(19) United States
(12) Patent Application Publication
(10) Pub. No.: US 2017/0363612 A1
(43) Pub. Date: Dec. 21, 2017

(54) COMPOSITIONS AND METHODS RELATING TO PROLIFERATIVE DISORDERS

(71) Applicant: Wayne State University, Detroit, MI (US)

(72) Inventors: Leon Carlock, Bloomfield Hills, MI (US); Maria Cypher, Magnolia, TX (US)

(21) Appl. No.: 15/532,366

(22) PCT Filed: Dec. 3, 2015
(86) PCT No.: PCT/US2015/063777

§ 371 Date: Jun. 1, 2017

Related U.S. Application Data
(60) Provisional application No. 62/087,023, filed on Dec. 3, 2014.

(51) Int. Cl. G01N 33/50; A61K 49/00; C12N 5/09; C12Q 1/68

(52) U.S. Cl. CPC G01N 33/5011; G01N 33/5017; C12Q 1/6897; A61K 49/0008; C12N 5/0693; C12N 2510/00

(57) ABSTRACT

Methods and compositions for drug discovery, analysis and treatment of a proliferative disorder characterized by abnormal cells in a mammalian subject are provided according to aspects of the present invention which include administering a pharmaceutically effective amount of a combination of: a cytotoxic agent, a SET agonist and a SET ribosome antagonist. Methods and compositions according aspect of the present invention incorporate agents effective to regulate and/or affect selective translation in a cell characterized by abnormal proliferation, such as a cancer cell, thereby promoting death of the cell.

### REFERENCE TO RELATED APPLICATION

This application claims priority from U.S. Provisional Patent Application Ser. No. 62/087,023, filed Dec. 3, 2014, the entire content of which is incorporated herein by reference.

### FIELD OF INVENTION

The present invention generally relates to methods and compositions for inhibition of abnormally proliferating cells. According to specific aspects, methods and compositions of the present invention relate to detecting and affecting selective translation selective translation in vitro and in vivo.

### BACKGROUND OF THE INVENTION

Cancer is characterized by abnormal, accelerated growth of epithelial, connective tissue, blood and lymph cells, as well as other rare cell types (e.g. glioma), that acquire the potential to spread to distant organs and cause premature patient death. In 2014, about 1.7 million new cancer cases will be diagnosed and 585,700 Americans will die, amounting to nearly 1,600 patients per day. This year, cancer will be the second most common cause of death in the US, exceeded only by heart disease, accounting for nearly 1 in every 4 deaths. There is a continuing need for compositions and methods relating to treatment of proliferative disorders, including cancer.

### SUMMARY OF THE INVENTION

This invention provides, in one aspect, a method for treating a proliferative disorder in a patient, comprising administering to the patient a therapeutically effective amount of a Selective Translation (SET) Therapeutic. The term “SET Therapeutic” as used herein refers to a cytotoxic agent in combination with a Selective Translation (SET) Combination Drug, delivered with a pharmaceutically acceptable carrier or excipient. A SET Therapeutic is administered to a patient in need thereof according to aspects of the present invention to prevent and/or treat a wide variety of neoplastic disorders, such as cancers, particularly drug resistant cancers and/or metastatic cancers.

A SET Combination drug includes an agonist of the SET response (SET agonist) and an antagonist of the SET Ribosome (SET ribosome antagonist).

Nonlimiting representative cancers that can be treated and/or prevented with this drug combination include drug resistant colorectal, breast, lymphoma, leukemia, melanoma, and prostate cancer. The nonlimiting list of cancers that can be treated with SET Therapeutics containing capecitabine or 5-FU/leucovorin, in pairwise combinations or as part of combination drugs such as CMF, FEC, FOLFIRI, CAPDXIRI, XELIRI, CAPDX, XELOX, CAPDXIRI, includes metastatic breast cancer, metastatic colon and rectal cancers, pancreatic cancer, anal cancer, gastric and esophageal cancers, cancers of the bile duct and gallbladder, cholangiocarcinoma, hepatocellular carcinoma, glioma, ependymoma, ovarian endometrial and cervical cancers, bladder cancer, metastatic renal cell carcinoma, non-small cell lung cancer, head and neck cancer, nasopharyngeal carcinoma, actinic (solar) keratoses and some types of basal cell carcinomas (Bowen's Disease). The nonlimiting list of cancers that can be treated with SET Therapeutics containing paclitaxel or docetaxel, in pairwise combinations or as part of combination drugs such as TCH, TC, AC, TIP, TPF, includes breast cancer, ovarian cancer, prostate cancer, testicular cancer, non-small cell lung cancer, small cell lung cancer, head and neck cancer, Kaposi's sarcoma, pancreatic cancer, biliary tract cancer, bladder cancer, endometrial cancer and gastric cancer. The nonlimiting list of cancers that can be treated with SET Therapeutics containing irinotecan or topotecan, in pairwise combinations or as part of combination drugs such as FOLFIRI, CAPDXIRI, and XELIRI, includes metastatic colon and rectal cancers, metastatic carcinoma of the ovary, Stage IV-B recurrent or persistent carcinoma of the cervix, small cell lung cancer, anaplastic astrocytomas, mixed malignant gliomas, oligodendrogliomas, non-small cell lung cancer, small cell lung cancer, neuroblastoma, breast cancer, leukemia and lymphoma either as monotherapies or in combination with other drugs. The nonlimiting list of cancers that can be treated with SET Therapeutics containing oxaliplatin, in pairwise combinations or as part of combination drugs such as FOLFOX, CAPDX, XELOX, and CAPDXIRI, includes adenocarcinoma of the pancreas, ampullary and periampullary carcinomas, adenocarcinoma of the anus, appendiceal carcinoma, metastatic colon and rectal cancers, ovarian cancer, esophageal carcinoma, gastric carcinoma, small bowel carcinoma, testicular cancer, chronic lymphocytic leukemia, non-Hodgkin's lymphoma, peripheral T-cell lymphomas, large B-cell lymphoma, and gallbladder cancer. The nonlimiting list of cancers that can be treated with SET Therapeutics containing cyclophosphamide, in pairwise combinations or as part of combination drugs such as AC, AP, CMF, and FEC, includes carcinoma of the breast, neuroblastoma (disseminated disease), retinoblastoma, adenocarcinoma of the ovary, malignant lymphomas (Stages III and IV of the Ann Arbor staging system), Hodgkin's disease, lymphocytic lymphoma (nodular or diffuse), mixed-cell type lymphoma, histiocytic lymphoma, Burkitt's lymphoma, multiple myeloma, chronic lymphocytic leukemia, chronic granulocytic leukemia, acute myelogenous and monocytic leukemia, acute lymphoblastic (stem-cell) leukemia in children.

TCH: paclitaxel, carboplatin and trastuzumab; TC: docetaxel and cyclophosphamide; AC: doxorubicin and cyclophosphamide; TAC: docetaxel and doxorubicin; AP: paclitaxel and doxorubicin with cyclophosphamide (Cytoxan) 500 mg/m2 iv dl; TIP: paclitaxel, ifosfamide and cisplatin; TPF: docetaxel, cisplatin and fluorouracil (5-FU); GTX: gemcitabine, capecitabine and docetaxel; CMF: cyclophosphamide, methotrexate, and 5-FU; FEC: 5-FU, epirubicin, and cyclophosphamide; XELOX (also called CAPDX): capecitabine combined with oxaliplatin; XELIRI: capecitabine combined with irinotecan; CAPDXIRI: capecitabine, oxaliplatin, and irinotecan; FL (also known as Mayo): 5-FU and leucovorin (folinic acid); FOLFOX: 5-FU/, leucovorin, and oxaliplatin; FOLFIRI: 5-FU, leucovorin, and irinotecan (several drugs, such as monoclonal antibodies, are sometimes added to FOLFIRI); GTX: gemcitabine, capecitabine and docetaxel; PEXG: gemcitabine hydrochloride, cisplatin, epirubicin hydrochloride, and capecitabine; FOLFIRINOX: 5-FU, leucovorin, irinotecan, and oxaliplatin; ECF: epirubicin, cisplatin, and 5-FU; TPF: docetaxel, cisplatin and 5-FU.

Treatment regimens known for administration of drug combinations TCH, TC, AC, TAC, AP, TIP, TPF, GTX, CMF, FEC, XELOX, XELIRI, CAPDXIRI, FL, FOLFOX, FOLFIRI, GTX, PEXG, FOLFIRINOX, ECF, TPF can be used in conjunction with administration of a SET Therapeutic to a subject or varied depending on the characteristics of the subject and clinical assessment of the disease to be treated.

In one aspect, a SET Therapeutic includes a cytotoxic agent including a compound that is converted to 5-fluorouracil (5-FU) in the body of the patient. Capecitabine is an example of a cytotoxic agent converted to 5-FU in the body of a patient.

According to aspects of the present invention the cytotoxic agent includes one or more of: capecitabine, 5-FU/leucovorin, paclitaxel, docetaxel, cyclophosphamide, topotecan, irinotecan, and oxaliplatin.

According to aspects of the present invention, an included SET Agonist is one or more of: a phorbol ester, a derivative of a phorbol ester, a bryostatin, and a polyoxyl hydrogenated castor oil.

According to aspects of the present invention, an included SET Ribosome Antagonist is anisomycin, cycloheximide, and/or emetine.

Methods of enhancing the efficacy of a cytotoxic agent in a subject being treated with the cytotoxic agent are provided according to aspects of the present invention. In this aspect, the SET Combination Drugs stimulate cell cycle progression and block the recovery of drug resistant tumors after cytotoxic injury, which promotes cell death.

According to aspects of the present invention, a SET Combination drug is simultaneously administered with the cytotoxic agent. According to aspects of the present invention, a SET Combination drug is administered to a patient at a different time from the administration of the cytotoxic agent to the patient. Further, the SET agonist and SET ribosome antagonist components of a SET Combination drug are optionally administered together or separately, and together with, or separately from the cytotoxic agent. In a preferred aspect, the cytotoxic agent is administered prior to the SET Combination drug.

According to aspects of the present invention, in which a SET Combination drug is administered to a patient at a different time from the administration of the cytotoxic agent to the patient, the SET combination drug is preferably administered within 10 minutes, 30 minutes, 1 hour, 2 hours, 3 hours, 4, hours, 8 hours, 12, hours, 24 hours, 48 hours, 72 hours, 4 days, 5 days, 6 days or 7 days after administration of the cytotoxic agent. Where the SET agonist and SET ribosome antagonist components of a SET Combination drug are administered separately from each other, they are preferably administered within 10 minutes, 30 minutes, 1 hour, 2 hours, 3 hours, 4, hours, 8 hours, 12, hours, 24 hours, 48 hours, 72 hours, 4 days, 5 days, 6 days or 7 days of each other.

According to aspects of the present invention, the SET Combination Drugs are simultaneously administered with 5-FU/leucovorin, capecitabine, cyclophosphamide, topotecan, irinotecan, oxaliplatin, docetaxel, and/or paclitaxel to a patient.

According to aspects of the present invention, the SET Combination drug is administered to a patient at a different time than 5-FU/leucovorin, capecitabine, cyclophosphamide, topotecan, irinotecan, oxaliplatin, docetaxel, and/or paclitaxel is administered to the patient.

The SET Therapeutic can be administered by any pharmaceutically acceptable route. According to aspects of the present invention, a SET Therapeutic is administered to a patient by an oral and/or parenteral route. According to aspects of the present invention, a SET Therapeutic is administered to a patient by an intravenous or subcutaneous route.

Methods for treatment of a proliferative disorder characterized by abnormal cells in a mammalian subject are provided according to aspects of the present invention which include administering a pharmaceutically effective amount of a combination of: a cytotoxic agent, a SET agonist and a SET ribosome antagonist. The abnormal cells include both mitotic abnormal cells and non-mitotic abnormal and wherein both abnormal cells and non-mitotic abnormal cells are induced to die due to the administering of the pharmaceutically effective amount of a combination of: a cytotoxic agent, a SET agonist and a SET ribosome antagonist, wherein the combination promotes increased abnormal cell death in G2 phase compared to administration of the cytotoxic agent alone.

Methods for treatment of a proliferative disorder characterized by abnormal cells in a mammalian subject are provided according to aspects of the present invention wherein the combination of a cytotoxic agent, a SET agonist and a SET ribosome antagonist is effective such that a lower dose of the cytotoxic agent is required to kill the abnormal cells compared to treatment by administering the cytotoxic agent without the SET agonist and the SET ribosome antagonist.

The cytotoxic agent is selected from the group consisting of: capecitabine, cyclophosphamide, topotecan, paclitaxel, 5-FU/leucovorin, docetaxel, irinotecan, and oxaliplatin, a pharmaceutically acceptable salt thereof and a combination of any two or more thereof according to aspects of methods of treatment of the present invention.

Methods for treatment of a proliferative disorder characterized by abnormal cells in a mammalian subject are provided according to aspects of the present invention in which the SET agonist is a stimulator of G2 progression. According to further aspects of the present invention, the SET agonist is selected from the group consisting of: a polyoxyl hydrogenated castor oil, a phorbol ester, a bryostatin, a pharmaceutically acceptable salt thereof and a combination of any two or more thereof.

According to further aspects of the present invention, the polyoxyl hydrogenated castor oil is selected from the group consisting of: polyoxyl 30 hydrogenated castor oil, polyoxyl 35 hydrogenated castor oil, polyoxyl 40 hydrogenated castor oil, polyoxyl 50 hydrogenated castor oil, polyoxyl 60 hydrogenated castor oil and a combination of any two or more thereof.

According to further aspects of the present invention, the polyoxyl hydrogenated castor oil is polyoxyl 35 hydrogenated castor oil, polyoxyl 40 hydrogenated castor oil, or a combination of polyoxyl 35 hydrogenated castor oil and polyoxyl 40 hydrogenated castor oil.

According to further aspects of the present invention, the bryostatin is bryostatin 1 and/or bryostatin 2; or a pharmaceutically acceptable salt thereof.

According to further aspects of the present invention, the phorbol ester is 12-O-tetradecanoylphorbol-13-acetate or a pharmaceutically acceptable salt thereof.

A SET ribosome antagonist administered according to aspects of the present invention inhibits protein synthesis by SET Ribosomes. According to aspects of the present invention, the SET ribosome antagonist is selected from the group consisting of: anisomycin, cycloheximide, emetine, a pharmaceutically acceptable salt thereof and a combination thereof.

Methods for treatment of a proliferative disorder characterized by abnormal cells in a mammalian subject are provided according to aspects of the present invention which include administering a pharmaceutically effective amount of a combination of: 1) 5-fluorouracil/leucovorin, capecitabine, cyclophosphamide, irinotecan, topotecan, paclitaxel, docetaxel, oxaliplatin, a pharmaceutically acceptable salt thereof or a combination of any two or more thereof; 2) polyoxyl 35 hydrogenated castor oil polyoxyl, 40 hydrogenated castor oil or a combination of both thereof; and 3) emetine, cycloheximide, anisomycin, a pharmaceutically acceptable salt of any thereof or a combination of any two or more thereof. The abnormal cells include both mitotic abnormal cells and non-mitotic abnormal and wherein both abnormal cells and non-mitotic abnormal cells are induced to die due to the administering of the pharmaceutically effective amount of 1), 2) and 3).

Methods for treatment of a proliferative disorder characterized by abnormal cells in a mammalian subject are provided according to aspects of the present invention wherein the combination of: 1) 5-fluorouracil/leucovorin, capecitabine, cyclophosphamide, irinotecan, topotecan, paclitaxel, docetaxel, oxaliplatin, a pharmaceutically acceptable salt thereof or a combination of any two or more thereof; 2) polyoxyl 35 hydrogenated castor oil polyoxyl, 40 hydrogenated castor oil or a combination of both thereof; and 3) emetine, cycloheximide, anisomycin, a pharmaceutically acceptable salt of any thereof or a combination of any two or more thereof, is effective such that a lower dose of cytotoxic agent selected from: 5-fluorouracil/leucovorin, capecitabine, irinotecan, topotecan, paclitaxel, docetaxel, oxaliplatin, cyclophosphamide, a pharmaceutically acceptable salt thereof or a combination of any two or more thereof, is required to kill the abnormal cells compared to treatment by administering the cytotoxic agent without the 2) polyoxyl 35 hydrogenated castor oil polyoxyl, 40 hydrogenated castor oil or a combination of both thereof; and 3) emetine, cycloheximide, anisomycin, a pharmaceutically acceptable salt of any thereof or a combination of any two or more thereof.

Methods for treatment of a proliferative disorder characterized by abnormal cells in a mammalian subject are provided according to aspects of the present invention wherein the combination of a cytotoxic agent, SET agonist and SET ribosome antagonist is any one of the combinations shown as Ref Nos: 1-96 in Table 13, including the combination of a cytotoxic agent, SET agonist and SET ribosome antagonist shown therein as Ref No: 1, 2, 3, 4, 5, 6, 7, 8, 9,10, 11, 12, 13,14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95 or 96; any one or more of which is specifically contemplated.

Methods for treatment of a proliferative disorder characterized by abnormal cells in a mammalian subject are provided according to aspects of the present invention wherein the subject is human.

Methods for treatment of a proliferative disorder characterized by abnormal cells in a mammalian subject are provided according to aspects of the present invention wherein the proliferative disorder is drug-resistant cancer.

Methods for treatment of a proliferative disorder characterized by abnormal cells in a mammalian subject are provided according to aspects of the present invention wherein the proliferative disorder is metastatic cancer.

Methods for treatment of a proliferative disorder characterized by abnormal cells in a mammalian subject are provided according to aspects of the present invention wherein the proliferative disorder is selected from the group consisting of: breast cancer, metastatic breast cancer, colon cancer, metastatic colon cancer, anal cancer, metastatic rectal cancer, pancreatic cancer, gastric cancer, esophageal cancer, bile duct cancer, gallbladder cancer, cholangiocarcinoma, hepatocellular carcinoma, glioma, ependyoma, metastatic ovarian cancer, endometrial cancer, cervical cancer, recurrent or persistent carcinoma of the cervix, bladder cancer, renal cell carcinoma, metastatic renal cell carcinoma, non-small cell lung cancer, head and neck cancer, nasopharyngeal carcinoma, ovarian cancer, retinoblastoma, neuroblastomas, anaplastic astrocytomas, mixed malignant gliomas, oligodendrogliomas, prostate cancer, adenocarcinoma of the pancreas, ampullary and periampullary carcinomas, adenocarcinoma of the anus, adenocarcinoma of the ovary, appendiceal carcinoma, testicular cancer, small cell lung cancer, small bowel carcinoma, leukemia, chronic lymphocytic leukemia, lymphoma, mixed cell type lymphoma, non-Hodgkin's lymphoma, peripheral T-cell lymphomas, large B-cell lymphoma, Kaposi's sarcoma, malignant lymphomas (Stages III and IV of the Ann Arbor staging system), Hodgkin's disease, lymphocytic lymphoma (nodular or diffuse), mixed-cell type lymphoma, histiocytic lymphoma, Burkitt's lymphoma, multiple myeloma, chronic lymphocytic leukemia, chronic granulocytic leukemia, acute myelogenous and monocytic leukemia, acute lymphoblastic (stem-cell) leukemia in children, biliary tract cancer, basal cell carcinoma, restenosis, scarring and actinic ketatoses.

Methods for treatment of a proliferative disorder characterized by abnormal cells in a mammalian subject are provided according to aspects of the present invention wherein the cytoxic agent, the SET agonist and the SET ribosome antagonist are administered simultaneously.

Methods for treatment of a proliferative disorder characterized by abnormal cells in a mammalian subject are provided according to aspects of the present invention wherein the cytotoxic agent, the SET agonist and the SET ribosome antagonist are administered at different times.

Methods for treatment of a proliferative disorder characterized by abnormal cells in a mammalian subject are provided according to aspects of the present invention wherein the SET agonist and the SET ribosome antagonist are administered together in a pharmaceutical formulation.

Methods for treatment of a proliferative disorder characterized by abnormal cells in a mammalian subject are provided according to aspects of the present invention wherein the SET agonist and the SET ribosome antagonist are administered orally together in a pharmaceutical formulation.

Methods for treatment of a proliferative disorder characterized by abnormal cells in a mammalian subject are provided according to aspects of the present invention wherein the further includes an adjunct therapeutic treatment.

Optionally, the adjunct therapeutic treatment includes radiation treatment of the subject.

In a further option, the adjunct therapeutic treatment comprises administration of one or more additional chemotherapeutic drugs.

Methods for treatment of a proliferative disorder characterized by abnormal cells in a mammalian subject are provided according to aspects of the present invention wherein the cytotoxic agent is administered by injection.

Methods for treatment of a proliferative disorder characterized by abnormal cells in a mammalian subject are provided according to aspects of the present invention wherein the cytotoxic agent is administered intravenously.

Methods for treatment of a proliferative disorder characterized by abnormal cells in a mammalian subject are provided according to aspects of the present invention wherein an abnormal cell of the subject having the proliferative disorder characterized by abnormal cells is contacted with the cytotoxic agent prior to being contacted with the SET agonist or a SET ribosome antagonist.

Methods for treatment of cancer in a mammalian subject are provided according to aspects of the present invention wherein a cancer cell of the subject having cancer is contacted with the cytotoxic agent prior to being contacted with the SET agonist or a SET ribosome antagonist.

Methods for treatment of a proliferative disorder characterized by abnormal cells in a mammalian subject are provided according to aspects of the present invention wherein an abnormal cell of the subject having the proliferative disorder characterized by abnormal cells is contacted with the cytotoxic agent prior to being contacted with the SET agonist or a SET ribosome antagonist.

Methods for treatment of a proliferative disorder characterized by abnormal cells in a mammalian subject are provided according to aspects of the present invention which include administering a pharmaceutically effective amount of a combination of: a cytotoxic agent, a SET agonist and a SET ribosome antagonist, wherein the dose of the SET agonist is at the concentration that produces a maximal SET ribosome activity and the SET ribosome antagonist is at the concentration that produces an IC100 of the SET ribosome activity in the range of 1/2500-1/5000 of the LD50. The abnormal cells include both mitotic abnormal cells and non-mitotic abnormal and wherein both abnormal cells and non-mitotic abnormal cells are induced to die due to the administering of the pharmaceutically effective amount of a combination of: a cytotoxic agent, a SET agonist and a SET ribosome antagonist, wherein the dose of the SET agonist is at the concentration that produces a maximal SET ribosome activity and the SET ribosome antagonist is at the concentration that produces an IC100 of the SET ribosome activity in the range of 1/2500-1/5000 of the LD50.

Pharmaceutical compositions are provided according to aspects of the present invention which include a SET agonist and a SET ribosome antagonist.

Pharmaceutical compositions are provided according to aspects of the present invention which include a SET agonist and a SET ribosome antagonist, wherein the SET agonist is a stimulator of G2 phase progression.

Pharmaceutical compositions are provided according to aspects of the present invention which include a SET agonist and a SET ribosome antagonist, wherein the SET agonist is selected from the group consisting of: a polyoxyl hydrogenated castor oil, a phorbol ester, a bryostatin, a pharmaceutically acceptable salt of any thereof, and a combination of any two or more thereof.

Pharmaceutical compositions are provided according to aspects of the present invention which include a SET agonist and a SET ribosome antagonist, wherein the polyoxyl hydrogenated castor oil is selected from the group consisting of: polyoxyl 30 hydrogenated castor oil, polyoxyl 35 hydrogenated castor oil, polyoxyl 40 hydrogenated castor oil, polyoxyl 50 hydrogenated castor oil, polyoxyl 60 hydrogenated castor oil, and a combination of any two or more thereof.

Pharmaceutical compositions are provided according to aspects of the present invention wherein the SET agonist is selected from bryostatin 1, bryostatin 2; a pharmaceutically acceptable salt of either thereof, and a combination of any two or more thereof.

Pharmaceutical compositions are provided according to aspects of the present invention which include a SET agonist is 12-O-tetradecanoylphorbol-13-acetate or a pharmaceutically acceptable salt thereof.

A SET ribosome antagonist inhibits protein synthesis by SET Ribosomes according to aspects of the invention as described herein.

Pharmaceutical compositions are provided according to aspects of the present invention which include a SET ribosome antagonist selected from the group consisting of: anisomycin, cycloheximide, emetine, a pharmaceutically acceptable salt of either thereof and a combination of any two or more thereof.

Pharmaceutical compositions are provided according to aspects of the present invention which include polyoxyl 35 hydrogenated castor oil and anisomycin or a pharmaceutically acceptable salt thereof.

Pharmaceutical compositions are provided according to aspects of the present invention which include polyoxyl 35 hydrogenated castor oil and emetine or a pharmaceutically acceptable salt thereof.

Pharmaceutical compositions are provided according to aspects of the present invention which include polyoxyl 35 hydrogenated castor oil and cycloheximide or a pharmaceutically acceptable salt thereof.

Pharmaceutical compositions are provided according to aspects of the present invention which are formulated for oral administration to a subject.

Derivatives of cytotoxic agents, SET agonists and/or SET ribosome antagonists are useful in compositions and methods according to aspects of the present invention and are specifically contemplated for inclusion therein. The term “derivative” refers to a modified composition which retains an identifiable structural relationship with the unmodified composition and which retains the function of the unmodified composition or has improved functionality relative to the unmodified composition.

According to aspects of the present invention, methods and compositions include an expression cassette encoding a TR element. According to aspects of the present invention, the encoded TR element is selected from a human or a mouse TR element. According to preferred aspects of the present invention, the TR element is selected from those encoded by SEQ ID NO:1, SEQ ID NO:2, SEQ ID NO:3, SEQ ID NO:4, SEQ ID NO:17, SEQ ID NO:18, SEQ ID NO:19, SEQ ID NO:20 or a variant of any thereof, wherein the encoded TR element confers selective translation on an operably linked coding sequence in an mRNA.

Methods of identifying an agent effective as a component of a SET Combination drug for treatment a proliferative disease according to aspects of the present invention include providing a cell characterized by a TR Class 3 outlier SET response, wherein the cell comprises an expression cassette encoding a TR element and a reporter and wherein the expression cassette is stably integrated into the genome of the cells contacting the cell with a test substance; and measuring the effect of the test substance on protein synthesis from a SET ribosome compared to a control, wherein inhibition of protein synthesis from a SET ribosome by the test substance identifies the substance as an agent effective as a component of a SET Combination drug for treatment a proliferative disease.

Methods of identifying an agent effective as a component of a SET Combination drug for treatment a proliferative disease according to aspects of the present invention include providing a cell characterized by a TR Class 3 outlier SET response, and further characterized by in vitro ability to grow in suspension cultures as nonadherent 3D and/or the ability to initiate and grow into a primary xenogeneic tumor in vivo, wherein the primary xenogeneic tumor can be dissected into subfragments and propagated as a secondary tumor;

Isolated non-naturally occurring, TR Class 4 cells characterized by a TR Class 3 outlier SET response are provided according to aspects of the present invention.

Isolated non-naturally occurring, TR Class 4 cells characterized by a TR Class 3 outlier SET response, further characterized by an in vitro ability to grow in suspension cultures as nonadherent 3D structures and the ability to initiate and grow into a primary xenogenic tumor in vivo, that can be dissected into subfragments and propagated as a secondary tumor are provided according to aspects of the present invention.

Methods of generating a metastatic cancer cell line model are provided according to aspects of the present invention which include introducing an expression cassette encoding a TR element and a reporter into a cell, producing a parental population of cells wherein the expression cassette is stably integrated into the genome of the cells; isolating subclones of the parental population; administering a SET agonist to a population of cells of each subclone to induce a SET TR response in the population of cells of each subclone; assaying the TR SET response in the population of cells of each subclone by detecting expression of the reporter; ranking the TR SET response of each subclone compared to each other subclone, establishing a range of TR SET responses characterized by an average response; selecting the subclones characterized by detectable increases in expression of the reporter of at least two standard deviations greater than the mean response, thereby defining the selected subclones as TR Class 3 SET response subclones; administering a SET agonist to a population of cells of each TR Class 3 SET response subclone to induce a SET TR response in the population of cells of each TR Class 3 SET response subclone; assaying the TR SET response in the population of cells of each TR Class 3 SET response subclone by detecting expression of the reporter; ranking the TR SET response of each TR Class 3 SET response subclone compared to each other TR Class 3 SET response subclone, establishing a range of TR SET responses characterized by an average response; selecting the TR Class 3 SET response subclones characterized by detectable increases in expression of the reporter of at least two standard deviations greater than the mean response, thereby defining the selected TR Class 3 SET response subclones as TR Class 3 SET response outliers; administering one or more toxins to cells of one or more subclones characterized as a TR Class 3 SET response outliers; detecting a response of the cells of the one or more subclones characterized as a TR Class 3 SET response outliers indicative of drug and stress resistance due to elevated SET ribosome activity in the cells of the subclone, thereby determining that the cells are TR Class 4 cells; and thereby generating a metastatic cancer cell line model.

Methods of generating a metastatic cancer cell line model are provided according to aspects of the present invention which further include culturing the TR Class 4 cells under low density conditions for at least 50 cell cycles, generating TR Class 4 subclones and capable of low density colony formation; selecting the TR Class 4 subclones capable of low density colony formation; administering a SET agonist to a population of cells of each TR Class 4 subclone capable of low density colony formation to induce a TR SET response; assaying the SET response in the population of cells of each TR Class 4 subclone capable of low density colony formation to induce a TR SET response by detecting expression of the reporter; ranking the TR SET response of each TR Class 4 subclone capable of low density colony formation compared to each other TR Class 4 subclone capable of low density colony formation establishing a range of SET responses characterized by an average response; selecting the TR Class 4 subclones capable of low density colony formation and characterized by detectable increases in expression of the reporter of at least two standard deviations greater than the mean response.

Methods of generating a metastatic cancer cell line model are provided according to aspects of the present invention which further include culturing the TR Class 4 cells under nonadherent low density culture conditions and selecting subclones of the TR Class 4 cells that grow as suspended aggregates, thereby selecting subclones of TR Class 4 cells capable of ex vivo tumorsphere formation with 10 or fewer cells initiating the tumorsphere; administering one or more toxins to cells of the TR Class 4 subclones capable of ex vivo tumorsphere formation with 10 or fewer cells initiating the tumorsphere response; detecting a response of the cells of the TR Class 4 subclones capable of ex vivo tumorsphere formation with 10 or fewer cells initiating the tumorsphere indicative of drug and stress resistance due to elevated SET ribosome activity in the cells of the subclone, thereby determining that the cells of the TR Class 4 subclones are capable of ex vivo tumorsphere formation with 10 or fewer cells, characterized by a TR Class 4 TR SET response.

Methods of identifying an agent effective to promote or inhibit G2 progression in vivo are provided according to aspects of the present invention which include providing a cell of a TR Class 4 cell line characterized by a TR Class 3 outlier SET response, wherein the cell comprises a TR nucleic acid expression cassette encoding a TR element and a reporter; administering the cell to a non-human animal, producing a xenograft tumor in the non-human animal; administering a test substance to the non-human animal; and measuring the effect of the test substance on the SET response, wherein an increase in a SET response identifies the agent as a SET agonist effective to promote G2 progression in vivo.

Methods of identifying an agent effective to promote or inhibit G2 progression in vivo are provided according to aspects of the present invention which further include administering a SET agonist to the non-human animal to promote G2 progression in vivo, wherein a decrease in the SET response identifies the agent as a SET antagonist effective to inhibit G2 progression in vivo.

Methods of identifying an agent effective to promote or inhibit G2 progression in vivo are provided according to aspects of the present invention which further include measuring the effect of the test substance on the xenograft tumor.

The non-human animal is any suitable animal. According to aspects of the present invention, the non-human animal is a rodent, rabbit, monkey or other non-human primate. According to aspects of the present invention, the non-human animal is a rat or mouse.

Methods of identifying an agent effective to promote or inhibit G2 progression in vivo according to aspects of the present invention include providing a cell of a TR Class 4 cell line characterized by a TR Class 3 outlier SET response, wherein the cell comprises a TR nucleic acid expression cassette encoding a TR element and a reporter, the expression cassette stably integrated into the genome of the cell; administering the cell to a non-human animal, producing a xenograft tumor in the non-human animal; administering a test substance to the non-human animal; measuring the effect of the test substance on the xenograft tumor; and measuring the effect of the test substance on the SET response, wherein an increase in a SET response identifies the agent as a SET agonist effective to promote G2 progression in vivo.

Methods of identifying an agent effective to promote or inhibit G2 progression in vivo according to aspects of the present invention include providing a cell of a TR Class 4 cell line characterized by a TR Class 3 outlier SET response, wherein the cell comprises a TR nucleic acid expression cassette encoding a TR element and a reporter, the expression cassette stably integrated into the genome of the cell; administering the cell to a non-human animal, producing a xenograft tumor in the non-human animal; administering a test substance to the non-human animal; administering a SET agonist to the non-human animal; and measuring the effect of the test substance on a SET response of the cell, wherein a decrease in the SET response identifies the agent as a SET antagonist effective to inhibit G2 progression in vivo.

Methods of identifying an agent effective to promote or inhibit G2 progression in vivo according to aspects of the present invention optionally further include measuring the effect of the test substance on the xenograft tumor.

### BRIEF DESCRIPTION OF THE DRAWINGS

FIG. 1A is a schematic showing the sequence elements within the TR Expression Cassette that prevent Cap-dependent translation and regulate SET ribosome translation. TR Expression Cassette has been derived from the mammalian proteolipid protein (pip) gene. It contains multiple upstream start codons, stop codons (shown as arrows), and short open reading frames (uORFs 1-9, shown as boxes) that prevent ribosomal scanning from the 5′ cap structure to the reporter gene start codon. Site directed mutagenesis defined an RNA segment in exon 4 that acts as a ribosome loading site for translation of the internal PIRP ORF (TR IRES Table 1). This site contains an 18S RNA complementary sequence that is strongly homologous to the sequence that directs ribosome loading in the Gtx IRES (alignment shown in Table 1). While the sequences in exons 5 and 6 appear to be nonessential for internal translation initiation, the 3′ terminus of the gene cassette (exon 7) contains a key regulator of the IRES function (TR Regulator Table 1). Deletions and point mutations in this region affect the fidelity of start codon selection and stress-specificity of the TR IRES translation, presumably due to disruption of the RNA secondary structure (summarized in Table 2). The regulator sequence also contains a distinct 18S RNA complementary sequence that is highly homologous to the caliciviral translational termination-reinitiation motif (alignment shown in Table 1), which means that reporter gene translation occurs by a reinitiation mechanism.

FIG. 1B shows a SET time course measuring the secreted Gaussia luciferase (gLUC) reporter protein released from the HEK293 hTRdm-gLUC#79 cell line treated continuously for 6 hr with 100 nM 12-O-tetradecanoylphorbol-13-acetate (TPA). Statistical analysis (Student's two-tailed tTest) found a significant SET increase at 2 hours post-treatment when Cap-dependent translation had declined (arrow). The timing of gLUC protein synthesis shows that rapidly replicating cells do not exhibit SET from the TR Expression Cassette in the Gl/S or early S cell cycle phases but activate the SET Ribosome during late S (>2 hours post-treatment). Increasing SET Ribosome activity was observed as cells enter the G2 cell cycle phase (3.5-6 hours) establishing that gLUC synthesis, transport, and secretion are not hindered by TPA activation of SET.

FIG. 2 shows heat shock regulation of Cap-dependent and SET ribosome-specific translation. HEK293 derived cell lines that express the firefly luciferase (fLuc) reporter either constitutively (CMV lines) or as a part of the TR Expression Cassette (hTR and mTR lines) were continuously heated at 42° C. for 6 hours (FIG. 2A) or 3 hours (FIG. 2B) and assayed for fLuc activity at hourly intervals. As expected, continuous lethal heat treatment blocks the Cap-Dependent fLuc expression in the CMV lines, and fLuc activity continues to drop throughout the assay as a result of continued turnover. The timing of the Cap-Dependent translation inhibition closely correlates with a time-dependent decrease in survival of mice treated with a lethal temperature (41° C.), which reaches statistical significance within 45 min. In contrast, the SET-dependent fLuc expression in the TR lines becomes detectable within 2 hr and continues to increase throughout the assay period. FIG. 2B shows that SET induction occurs between 1-2 hours of heat exposure. By 4 hr post-treatment, the TR cell line SET responses segregate into the previously defined TR SET Classes based upon an inherent thermal viability (resistance to lethal heat shock). A subset of TR Class 3 cells (termed the TR Class 4; hTRdm-fLUC#122) exhibit a statistically significant increase in heat induced SET activity compared to the Class 3 mean and exhibit enhanced cell viability at 8 hr post treatment (as measured by the Trypan blue staining). These results illustrate that ex vivo and in vivo thermal viability correlates with the presence of a distinct population of ribosomes capable of recovery protein synthesis (termed the SET Ribosomes).

FIG. 3 shows thermal regulation of Cap-dependent and SET ribosome translation.

Abbreviations: TPA: 12-O-tetradecanoylphorbol-13-acetate; Tax: Paclitaxel; MG: MG132; Cal: Calcium Ionophore A23187; Topo: Topotecan.

FIGS. 3A and 3B show trend plots demonstrating the TR Class-specific SET responses to the five Reference Standard Reagents (Table 3) at low ambient (23° C.) and high (42° C.) temperatures. HEK293 derived cell lines that express the fLuc reporter either constitutively (CMV lines) or as a part of the TR Expression Cassette (hTR and mTR lines) were treated with Reference Standard Reagents (full names and doses are summarized in Table 3), incubated at designated temperatures for 6 hours, and assayed for fLuc activity. In the CMV cell line, where the fLuc reporter is translated by the Cap-dependent Ribosome, all Reference Standard responses are repressed by heat and cold. In the TR cell lines, where the fLuc reporter is translated by the SET Ribosome, some Reference Standard responses show unpredictable changes that differ depending on the TR SET Class. TR Class 2 hTRdm-fLUC#8 cells show the lowest activation of the SET Ribosome in response to heat (FIG. 3A). While the 42° C. trend line shows the same profile as the 37° C., only TPA, TPA+Tax, and TPA+Cal demonstrate an enhanced fLuc activity compared to the untreated samples. Although the Tax response doesn't rise above the baseline, it doesn't drop at 42° C. like at 23° C. and 37° C., which is consistent with the 42° C. Tax peak in the TR Class 3 trend plots. The effects of cold treatment in this cell line are much less dramatic, affecting the magnitude, rather than the nature of the Reference Standard responses. In contrast to the hTRdm-fLUC#8 cell line, SET Ribosomes in the TR Class 3 mTRdm-fLUC#12 and hTRdm-fLUC#122 cells are less active at 23° C. than at 37° C. and 42° C. Heat has a stimulatory effect on the TPA, TPA+Tax, TPA+Cal, Tax, Tax+Cal, and Cal responses. As before, one of the two TR Class 3 cell lines exhibited enhanced SET Ribosome activity at 37° C. and 42° C. Only at 23° C. do the TR Class 2-3 responses show similar SET magnitude and repression of the Tax, Tax+Cal, and Cal responses.

As shown in FIG. 3B, the 23° C. SET trend plot shows that in the hTR and mTR cell lines SET ribosome activation by TPA is retained at 23° C., while the Tax response appears to require higher temperatures. Although the Cal response could not be easily detected due to the difference in magnitude between the high and low temperature responses, it can be observed, but in a much diminished form. The CMV trend line shows that in contrast to the SET Ribosome, the Cap-dependent Ribosome is completely inactivated by ambient temperature.

FIG. 4 shows that inactivation of mTORC1 by rapamycin stimulates SET Ribosome activity.

Abbreviations: T/T: 12-O-tetradecanoylphorbol-13-acetate+Taxol; T/T/R: 12-O-tetradecanoylphorbol-13-acetate+Taxol+Rapamycin.

FIG. 4 shows a rapamycin dose response chart for the MCF7 derived cell lines that express the fLuc reporter either constitutively (CMV lines) or as a part of the TR Expression Cassette (hTR and mTR lines). Rapamycin inhibits the Growth Ribosome activity by blocking mTORC1 (a protein complex that functions as a nutrient/energy/redox sensor and controls cell growth in the G1 cell cycle phase). It also affects the activity of mTORC2 (a complex involved in stress signaling during the G2 cell cycle phase), but only at high concentrations and after prolonged exposure. To tease apart the growth and stress responses, cells were treated with varying doses of rapamycin, incubated at 37° C. for 6 hours, and assayed for fLuc activity. Surprisingly, the CMV cell line, where the fLuc reporter is translated by the Growth Ribosome, only showed a modest block in protein synthesis. The fLuc expression goes up slightly relative to the untreated cells, and shows little change over the range of rapamycin doses tested. In contrast, the mTR and hTR cells, where the fLuc reporter is translated by the SET Ribosome, respond in accordance with the TR Class structure. Class 1 hTRdm-fLUC#6 responds similarly to the CMV cell lines. Of the Class 3 cells (mTRdm-fLUC#27, mTRdm-fLUC#45), the mTRdm-fLUC#8 line showed the greatest increase in fLuc expression (a putative TR Class 4 responder). The magnitude of SET activation was steady at rapamycin concentrations between 1 nM and 20 nM, with a spike in fLuc activity in the 50 nM rapamycin samples, and followed by a drop in SET activity in the 100 nM-1 uM samples. This drop in fLuc activity correlates with complete mTORC1 (but not mTORC2) inactivation, which establishes a link between the SET Ribosome, mTORC2 stress signaling, and the G2 cell cycle phase.

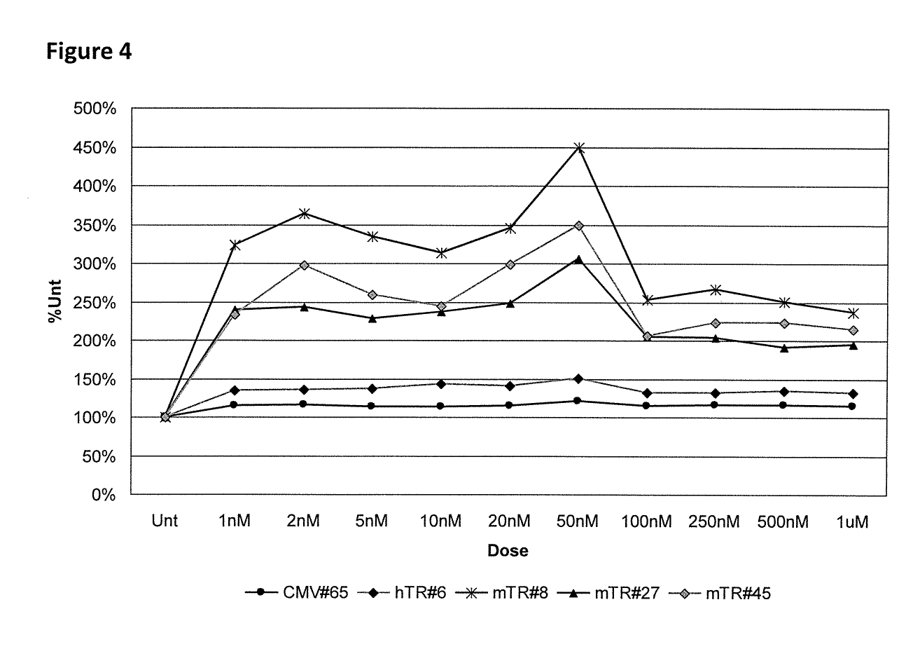

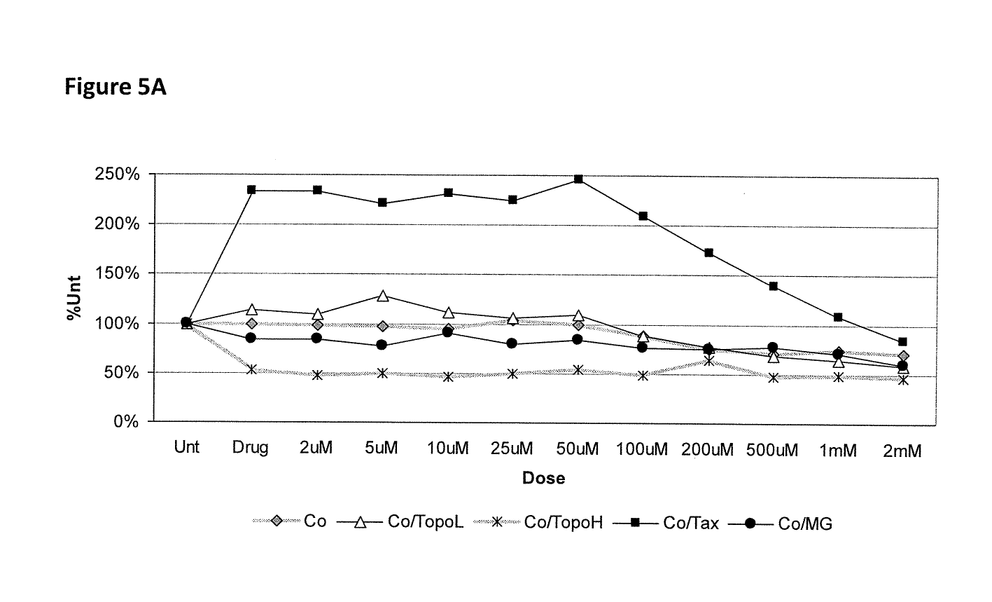

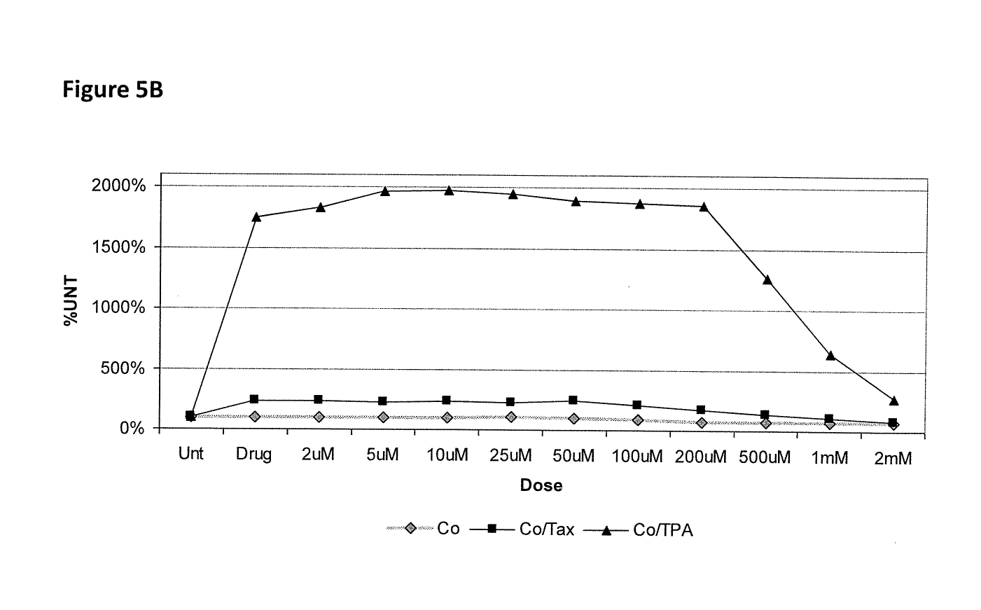

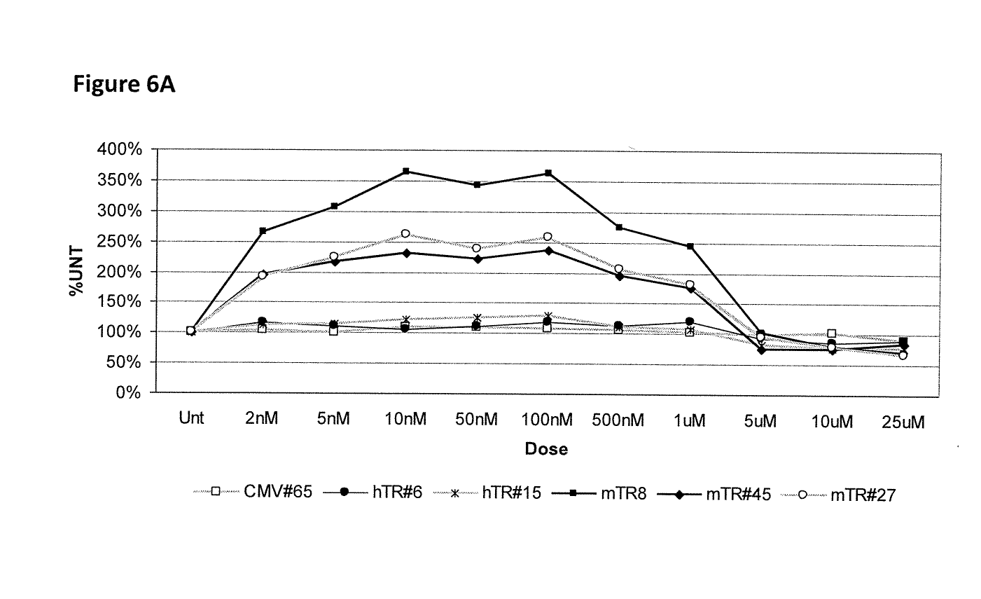

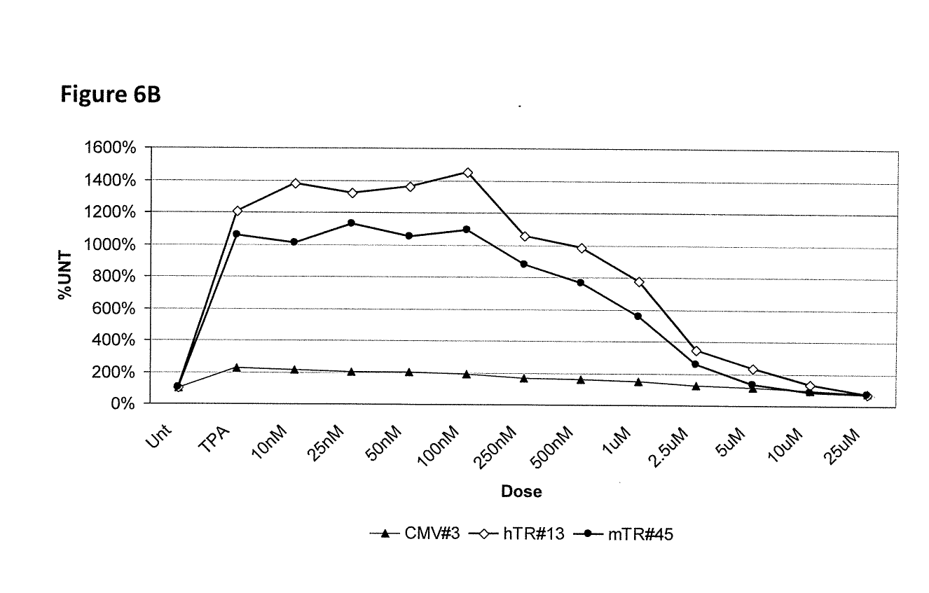

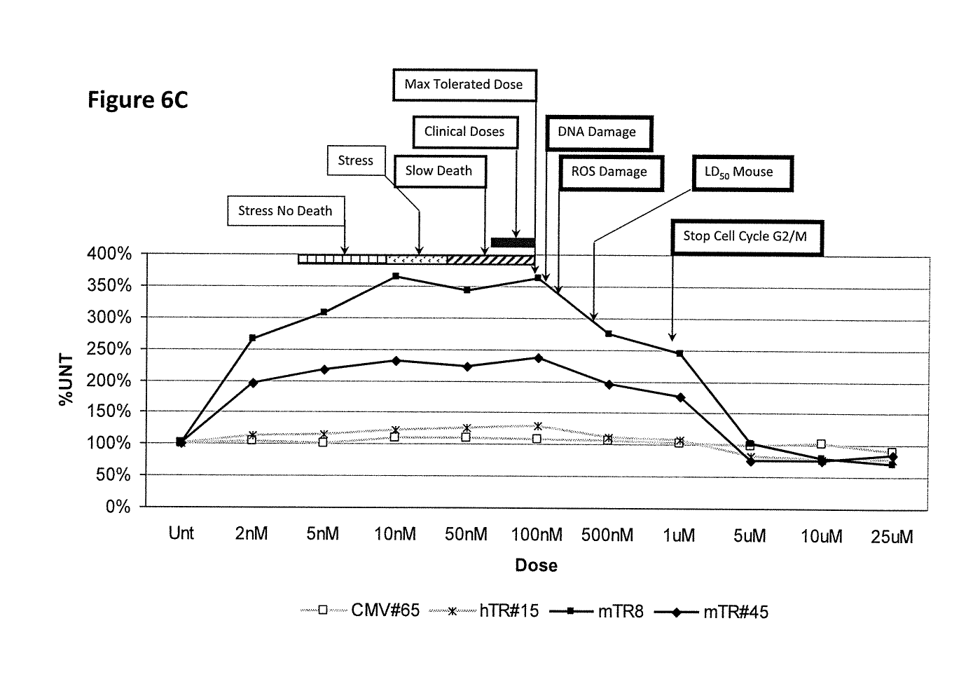

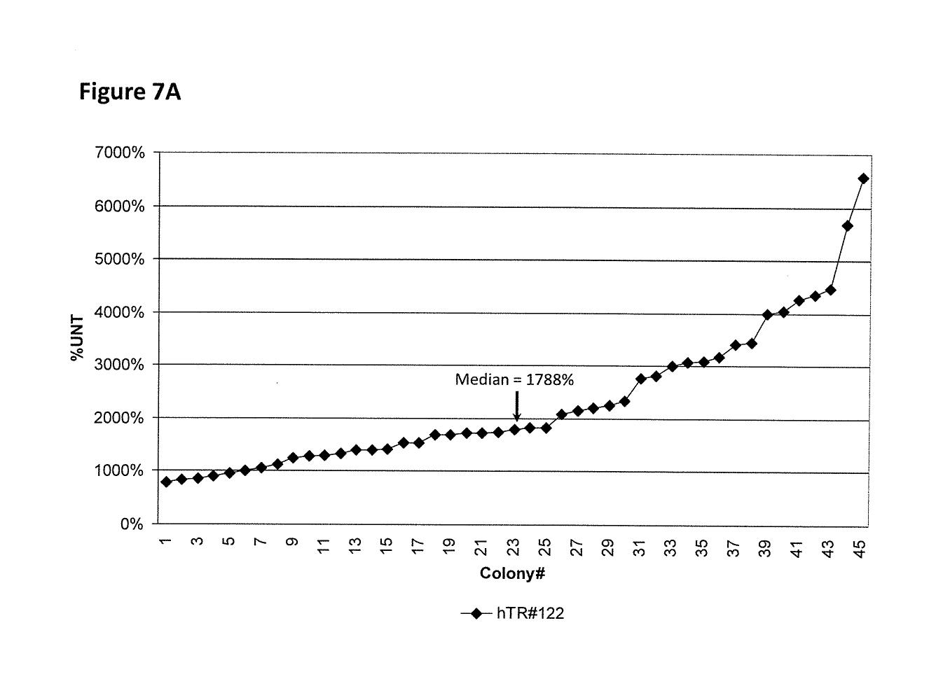

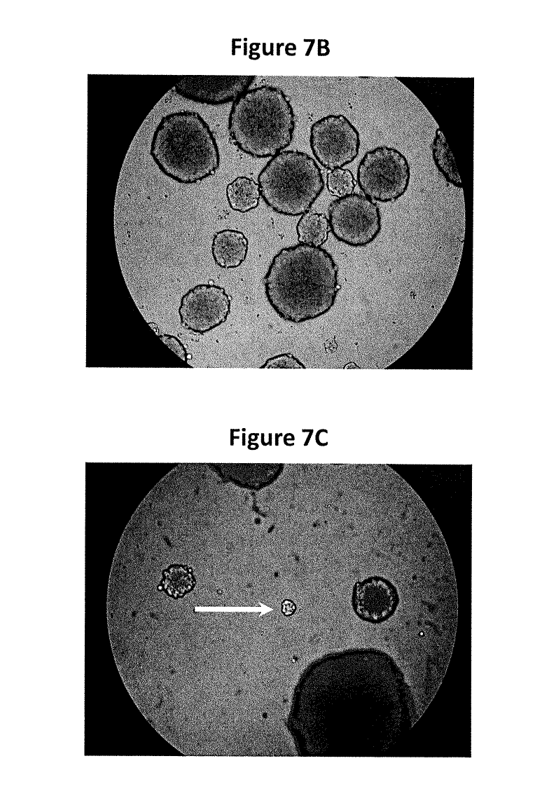

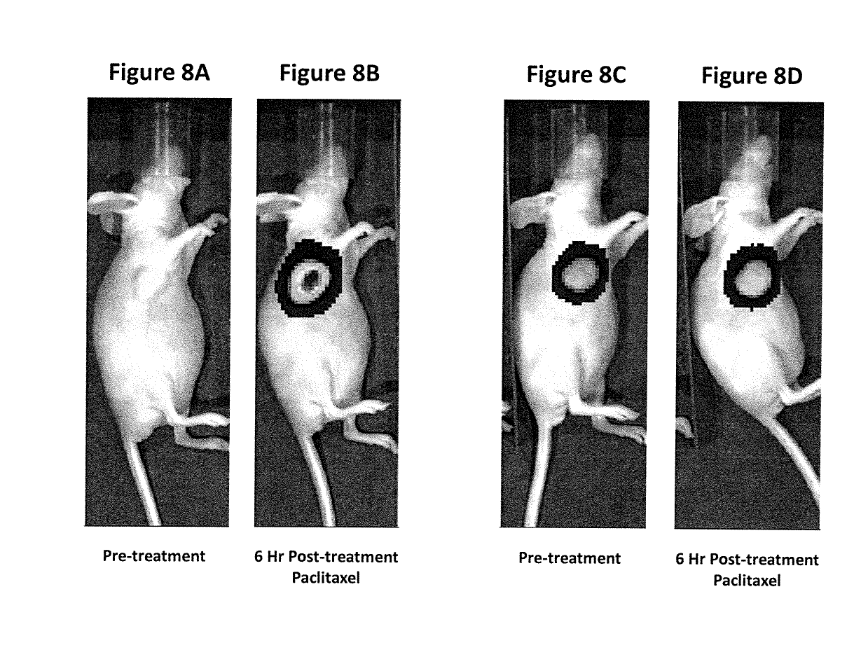

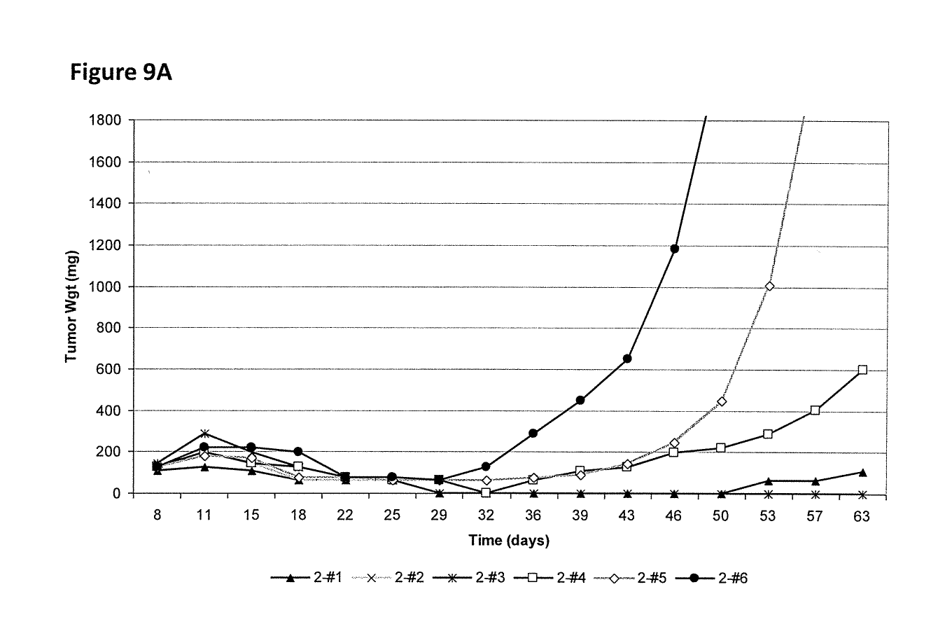

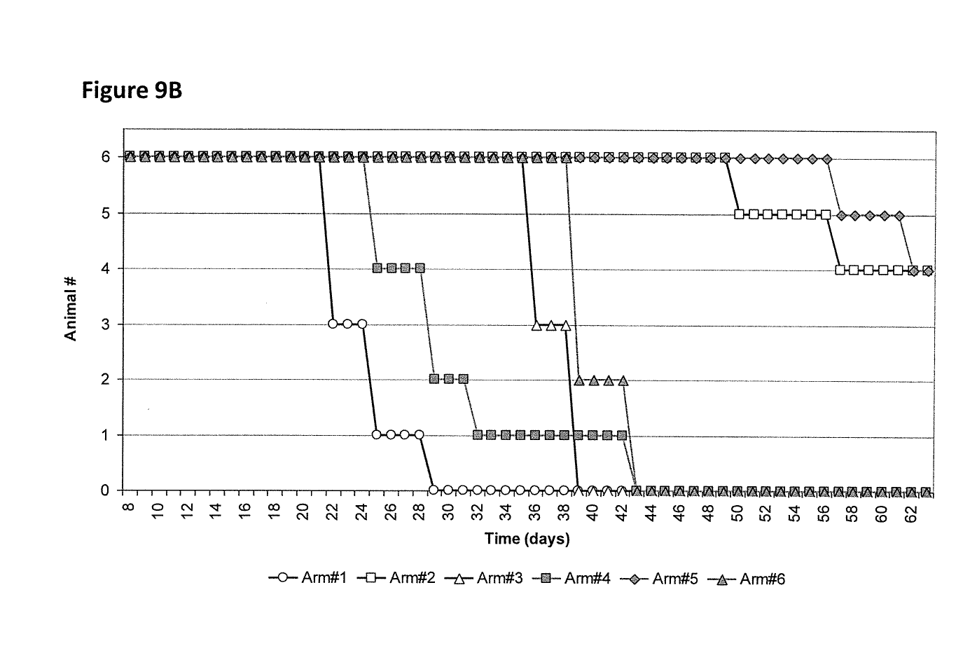

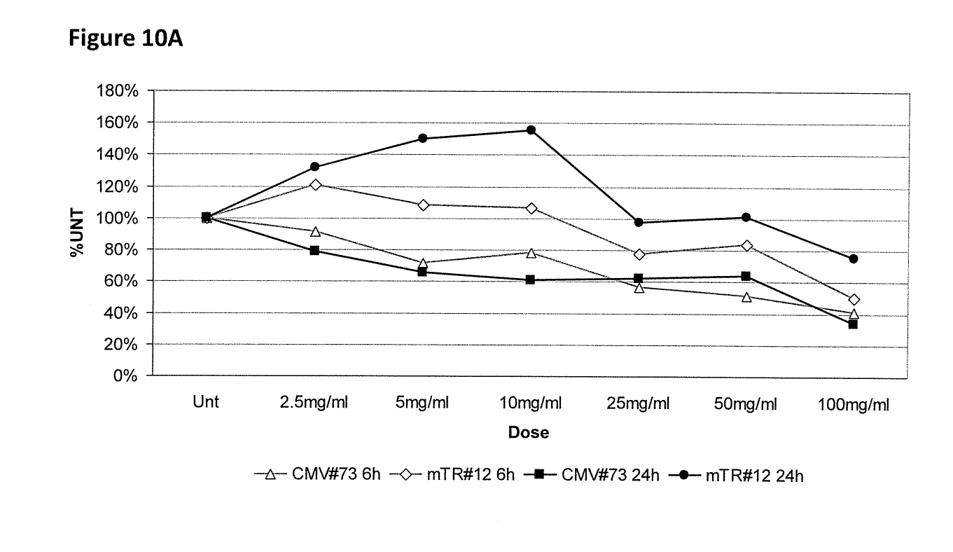

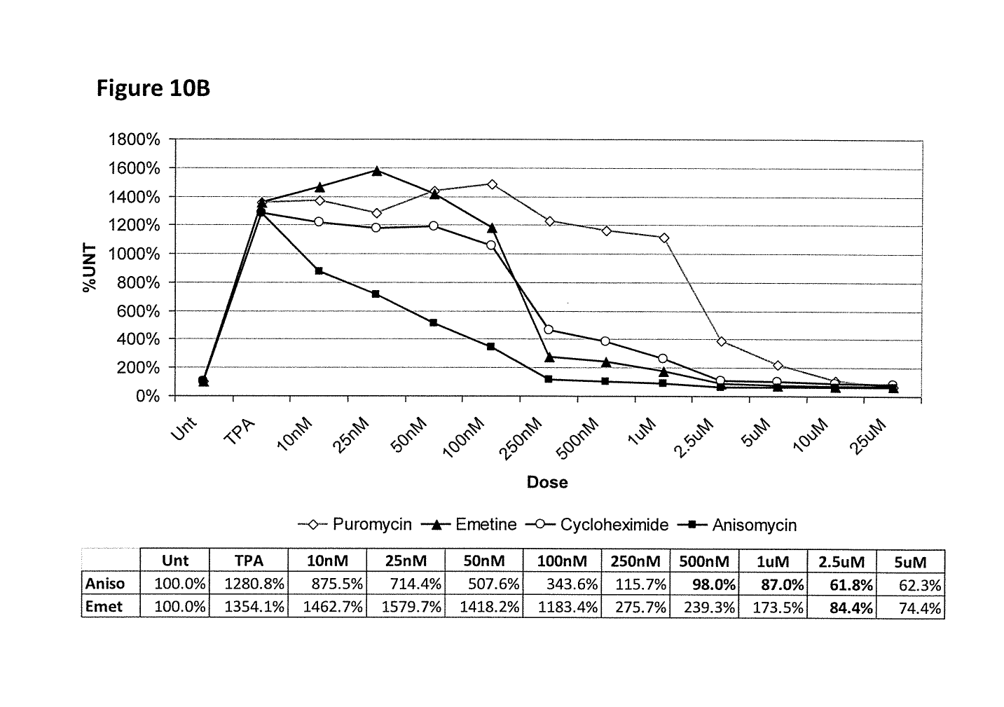

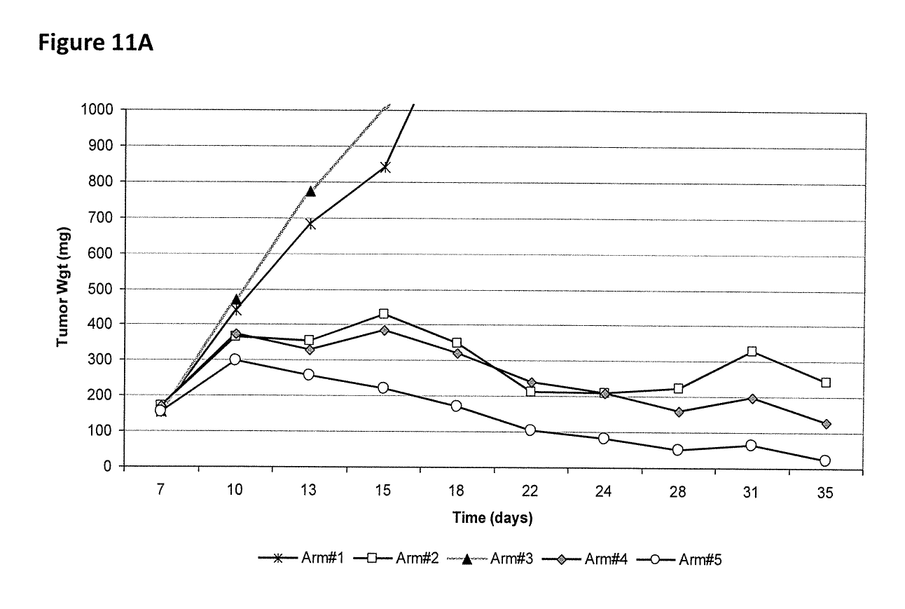

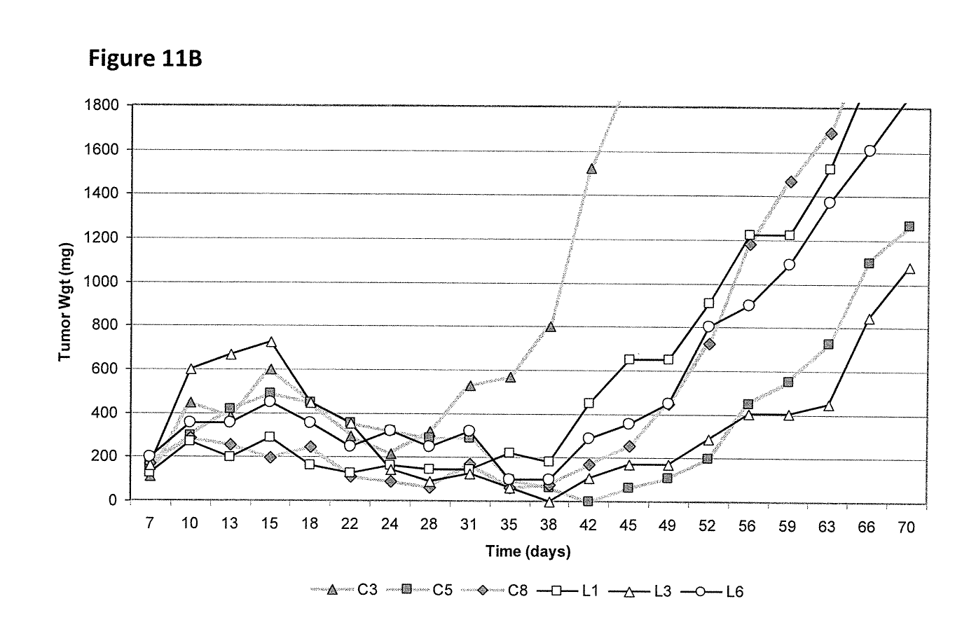

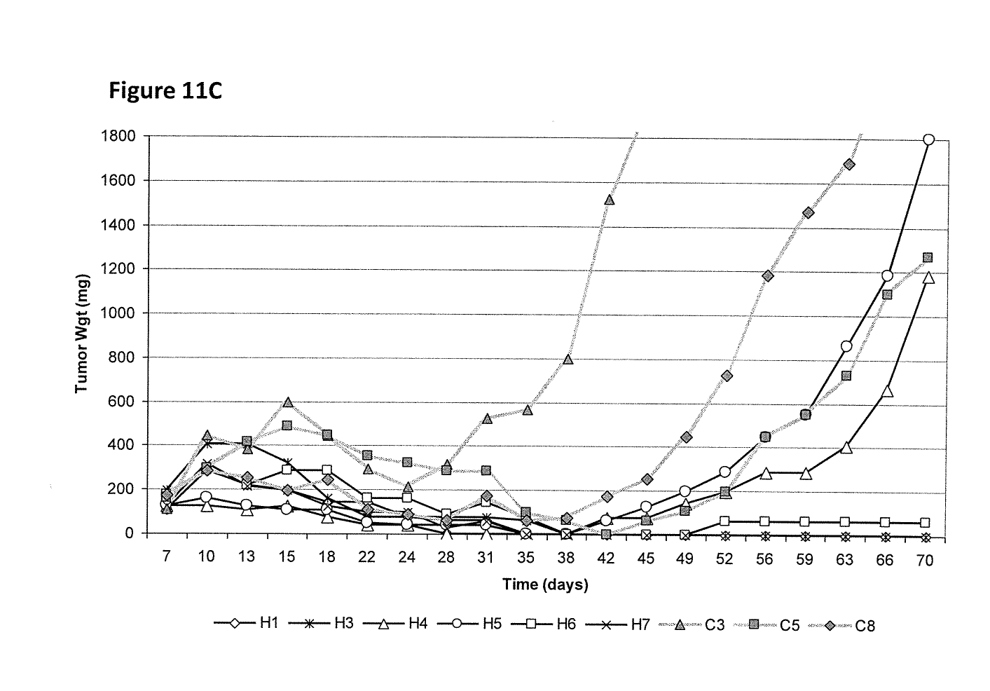

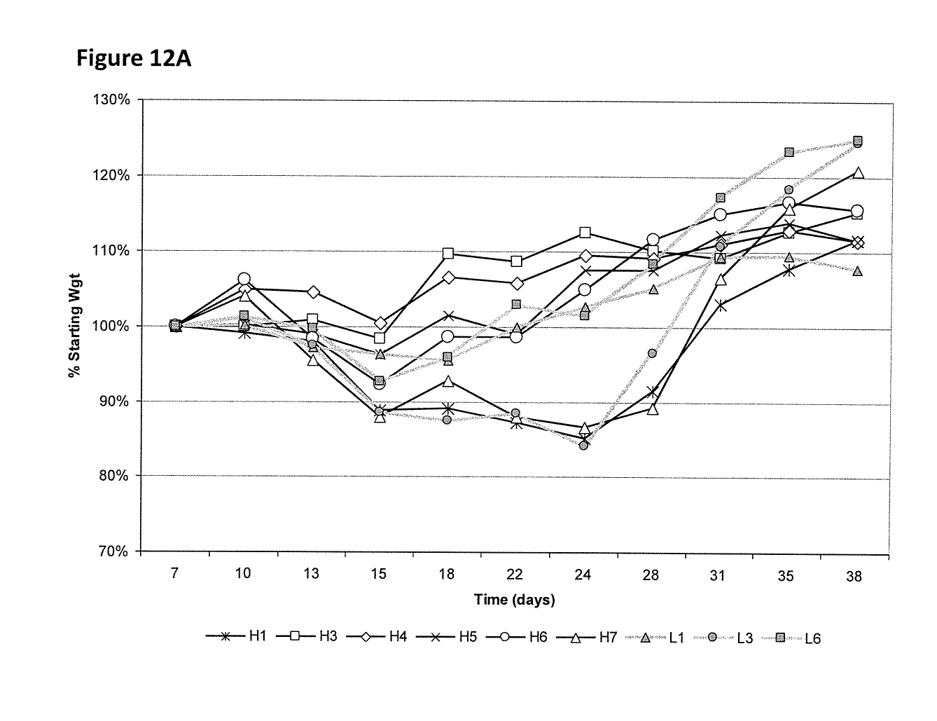

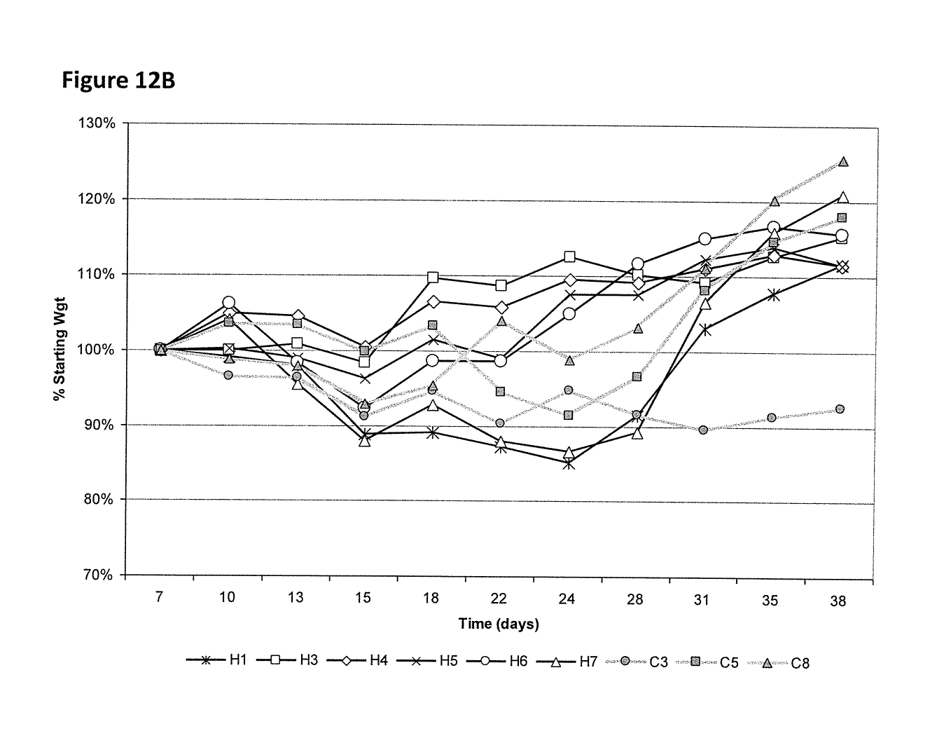

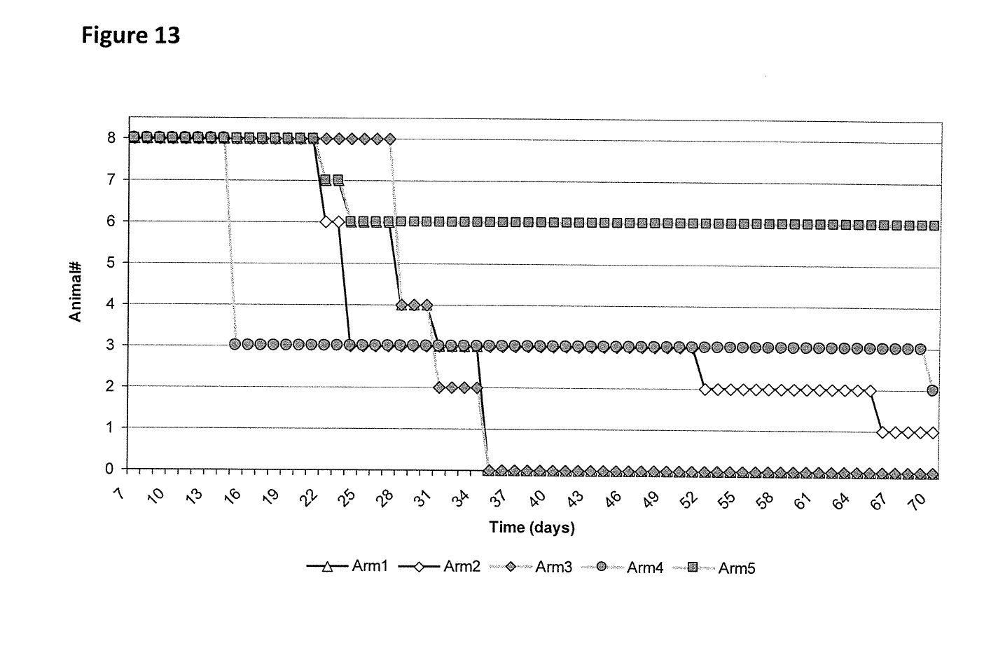

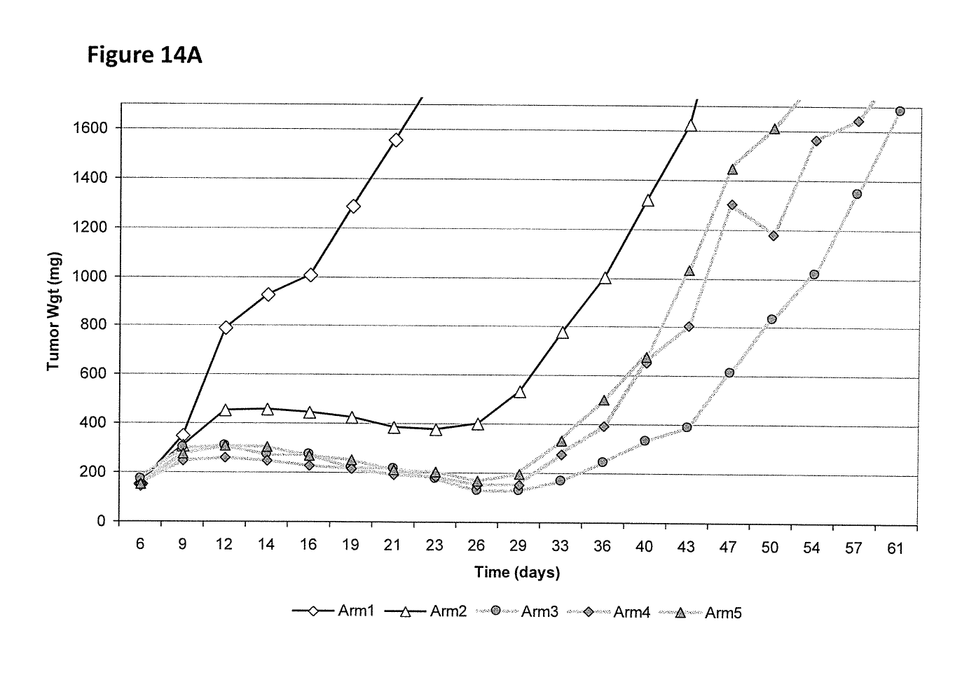

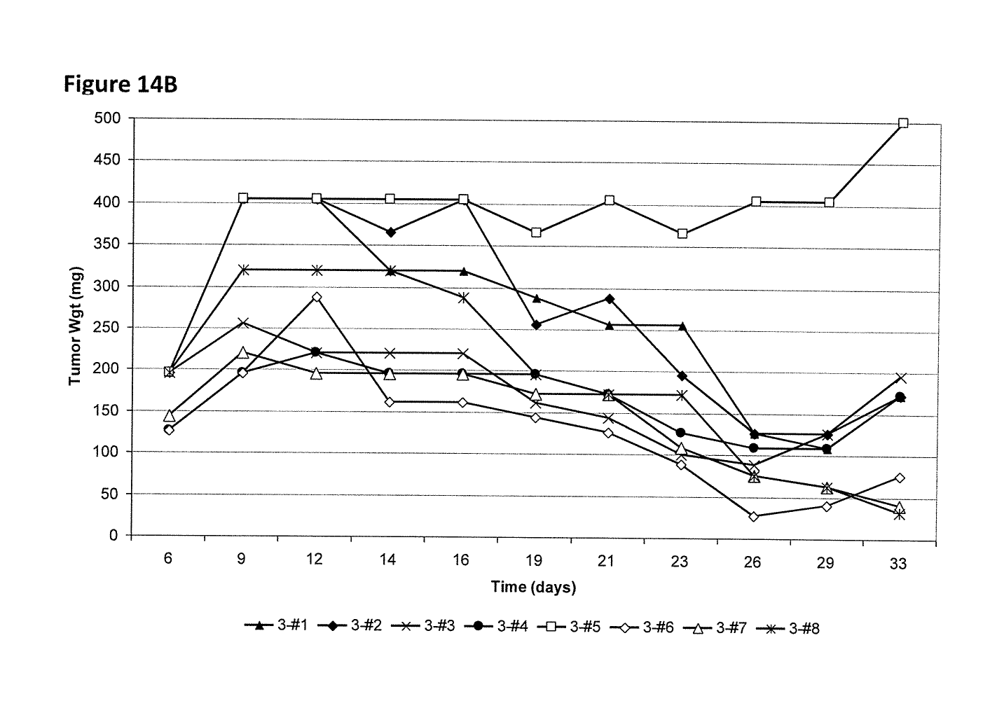

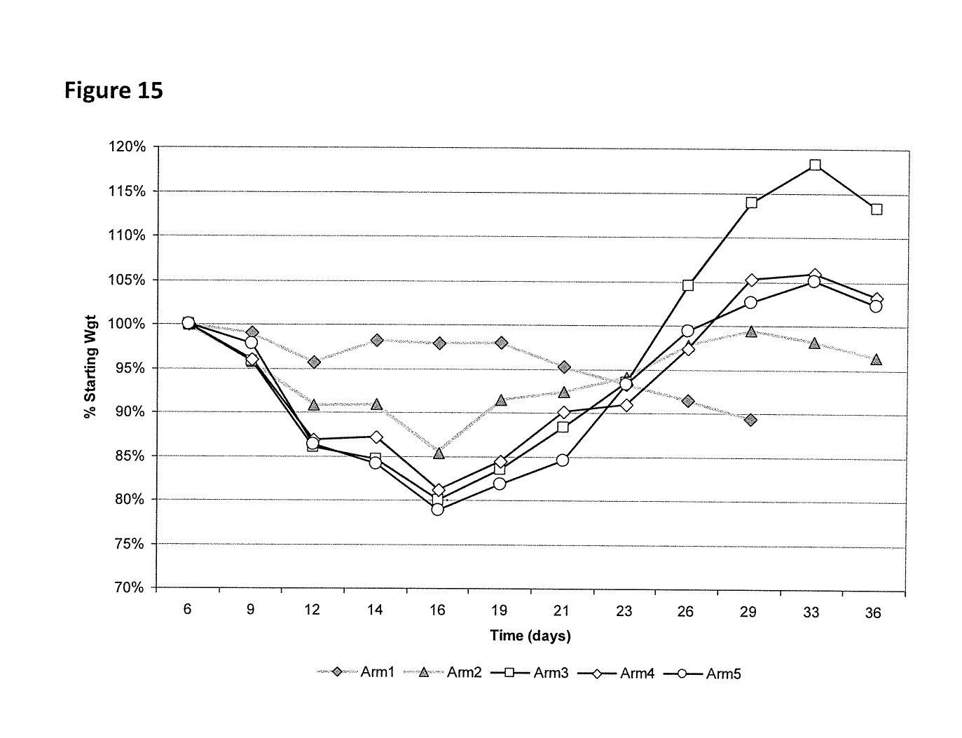

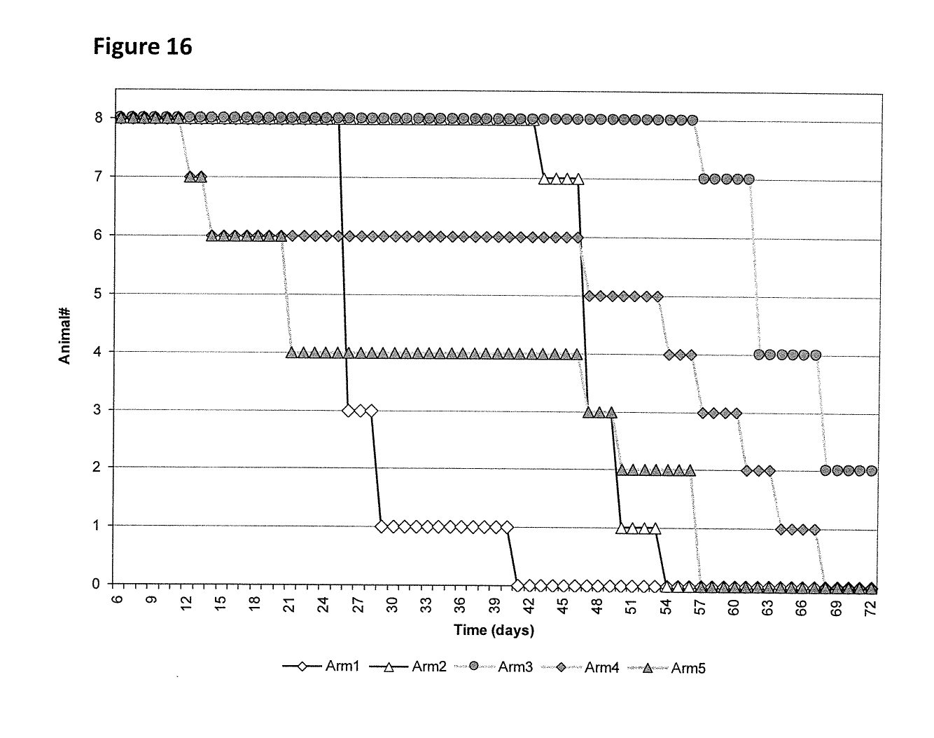

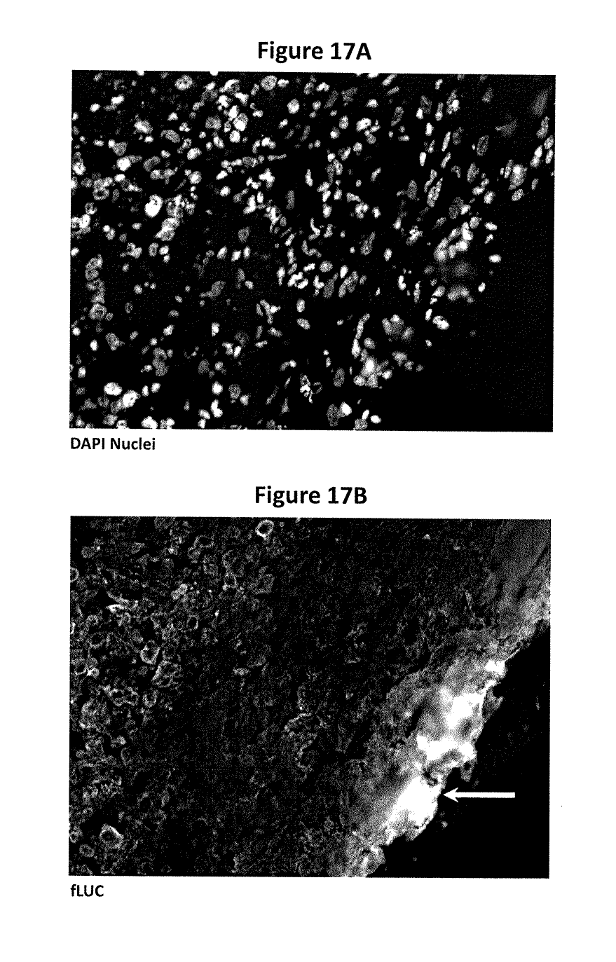

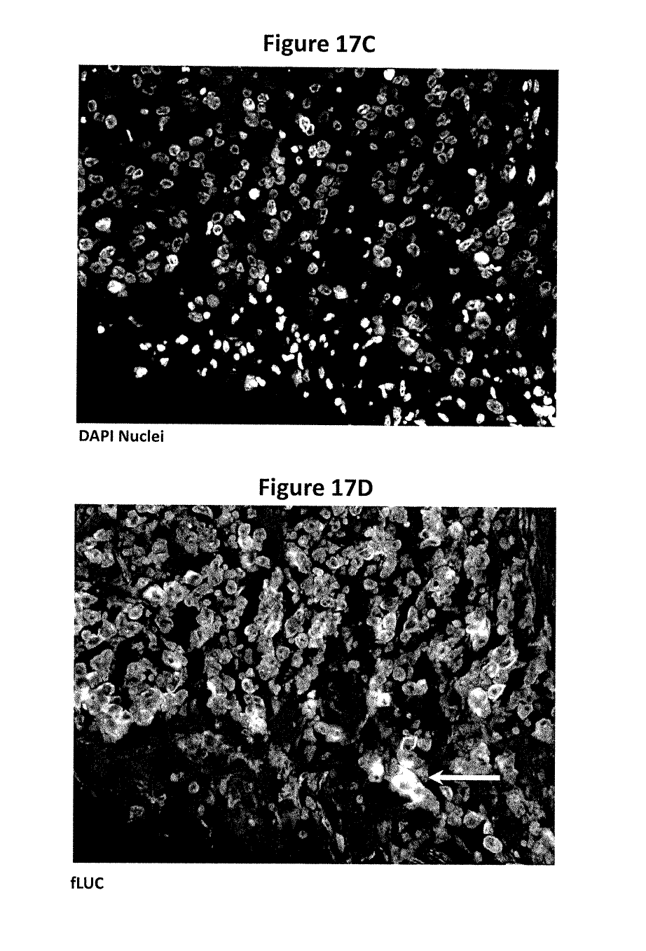

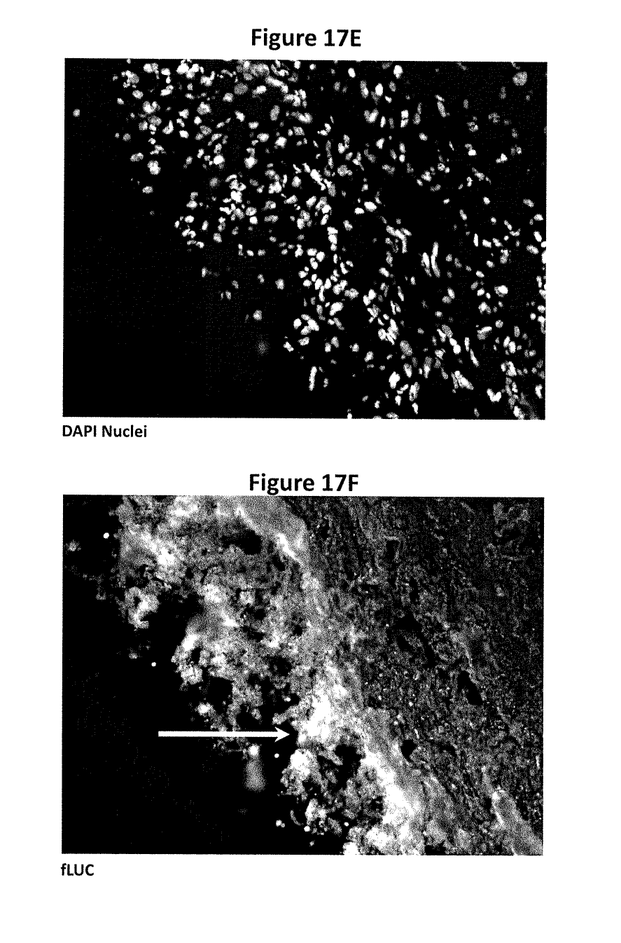

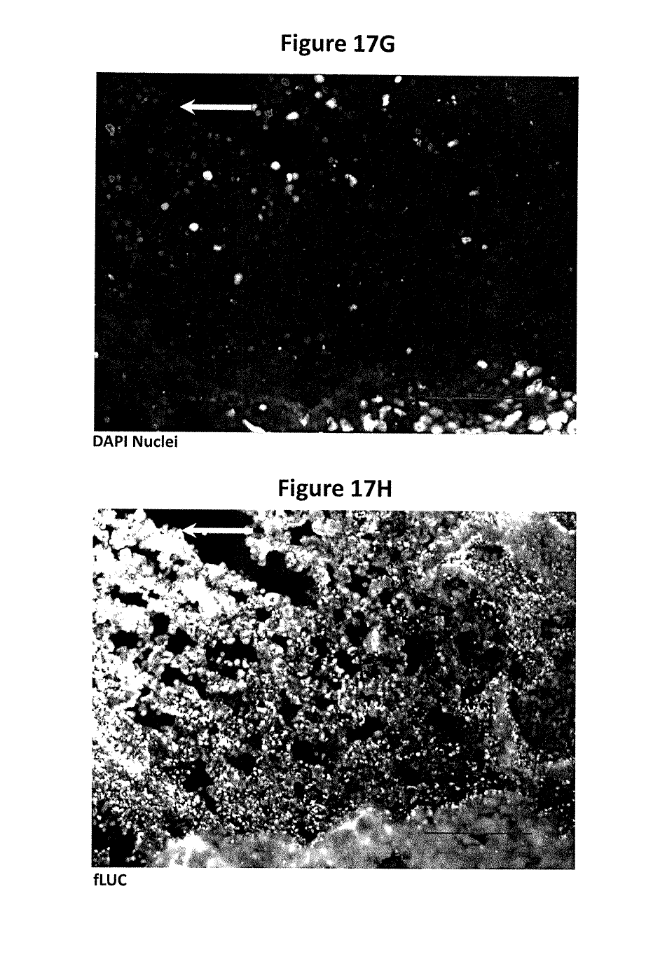

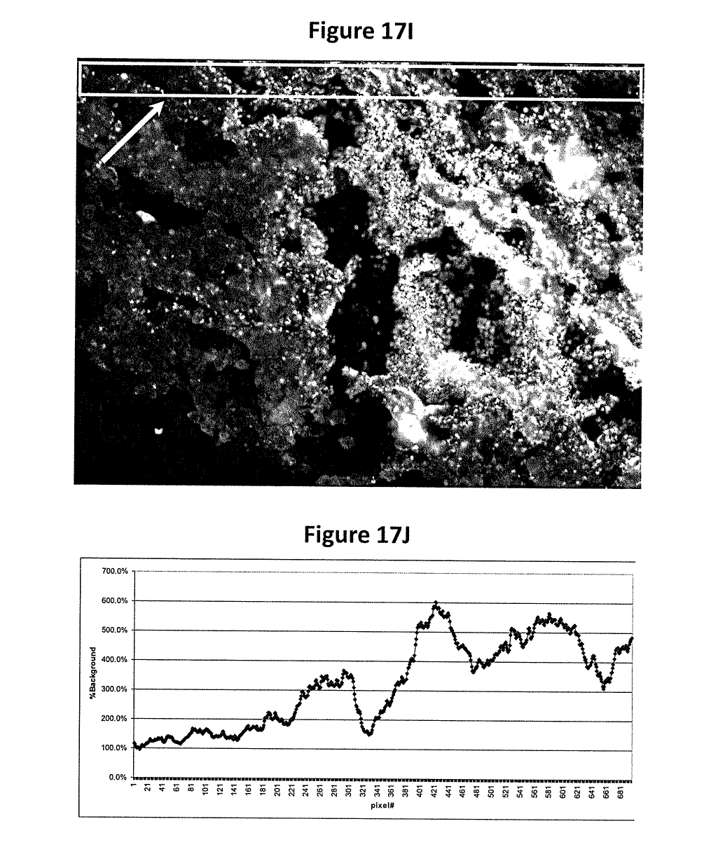

In other studies, the effects of different rapamycin concentrations were measured in combination with the TPA+Taxol Reference Standard response in the MCF7 derived cell lines that express the fLuc reporter either constitutively (CMV lines) or as a part of the TR Expression Cassette (hTR and mTR lines). This reference drug combination was selected because of its particularly strong SET induction in MCF7 cells. Cells were treated with 100 nM TPA, 500 nM paclitaxel, and varying concentrations of rapamycin, incubated at 37° C. for 6 hours, and assayed for fLuc activity. Since SET induction by the TPA+Taxol Reference Standard Reagents is undetectable in the CMV lines (which only show Growth Ribosome responses) and negligible in the TR Class 1 hTRdm-fLUC#6 line, the most useful information comes from the TR Class 3 (mTRdm-fLUC#27 and mTRdm-fLUC#45) and TR Class 4 (mTRdm-fLUC#8) cells, where Rapamycin causes a dose-dependent super-induction of SET in the TPA+Taxol treated samples up to the 50 nM rapamycin dose, followed by a SET decline at rapamycin concentrations of 100 nM and higher. These results are consistent with those in shown in FIG. 4 and confirm a link between the SET Ribosome and mTORC2.

FIGS. 5A and 5B show use of TR Modifier Assays to detect selective regulation of the SET Ribosome by Cobalt.

Abbreviations: TPA: 12-O-tetradecanoylphorbol-13-acetate; TopoL: low dose Topotecan; TopoH: high dose Topotecan; CoCi: Cobalt chloride.

FIG. 5A shows a cobalt(II) dose response chart for the TR Class 3 HEK293 mTRdm-fLUC#12 cell line and illustrates the effects of different cobalt(II) concentrations on Taxol, MG132, and Topotecan (high and low dose) Reference Standard responses. FIG. 5B shows a similar chart for the different cobalt doses combined with the TPA Reference Standard Reagent. Soluble cobalt(II) is widely used for treating anemia and as a research reagent that mimics hypoxia associated with cancer, stroke and cardiac ischemia. Prolonged exposure to cobalt(II) causes heavy metal toxicity which blocks DNA replication and cell cycle progression. Cells were treated with the varying concentrations of CoCl₂, alone or in combination with the Reference Standard Reagents (full names and doses are shown in Table 3), incubated at 37° C. for 6 hours, and assayed for fLuc activity. When applied alone (FIG. 5A), none of the cobalt doses tested induced the SET Ribosome. However, doses >50 μM repressed the background SET Ribosome activity in a dose dependent manner. This SET Ribosome blocking effect was much more pronounced when cobalt was combined with the Reference Standard Reagents known to activate SET (Taxol and TPA). Taxol response was inhibited starting at 50 μM CoCl₂, while TPA activation (FIG. 5B) was inhibited starting at 200 μM CoCl₂. The available toxicity data shows that the 50 μM-200 μM cobalt concentrations correlate with chronic toxicity in humans affecting multiple organs after long exposure times, while doses above 200 μM are associated with production of reactive oxygen species by the mitochondria and bacterial/animal death (mouse and rat LD50). Thus, the ability of a drug or chemical to block SET Ribosome activation may be a good predictor of in vivo toxicity and side effects.

(0090) FIG. 6A shows a class dependent SET ribosome regulation by Topotecan. In this figure, a topotecan dose response assay was performed on the MCF7 derived cell lines that express the fLuc reporter either constitutively (CMV lines) or as a part of the TR Expression Cassette (hTR and mTR lines). Topotecan is a mature First-Line oral therapeutic that disrupts Topoisomerase I protein function, DNA replication and cell cycle progression which is commonly used to treat ovarian, cervical, and small cell lung cancer. Cells were treated with varying doses of topotecan, incubated at 37° C. for 6 hours, and assayed for fLuc activity. In the CMV cell line, where the fLuc reporter is translated by the Growth Ribosome, topotecan had little effect on protein synthesis at doses <500 nM. The mTR and hTR cells, where the fLuc reporter is translated by the SET Ribosome, respond in accordance with the TR Class structure. Class 1 hTRdm-fLUC#6 and hTRdm-fLUC#15 respond similarly to the CMV control line. TR Class 3 (mTRdm-fLUC#27 and mTRdm-fLUC#45) and TR Class 4 (mTRdm-fLUC#8) show maximum SET value at 10 nM-100 nM topotecan which was followed by a decline below the SET maximum (produced by >100 nM-5 uM topotecan doses) and a complete SET Ribosome block (5 uM and higher topotecan concentrations) that is exemplified by no SET protein synthesis and reporter protein turnover for 6 hr. This high dose SET inhibition correlates with a known concentration dependent block of DNA replication at an intra-S cell cycle checkpoint which effectively blocks Cap-dependent and SET Ribosome activity.

FIG. 6B illustrates the effects of different topotecan concentrations on the TPA Reference Standard response in the HEK293 derived cell lines that express the fLuc reporter either constitutively (CMV3 line) or as a part of the TR Expression Cassette (hTRdm-fLUC#13 and mTRdm-fLUC#45 lines). Cells were treated with 100 nM TPA and varying concentrations of topotecan, incubated at 37° C. for 6 hours, and assayed for fLuc activity. Neither TPA nor topotecan had a pronounced effect on Cap-dependent Ribosome activity in the CMV control cells. While low doses of topotecan produce only mild SET superinduction in the hTR and mTR cell lines compared to that caused by TPA alone, doses between 100 nM and 5 uM produced a decline in SET activation with doses >5 uM completely blocking SET Ribosome activity as in FIG. 6A

FIG. 6C shows how TR SET ribosome activity induced by Topotecan correlates with in vivo toxicity. Comparing the results in FIG. 6A to the considerable preclinical and clinical drug dosing and toxicity data available for topotecan revealed a strong correlation between the topotecan dose, SET response, DNA replication injury, and chronic/acute toxicity. Low doses (<10 nM) that produced a rapid SET Ribosome induction are associated with cell stress but not death. Doses that result in maximal SET plateau (10 nM to 100 nM) correlate with chronic ex vivo and in vivo toxicity, such as slow death of cultured cells, human clinical treatment doses and the human Maximum Tolerated Dose (MTD). High doses (100 nM-5 uM) that cause SET decline are invariably associated with acute ex vivo and in vivo toxicity (immediate G2/M cell cycle block in cultured cells and the mouse LD50). Finally, the highest dose range (5 uM to 25 uM) that blocks SET Ribosome induction is characterized by momentous cell death associated with an intra-S checkpoint. Ex vivo studies find that these doses produce immediate lethality (<24 hr) if the drug remains in constant contact with cells; however, protocols removing the drug before ˜6 hr result in delayed but significant apoptotic cell death (90-95%) within 72 hr. TR Class 4 mTRdm-fLUC#8 cells exhibit the greatest SET induction and enhanced resistance to drug toxicity, as defined by standard cell viability assays and higher drug concentrations needed to completely block SET Ribosome activity.

FIG. 7 shows a key step for identifying a TR metastatic cancer cell line model.

Advanced and aggressive tumors are thought to contain a unique population of cancer cells that exhibit stem cell traits, such as an ability for self-renewal, the capacity to evolve and give rise to novel stem cell progeny, enhanced resistance to cell damage, and a tumor initiating capacity. Although cancer stem cells (CSCs) represent a small fraction of any tumor, they constitute the population needed to create distant, heterogeneous metastases. Because a high TR Class number (and elevated SET Ribosome activity) correlates with increased G2/M damage repair potential, improved cell viability, and drug resistance; multiple mTR and hTR cell lines were used to compare SET Ribosome responses with established in vitro and in vivo CSC properties. By example, a TR Metastatic Cancer Cell model will exhibit a series of measurable traits including: (1) it was derived from a small outlier population of a parental TR cell line (top 1-5% SET induction), (2) it demonstrated drug and stress resistance that correlated with a statistically elevated SET Ribosome activity in cell-based TR assays (termed a Class 4 response), (3) it exhibited Clonal Evolution that resulted in highly significant changes in SET Ribosome activity (creating a novel TR Outlier response) as a result of low density selective growth, such as repeated single cell colony formation and the generation of nonadherent tumorspheres from a small number of cells, (4) it displayed in vivo tumor initiating activity following serial xenotransplantation into nude mice, (5) it formed xenogenic tumors that exhibited in vivo regulation of SET-specific translation from the TR expression cassette, and (6) it formed xenogenic tumors with an elevated growth rate and resistance to cytotoxic drug treatment. For example, a TR metastatic colorectal cancer (CRC) cell model clone would be isolated from a parental CRC cell line (such as HCT116) and exhibit each of these traits. As shown in subsequent sections, one example of a TR metastatic CRC cell model is hTRdm-fLUC#32.

FIG. 7A shows how the magnitude of SET Ribosome activity correlates with genetic instability in tumor cells grown in an in vitro culture model of metastatic growth. Every human tumor is composed of many distinct cell subpopulations that exhibit unique biological properties (tumorgenicity, metastatic potential, drug resistance, etc.). These populations can inter-convert during tumor progression as a result of genetic instability, which allows parts of the tumor to repair replication damage produced by antineoplastic drugs and regrow upon completion of the treatment cycle. Although current technology can detect tumor cell conversion, the process is lengthy, expensive, and employs cell-specific biomarkers that preclude widespread correlations between cancer types. To investigate the relationship between the TR SET response and tumor cell conversion events associated with an in vitro model of metastatic growth, the FIEK293 TR Cell Panel lines were plated at low density and allowed to form colonies (˜2 months; 50 growth cycles selecting for elevated cell adherence and colony formation ability), and then used to generate a daughter subclone cell panel for each line. The new subclones were treated with 100 nM TPA, incubated for 6 hours, assayed for fLuc activity, and the subcloned cell responses were compared to the parental cell lines. FIG. 7A shows the ranking plot of the fLuc responses to the TPA Reference Standard Reagent in the daughter panel generated from the hTRdm-fLUC#122 (TR Class 4) parental line. The wide range of responses in the daughter cells shows that tumor cell conversion had altered the inheritable SET activity, which means that the SET ribosome can adapt during the selection process and display genetic heterogeneity, also known as Clonal Evolution. The arrow indicates the median response, showing that subclone numbers were roughly equal on both sides. However, it is particularly important that the TR Class 4 hTRdm-fLUC#122 line generated a daughter cell with an outlier SET induction activity (median response 1788% compared to an outlier daughter subclone with a 6568% response or a 3.7-fold increase in SET induction). The slope of the line reflects the degree of heterogeneity, with the TR Class 2 CMV#74 and TR Class 3 mTRdm-fLUC#12 showing the greatest instability. These results provide convincing evidence that SET responses propagated in stable cell lines provide a simple, functional biomarker for tumor cell conversion. The lower frequency of conversion events in high and low TR SET Classes might be explained by the difference in G2/M cell cycle phase recovery potential in these cell populations. For example, low TR Class cells exhibit minimal SET Ribosome activity and would be easily killed at an S or G2/M checkpoint during toxic treatment, whereas the high TR Class cells express elevated SET Ribosome activity which correlates with stress resistance, high viability and an ability to recover from replication damage. Therefore, tumor cell conversion is not an absolute gauge of survival potential but a measure of the frequency of survival to a given stressor. The ability of the TR Class 4 cell line to exhibit correlate conversion activity with recovery, viability and drug resistance validates the importance of new therapeutics designed to reduce tumor cell recovery potential. In a second in vitro cell culture model of metastatic growth, FIGS. 7B and 7C show a HCT116 TR Class 4 TR SET cell line that readily formed tumorspheres and exhibited anchorage independent growth. While a high TR SET response is not absolutely required for tumorsphere formation (TR Class 1-3 cells often display this trait, see Table 4), a true metastatic cell candidate must exhibit nonadherent growth which is defined as growth on non-coated tissue culture dishes, whether attached or unattached (FIGS. 7B and 7C). As shown, tumorspheres form within a few passages (free floating cell masses), and exhibit de novo clonal tumorsphere activity (nonadherent cell growth from single cells). FIG. 7C shows a clonal tumorsphere, marked with an arrow, that contained fewer than 10 nuclei (determined using DAPI nuclear staining).

As shown in Table 4, the HCT116 cell panel was grown as tumorspheres for 32 days in untreated tissue culture dishes and replated on standard tissue culture dishes for 14 days prior to performing a TR SET Assay using the TPA TR SET Reference

Standard. In this study, three cell lines displayed enhanced SET induction levels consistent with an outlier TR SET response (mTRdm-fLUC#25, #28 and #75). For these putative TR metastatic cancer cell models, the TR SET responses increased to 19,413% -26,675% of an untreated control (15-fold to 79-fold).

FIGS. 8A-8D and Table 5 test a Class 4 TR SET cell line for a Tumor Initiation Phenotype. To examine the ability of the TR Class 4 HCT116 hTRdm-fLUC#32 cell line to form tumors in nude mice (nu/nu), ten animals were implanted with either HCT116 hTRdm-fLUC#32 or parental HCT116 cells (5×10e6 cells) and tested for tumor growth, defined as time to 750 mg. The parental HCT116 cell exhibited a range of tumor growth responses including one aggressive tumor and one no-take implant (time to 750 mg was 8.6 days). In contrast, the growth of the ten TR cell tumors did not show significant group variability (time to 750 mg was 8.8 days). These results were consistent with the fact that the TR cells were cloned from the parental HCT116. In a second study, 30-60 mg tumor fragments were cut from the cell derived tumors and implanted bilaterally into 6 nude mice, generating 12 tumor events (Table 5). The final size of bilateral implants (Day 21 of the study) was variable. For example, the HCT116 3R implant grew to 2138 mg, while the 3L implant was only 138 mg. Additionally, there was a significant size difference between the large tumors and the small tumors in each study arm. The two largest parental HCT116 tumors differed significantly from the remaining ten tumors (p=0.00034, 2-tailed t-Test). Similarly, the four largest HCT116 hTRdm-fLUC#32 tumors differed significantly from the remaining eight implants (p=0.00001, 2-tailed t-Test). Whereas no significant difference in tumor size was observed between the two arms, the tumor size distribution was skewed so that the HCTI 16 hTRdm-fLUC#32 cells were larger than the HCT116 control (Table 5). For example, 4 of 12 TR implants produced tumors larger than 1.25 g compared to 2 of 12 control samples. Similarly, 6 of 12 TR implants were larger than 550 mg compared to 4 of 12 HCT116 tumors. The enhanced HCTI 16 hTRdm-fLUC#32 tumor growth rate in vivo verified that it was an appropriate choice for the TR SET Metastatic Tumor Model. FIG. 8A-8D and Table 6 provide further support for the hTRdm-fLUC#32 cell line as a TR metastatic Colorectal Cancer (CRC) cell model. In this study, 58 nude mice were injected with the HCT116 hTRdm-fLUC#32 or parental HCT116 cells and the tumors were grown to ˜125 mg (Day 8 of the study). The animals were organized in six arms (6 animals per arm or n=36) with the remaining 22 animals triaged as controls. In a first effort, the TR SET animals (n=18) were assayed for SET Ribosome activity by noninvasive bioluminescent imaging for the fLUC reporter activity prior to test agent injection (Pre-treatment animals in FIGS. 8A and 8C and Table 6). Animals were allowed to recover, injected with vehicle in Arm 1 (polyoxyl 35 castor oil or cremophor EL, 0.5 mg/kg or 75.8 mg/sq m/day), 120 mg/kg cyclophosphamide in Arm 3 (a chemotherapy drug that has no effect on HCT116 tumors), or 20 mg/kg/day paclitaxel (taxol) dissolved in CremophorEL in Arm 2, and retested for fLUC activity after 6 hrs (to mimic the timing of the cell based TR SET assay). FIGS. 8A-8D and Table 6 shows examples of an unexpected chronic, G2 cell cycle phase stress in small tumors (˜125 mg). The Pre-treatment bioluminescence levels resolved into two distinct tumor types: a Low Stress group (low SET Ribosome activity was exhibited by 8 of 18 tumors), exemplified by FIG. 8A; and a High Stress group (high SET Ribosome activity expressed by 10 of 18 tumors), represented by FIG. 8C. As shown in Table 6, when the animals were re-imaged 6 hrs after treatment, the Low Stress tumors significantly increased fLUC activity compared to pre-treatment expression levels (range 7192% to 46600%). Surprisingly, this occurred not only in the paclitaxel/cremophorEL treated arm, but also in cremphorEL and cyclophosphamide tumors. In contrast, High Stress tumors (FIG. 8C-8D) were incapable of fLUC super-induction (i.e. unable to activate the SET Ribosome). A subsequent study found that the frequency of the Pre-treatment G2 cell cycle phase stress correlated with tumor size. As shown in Table 6, nine of the eleven triaged animals containing HCT116 hTRdmfLUC-#32 tumors were assigned to 3 test arms and assayed for bioluminescence as before. Due to the delay in processing these animals, tumor size had increased to ˜500 mg and only 1 of 9 tumors exhibiting low SET Ribosome activity. Since this tumor exhibited a significant SET induction (20500%) when treated with paclitaxel/cremophorEL, activation of the SET Ribosome was not affected by tumor size.

FIGS. 9A and 9B show that the TR metastatic CRC cell model hTRdm-fLUC#32 exhibited stress-dependent drug resistance to Paclitaxel. To assay how activation of the SET ribosome might regulate in vivo tumor recovery and growth, tumor size was monitored in each test arm for a total of 63 days. Animals were sacrificed for tumor burden (>2 g) or at the end of the trial (63 days).

As expected, polyoxyl 35 castor oil (cremophor EL) had no effect on tumor growth and recovery. Cyclophosphamide treatment slowed tumor growth compared to cremophorEL control, but did not result in tumor regression. In the taxol arm, a strong correlation between the pre-treatment G2 cell cycle phase tumor stress and the apparent therapeutic index of paclitaxel/polyoxyl 35 castor oil was observed (FIG. 9A and Table 6). Although the three Low Stress tumors (animals #4, #5 and #6) exhibited growth arrest and modest tumor regression (˜33% size decrease) during the 10-day paclitaxel treatment period (FIG. 9A), 14-18 days after treatment was discontinued, each tumor had increased in size. Two of the three Low Stress animals were sacrificed for tumor burden on Day 50 and Day 57, and the remaining tumor was >600 mg and growing rapidly on Day 63. In contrast, a significant decrease in the size of the High Stress tumors was observed during paclitaxel treatment (>59% size decrease). This trend continued until day 29, when the tumors became too small to measure (<50 mg size). For this group, only 1 animal exhibited any tumor regrowth, resulting in a 100 mg tumor on Day 63. Necropsy found no obvious tumors in the 2 remaining High Stress animals at the end of the trial period. Given that all tumors were derived from the same drug resistant TR metastatic CRC cell model (the hTRdm-fLUC#32 cell line), it is apparent that Pre-treatment SET ribosome activation, produced by G2 cell cycle phase translation, plays a major role in regulating in vivo tumor response to the First Line oncology drug Paclitaxel.

FIG. 9B shows that the TR metastatic CRC cell model exhibits enhanced cell growth that correlates with decreased animal survival. Preclinical in vivo Survival is a function of spontaneous animal death, animal wasting (animal sacrifice after >20% total weight loss) and maximum allowed tumor burden (animal sacrifice after tumor size is >2 g). In this particular trial, all animals were sacrificed due to tumor burden. This panel shows a Kaplan-Meier graph, where the animal number (% Survival) is plotted versus day of trial (time) and provides an estimate of the Survival Function for each treatment arm. While cyclophosphamide had some effect on animal survival compared to cremophorEL control, all of the animals were sacrificed due to tumor burden well before the end of the trial. Only the paclitaxel/cremophorEL treated arms showed prolonged animal survival (4 of 6 animals survived to day 63). It is important to note that the TR metastatic tumor cell model derived tumors (Arms 1-3) grew more aggressively than the parental HCT116 derived tumors, which resulted in earlier animal sacrifice across all treatment arms. Together the tumor and animal survival results show that in addition to paclitaxel and cremophorEl, an undefined tumor stressor is needed to induce a chronic G2 checkpoint which correlates with an enhanced tumor response and prolonged animal survival.

FIG. 10A shows the use of the TR SET Assay to examine the in vitro ability of an in vivo SET Agonist to activate the SET Ribosome. As shown in Arm 1 of Table 6, 0.5 mg/kg cremophor (oral dose equivalent to 62.5 mg/ml) induces SET in xenogenic tumors 6 hours after intravenous (IV) delivery. To examine this in vivo response in a cell based TR assay, cremophorEL doses ranging from 2.5 mg/ml-100 mg/ml were continuously applied to HEK293 mTRdm-fLUC#12 (a potential TR metastatic CRC cell model) and CMV-fLUC#73 cells for 6 hours and 24 hours. In contrast to the >2000% SET increase produced by 100 nM TPA, unexpectedly mTRdm-fLUC#12 cells treated with cremophorEL exhibited only a 60% SET increase at 24 hours (2.5-10 mg/ml). Furthermore, the CMV response shows that cremophorEL is an inhibitor of the Cap-dependent ribosome. These results mean that cremophorEL can activate the SET ribosome in vivo but application to cultured cells does not induce a G2 cell cycle checkpoint. Although surfactants are commonly used to solubilize hydrophobic drugs, cremophorEL is not an inert vehicle and produces many in vivo biological effects. This study provides evidence that a cell model can exhibit an in vivo drug response that may not be observed in vitro.

As shown in Table 3, a number of SET Antagonists have been identified; however, the majority of these agents simply prevent cell cycle progression in S phase and prevent SET Ribosome activation in G2. FIG. 10B shows the in vitro TR Assay results examining SET antagonists that can bind directly to the SET Ribosome which will selectively block G2 translation at subtoxic doses and prevent cell cycle progression. This effect should mimic the effect of endogenous stress on the apparent therapeutic index of oncology drugs. Multiple compounds have been tested for their ability to block SET Ribosome activation using the TPA Reference Standard Response Modifier Assay, in which the high TR Class HEK293 cells were treated with 100 nM TPA and varying concentrations of candidate SET Ribosome blockers, incubated at 37° C. for 6 hours, and assayed for fLuc activity (for example FIG. 5B).

For this study, 4 compounds were selected that bind to different ribosome structures. Anisomycin binds to the 60S ribosomal subunit at the A site, which is the point of entry for the aminoacyl tRNA (except for the first aminoacyl tRNA, which enters at the P site). Puromycin interacts with both 40S and 60S subunits at the P site, where the peptidyl tRNA is formed in the ribosome. Cycloheximide binds to the 60S subunit at the E site, which is the exit site of the uncharged tRNAs after they discharge their amino acid to the growing peptide chain. Emetine binds at a ribosome shelf structure adjacent to the E site, but unlike cycloheximide it binds to the 40S subunit rps14 ribosomal protein. Of these test compounds, only emetine exhibits significant water solubility, which required a solvent such as DMSO for the high dose assays.

FIG. 10B illustrates the effects of different concentrations of the candidate SET Antagonists on the TPA Reference Standard response in the Class 3 HEK293 hTRdm-fLUC#13 cell line. The most dramatic result was the detection of a linear dose-dependent inhibition of SET by low dose anisomycin (SET ribosome activity steadily decreased between 10 nM and 250 nM concentrations with an IC50 of ˜35 nM, and was completely blocked by doses >500 nM). The same treatments had minimal effect on Cap-Dependent translation in the HEK293 CMV#3 line. Therefore, anisomycin must inhibit DNA replication and cell cycle progression at S/G2 by activation of the p38MAPK stress kinase, which interacts with the PKC signaling system. Emetine and cycloheximide inhibited SET at doses between 50 nM and 1 uM, followed by a SET Blocking activity at doses above 2.5 uM. Similarly to anisomycin, emetine is also known to interact with stress kinases. Puromycin was the least efficient at SET inhibition, acting between 1 uM and 2.5 uM doses, which are known to be toxic due to disruption of polysomal structures.

When selecting an optimal Biologically Effective Dose for a SET Antagonist, the lowest dose that resulted in complete and immediate inhibition of SET Ribosome activity was determined (an IC100). Given the known fLUC protein half-life, any treatment that immediately blocks SET protein synthesis will result in ˜15% decrease in fLuc activity within 6 hr (the timing of a standard TR SET assay), which means that continuing protein synthesis is required to produce >85% fLUC activity. In contrast, any treatment that increases fLUC protein turnover will result in <85% fLUC activity at 6 hr. FIG. 10B shows that 500 nM anisomycin treatment results in 98% fLUC activity compared to the untreated control. Given that 15% of the fLUC has degraded during this assay, this value represents either ˜15% residual translation for 6 hr or a short burst of protein synthesis prior to a translational block. In contrast, 1 uM anisomycin results in an immediate block or IC100 (87% fLUC activity), which contrasts with higher doses that seem to exhibit enhanced protein degradation (˜2× the expected fLUC turnover rate). Therefore, 500 nM anisomycin is an effective (but incomplete) SET Inhibitor, while 1 uM anisomycin produces a complete or IC100 SET Antagonist response. Further dose increases enhance not only the SET Antagonist activity, but may also promote protein degradation (which may negatively affect normal cell recovery and produce unpredictable systemic side effects). However, many cell based effects may not be observed in vivo. Therefore, the first animal trial examined two anisomycin doses to test for a sub-IC100 dose effect in animals. As shown in Table 7, a 500 nM equivalence dose (Low Dose=0.000027 mg/kg/day) and a 1 uM equivalence dose (High Dose=0.000054 mg/kg/day) were selected. In a subsequent trial, the 1 uM equivalence dose) was compared to a 2.5 uM equivalence dose (Very High anisomycin dose) and a 2.5 uM equivalence dose of emetine to test for a preferred dosing regimen

FIGS. 11A-11C, Table 8, and Table 9 show the first Xenogenic Animal Trial results for a SET Combination Drug. To test for the ability of SET Blocker drugs to improve the efficacy of the First Line anti-CRC oncology drug Capecitabine, the HCT116 hTRdm-fLUC#32 metastatic CRC tumor cell model was injected into nude mice (athymic nude-Foxn 1 nu), allowed to form tumors, and were treated orally QD with various SET Component combinations using five treatment Arms (Table 7). The treatment was started when the tumors reached ˜125 mg in size (Day 7 of the study) and applied daily for 18 days, after which the animals were monitored for additional 45 days. The drug concentrations for each arm of the study are listed in Table 7. Whole body weights and tumor weights were measured using standard sizing procedures. Animals were sacrificed due to wasting (after >20% total weight loss) or for tumor burden (>2 g).

FIG. 11A, Table 8 and Table 9 show that a SET Combination drug will induce significant tumor regression when applied with high dose Capecitabine. Neither the Arm 1 vehicle (cremophorEL), nor the Arm 2 anisomycin/cremophorEL treatment had any effect on tumor growth, but a statistically significant weight gain was observed on Day 10 in Arm 2 animals (Table 9). Even though the Arm 3 capecitabine (Cape) dose selected for this study (500 mg/kg/day, 1500 mg/sq m/day) was in the cytotoxic range (78% of a standard human dose), it only produced one spontaneous death. However, as shown in Table 9, this toxicity did result in significant animal weight loss and sacrifice for >20% weight loss (four animals in Arm #3, five animals in Arm #4, and two animals in Arm #5). Even though this capecitabine dose did not produce excessive tumor regression (mean size reduction of 56%, Table 8), it did significantly delay (Day 10 to Day 35) tumor growth compared to Arms #1 and #2. The addition of low dose anisomycin and cremophorEL in Arm #4 did not significantly improve the capecitabine tumor responses. In contrast, the addition of high dose anisomycin/cremophorEL in Arm #5 resulted in considerable tumor size regression in every animal (FIG. 11A, mean size regression of 76.7%, Table 8). Correlating tumor responses in the Arm #3 and Arm #5 detected a statistically significant (2-tailed tTest) size difference that first reached significance during the treatment period (Day 15, p=0.0047) and continued until animal sacrifice in Arm #3 prevented further statistical analysis (after Day 31). Tumor regression in Arm #5 continued after treatment, and the tumors reached a size minimum at Day 38, when each tumor was smaller than the minimum measurable size (a theoretical cure).

FIG. 11B shows that capecitabine-dependent xenogenic tumor responses in the first animal study resolved into 3 types of delayed tumor growth responses (exemplified by Arm 3 animals #3, #5 and #8) and that the low dose anisomycin treatment (Arm 4) did not produce any novel tumor responses when compared to capecitabine treated animals. The apparent difference is due to a particularly aggressive tumor in animal C3 (FIG. 11B). FIG. 11C shows a similar comparison of individual Arm #3 and Arm #5 tumors. In contrast to the three capecitabine-dependent growth delays shown in FIG. 11B, the Arm 5 animals exhibited tumor regrowth patterns that correlated with either the lowest regrowth rate or a new tumor response where the tumors did not exhibit any significant regrowth (exemplified by animals #1, #3, #6 and #7). Of the six Arm #5 animals that survived the treatment cycle, all were alive on Day 70 (since none of the tumors had reached a 2 g size limit). Only three tumor regrowth events were detected, with animal H5 exhibiting the greatest regrowth activity (<1900 mg on Day 70), which was very similar to animal C5 from Arm #3 (the most favorable capecitabine response). For animals H1, H3 and H7, the post-mortem examination established that the TR metastatic tumor cells had been completely killed by the treatment (no visible tumor and only minor scarring at the cell implantation site), showing a maximal tumor regression of ˜450 mg by Day38 (animal H3) and a mean tumor regression of 76.7% for Arm 5 (Table 8). These results validated the Adjunct activity of the SET Regulatory components in combination with capecitabine, established a Preclinical Biological Effective Dose (BED) of 0.000054 mg/kg/day or 0.00016 mg/sq m/day for anisomycin, and warranted further investigation of using SET Combination drugs in improving the efficacy of First line oncology drugs.

FIGS. 12A, 12B and Table 9 show dose dependent reversible weight loss associated with the SET Combination drug. Whole animal weights recorded throughout the study were normalized by subtracting the weight of the tumor and expressed as percentages of the starting weight (Day 7 of the study).

Terms and Abbreviations: Control drug (Con): vehicle (cremophorEL), or Arm #1; C or Cape: capecitabine, or Arm #3; C+L, L, or C+LD: capecitabine/low dose anisomycin/cremophorEL, or Arm #4; C+H, H, or C+HD: capecitabine/high dose anisomycin/cremophorEL, or Arm #5 (Table 7).

FIG. 12A compares weight changes in individual animals treated with the low dose (Arm 4) and high dose (Arm 5) SET Combination drugs. This chart exemplifies the animal weight dynamics through the drug treatment and subsequent animal recovery phases of mice through Day 38 of the study. In summary, animals responded to treatment by exhibiting either weight loss (significant weight losses in Arms 4 and 5, Table 9) followed by a recovery phase that resulted in a statistically significant weight gain after day 31 or a control animal weight pattern, which produces a biphasic weight response. Given that this weight loss appears to be nontoxic, since both of these animals recover and catch up with the others by the end of the study. This biphasic response was not obvious when Arm 3 capecitabine animals were compared to the Arm 5 high dose anisomycin animals (FIG. 12B), which means that animals exhibit a systemic SET Combination drug response that can reversibly affect animal weight and promote a subsequent weight gain. The capecitabine treated mice (C8 and C5) appear to fall right in the middle of the two C+HD weight responses, while animal C3 never gained weight. Table 9 shows that the weights of Arm 1 vehicle (cremophorEL) and Arm 3 capecitabine treated animals did not change significantly through the treatment period. Most animals displayed modest average weight changes at the early treatment stages, but then slowly lost weight as their general health declined due to tumor growth. Detailed analysis showed that the capecitabine treated animals fell into two categories. Weight changes in animals C6, C5, C8, and C3 were similar to vehicle controls, while C1, C2, C4, and C7 lost weight during the treatment period and were sacrificed between Day 22 and Day 24. The addition of low dose anisomycin/cremophorEL appeared to enhance the weight loss effect (significant weight loss observed on days 10, 13 and 15, Table 9), resulting in the sacrifice of 5 animals (C+L 2, 4, 5, 7, and 8) on Day 18, even though it had no effect on tumor size regression. The three surviving animals (C+L 1, 3, and 6) looked similar to vehicle controls or the high dose response (FIG. 12A). In contrast, the addition of high dose anisomycin/cremophorEL seemed to have a protective effect, since only 2 animals (C+H 2 and 8 were sacrificed due to wasting on Days 22 and 24). Therefore, it seems that doses of the SET Combination Drug components must be selected that maximize the protective weight loss effect treatment dose, as low dose weight loss might have unexpected clinical consequences that have little to do with the drug therapeutic activity.

FIG. 13 shows that a preferred SET Combination drug (Arm 5) will enhance animal survival when applied with high dose Capecitabine. Preclinical in vivo Survival is a function of spontaneous animal death, animal wasting (animal sacrifice after >20% total weight loss) and maximum allowed tumor burden (animal sacrifice after tumor size is >2 g). This figure shows a Kaplan-Meier graph, where animal number (% Survival) is plotted versus day of trial (Time) and provides an estimate of the overall survival function for each treatment Arm.

Vehicle (Arm #1) and the anisomycin/cremophorEL (Arm #2) treatments did not affect survival, and all animals were sacrificed by Day 36 (as a result of tumor burden). Although the Low Dose anisomycin SET Combination Drug Arm #4 exhibited an early loss of five animals (due to weight loss) on Day 15, a similar survival decline was detected in the capecitabine Arm #3 (a total of five animals sacrificed on Day 22 and 24). So by the end of the treatment (Day 24), there was no difference in animal wasting in treatment Arms #3 and #4. While a slight variation in tumor growth resulted in one surviving animal in Arm #3 compared to two live animals in Arm #4 on Day 70, this difference was insignificant since the tumor in animal L6 was 1.8 g, so it would have been sacrificed on Day 71. In summary, all tumor and animal responses show that the 0.000027 mg/kg/day or 0.00008 mg/sq m/day anisomycin dose was below the Biological Effective Dose and provided no Adjunct Drug activity for capecitabine.

In contrast, only two animals exhibited capecitabine toxicity in the High Dose anisomycin SET Combination Drug Arm #5 and were sacrificed during the treatment period (Day 22 and 24). This low rate of animal wasting (only 40% of capecitabine controls) is consistent with animal weight gain prior to the end of the treatment (see FIG. 12A). The unexpected weight gain resulted in no animal loss due to weight decline for the remainder of the study (a 100% survival rate). Even though there was a significant delay in tumor regrowth in Arm #5 (see FIG. 11C), the survival function clearly shows that this delay equates with enhanced animal survival, the preferred metric for human drug responses (Increased Overall Patient Survival).

While it is currently impossible to correlate animal and human survival, this Preclinical trial clearly demonstrates that animals treated with 0.5 mg/kg/day CremophorEL and 0.000054 mg/kg/day anisomycin for an 18 day cycle exhibited an Adjunct or Concurrent Sensitizing Drug response that improved the Therapeutic Index of the high dose capecitabine (500 mg/kg/day) therapy.

FIGS. 14A, 14B, Tables 10, and 11 describe a second Xenogenic animal trial that establishes that two SET Combination drugs induce tumor regression and delayed tumor regrowth when applied with a low dose, subtherapeutic level of capecitabine. For the second animal trial, HCTI16 hTRdm-fLUC#32 metastatic tumor cells were injected into nude mice (athymic nude-Foxnlnu), allowed to form tumors, and triaged into 5 treatment Arms (Table 10). The treatment was started when the tumors reached ˜125 mg in size (Day 6 of the study) and applied daily for 10 days, after which the animals were monitored for additional 56 days. The drug concentrations for each arm of the study are listed in Table 10. Whole body weights and tumor weights were measured using standard sizing procedures. Animals were sacrificed due to wasting (after >20% total weight loss) or for tumor burden (>2 g).

Terms and Abbreviations: Vehicle: cremophor or Arm #1; C or Cape: capecitabine or Arm #2; C+E: capecitabine/emetine/cremophorEL or Arm #3; C+H: capecitabine/high dose anisomycin/cremophorEL (same as in the first animal trial) or Arm #4; C+VH: capecitabine/very high dose anisomycin/cremophorEL or Arm #5.

FIG. 14A shows average tumor weights over the course of the trial. As expected, Arm 1 vehicle control (CremophorEL) treated tumors displayed linear growth, and all animals in this arm were sacrificed for tumor burden between Day 25 and Day 40. In an attempt to reduce animal sacrifice due to weight loss, the capecitabine dose chosen for this trial (400 mg/kg/day or 1200 mg/sq m/day) was in the cytostatic (35% of the standard human dose), rather than the cytotoxic range of the first study. Consequently, capecitabine only produced minor tumor regression in this trial (Table 11; mean tumor size reduction of 35.2%), and tumor growth resumed almost immediately after treatment was terminated. Correlating tumor regression in the Arm #2 animals (capecitabine) with Arm #3, Arm #4, and Arm #5 detected statistically significant (2-tailed tTest, p<0.05) tumor size differences that first reached significance before the end of the treatment period (day 14) and continued until animal sacrifice made statistical analysis impossible. Therefore, all three SET Combination drug arms demonstrated reduced tumor growth, extensive tumor regression, and delayed tumor re-growth compared to the capecitabine controls. Paradoxically, as shown in FIG. 14A and Table 11, there was no benefit to using a very high anisomycin dose over a lower dose anisomycin, since the tumor responses in Arm #4 and Arm #5 were equivalent, which confirms that the preferred Preclinical Biological Effective Dose (BED) for anisomycin is 0.000054 mg/kg/day. As shown in FIG. 14B and Table 11, the greatest tumor regression and slowest tumor re-growth were produced by the combination of low dose capecitabine, emetine, and cremophorEL (Arm #3). Although 7 of 8 Arm 5 animals exhibited significant tumor effects (maximal tumor regressions of 90%), there were no “cure” events (tumors regressed below detectable size and never regrew). Nonetheless, both xenogenic tumor animal studies showed that multiple SET Combination Drugs (unique compositions and dosings) functioned as adjunct drugs that can enhance the therapeutic index of a highly toxic First Line oncology drug, even at subtherapeutic concentrations.

FIG. 15 shows that the SET Combination drugs induce a monophasic weight change profile. Whole animal weights recorded throughout the study were normalized by subtracting the weight of the tumor and expressed as percentages of the starting weight (Day 6 of the study).

Terms and Abbreviations: Vehicle: cremophor or Arm #1; C or Cape: capecitabine or Arm #2; C+E: capecitabine/emetine/cremophorEL or Arm #3; C+H: capecitabine/high dose anisomycin/cremophorEL (same as in the first study) or Arm #4; C+VH: capecitabine/very high dose anisomycin/cremophorEL or Arm #5; C+H7: animal 7 from the C+HD arm, first study; C+H1: animal 1 from the C+HD arm, first study; C+L3: animal 3 from the C+LD arm, first study.

FIG. 15 shows the average % weight changes for each arm up to Day 36 of the study. As with the first animal trial, the weights of Arm 1 vehicle (cremophorEL) treated animals did not change significantly. Most animals continued growing at the early stages, but then slowly lost weight as their general health declined due to tumor growth (i.e. cachexia). However, in contrast to high dose capecitabine treated animals in the first xenogenic animal study, where some animals were sacrificed for catastrophic weight loss, the low dose capecitabine (400 mg/kg/day) treated animals in Arm #2 did not exhibit sufficient weight change to warrant sacrifice. As before, capecitabine treated animals showed a concerted weight drop during treatment followed by a rebound to the pre-treatment weight. As in the first animal study, the SET Combination Drugs of Arms #3, #4, and #5 enhance this effect. The weight loss was greatest in the Arm 5 capecitabine/very high dose anisomycin/cremophorEL treated arms, where 4 of 8 animals were sacrificed for weight loss between Days 11 and 20. In contrast, for the lower anisomycin dose Arm #4 animals, only 2 of 6 animals were sacrificed which was the same animal number as in the first study. While no animals were lost to wasting in capecitabine/emetine/cremophorEL treated Arm #3, their average weight loss during the treatment period was significantly lower (Days 12-14; 2-tailed tTest p=0.022) than that of the capecitabine treated mice in Arm #2. As shown in FIG. 15, all of the SET Combination drug treated animals displayed weight gains (above starting weight levels) by Day 29; however, the weight gain in Arm #3 animals surpassed all other arms by Day 26 and reached statistical significance by Day 29 (2-tailed tTest, p=0.0006). As in the first animal study, emetine and anisomycin appear to have a protective effect and induce a reversible weight change that results in rapid animal weight gain within a week after treatment stops.

When the average % starting weight change for the first and second animal studies are compared (FIGS. 12A and 15), the weight loss and weight gain trends are remarkably similar. Therefore, the reversible weight changes observed in both studies might be a common response when SET Combination drug are delivered with capecitabine. Since this weight loss is rapidly reversible (not produced by a toxic side effect since animal weight rebounds by 38.2% in 17 days) and has nothing to do with the tumor responses, it seems to be a treatment feature that will require monitoring to prevent weight changes from becoming a critical therapeutic problem.

FIG. 16 shows that SET Combination drugs enhance animal survival when applied with low dose capecitabine. Preclinical in vivo Survival is a function of spontaneous animal death, animal wasting (animal sacrifice after >20% total weight loss) and maximum allowed tumor burden (animal sacrifice after tumor size is >2 g). This figure shows a Kaplan-Meier graph, where animal number (% Survival) is plotted versus day of trial (Time) and provides an estimate of the overall survival function for each treatment Arm.

Vehicle (Arm #1, cremophorEL) treatment did not affect survival and all animals were sacrificed by Day 40 as a result of tumor burden (sacrifice mean of 26 days). The loss of capecitabine treated animals (Arm #2) had not begun until Day 43, after which their loss due to tumor burden was fairly rapid (5 animals by Day 47, 2 more by Day 50, and the last animal on Day 54) (sacrifice mean of 47 days). Although most Arm #2 animals lost weight during the treatment period (FIG. 15), none reached the >20% weight loss threshold required for animal sacrifice. In contrast, both anisomycin treated arms had significant animal loss due to weight loss. In the very high anisomycin/capecitabine/cremophorEL Arm #5, 4 animals were sacrificed for weight loss between Day 11 and Day 20, with the remainder being terminated for tumor burden between Days 46 and 57 (sacrifice mean of 47 days). Thus, despite its effects on tumor size (shown in FIG. 14A and Table 11), the 0.00013 mg/kg/day anisomycin dose combined with low dose capecitabine did not provide any statistically significant Overall Survival benefit over the capecitabine monotherapy.

In contrast, despite the loss of 2 animals to weight loss (Days 12-14), the 0.000054 mg/kg/day anisomycin dose in Arm #4 did provide a significant Overall survival benefit (sacrifice mean of 57 days, an increase of 121% compared to Arms 2 and 5). The mice in this group were sacrificed for tumor burden between Days 47 and 68, confirming that the 0.000054 mg/kg/day treatment is a preferred anisomycin dose (BED) when combined with capecitabine. However, the greatest Overall Survival benefit was seen in Arm #3, where low dose capecitabine was combined with 0.00013 mg/kg/day emetine and 0.5 mg/kg/day CremophorEL (sacrifice mean of 68 days, an increase of 145% compared to Arms 2 and 5). Although there was significant weight loss in Arm #3 during the treatment period, none of the animals reached the >20% weight cutoff (FIG. 15). Moreover, the first animal in this arm was sacrificed for tumor burden on Day 57, after the sacrifice mean of Arms #1, #2, and #5 and at the sacrifice mean of Arm #4. As shown in FIG. 16, two of the animals in Arm #3 were alive at the end of the study period. These Preclinical trials clearly demonstrate that animals treated with 0.5 mg/kg/day CremophorEL and 0.000054 mg/kg/day anisomycin or 0.5 mg/kg/day CremophorEL and 0.00013 mg/kg/day emetine exhibit an Adjunct Drug response that improves the Therapeutic Index of capecitabine at various doses.

FIGS. 17A-17J and Table 12 show immunostaining studies of hTRdm-fLUC#32 tumors that examined tumor and immune cell responses during chemotherapy treatment. Tumors from the first animal study (Table 7) were dissected from animals sacrificed for weight loss (Arm 2 animals #1 on day 24 and #7 on day 22; Arm 4 animal #5 on day 18; Arm 5 animal #2 on day 24 and animal #8 on day 22). Tumors were flash frozen, fixed, sectioned, and for multi-epitope detection of cellular and recombinant proteins, a mixture of fluorescently labeled and unlabeled antibodies were used to detect 4 macrophage marker proteins (biotin-labeled anti-mouse MHC class II molecules IA/IE, Alexa-647-labeled anti-mouse CD1 1 b/Mac-1, Alexa-488-labeled anti-mouse F4/80, and Alexa-647-labeled anti-mouse CD68) and the TR reporter protein (anti-firefly luciferase). To detect unlabeled primary antibodies, an Alexa-555-labeled secondary antibody or PE-labeled streptavidin were used. Nuclear DNA staining with the DAPI dye is used to detect viable tumor cells.

It has been known for more than 150 years that human solid tumors exhibit asynchronous, non-exponential growth due in large part to a multi-layer structure that contains an outer proliferative/mitotic layer, a non-mitotic cell layer, and an inner necrotic core. The proliferative cell layer (<10 cells thick) directly contacts the tumor microenvironment so that passive diffusion infuses cells with nutrients, oxygen, and growth stimulators. Inside the mitotic layer is a compressed cell stratum that has reduced vascularization/blood flow and an inherent resistance to passive diffusion (i.e. tumor interstitial fluid pressure). In addition to reducing drug diffusion and metabolic activity, this high cell density produces a “Contact Inhibited or CI” phenotype that arrests cells at a G1 /S checkpoint and prevents cell cycle progression. Since these nonmitotic CI cells cannot begin DNA replication, they are intrinsically resistant to the action of S phase cytotoxic chemotherapeutics. However, tumor regrowth requires a CI tumor cell to reenter the cell cycle to replace mitotic cells killed by drug damage. This should produce unexpected G2-specific SET in tumor cells that can only be detected using the TR expression system. Moreover, animal respond to apoptotic cell debris in regressing tumors by activating phagocytic immune cells (for nude mice these immune cells are only produced by the innate immune system). Immunostaining will be used to define the subtypes of tumor associated macrophage (TAM), their tumor distribution, and association with dying tumor cells.

Abbreviations: fLUC: firefly luciferase; DAPI: 4′,6-diamidino-2-phenylindole.

Correlating the nuclear staining of FIG. 17A with the G2-specific fLUC expression in FIG. 17B (tumor isolated from Arm 2 animal #1, treated with capecitabine for 16 days, Table 7) confirmed that capecitabine induced a G2/M checkpoint and activated SET Ribosome translation (fLUC expression) in a narrow strip of peripheral mitotic cells (white arrow FIG. 17B, Layer 1). By counting the number of sequential nuclei extending from the tumor surface, Layer 1 was shown to have an average thickness of 3.4 cells (Table 12). Surprisingly, Layer 1 cells exhibited minimal staining macrophage epitopes but was bordered by an inner cell layer (Layer 2) that contained a dense concentration of F4/80 stained macrophages (6.4 cells thick). In general, the F4/80+ macrophages in this layer did not stain for the other immune or fLUC proteins and appeared to be contained within and established a boundary for the mitotic cell layer (9.8 cells thick). Extending into the tumor were individual F4/80+ cells that penetrated the tumor at an average depth of 16.6 cells (Layer 3, total depth from surface of 26.4 cells). While the border of Layers 2/3 contained a modest number of fLUC positive cell bodies, minimal staining was observed between Layer 3 and the necrotic core (cell remnants with minimal nuclear DAPI staining). These results are consistent with the capecitabine mode of action, the expected multi-layer structure of solid tumors, and activation of a specific subclass of F4/80+innate immune cells by dying cells. Identical tumor and immune cell responses were observed in a second tumor processed from Arm 2 animal #7 that had been treated for 14 days.

FIG. 17C and FIG. 17D (tumor isolated from Arm 4 animal #5, treated with capecitabine, low dose anisomycin, and cremophorEL for 10 days, Table 7) established that the SET Combination drug activated uniform, G2-specific fLUC expression in tumor cells extending from Layer 3 to the necrotic core (white arrow FIG. 17D). In FIG. 17C, the Layer 2 macrophages are exemplified by bright, small nuclei that do not stain for the fLUC antigen. In contrast to the capecitabine tumor, the majority of the Layer 2 immune cells displayed selective staining for the CD68 marker protein (CD68+F4/80−) and a minor fraction of co-stained or lightly stained F4/80 macrophages. Moreover, the CD68+F4/80− immune cells penetrated throughout the entire tumor, including the necrotic core. Since the tumors in Arm 4 did not display significant tumor responses or improved animal survival, the SET Agonist cremophorEL stimulated G2-specific SET throughout the tumor and activated a distinct CD68+F4/80− macrophage subtype. Although any tumor or animal effects (other than weight changes) produced by the low dose anisomycin remain unclear, this study does show that the CI cells mapping from Layer 3 to the necrotic core are forced by the SET Combination drug to reenter the cell cycle and begin expression of the G2-specific fLUC reporter protein, which was not observed in the Arm 2 capecitabine monotherapy tumors.

FIGS. 17E-17F and Table 12 (tumor isolated from Arm 5 animal #2, treated with capecitabine, high dose anisomycin, and cremophorEL for 16 days) show significant changes in the tumor Layer structure. FIG. 17F confirmed that G2-specific translation of the fLUC reporter protein was present in Layer 1 (white arrow); however, the average thickness had increased to 7.8 cells compared to the Arm 2 tumors (2-tailed tTest p=0.008, Table 12). Furthermore, this Layer was highly disorganized and contained small, subcellular fLUC+ bodies that mapped to the tumor periphery. Significantly, in contrast to the Arm 4 tumor, internal tumor cells did not display significant fLUC staining except in the necrotic core. Similar size increases were also observed in Layer 2 (average thickness of 7.8 cells) and Layer 3 (average thickness of 18.6 cells). In contrast to the mitotic cell layer of the Arm 2 tumors, this Arm 5 tumor exhibited a combined size for Layers 1/2 of 15.6 cells (>50% size increase). As before, an abundance of CD68+F4/80− macrophages were observed in Layers 2 and 3 that had penetrated to the necrotic core. These results are consistent with an increase in the number of G2 phase tumor cells and the appearance of small fLUC+ bodies within the dying mitotic cell layer (tumor regression of 33.9%). This supports the ability of the SET Combination drug to enhance capecitabine-induced apoptotic cell death, as well as, promote an invasive CD68+F4/80− macrophage response while also reducing G2-specific translation in the CI cell layer.

FIGS. 17G-17H (tumor isolated from Arm 5 animal #8, treated with capecitabine, high dose anisomycin, and cremophorEL for 14 days) show unexpectedly high levels of fLUC expression and cell death in CI and necrotic core cells. FIG. 17G shows DAPI staining over a tumor section spanning from the proximal Cl/necrotic layer (detectable DAPI stained nuclei) into the necrotic core (minimal DAPI staining). FIG. 17H demonstrates that this tissue section contains a high density of fLUC+ bodies that localize to cells containing no detectable DAPI staining (white arrows in FIGS. 17G and 17H). Surprisingly, this data shows that the SET Combination drug activates an unexpectedly high metabolic activity in supposedly dead cells. Moreover, the SET Combination drug stimulates cell cycle progression to the G2 phase and enhances cell death at the center of a treated tumor. Given that this tumor had undergone a 59.8% size regression, these results support the idea that this drug kills mitotic cells at the tumor surface and non-mitotic cells within the necrotic core.

FIGS. 171-17J (tumor from Arm 5 animal #8) shows the quantitation of fluorescent fLUC+staining across the interior of a tumor using the ImageJ software. A fluorescence density map was produced by drawing 15 boxes (35×695 pixels, 0.64 um/px) on FIG. 17I and measuring the fluorescence intensity for each of the 695 pixels. The darkest necrotic cell layer pixel was adjusted to 100% background and the total fluorescence for each pixel was compared to that value (FIG. 17J). This density map shows that fLUC staining intensity increased by 500%-600% in cells that exhibit minimal DAPI staining (the necrotic core) compared to adjacent DAPI+ cells. This result is consistent with a highly significant and selective increase in SET of the fLUC reporter protein (and G2-specific apoptotic cell death) in cells that are assumed to be nonmitotic and metabolically inactive.

### DETAILED DESCRIPTION OF THE INVENTION

Throughout the specification, several terms are employed that are defined in the following paragraphs.

The singular terms “a,” “an,” and “the” are not intended to be limiting and include plural referents unless explicitly stated otherwise or the context clearly indicates otherwise.

The terms “comprising,” “comprises” and “comprise” as used herein are synonymous with “including,” “includes” and “include,” respectively, and do not exclude additional elements.

The term “proliferative disorder” as used herein refers to pathological as well as benign conditions characterized by undesirable cell proliferation, including cancer.

The term “cancer” in a mammal refers to a physiological condition that is characterized by the presence of cells possessing characteristics typical of cancer cells, such as uncontrolled proliferation, immortality, metastatic potential, rapid growth and proliferation, anchorage-independent growth, and certain distinct morphological features. Often, a collection of cancer cells will localize into a “tumor”, but such cancer cells may also exist alone within an animal, or may circulate in the blood as independent cells.

“Metastatic cancer” is cancer that has spread from a place or origin to another spot in the body. A tumor formed by metastatic cells is called a “metastatic” tumor or a “metastasis”. The process by which cancer cells spread to other parts of the body is termed “metastasis”.

Cancer examples, include, but are not limited to carcinoma, lymphoma, blastoma, sarcoma, and leukemia. More particularly, examples of such cancer include colorectal cancer, squamous cell carcinoma, small-cell lung cancer, non-small cell lung cancer, pancreatic cancer, glioblastoma multiform, esophageal/oral cancer, cervical cancer, ovarian cancer, endometrial cancer, prostate cancer, bladder cancer, head and neck cancer, hepatoma, and breast cancer.

The term “proliferative disorder” also encompasses disorders of cell division and abnormal or undesirable proliferation of non-cancerous cells and such conditions are treated by administration of the compositions of this invention. Such proliferative disorders include, for example, EBV-induced lymphoproliferative disease and lymphoma, neointimal hypoplasia (e.g. in patients with athlerosclerosis and undergoing balloon angioplasty), proliferative effects secondary to diabetes, psoriasis, benign tumors (e.g. angiomas, fibromas, and myomas), and myeloproliferative disorders (e.g. polycytemiavera).

The terms “subject,” “individual” and “patient” are used interchangeably to refer to a human or any other mammal, such as a mouse, rat, guinea pig, rabbit, dog, cat, sheep, cow, horse or non-human primate.

The terms “subject,” “individual” and “patient” refer to an individual that can be afflicted with or is susceptible to a neoplastic disorder (e.g. cancer) but may or may not have the disease or disorder. For example, the terms “subject,” “individual” and “patient” may be an individual that presents one or more symptoms indicative of a neoplastic disorder, has one or more risk factors, or is being screened for a neoplastic disorder. The terms also apply to individuals that have previously undergone therapy for a proliferative disorder. In a preferred aspect, the subject is a human being.

The term “treatment,” “protocol,” “method of treating,” “procedure,” “therapy” or their equivalents, as used herein to refer to a method, composition, or process aimed at: (1) delaying or preventing the onset or relapse of a medical condition, disease or disorder; (2) slowing or stopping the progression, aggravation, or deterioration of the symptoms of a condition; (3) ameliorating the symptoms of the condition; and/or (4) curing the condition. Treating cancer or a proliferative cellular disease does not necessarily imply that all proliferative cells will be eliminated, that the number of proliferative cells will be reduced, or that the symptoms of a condition will be alleviated. Often, treatments will be performed even with a low likelihood of success, but which, given the medical history and estimated survival expectancy of the patient is nevertheless deemed potentially beneficial. The treatment may be administered prior to the onset of the condition, for a prophylactic or preventive activity, or it may be administered after the initiation or onset of a condition, for a therapeutic action.

The term “substance” as used herein refers to a matter of defined chemical composition and is used herein interchangeably with the terms “compound” and “drug”. As used herein, the terms “cytotoxic agent,” “chemotherapeutic,” “antineoplastic,” “therapeutic agent,” “cytotoxic oncology drug” and “anticancer drug” are used interchangeably to refer to a substance, molecule, compound, composition, agent, factor, process or composition that provides treatment for various forms of proliferative disorders, cancer and proliferative cellular diseases. Cytotoxic oncology drugs are conventionally classified as “cytotoxic antineoplastics” e.g. nucleoside analogues, antifolates, other antimetabolites, Topoisomerase I inhibitors, anthracyclines, podophyllotoxins, taxanes, vinca alkaloids, alkylating agents, platinum compounds, and miscellaneous compounds or “targeted antineoplastics” e.g. monoclonal antibodies, tyrosine kinase inhibitors, mTOR inhibitors, retinoids, immunomodulatory agents, histone deacetylase inhibitors, and other agents. Cytotoxic oncology drugs directly or indirectly inhibit cancer cell growth or kill cancer cells.

The terms “standard of care”, “first-line therapy”, “induction therapy”, primary therapy”, and “primary treatment” are used herein to define the first treatment option(s) a clinician should follow for a certain type of patient, illness, or clinical circumstance. Since disease treatment is complex, a given first-line therapy will not necessarily be the only standard of care option. The terms “adjunct drug” and “adjuvant drug” are used interchangeably herein and refer to any additional substance, treatment, or procedure that is added to a primary therapy, treatment, or procedure that enhances the efficacy, safety or facilitates the performance of a primary therapy, treatment or procedure. Functionally, an adjunct drug may or may not display treatment or therapeutic activity when applied without the primary or main substance, treatment, or procedure.

As used herein, the terms “biologically effective dose and/or amount,” “treatment effective dose/amount,” and “effective dose/amount”, refer to any quantity of a substance, composition, or treatment process that elicits a desired biological, medicinal or therapeutic response in a tissue, organ system or subject. For example, a desirable response may include one or more preferred outcomes of a treatment paradigm.

The terms “pan-resistance,” “extreme-drug resistance,” and “cross-drug resistance” are used interchangeably herein to define a cellular phenotype characterized by a generalized resistance to oncology therapeutic drugs and processes. This process is distinguished from multi-drug resistance which is used to describe the over-expression of drug transporter systems.

As used herein, the terms “coadministration” and “combination” refer to the administration of two or more drugs that exhibit biologically effective or therapeutic activity in a subject. Coadministered or combination drugs can be simultaneously or sequentially delivered. The two or more biologically effective or therapeutically active substances can be delivered as single or independent compositions.

A “pharmaceutical composition” is herein defined as comprising an amount of a drug and at least one “physiologically acceptable carrier” or “excipient”. A pharmaceutical composition can include various additional ingredients to aid or improve formula activity, such as bioavailability, pharmacokinetics or pharmacodynamics, as well as one or more therapeutic agents. As used herein, the terms “physiologically acceptable carrier” and “excipient” refer to an agent that does not interfere with the therapeutic effectiveness or biological process of any active pharmaceutical ingredient and which is not excessively toxic to a subject at the administered concentration. The term excipient is exemplified by, but not limited to, diluents, bulking agents, antioxidants or other stabilizers, dispersants, solvents, dispersion medium, coatings, antibacterial agents, isotonic agents, absorption delaying or enhancing agents, and the like. The use of such excipients for the formulation of pharmaceutically active substances is well known in the art, see for example, “Remington's Pharmaceutical Sciences”, E. W. Martin, 18th Ed., 1990, Mack Publishing Co.: Easton, Pa., which is incorporated herein by reference in its entirety.

The terms “cap-dependent ribosome” and “growth ribosome” refer to the eukaryotic ribosome and associated initiation factors that interact with selective structures at the 5′ end of an mRNA and initiate eukaryotic translation by binding and scanning to a preferred translation initiation codon in growing and proliferating cells (Cap-dependent translation). The term “Selective Translation” (SET) refers to all cellular translational activity not produced by a Cap-dependent translation process or translation that results from the inhibition of Cap-dependent translation. The “SET Ribosome” is the eukaryotic ribosome and any associated protein or complex needed to generate all cellular translational activity not produced by a Cap-dependent translation process or translation that results from the inhibition of Cap-dependent translation. The SET Ribosome directs the selective synthesis of proteins (SET) during the late S and G2 cell cycle phases. During SET, the SET ribosome has the ability to initiate translation from internal mRNA sequences termed an “Internal Ribosome Entry Sequence” (IRES; directs the binding of the 40S ribosome subunit to a specific mRNA sequence) and to reinitiate translation using mRNA “Reinitiation Sequences”, exemplified by the regulatory sequences in the TR expression cassette. The Translation Regulated (TR) technology is based upon specific RNA sequences and mRNA secondary structures within the TR expression cassette (derived from the mammalian proteolipid protein gene) that bind to and orient the 40S ribosome subunit (without any interaction with the 5′ mRNA Cap structure) so that the translation initiation codon of an operably linked reporter gene is positioned in the ribosome decoding center for translation initiation.

The term “SET Agonist” refers to an agent or treatment that increases SET of the TR mRNA by activating the SET Ribosome, produces a SET Agonist “response”, and induces cell cycle progression to the late S/G2 phases, while simultaneously inhibiting Cap-dependent translation. A SET Agonist “response” is defined as an outlier TR Assay result (detected by any TR Assay format, such as a Cell Count 15-Reagent Assay) that is >2 standard deviations above the mean of all cumulative SET responses. By way of example, treating the HCT116 mTRdm-fLUC cell lines with the 5 Reference Standards (TPA, Tax, Cal, MG132, and cAMP) established that TPA-Tax was a SET Agonist since this TR Assay mean was >3 standard deviations above the mean of each 5 Reference Standard response.

The term “SET Ribosome Antagonist” refers to an agent (that binds to or acts on the SET Ribosome) that, when delivered in combination with the SET Agonist, completely blocks SET Agonist activity and SET Ribosome translation at an IC100 dose that is 0.02% of the LD50 concentration for rodents. By way of example, an IC100 dose or 100% Inhibitor Concentration is detected by a TR Assay (such as a Cell Count Dose-dependent Modifier Assay) that tests for a specific SET Antagonist dose that inactivates a known SET Agonist (such as TPA).

The term “gene” refers to a nucleic acid (e.g., DNA) sequence that includes coding sequences necessary for the production of a polypeptide or precursor or RNA (e.g., tRNA, siRNA, rRNA, etc.). The polypeptide can be encoded by a full length coding sequence or by any portion of the coding sequence so long as the desired activity or functional properties, such as enzymatic activity, ligand binding, signal transduction, etc., of the full-length or fragment are retained. The term also encompasses the coding region of a structural gene and the sequences located adjacent to the coding region on both the 5′ and 3′ ends, such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region, which may be interrupted with non-coding sequences termed “introns” or “intervening regions” or “intervening sequences.” Introns are removed or “spliced out” from the nuclear or primary transcript, and are therefore absent in the messenger RNA (mRNA) transcript. The mRNA functions during translation to specify the sequence or order of amino acids in a nascent polypeptide.

The term “expression vector” refers to both viral and non-viral vectors comprising a nucleic acid expression cassette.

The term “expression cassette” is used to define a nucleotide sequence containing regulatory elements operably linked to a coding sequence that result in the transcription and translation of the coding sequence in a cell.

A “mammalian promoter” refers to a transcriptional promoter that functions in a mammalian cell that is derived from a mammalian cell, or both.

A “mammalian minimal promoter” refers to a ‘core’ DNA sequence required to properly initiate transcription via RNA polymerase binding, but which exhibits only token transcriptional activity in the absence of any operably linked transcriptional effector sequences.

The phrase “open reading frame” or “coding sequence” refers to a nucleotide sequence that encodes a polypeptide or protein. The coding region is bounded in eukaryotes, on the 5′ side by the nucleotide triplet “ATG” that encodes the initiator methionine and on the 3′ side by one of the three triplets which specify stop codons (i.e., TAA, TAG, and TGA).

“Operably linked” is defined to mean that the nucleic acids are placed in a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous. However, enhancers do not have to be contiguous. Linking is accomplished by ligation at convenient restriction sites. If such sites do not exist, the synthetic oligonucleotide adaptors or linkers are used in accord with conventional practice.

“Recombinant” refers to the results of methods, reagents, and laboratory manipulations in which nucleic acids or other biological molecules are enzymatically, chemically or biologically cleaved, synthesized, combined, or otherwise manipulated ex vivo to produce desired products in cells or other biological systems. The term “recombinant DNA” refers to a DNA molecule that is comprised of segments of DNA joined together by means of molecular biology techniques.

“Transfection” is the term used to describe the introduction of foreign material such as foreign DNA into eukaryotic cells. It is used interchangeably with “transformation” and “transduction” although the latter term, in its narrower scope refers to the process of introducing DNA into cells by viruses, which act as carriers. Thus, the cells that undergo transfection are referred to as “transfected,” “transformed” or “transduced” cells.

The term “plasmid” as used herein, refers to an independently replicating piece of DNA. It is typically circular and double-stranded.

A “reporter gene” refers to any gene the expression of which can be detected or measured using conventional techniques known to those skilled in the art.

The term “regulatory element” or “effector element” refer to a transcriptional promoter, enhancer, silencer or terminator, as well as to any translational regulatory elements, polyadenylation sites, and the like that regulate ribosome activity or mRNA maturation. Regulatory and effector elements may be arranged so that they allow, enhance or facilitate selective production of a mature coding sequence that is subject to their regulation.

The term “vector” refers to a DNA molecule into which foreign fragments of DNA may be inserted. Generally, they contain regulatory and coding sequences of interest. The term vector includes but is not limited to plasmids, cosmids, phagemids, viral vectors and shuttle vectors.

A “shuttle” vector is a plasmid vector that is capable of prokaryotic replication but contains no eukaryotic replication sequences. Viral DNA sequences contained within this replication-deficient shuttle vector direct recombination within a eukaryotic host cell to produce infective viral particles.

The terms “stress” and “toxicity” are used to refer to the disturbance of the natural biochemical and biophysical homeostasis of the cell. Whereas stress generally leads to recovery of cellular homeostasis, a toxic response eventually results in cell death.

Methods according to aspects of the present invention for treating a proliferative disorder include administering to a mammal a Selective Translation (SET) Therapeutic that includes a cytotoxic drug and a SET Combination drug. Methods according to aspects of the present invention for treating a proliferative disorder include administering to a human subject a SET Therapeutic that includes a cytotoxic drug and a SET Combination drug.

Compositions according to aspects of the present invention include a cytotoxic agent and a SET Combination drug.

Compositions according to aspects of the present invention include capecitabine and a SET Combination drug.

A SET Combination drug includes an activator of the SET response and an inhibitor of SET ribosome activity.

According to aspects of the present invention, an included activator of the SET response is a protein kinase C activator, such as, but not limited to a phorbol ester.

According to aspects of the present invention, an included SET ribosome Antagonist is the translational regulator emetine.

Optionally, one or more additional anti-cancer treatments, such as administration of one or more additional cytotoxic drugs, radiotherapy, photodynamic therapy, surgery or other immunotherapy, can be combined with a SET Therapeutic to treat a proliferative disorder in a patient.

According to aspects of the present invention, an expression cassette includes an upstream transcriptional effector sequence which regulates gene expression. In one aspect, the transcriptional effector sequence is a mammalian promoter. In addition, the transcriptional effector can also include additional promoter sequences and/or transcriptional regulators, such as enhancer and silencers or combinations thereof. These transcriptional effector sequences can include portions known to bind to cellular components which regulate the transcription of any operably linked coding sequence. For example, an enhancer or silencer sequence can include sequences that bind known cellular components, such as transcriptional regulatory proteins. The transcriptional effector sequence can be selected from any suitable nucleic acid, such as genomic DNA, plasmid DNA, viral DNA, mRNA or cDNA, or any suitable organism (e.g., a virus, bacterium, yeast, fungus, plant, insect or mammal). It is within the skill of the art to select appropriate transcriptional effector sequences based upon the transcription and/or translation system being utilized. Any individual regulatory nucleic acid sequence can be arranged within the transcriptional effector element in a wild-type arrangement (as present in the native genomic order), or in an artificial arrangement. For example, a modified enhancer or promoter sequence may include repeating units of a regulatory nucleic acid sequence so that transcriptional activity from the vector is modified by these changes.

In one aspect, a promoter included in a TR nucleic acid expression cassette or control nucleic acid expression cassette is selected from constitutive, tissue specific, and tumor specific promoters.

A constitutive promoter included in a TR nucleic acid expression cassette or control nucleic acid expression cassette can be selected, e.g., from Rous sarcoma virus (RSV) long terminal repeat (LTR) promoter, cytomegalovirus immediate early gene (CMV) promoter, simian virus 40 early (SV40E) promoter, cytoplasmic beta-actin promoter, adenovirus major late promoter, and the phosphoglycerol kinase (PGK) promoter. According to one aspect, a constitutive promoter included in a TR nucleic acid expression cassette or control nucleic acid expression cassette is a CMV promoter. According to one aspect, a constitutive promoter included in a TR nucleic acid expression cassette or control nucleic acid expression cassette is an SV40E promoter.

A tissue specific promoter included in a TR nucleic acid expression cassette or control nucleic acid expression cassette can be selected, e.g., from the transferrin (TF), tyrosinase (TYR), albumin (ALB), muscle creatine kinase (CKM), myelin basic protein (MBP), glial fibrillary acidic protein (GFAP), neuron-specific enolase (NSE), and synapsin I (SYN1) promoters. According to one aspect, a tissue specific promoter included in a TR or control expression cassette is a synapsin I (SYN1) promoter. In another preferred aspect, a tissue specific promoter included in a TR or control expression cassette is the ALB promoter.

A tumor specific promoter included in a TR nucleic acid expression cassette or control nucleic acid expression cassette can be selected, e.g., from vascular endothelial growth factor (VEGF), a VEGF receptor (i.e. KDR, E-selectin, or endoglin), alpha-fetoprotein (AFP), carcinoembryonic antigen (CEA), erbB2 (v-erb-b2 erythroblastic leukemia viral oncogene homolog 2), osteocalcin (bone gamma-carboxyglutamate protein, BGLAP), SLP1 (secretory leukoproteinase inhibitor or antileukoproteinase 1), hypoxia-response element (HRE), L-plastin (lymphocyte cytosolic protein 1) and hexokinase II (HK2). In a preferred aspect, a tumor specific promoter included in a TR nucleic acid expression cassette or control nucleic acid expression cassette is an alpha fetoprotein (AFP) promoter. In another preferred aspect, a tumor specific promoter included in a TR nucleic acid expression cassette or control nucleic acid expression cassette is a SLP1 promoter.

According to aspects of the present invention, a specific transcriptional effector element is isolated and then operatively linked to a minimal promoter in a TR nucleic acid expression cassette or control nucleic acid expression cassette producing an expression cassette whose transcriptional activity is dependent upon a single or limited type of cellular response (e.g., a heat shock response or metal-regulated element).

According to aspects of the present invention, a TR nucleic acid expression cassette or control nucleic acid expression cassette can include species-specific transcriptional regulatory sequences. Such DNA regulatory sequences can be selected on the basis of the cell type into which the expression cassette will be inserted and can be isolated from prokaryotic or eukaryotic cells, including but not limited to bacteria, yeast, plant, insect, mammalian cells or from viruses. In such example, a mammalian promoter would be selected to express a nucleic acid of choice in a mammalian cell.

An open reading frame nucleic acid sequence encoding a reporter protein is positioned 3′ with respect to the nucleic acid encoding the TR element in a TR nucleic acid expression cassette or positioned 3′ with respect to the constitutive promoter in a control nucleic acid expression cassette. The nucleic acid sequence encoding a reporter protein can be either a full genomic sequence (e.g., including introns), synthetic nucleic acid or a cDNA copy of a gene encoding the reporter protein. In a preferred aspect, a cDNA sequence encoding a reporter protein is included in a TR nucleic acid expression cassette or control nucleic acid expression cassette due to the reduction in genomic complexity provided by removal of mRNA splice sites.

Techniques for inserting the nucleic acid sequence encoding a reporter protein and the nucleic acid sequence encoding the TR element into a TR nucleic acid expression cassette are known in the art, and include, ligating the sequences, directly or via a linker, so that they are under the control of the regulatory elements included in the expression cassette. One or more linkers providing a restriction endonuclease site can be added to any of the nucleic acid sequences to be included in the expression cassette to facilitate correct insertion of the sequences.

As described herein, a reporter gene encodes a reporter that confers on the cell in which it is expressed a detectable biochemical or visually observable (e.g., fluorescent) phenotype. The reporter protein can also include a fused or hybrid polypeptide in which another polypeptide is fused at the N-terminus or the C-terminus of the polypeptide or fragment thereof. A fused polypeptide is produced by cloning a nucleic acid sequence (or a portion thereof) encoding one polypeptide in-frame with a nucleic acid sequence (or a portion thereof) encoding another polypeptide. Techniques for producing fusion polypeptides are known in the art, and include, ligating the coding sequences encoding the polypeptides so that they are in-frame and translation of the fused polypeptide is under the control of the regulatory elements included in the expression cassette.

Non-limiting examples of reporters encoded in an expression cassette described herein include proteins which are antigenic epitopes, bioluminescent proteins, enzymes, fluorescent proteins, receptors, and transporters.

One commonly used class of reporter genes encodes an enzyme or other biochemical marker, which, when expressed in a mammalian cell, cause a visible change in the cell or the cell environment. Such a change can be observed directly, can involve the addition of an appropriate substrate that is converted into a detectable product or the addition and binding of a metabolic tracer. Examples of these reporter genes are the bacterial lacZ gene which encodes the β-galactosidase (β-gal) enzyme, the

Chloramphenicol acetyltransferase (CAT) enzyme, Firefly luciferase (Coleoptera beetle), Renilla luciferase (sea pansy), Gaussia luciferase, Herpes Simplex 1 thymidine kinase (HSV1-TK) and the mutant Herpes Simplex 1 thymidine kinase (HSV1-sr39tk) genes. In the case of 13-gal, incubation of expressing cells with halogen-derivatized galactose results in a colored or fluorescent product that can be detected and quantitated histochemically or fluorimetrically. In the case of CAT, a cell lysate is incubated with radiolabeled chloramphenicol or another acetyl donor molecule such as acetyl-CoA, and the acetylated chloramphenicol product is assayed chromatographically. Other useful reporter genes encode proteins that are naturally fluorescent, including the (green fluorescent protein (GFP), enhanced yellow fluorescent protein (EYFP), or monomeric red fluorescent protein (mRFP1).

A reporter encoded by a nucleic acid in an expression cassette can be selected from luciferase, GFP, EYFP, mRFP1, β-Gal, and CAT but any other reporter gene known in the art can be used. According to preferred aspects, the reporter encoded by a nucleic acid in an expression cassette is Firefly Luciferase. In another preferred aspect, the reporter encoded by a nucleic acid in an expression cassette is Renilla Luciferase. In still another preferred aspect, the reporter encoded by a nucleic acid in an expression cassette is Gaussia Luciferase.

One skilled in the art will readily recognize that any polyadenylation (polyA) signal can be incorporated into a 3′ untranslated (3′UTR) element of a TR nucleic acid expression cassette or control nucleic acid expression cassette described herein. Examples of polyA sequences useful for the present invention include the SV40 early and late gene, the HSV-TK, and human growth hormone (hGH) sequences. According to a preferred aspect, the polyA sequence is the SV40 early gene sequence.

According to aspects of expression cassettes of the present invention, the 3′UTR can include one or more elements which regulate gene expression by altering mRNA stability. Typically, mRNA decay is exemplified by the loss of the mRNA polyA tail, recruitment of the deadenylated RNA to the exosome, and ribonuclease (RNAse) degradation. In select mRNAs, this process is accelerated by specific RNA instability elements that promote the selective recognition of a mRNA by cellular degradation systems. In this invention, the expression cassette mRNA can contain elements such as the 3′UTR AU-rich element (“ARE”) sequences derived from mRNA species encoding cellular response/recovery genes.

Examples of ARE sequences optionally included in an expression cassette according to aspects of the present invention are 3′UTR sequences from the c-fos, the granulocyte-macrophage colony stimulating factor (GM-CSF), c-jun, tumor necrosis factor alpha (TNF-α), and IL-8 mRNAs. According to preferred aspects, the ARE sequences from the c-fos gene are included in a TR nucleic acid expression cassette or control nucleic acid expression cassette.

A TR nucleic acid expression cassette or control nucleic acid expression cassette can also include a 5′ untranslated region (5′UTR), which is located 3′ to the promoter, and 5′ to the sequence encoding the TR element in a TR nucleic acid expression cassette. In some aspects of expression cassettes, such a region includes an mRNA transcription initiation site. In other aspects of expression cassettes, the 5′ untranslated region includes an intron sequence, which directs mRNA splicing and is required for the efficient processing of some mRNA species in vivo. A general mechanism for mRNA splicing in eukaryotic cells is defined and summarized in Sharp (Science 235: 736-771, 1987). There are four nucleic acid sequences which are necessary for mRNA splicing: a 5′ splice donor, a branch point, a polypyrimidine tract and a 3′ splice acceptor. Consensus 5′ and 3′ splice junctions (Mount, Nucl. Acids. Res. 10:459-472, 1992 and branch site sequences (Zhuang et al., PNAS 86:2752-2756, 1989, are known in the art.

A TR nucleic acid expression cassette or control nucleic acid expression cassette can also include one or more 5′ UTR sequences which include one or more natural introns which exist in a native gene sequence or an artificial intron, such as the human beta-globin-immunoglobulin sequence present in the pAAV-MCS vector (Stratagene).

A TR nucleic acid expression cassette or control nucleic acid expression cassette can include one or more of the following: a sequence of between about 15-50 nucleotides located 5′ to the promoter, that includes one or more restriction sites for insertion of the expression cassette into a plasmid, shuttle vector or viral vector; a sequence of between about 15-50 nucleotides located 3′ to the sequence encoding the TR element or constitutive promoter and 5′ to the reporter sequence, that includes one or more restriction sites for insertion and operative linkage of the sequence encoding the TR element or constitutive promoter and the sequence encoding the reporter; a sequence of between about 15-50 nucleotides located 3′ to the reporter sequence and 5′ to the polyadenylation signal, that includes one or more restriction sites for insertion and operative linkage of the ORF sequence and the polyadenylation sequence; and a sequence of between about 15-50 nucleotides located 3′ to the polyadenylation sequence, that includes one or more restriction sites for insertion of the nucleic acid expression cassette into a plasmid, shuttle vector or viral vector.

A TR nucleic acid expression cassette or control nucleic acid expression cassette described herein can be inserted into plasmid or viral (“shuttle”) vectors depending upon the host cell which is used to replicate the expression cassette. In general, a TR nucleic acid expression cassette or control nucleic acid expression cassette is inserted into an appropriate restriction endonuclease site(s) in a vector using techniques known in the art. Numerous vectors useful for this purpose are generally known such as described in Miller, Human Gene Therapy 15-14, 1990; Friedman, Science 244:1275-1281, 1989; Eglitis and Anderson, BioTechniques 6:608-614, 1988; Tolstoshev and Anderson, Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechniques 7:980-990, 1989; and Le Gal La Salle et al., Science 259:988-990, 1993; and Johnson, Chest 107:77S-83S, 1995.

A plasmid vector is selected in part based upon the host cell that is to be transformed with the plasmid. For example, the presence of bacterial or mammalian selectable markers present in the plasmid, the origin of replication, plasmid copy number, an ability to direct random or site specific recombination with chromosomal DNA, etc. can influence the choice of an appropriate vector. A bacterial plasmid, such as pBluescript II, pET14, pUC19, pCMV-MCS and pCMVneo, can be employed for propagating an expression cassette of the present invention in bacterial cells. In a preferred aspect, a plasmid is the pCMVneo vector. In another preferred aspect, the plasmid is the pBluescript II vector.

In another aspect, a TR nucleic acid expression cassette or control nucleic acid expression cassette is inserted into a mammalian or viral shuttle vector. Whereas mammalian shuttle vectors contain mammalian selectable markers and provide for the isolation of cells containing stable genomic integrants, viral shuttle vectors provide for the reconstitution of a viral genome using recombination or genetic complementation. In some aspects, a mammalian shuttle vector is selected from the pCMV, pEYFP-N1, pEGFP-N1, or pEGFP-C1 plasmids. In a preferred aspect, the mammalian shuttle vector is pEYFP-N1. In some aspects, a viral shuttle vector is selected from the pAAV-MCS (Adeno-associated Virus serotype 2 or AAV2 genome) or pBac-1, pBacPAK8/9 (Autographa californica baculovirus genome) plasmids. In one preferred aspect, the viral shuttle vector is pAAV-MCS. In another preferred aspect, the viral shuttle vector is the pBac-1 plasmid.

To insure efficient delivery of a TR nucleic acid expression cassette or control nucleic acid expression cassette to a particular cell, tissue or organ, it can be incorporated into a non-viral delivery system, which facilitates cellular targeting. For example, a mammalian shuttle plasmid that includes a TR nucleic acid expression cassette or control nucleic acid expression cassette of the present invention may be encapsulated into liposomes. Liposomes include emulsions, foams, micelles, insoluble monolayers, liquid crystals, phospholipid dispersions, lamellar layers and the like. The delivery of DNA sequences to target cells using liposome carriers is well known in the art as are methods for preparing such liposomes.

Viruses useful in the practice of the present invention include recombinantly modified enveloped or non-enveloped DNA and RNA viruses, preferably selected from the baculoviridiae, parvoviridiae, picornoviridiae, herpesviridiae, poxviridiae, and adenoviridiae viruses. According to aspects, the recombinant virus is a baculoviridiae virus. In a preferred aspect, the baculovirus is an Autographa californica derivative virus. In other embodiments, the virus is a parvoviridiae virus. In a preferred aspect, the adeno-associated virus (“AAV”) is an AAV serotype 2. In another aspect, the AAV is an AAV serotype 1.

The viral genomes are preferably modified by recombinant DNA techniques to include a TR nucleic acid expression cassette or control nucleic acid expression cassette of the present invention and may be engineered to be replication deficient, conditionally replicating or replication competent. For example, it may prove useful to use a conditionally replicating virus to limit viral replication to specific, regulated cell culture conditions.

Chimeric viral vectors which exploit advantageous elements of more than one “parent” virus properties are included herein. Minimal vector systems in which the viral backbone contains only the sequences needed for packaging of the viral vector and optionally includes a TR nucleic acid expression cassette or control nucleic acid expression cassette may also be produced and used in the present invention. It is generally preferred to employ a virus from the species to be treated, such as a human herpes virus when a human cell or a human cell line is transduced with it. In some instances, viruses which originated from species other than the one which is to be transduced therewith can be used. For example, adeno-associated viruses (AAV) of serotypes derived from non-human sources may be useful for treating humans because the non-human serotypes should not be immediately recognized by natural or pre-existing human antibodies. By minimizing immune responses to the vectors, rapid systemic clearance of the vector is avoided and the duration of the vector's effectiveness in vivo is increased.

A TR nucleic acid expression cassette or control nucleic acid expression cassette in any of the mammalian shuttle vectors described above can be transformed into a mammalian cell. A shuttle vector can be introduced into the host cell by any technique available to those of skill in the art. These include, but are not limited to, chemical transfection (e.g., calcium chloride method, calcium phosphate method), lipofection, electroporation, cell fusion, microinjection, and infection with virus (Ridgway, A. “Mammalian Expression Vectors” Ch.24, pg. 470-472, Rodriguez and Denhardt, Eds., Butterworths, Boston Mass. 1988).

A Translation Regulated or TR element encoded by a DNA sequence included in an expression cassette and/or integrated into the genome of a stable cell line according to aspects of the present invention according to aspects of the present invention is an internal ribosome entry site (IRES), which can be distinguished from other IRESs by (a) its nucleic acid sequence context and (b) the cellular activity which regulates translation (US Published Patent Application Nos. 2006/0173168, which is hereby incorporated by reference). The combination of these two features forms a basis for selective translation of downstream coding sequences in stressed and/or dying mammalian cells that are operably linked to this IRES sequence. Thus, the present invention contemplates the use of any mammalian IRES as the TR element, which is selectively expressed in stressed and/or dying cells.

In some embodiments, the IRES element of this invention has cap-independent translational activity which localizes within the ORF of the mammalian Proteolipid Protein (PLP) gene or the DM20 splice variant thereof. In its native context, PLP IRES activity resides within a multicistronic RNA containing several upstream ORFs (“uORFs”) which effectively block ribosome scanning to internal AUG codons in normal cells. However, exposure of cells to toxic agents results in ribosome binding and translation from specific internal RNA sequences so that an internal amino acid sequence is translated from the 3′ end of the pip ORF (e.g. the PIRP-M and PIRP-L peptides).

A nucleic acid sequence encoding a TR element derived from a gene encoding the proteolipid protein (PLP) or DM20 isoform of proteolipid protein of any mammalian species is included in a TR expression cassette and/or integrated into the genome of a stable cell line according to aspects of the present invention. Nucleic acid sequences encoding PLP and DM20 are characterized by a high degree of identity between mammalian species. Human and mouse PLP and DM20 DNA sequences encoding a TR element are described in detail herein.

Mouse PLP and human PLP DNA sequences, SEQ ID NOs: 6 and 8, respectively, are highly related and characterized by 96.5% identity. Mouse DM20 and human DM20 DNA sequences, SEQ ID NOs: 5 and 7, respectively, are highly related and characterized by 96% identity.

PLP DNA sequences are highly related to DM20 DNA sequences, although DM20 is characterized by a 104 nucleotide deletion compared to PLP DNA sequences (alternative mRNA splicing). Using the human PLP DNA sequence (SEQ ID NO: 8) as a reference, human DM20 DNA sequence is 87.5% identical and the mouse DM20 is 84.3% identical. TR element encoding sequence SEQ ID NO:1 has 83.1% identity to human PLP DNA sequence (SEQ ID NO: 8). Thus, variants of TR element encoding sequences included in expression cassettes according to aspects of the present invention have 83% identity or more to SEQ ID NO: 8 when exon 3b is present.

Exon 5 is optionally excluded from DNA sequences encoding a TR element in an expression cassette and/or integrated into the genome of a stable cell line according to aspects of the present invention. Using the human PLP DNA sequence (SEQ ID NO: 8) as a reference, human DM20 DNA sequence excluding exon 5 is 78.7% identical and the mouse DM20 excluding exon 5 is 75.4% identical. Deletion of exon 5 from TR element encoding sequence SEQ ID NO:1 produces a DNA sequence with 74.2% identity to human PLP DNA SEQ ID NO: 8. Thus, variants of TR element encoding sequences included in expression cassettes according to aspects of the present invention have 74.2% identity or more to SEQ ID NO: 8 when exons 3b and 5 are deleted.

A DNA sequence included in a TR element expression cassette and/or integrated into the genome of a stable cell line according to aspects of the present invention encoding a TR element does not encode any expressed protein or peptide such that conserving one or more amino acid codons in the TR element encoding sequences is not implicated in analysis of DNA sequences encoding TR elements.

SEQ ID NO:17 is a proteolipid protein mutant consensus sequence encoding a TR element useful in compositions and methods according to aspects of the present invention. SEQ ID NO:17 is characterized by nucleotide T at nucleotide position 722; nucleotide A at nucleotide position 772; a first 18S rRNA binding site is encoded at nucleotide position 503-526; and a second 18S rRNA binding site is encoded at nucleotide position 796-822, wherein the encoded TR element confers selective translation on an operably linked coding sequence in an mRNA. Variants of SEQ ID NO:17 include these features and are further characterized as having at least 95%, 96%, 97%, 98%, 99% or greater identity to full-length SEQ ID NO:17, wherein the encoded TR element confers selective translation on an operably linked coding sequence in an mRNA.

SEQ ID NO:18 is a DM20 proteolipid protein consensus sequence encoding a TR element useful in compositions and methods according to aspects of the present invention. SEQ ID NO:18 is characterized by nucleotide T at nucleotide position 617; nucleotide A at nucleotide position 667; and further characterized in that a first 18S rRNA binding site is encoded at nucleotide position 398-422; and a second 18S rRNA binding site is encoded at nucleotide position 691-716, wherein the encoded TR element confers selective translation on an operably linked coding sequence in an mRNA. Variants of SEQ ID NO:18 include these features and are further characterized as having at least 95%, 96%, 97%, 98%, 99% or greater identity to full-length SEQ ID NO:18, wherein the encoded TR element confers selective translation on an operably linked coding sequence in an mRNA.

SEQ ID NO:19 is a proteolipid protein consensus sequence encoding a TR element useful in compositions and methods according to aspects of the present invention. SEQ ID NO:19 is characterized by nucleotide T at nucleotide position 648; nucleotide A at nucleotide position 698; a first 18S rRNA binding site is encoded at nucleotide position 503-526; and a second 18S rRNA binding site is encoded at nucleotide position 722-748, wherein exon 5 is deleted, and wherein the encoded TR element confers selective translation on an operably linked coding sequence in an mRNA. Variants of SEQ ID NO:19 include these features and are further characterized as having at least 95%, 96%, 97%, 98%, 99% or greater identity to full-length SEQ ID NO:19, wherein the encoded TR element confers selective translation on an operably linked coding sequence in an mRNA.

SEQ ID NO:20 is a DM20 proteolipid protein consensus sequence encoding a TR element useful in compositions and methods according to aspects of the present invention. SEQ ID NO:20 is characterized by nucleotide T at nucleotide position 543; nucleotide A at nucleotide position 593; and further characterized in that a first 18S rRNA binding site is encoded at nucleotide position 398-422; and a second 18S rRNA binding site is encoded at nucleotide position 617-642, wherein exon 5 is deleted, and wherein the encoded TR element confers selective translation on an operably linked coding sequence in an mRNA. Variants of SEQ ID NO:20 include these features and are further characterized as having at least 95%, 96%, 97%, 98%, 99% or greater identity to full-length SEQ ID NO:20, wherein the encoded TR element confers selective translation on an operably linked coding sequence in an mRNA.

SEQ ID NO:1 encodes a TR element (mTRdm) derived from the mouse gene encoding the DM20 isoform of proteolipid protein. SEQ ID NO:1 is characterized by nucleotide T at nucleotide position 617; nucleotide A at nucleotide position 667; mutation of nucleotide I from A to T; mutation of nucleotide 4 from G to A; mutation of nucleotide 6 from C to T; mutation of nucleotide 7 from T to G; mutation of nucleotide 8 from T to A; mutation of nucleotide 17 from G to A; mutation of nucleotide 18 from T to G; mutation of nucleotide 21 from T to A; mutation of nucleotide 27 from A to T; mutation of nucleotide 511 from A to T; and mutation of nucleotide 598 from A to T, all relative to the wild-type mouse DM20 (mDM) DNA sequence of SEQ ID NO:5; and further characterized by a first 18S rRNA binding site encoded at nucleotide position 398-422; and a second 18S rRNA binding site encoded at nucleotide position 691-716, wherein the encoded TR element confers selective translation on an operably linked coding sequence in an mRNA.

Variants of the TR element encoded by SEQ ID NO:1 are encoded by a DNA sequence of 726 nucleotides characterized by nucleotide T at nucleotide position 617; nucleotide A at nucleotide position 667; and further characterized in that any or all of nucleotides 1, 2 and 3 are mutated such that nucleotides 1, 2 and 3 are not ATG; any or all of nucleotides 27, 28 and 29 are mutated such that nucleotides 27, 28 and 29 are not ATG; any or all of nucleotides 511, 512 and 513 are mutated such that nucleotides 511, 512 and 513 are not ATG; any or all of nucleotides 598, 599 and 600 are mutated such that nucleotides 598, 599 and 600 are not ATG; any or all of nucleotides 2, 3 and 4 are mutated such that nucleotides 2, 3 and 4 are a stop codon; any or all of nucleotides 6, 7 and 8 are mutated such that nucleotides 6, 7 and 8 are a stop codon; any or all of nucleotides 16, 17 and 18 are mutated such that nucleotides 16, 17 and 18 are a stop codon; any or all of nucleotides 19, 20 and 21 are mutated such that nucleotides 19, 20 and 21 are a stop codon; all mutations relative to SEQ ID NO: 5; a first 18S rRNA binding site is encoded at nucleotide position 398-422; and a second 18S rRNA binding site is encoded at nucleotide position 691-716; and further characterized by having at least 83%, 84%, 85%, 86%, 87%, 88%, 89%, 90%, 91%, 92%, 93%, 94%, 95%, 96%, 97%, 98%, 99% or greater identity to SEQ ID NO:5 or by having at least 95%, 96%, 97%, 98%, 99% or greater identity to SEQ ID NO:5, wherein the encoded TR element confers selective translation on an operably linked coding sequence in an mRNA.

Variants of the TR element encoded by SEQ ID NO:1 are encoded by a DNA sequence of 726 nucleotides characterized by nucleotide T at nucleotide position 617; nucleotide A at nucleotide position 667; and further characterized in that nucleotide 1 is T; nucleotide 4 is A; nucleotide 6 is T; nucleotide 7 is G; nucleotide 8 is A; nucleotide 17 is A; nucleotide 18 is G; nucleotide 21 is A; nucleotide 27 is T; nucleotide 511 is T; nucleotide 598 is T, all mutations relative to SEQ ID NO: 5; a first 18S rRNA binding site is encoded at nucleotide position 398-422; and a second 18S rRNA binding site is encoded at nucleotide position 691-716, and further characterized by having at least 83%, 84%, 85%, 86%, 87%, 88%, 89%, 90%, 91%, 92%, 93%, 94%, 95%, 96%, 97%, 98%, 99% or greater identity to SEQ ID NO:5 or by having at least 95%, 96%, 97%, 98%, 99% or greater identity to SEQ ID NO:5, wherein the encoded TR element confers selective translation on an operably linked coding sequence in an mRNA.

Variants of the TR element encoded by SEQ ID NO:1 are encoded by a DNA sequence of 652 nucleotides characterized by nucleotide T at nucleotide position 543; nucleotide A at nucleotide position 593; and further characterized in that nucleotide 1 is T; nucleotide 4 is A; nucleotide 6 is T; nucleotide 7 is G; nucleotide 8 is A; nucleotide 17 is A; nucleotide 18 is G; nucleotide 21 is A; nucleotide 27 is T; nucleotide 511 is T; nucleotide 524 is T, and exon 5, nucleotides 518-591 are deleted, all mutations relative to SEQ ID NO: 5; a first 18S rRNA binding site is encoded at nucleotide position 398-422; and a second 18S rRNA binding site is encoded at nucleotide position 617-642, and further characterized by having at least 83%, 84%, 85%, 86%, 87%, 88%, 89%, 90%, 91%, 92%, 93%, 94%, 95%, 96%, 97%, 98%, 99% or greater identity to SEQ ID NO:5 or by having at least 95%, 96%, 97%, 98%, 99% or greater identity to SEQ ID NO:5, wherein the encoded TR element confers selective translation on an operably linked coding sequence in an mRNA.

Variants of the TR element encoded by SEQ ID NO:1 are encoded by a DNA sequence of 652 nucleotides characterized by nucleotide T at nucleotide position 543; nucleotide A at nucleotide position 593; and further characterized in that any or all of nucleotides 1, 2 and 3 are mutated such that nucleotides 1, 2 and 3 are not ATG; any or all of nucleotides 27, 28 and 29 are mutated such that nucleotides 27, 28 and 29 are not ATG; any or all of nucleotides 511, 512 and 513 are mutated such that nucleotides 511, 512 and 513 are not ATG; any or all of nucleotides 524, 525 and 526 are mutated such that nucleotides 524, 525 and 526 are not ATG; any or all of nucleotides 2, 3 and 4 are mutated such that nucleotides 2, 3 and 4 are a stop codon; any or all of nucleotides 6, 7 and 8 are mutated such that nucleotides 6, 7 and 8 are a stop codon; any or all of nucleotides 16, 17 and 18 are mutated such that nucleotides 16, 17 and 18 are a stop codon; any or all of nucleotides 19, 20 and 21 are mutated such that nucleotides 19, 20 and 21 are a stop codon; and exon 5, nucleotides 518-591 are deleted, all mutations relative to SEQ ID NO: 5; a first 18S rRNA binding site is encoded at nucleotide position 398-422; and a second 18S rRNA binding site is encoded at nucleotide position 617-642, and further characterized by having at least 83%, 84%, 85%, 86%, 87%, 88%, 89%, 90%, 91%, 92%, 93%, 94%, 95%, 96%, 97%, 98%, 99% or greater identity to SEQ ID NO:5 or by having at least 95%, 96%, 97%, 98%, 99% or greater identity to SEQ ID

NO:5, wherein the encoded TR element confers selective translation on an operably linked coding sequence in an mRNA.

SEQ ID NO:3 encodes a TR element (hTRdm) derived from the human gene encoding the DM20 isoform of proteolipid protein. SEQ ID NO:3 is characterized by nucleotide T at nucleotide position 617; nucleotide A at nucleotide position 667; and further includes mutation of nucleotide 1 from A to T; mutation of nucleotide 4 from G to A; mutation of nucleotide 6 from C to T; mutation of nucleotide 7 from T to G; mutation of nucleotide 8 from T to A; mutation of nucleotide 17 from G to A; mutation of nucleotide 18 from T to G; mutation of nucleotide 21 from T to A; mutation of nucleotide 27 from A to T; mutation of nucleotide 511 from A to T; mutation of nucleotide 598 from A to T, all mutations relative to the wild-type hDM DNA sequence of SEQ ID NO:7; a first 18S rRNA binding site is encoded at nucleotide position 398-422; and a second 18S rRNA binding site is encoded at nucleotide position 691-716, wherein the encoded TR element confers selective translation on an operably linked coding sequence in an mRNA.

Variants of the TR element encoded by SEQ ID NO:3 are encoded by a DNA sequence of 726 nucleotides characterized by nucleotide T at nucleotide position 617; nucleotide A at nucleotide position 667; and further characterized in that nucleotide 1 is T; nucleotide 4 is A; nucleotide 6 is T; nucleotide 7 is G; nucleotide 8 is A; nucleotide 17 is A; nucleotide 18 is G; nucleotide 21 is A; nucleotide 27 is T; nucleotide 511 is T;

nucleotide 598 is T, all mutations relative to SEQ ID NO:7; a first 18S rRNA binding site is encoded at nucleotide position 398-422; and a second 18S rRNA binding site is encoded at nucleotide position 691-716, and further characterized by having at least 83%, 84%, 85%, 86%, 87%, 88%, 89%, 90%, 91%, 92%, 93%, 94%, 95%, 96%, 97%, 98%, 99% or greater identity to SEQ ID NO:7 or by having at least 95%, 96%, 97%, 98%, 99% or greater identity to SEQ ID NO:7, wherein the encoded TR element confers selective translation on an operably linked coding sequence in an mRNA.

Variants of the TR element encoded by SEQ ID NO:3 are encoded by a DNA sequence of 726 nucleotides characterized by nucleotide T at nucleotide position 617; nucleotide A at nucleotide position 667; and further characterized in that any or all of nucleotides 1, 2 and 3 are mutated such that nucleotides 1, 2 and 3 are not ATG; any or all of nucleotides 27, 28 and 29 are mutated such that nucleotides 27, 28 and 29 are not ATG; any or all of nucleotides 511, 512 and 513 are mutated such that nucleotides 511, 512 and 513 are not ATG; any or all of nucleotides 598, 599 and 600 are mutated such that nucleotides 598, 599 and 600 are not ATG; any or all of nucleotides 2, 3 and 4 are mutated such that nucleotides 2, 3 and 4 are a stop codon; any or all of nucleotides 6, 7 and 8 are mutated such that nucleotides 6, 7 and 8 are a stop codon; any or all of nucleotides 16, 17 and 18 are mutated such that nucleotides 16, 17 and 18 are a stop codon; any or all of nucleotides 19, 20 and 21 are mutated such that nucleotides 19, 20 and 21 are a stop codon; all mutations relative to SEQ ID NO: 7; a first 18S rRNA binding site is encoded at nucleotide position 398-422; and a second 18S rRNA binding site is encoded at nucleotide position 691-716, and further characterized by having at least 83%, 84%, 85%, 86%, 87%, 88%, 89%, 90%, 91%, 92%, 93%, 94%, 95%, 96%, 97%, 98%, 99% or greater identity to SEQ ID NO:7 or by having at least 95%, 96%, 97%, 98%, 99% or greater identity to SEQ ID NO:7, wherein the encoded TR element confers selective translation on an operably linked coding sequence in an mRNA.

Variants of the TR element encoded by SEQ ID NO:3 are encoded by a DNA sequence of 652 nucleotides characterized by nucleotide T at nucleotide position 543; nucleotide A at nucleotide position 593; and further characterized in that nucleotide 1 is T; nucleotide 4 is A; nucleotide 6 is T; nucleotide 7 is G; nucleotide 8 is A; nucleotide 17 is A; nucleotide 18 is G; nucleotide 21 is A; nucleotide 27 is T; nucleotide 511 is T; nucleotide 524 is T, and exon 5, nucleotides 518-591 are deleted, all mutations relative to SEQ ID NO: 7; a first 18S rRNA binding site is encoded at nucleotide position 398-422; and a second 18S rRNA binding site is encoded at nucleotide position 617-642, and further characterized by having at least 83%, 84%, 85%, 86%, 87%, 88%, 89%, 90%, 91%, 92%, 93%, 94%, 95%, 96%, 97%, 98%, 99% or greater identity to SEQ ID NO:7 or by having at least 95%, 96%, 97%, 98%, 99% or greater identity to SEQ ID NO:7, wherein the encoded TR element confers selective translation on an operably linked coding sequence in an mRNA.

Variants of the TR element encoded by SEQ ID NO:3 are encoded by a DNA sequence of 652 nucleotides characterized by nucleotide T at nucleotide position 543; nucleotide A at nucleotide position 593; and further characterized in that any or all of nucleotides 1, 2 and 3 are mutated such that nucleotides 1, 2 and 3 are not ATG; any or all of nucleotides 27, 28 and 29 are mutated such that nucleotides 27, 28 and 29 are not ATG; any or all of nucleotides 511, 512 and 513 are mutated such that nucleotides 511, 512 and 513 are not ATG; any or all of nucleotides 524, 525 and 526 are mutated such that nucleotides 524, 525 and 526 are not ATG; any or all of nucleotides 2, 3 and 4 are mutated such that nucleotides 2, 3 and 4 are a stop codon; any or all of nucleotides 6, 7 and 8 are mutated such that nucleotides 6, 7 and 8 are a stop codon; any or all of nucleotides 16, 17 and 18 are mutated such that nucleotides 16, 17 and 18 are a stop codon; any or all of nucleotides 19, 20 and 21 are mutated such that nucleotides 19, 20 and 21 are a stop codon; and exon 5, nucleotides 518-591 are deleted, all mutations relative to SEQ ID NO: 7; a first 18S rRNA binding site is encoded at nucleotide position 398-422; and a second 18S rRNA binding site is encoded at nucleotide position 617-642, and further characterized by having at least 83%, 84%, 85%, 86%, 87%, 88%, 89%, 90%, 91%, 92%, 93%, 94%, 95%, 96%, 97%, 98%, 99% or greater identity to SEQ ID NO:7 or by having at least 95%, 96%, 97%, 98%, 99% or greater identity to SEQ ID NO:7, wherein the encoded TR element confers selective translation on an operably linked coding sequence in an mRNA.

SEQ ID NO:2 encodes a TR element (mTRp) derived from the mouse gene encoding proteolipid protein. SEQ ID NO:2 is characterized by nucleotide T at nucleotide position 722; nucleotide A at nucleotide position 772; and further includes mutation of nucleotide 1 from A to T; mutation of nucleotide 4 from G to A; mutation of nucleotide 6 from C to T; mutation of nucleotide 7 from T to G; mutation of nucleotide 8 from T to A; mutation of nucleotide 17 from G to A; mutation of nucleotide 18 from T to G; mutation of nucleotide 21 from T to A; mutation of nucleotide 27 from A to T, all mutations relative to the wild-type mPLP DNA sequence of SEQ ID NO:6; a first 18S rRNA binding site is encoded at nucleotide position 503-526; and a second 18S rRNA binding site is encoded at nucleotide position 796-822, wherein the encoded TR element confers selective translation on an operably linked coding sequence in an mRNA.

Variants of the TR element encoded by SEQ ID NO:2 are encoded by a DNA sequence of 831 nucleotides characterized by nucleotide T at nucleotide position 722; nucleotide A at nucleotide position 772; and further characterized in that nucleotide 1 is T; nucleotide 4 is A; nucleotide 6 is T; nucleotide 7 is G; nucleotide 8 is A; nucleotide 17 is A; nucleotide 18 is G; nucleotide 21 is A; nucleotide 27 is T; nucleotide 616 is T; and nucleotide 703 is T, all mutations relative to SEQ ID NO:6; a first 18S rRNA binding site is encoded at nucleotide position 503-526; and a second 18S rRNA binding site is encoded at nucleotide position 796-822, and further characterized by having at least 83%, 84%, 85%, 86%, 87%, 88%, 89%, 90%, 91%, 92%, 93%, 94%, 95%, 96%, 97%, 98%, 99% or greater identity to SEQ ID NO:6 or by having at least 95%, 96%, 97%, 98%, 99% or greater identity to SEQ ID NO:6, wherein the encoded TR element confers selective translation on an operably linked coding sequence in an mRNA.

Variants of the TR element encoded by SEQ ID NO:2 are encoded by a DNA sequence of 831 nucleotides characterized by nucleotide T at nucleotide position 722; nucleotide A at nucleotide position 772; and further characterized in that any or all of nucleotides 1, 2 and 3 are mutated such that nucleotides 1, 2 and 3 are not ATG; any or all of nucleotides 27, 28 and 29 are mutated such that nucleotides 27, 28 and 29 are not ATG; any or all of nucleotides 616, 617 and 618 are mutated such that nucleotides 616, 617 and 618 are not ATG; any or all of nucleotides 703, 704 and 705 are mutated such that nucleotides 703, 704 and 705 are not ATG; any or all of nucleotides 2, 3 and 4 are mutated such that nucleotides 2, 3 and 4 are a stop codon; any or all of nucleotides 6, 7 and 8 are mutated such that nucleotides 6, 7 and 8 are a stop codon; any or all of nucleotides 16, 17 and 18 are mutated such that nucleotides 16, 17 and 18 are a stop codon; any or all of nucleotides 19, 20 and 21 are mutated such that nucleotides 19, 20 and 21 are a stop codon; all mutations relative to SEQ ID NO: 6; a first 18S rRNA binding site is encoded at nucleotide position 503-526; and a second 18S rRNA binding site is encoded at nucleotide position 796-822, and further characterized by having at least 83%, 84%, 85%, 86%, 87%, 88%, 89%, 90%, 91%, 92%, 93%, 94%, 95%, 96%, 97%, 98%, 99% or greater identity to SEQ ID NO:6 or by having at least 95%, 96%, 97%, 98%, 99% or greater identity to SEQ ID NO:6, wherein the encoded TR element confers selective translation on an operably linked coding sequence in an mRNA.

Variants of the TR element encoded by SEQ ID NO:2 are encoded by a DNA sequence of 757 nucleotides characterized by nucleotide T at nucleotide position 648; nucleotide A at nucleotide position 698; and further characterized in that nucleotide 1 is T; nucleotide 4 is A; nucleotide 6 is T; nucleotide 7 is G; nucleotide 8 is A; nucleotide 17 is A; nucleotide 18 is G; nucleotide 21 is A; nucleotide 27 is T; nucleotide 616 is T; nucleotide 629 is T, and exon 5, nucleotides 623-696 are deleted, all mutations relative to SEQ ID NO: 6; a first 18S rRNA binding site is encoded at nucleotide position 503-526; and a second 18S rRNA binding site is encoded at nucleotide position 722-748, and further characterized by having at least 83%, 84%, 85%, 86%, 87%, 88%, 89%, 90%, 91%, 92%, 93%, 94%, 95%, 96%, 97%, 98%, 99% or greater identity to SEQ ID NO:6 or by having at least 95%, 96%, 97%, 98%, 99% or greater identity to SEQ ID NO:6, wherein the encoded TR element confers selective translation on an operably linked coding sequence in an mRNA.

Variants of the TR element encoded by SEQ ID NO:2 are encoded by a DNA sequence of 757 nucleotides characterized by nucleotide T at nucleotide position 648; nucleotide A at nucleotide position 698; and further characterized in that any or all of nucleotides 1, 2 and 3 are mutated such that nucleotides 1, 2 and 3 are not ATG; any or all of nucleotides 27, 28 and 29 are mutated such that nucleotides 27, 28 and 29 are not ATG; any or all of nucleotides 616, 617 and 618 are mutated such that nucleotides 616, 617 and 618 are not ATG; any or all of nucleotides 629, 630 and 631 are mutated such that nucleotides 629, 630 and 631 are not ATG; any or all of nucleotides 2, 3 and 4 are mutated such that nucleotides 2, 3 and 4 are a stop codon; any or all of nucleotides 6, 7 and 8 are mutated such that nucleotides 6, 7 and 8 are a stop codon; any or all of nucleotides 16, 17 and 18 are mutated such that nucleotides 16, 17 and 18 are a stop codon; any or all of nucleotides 19, 20 and 21 are mutated such that nucleotides 19, 20 and 21 are a stop codon; and exon 5, nucleotides 623-696 are deleted, all mutations relative to SEQ ID NO: 6; a first 18S rRNA binding site is encoded at nucleotide position 503-526; and a second 18S rRNA binding site is encoded at nucleotide position 722-748, and further characterized by having at least 83%, 84%, 85%, 86%, 87%, 88%, 89%, 90%, 91%, 92%, 93%, 94%, 95%, 96%, 97%, 98%, 99% or greater identity to SEQ ID NO:6 or by having at least 95%, 96%, 97%, 98%, 99% or greater identity to SEQ ID NO:6, wherein the encoded TR element confers selective translation on an operably linked coding sequence in an mRNA.

SEQ ID NO:4 encodes a TR element (hTRp) derived from the human gene encoding proteolipid protein. SEQ ID NO:4 is characterized by nucleotide T at nucleotide position 722; nucleotide A at nucleotide position 772; and further includes mutation of nucleotide 1 from A to T; mutation of nucleotide 4 from G to A; mutation of nucleotide 6 from C to T; mutation of nucleotide 7 from T to G; mutation of nucleotide 8 from T to A; mutation of nucleotide 17 from G to A; mutation of nucleotide 18 from T to G; mutation of nucleotide 21 from T to A; mutation of nucleotide 27 from A to T; mutation of nucleotide 616 from A to T; mutation of nucleotide 703 from A to T, all mutations relative to the wild-type hPLP DNA sequence of SEQ ID NO:8; a first 18S rRNA binding site is encoded at nucleotide position 503-526; and a second 18S rRNA binding site is encoded at nucleotide position 796-822, wherein the encoded TR element confers selective translation on an operably linked coding sequence in an mRNA.

Variants of the TR element encoded by SEQ ID NO:4 are encoded by a DNA sequence of 831 nucleotides characterized by a nucleotide T at nucleotide position 722; nucleotide A at nucleotide position 772; and further characterized in that nucleotide 1 is T; nucleotide 4 is A; nucleotide 6 is T; nucleotide 7 is G; nucleotide 8 is A; nucleotide 17 is A; nucleotide 18 is G; nucleotide 21 is A; nucleotide 27 is T; nucleotide 616 is T; nucleotide 703 is T, all mutations relative to SEQ ID NO:8; a first 18S rRNA binding site is encoded at nucleotide position 503-526; and a second 18S rRNA binding site is encoded at nucleotide position 796-822, and further characterized by having at least 83%, 84%, 85%, 86%, 87%, 88%, 89%, 90%, 91%, 92%, 93%, 94%, 95%, 96%, 97%, 98%, 99% or greater identity to SEQ ID NO:8 or by having at least 95%, 96%, 97%, 98%, 99% or greater identity to SEQ ID NO:8, wherein the encoded TR element confers selective translation on an operably linked coding sequence in an mRNA.

Variants of the TR element encoded by SEQ ID NO:4 are encoded by a DNA sequence of 831 nucleotides characterized by nucleotide T at nucleotide position 722; nucleotide A at nucleotide position 772; and further characterized in that any or all of nucleotides 1, 2 and 3 are mutated such that nucleotides 1, 2 and 3 are not ATG; any or all of nucleotides 27, 28 and 29 are mutated such that nucleotides 27, 28 and 29 are not ATG; any or all of nucleotides 616, 617 and 618 are mutated such that nucleotides 616, 617 and 618 are not ATG; any or all of nucleotides 703, 704 and 705 are mutated such that nucleotides 703, 704 and 705 are not ATG; any or all of nucleotides 2, 3 and 4 are mutated such that nucleotides 2, 3 and 4 are a stop codon; any or all of nucleotides 6, 7 and 8 are mutated such that nucleotides 6, 7 and 8 are a stop codon; any or all of nucleotides 16, 17 and 18 are mutated such that nucleotides 16, 17 and 18 are a stop codon; any or all of nucleotides 19, 20 and 21 are mutated such that nucleotides 19, 20 and 21 are a stop codon; all mutations relative to SEQ ID NO: 8; a first 18S rRNA binding site is encoded at nucleotide position 503-526; and a second 18S rRNA binding site is encoded at nucleotide position 796-822, and further characterized by having at least 83%, 84%, 85%, 86%, 87%, 88%, 89%, 90%, 91%, 92%, 93%, 94%, 95%, 96%, 97%, 98%, 99% or greater identity to SEQ ID NO:8 or by having at least 95%, 96%, 97%, 98%, 99% or greater identity to SEQ ID NO:8, wherein the encoded TR element confers selective translation on an operably linked coding sequence in an mRNA.

Variants of the TR element encoded by SEQ ID NO:4 are encoded by a DNA sequence of 757 nucleotides characterized by nucleotide T at nucleotide position 648; nucleotide A at nucleotide position 698; and further characterized in that nucleotide 1 is T; nucleotide 4 is A; nucleotide 6 is T; nucleotide 7 is G; nucleotide 8 is A; nucleotide 17 is A; nucleotide 18 is G; nucleotide 21 is A; nucleotide 27 is T; nucleotide 616 is T; nucleotide 629 is T, and exon 5, nucleotides 623-696 are deleted, all mutations relative to SEQ ID NO: 8; a first 18S rRNA binding site is encoded at nucleotide position 503-526; and a second 18S rRNA binding site is encoded at nucleotide position 722-748, and further characterized by having at least 83%, 84%, 85%, 86%, 87%, 88%, 89%, 90%, 91%, 92%, 93%, 94%, 95%, 96%, 97%, 98%, 99% or greater identity to SEQ ID NO:8 or by having at least 95%, 96%, 97%, 98%, 99% or greater identity to SEQ ID NO:8, wherein the encoded TR element confers selective translation on an operably linked coding sequence in an mRNA.

Variants of the TR element encoded by SEQ ID NO:4 are encoded by a DNA sequence of 757 nucleotides characterized by nucleotide T at nucleotide position 648; nucleotide A at nucleotide position 698; and further characterized in that any or all of nucleotides 1, 2 and 3 are mutated such that nucleotides 1, 2 and 3 are not ATG; any or all of nucleotides 27, 28 and 29 are mutated such that nucleotides 27, 28 and 29 are not ATG; any or all of nucleotides 616, 617 and 618 are mutated such that nucleotides 616, 617 and 618 are not ATG; any or all of nucleotides 629, 630 and 631 are mutated such that nucleotides 629, 630 and 631 are not ATG; any or all of nucleotides 2, 3 and 4 are mutated such that nucleotides 2, 3 and 4 are a stop codon; any or all of nucleotides 6, 7 and 8 are mutated such that nucleotides 6, 7 and 8 are a stop codon; any or all of nucleotides 16, 17 and 18 are mutated such that nucleotides 16, 17 and 18 are a stop codon; any or all of nucleotides 19, 20 and 21 are mutated such that nucleotides 19, 20 and 21 are a stop codon; and exon 5, nucleotides 623-696 are deleted, all mutations relative to SEQ ID NO: 8; a first 18S rRNA binding site is encoded at nucleotide position 503-526; and a second 18S rRNA binding site is encoded at nucleotide position 722-748, and further characterized by having at least 83%, 84%, 85%, 86%, 87%, 88%, 89%, 90%, 91%, 92%, 93%, 94%, 95%, 96%, 97%, 98%, 99% or greater identity to SEQ ID NO:8 or by having at least 95%, 96%, 97%, 98%, 99% or greater identity to SEQ ID NO:8, wherein the encoded TR element confers selective translation on an operably linked coding sequence in an mRNA.

A DNA sequence encoding a TR element and included in an expression cassette according to aspects of the present invention is derived from exons 1-7 of the PLP gene and/or DM20 gene. While not being bound to a particular theory, it is believed that the exons 1 through 4 are sufficient to encode a functional IRES activity based on mutational analysis data. Furthermore, it is believed that the TR regulatory system, which plays a role in stress/death-specific translation is located within exons 6 and/or 7.

The following mutations were made in wild-type human and mouse DNA sequences encoding PLP and DM20 shown herein as SEQ ID NOs: 5-8, producing mutant sequences: nucleotide 1 was mutated from A to T to remove the wild type AUG start codon in the myelin proteolipid protein PLP and DM20 cDNAs that directs the synthesis of the full length PLP and DM20 in order to prevent such synthesis from occurring; nucleotide 4 was mutated from G to A in order to create a stop codon in the second possible reading frame of the PLP and DM20 cDNAs to prevent full length synthesis thereof; nucleotides 6, 7 and 8 were mutated from C to T, T to G and T to A respectively to create a stop codon in the third possible reading frame of the PLP and DM20 cDNAs to prevent synthesis of the full length PLP and DM20; nucleotides 17 and 18 were mutated from G to A and T to G, respectively to create the first stop codon in the main (first) open reading frame of the PLP and DM20 cDNAs to prevent their full length synthesis; nucleotide 21 was mutated from T to A in order to create the second stop codon in the main (first) open reading frame of the PLP and DM20 cDNAs to prevent full length synthesis thereof; nucleotide 27 was mutated from A to T in order to remove the AUG codon from the third possible reading frame of the PLP and DM20 cDNAs to prevent out-of frame translation initiation in the absence of the wild type AUG codon; and the stop codon was deleted from the PLP and DM20 cDNAs to reduce interference with translation of the downstream open reading frame.

TR elements encoded by DNA sequences included in expression cassettes according to aspects of the present invention derived from PLP or DM20 do not direct translation of either PIRP-M or PIRP-L peptide. In addition to the above changes, the following mutations were introduced into the sequences encoding TR elements from the DM 20 variant of the cDNA: nucleotide 511 was mutated from A to T in order to remove the first in-frame internal AUG start codon in the DM20 variant that directs the synthesis of PIRP-M protein to prevent such synthesis from occurring; and nucleotide 598 was mutated from A to T to remove the second in-frame internal AUG start codon in the DM20 variant that directs the synthesis of PIRP-L protein in order to prevent such synthesis from occurring.

Similarly, the following mutations were introduced into the TR elements from the murine PLP variant of the cDNA: nucleotide 616 was mutated from A to T in order to remove the first in-frame internal AUG start codon in the PLP variant that directs the synthesis of PIRP-M protein to prevent such synthesis from occurring; and nucleotide 703 was mutated from A to T to remove the second in-frame internal AUG start codon in the PLP variant that directs the synthesis of PIRP-L protein in order to prevent such synthesis from occurring.

According to aspects of the present invention, a TR element is selected from a human or a mouse TR element. More preferably, the TR element is selected from those encoded by SEQ ID NO:1, SEQ ID NO:2, SEQ ID NO:3, SEQ ID NO:4, SEQ ID NO:17, SEQ ID NO:18, SEQ ID NO:19, SEQ ID NO:20 or a variant of any thereof, wherein the encoded TR element confers selective translation on an operably linked coding sequence in an mRNA.

According to aspects of the present invention, a DNA sequence encoding a TR element includes A) a PLP nucleotide sequence corresponding to at least nucleotides 1-831 of reference sequences SEQ ID NO: 2 or SEQ ID NO:4 and having at least 62% sequence identity thereto, or B) a DM20 nucleotide sequence corresponding to at least nucleotides 1-726 of reference sequences SEQ ID NO: 1 or SEQ ID NO:3 and having at least 62% sequence identity thereto; the DNA sequence encoding a TR element includes a polypyrimidine tract at one or more of SEQ ID NO: 2 or SEQ ID NO:4 PLP nucleotide positions 41-48, 50-56, 75-81, 150-156, 200-205, 227-244, 251-257, 270-274, 299-303, 490-494, 563-570, 578-582, 597-601, 626-632, 642-648, 669-674, 707-712, 755-761, 767-771, and 800-804, or at one or more positions corresponding thereto in SEQ ID NO: 1 or SEQ ID NO:3.

In one preferred embodiment, the sequence identity of (A) or (B) is at least or about 70%, and more preferably it is at least or about 80%.

According to aspects of the present invention, a DNA sequence encoding a TR element includes a GNRA sequence at one or more of SEQ ID NO: 2 or SEQ ID NO:4 PLP nucleotide positions 130-133, 142-145, 190-193, 220-223, 305-308, 329-332, 343-346, 572-575, 635-638, 650-653 and 683-686 or at one or more positions corresponding thereto in SEQ ID NO: 1 or SEQ ID NO:3.

In PLP/DM20 coding sequences, and TR elements encoded thereby or constructed therefrom, mutations can be made, without adverse effect on TR element function, at one or more positions corresponding to the following PLP/DM20 nucleotide positions of SEQ ID NO: 2 and SEQ ID NO:4/SEQ ID NO:1 and SEQ ID NO:3: 1, 2, 3, 4 to 21 (including deletion of all of part of this segment), 25, 26, 314, 332, 560/455, 614/509, 623/518 to 696/591 (including deletion of all or part of this segment, which removes exon 5), 616/511, 703/598, 806/701, 811/706, 817/712, 818/713, and 827/722. In various embodiments, other nucleobases than the foregoing can be conserved in PLP/DM20 coding sequences.

In PLP/DM20 coding sequences, and TR elements encoded thereby or constructed therefrom, mutations can be made, without adverse effect on TR element function, at one or more positions corresponding to the following PLP/DM20 nucleotide positions of SEQ ID NO: 2 and SEQ ID NO:4/SEQ ID NO:1 and SEQ ID NO:3: 1, 4, 6, 7, 8, 17, 18, 21, 25, 26, 27, 314, 332, 560/455, 616/511, 703/598, 806/701, 811/706, 817/712, 818/713, and 827/722. In some embodiments, these mutations can be one or more of: 1t, 4a, 6t, 7 g, 8a, 17a, 18 g, 21a, 25 g, 26c, 27t, 314 g, 332 g, 560/455c, 616/511t, 703/598t, 806/701g, 811/706t, 817/712a, 818/713a, and 827/722 g.

In addition, insertions, e.g., insertions of up to or about 5 nucleotides, can be made at PLP position 614/509, with no adverse effect on IRES function. In addition, fusions to position 831/726, e.g., in-frame fusions thereto of reporter or other target gene coding sequences, do not exhibit any adverse effect on TR element function.

In another embodiment, the TR element of the present invention is derived from a vertebrate PLP or DM20 sequence other than a human or a mouse. In some embodiments, this can be a primate, rod equine, bovine, ovine, porcine, canine, feline, lapine, marsupial, avian, piscine, amphibian, or reptilian sequence. In various embodiments, a vertebrate sequence can be a native sequence, whether wild-type or variant; in some embodiments, a vertebrate sequence can be a wild-type sequence.

As used herein in regard to PLP/DM20 sequences, “mammalian consensus sequence” refers to the DNA sequence SEQ ID NO: 9. The “mammalian consensus sequence” refers to the PLP or DM20 sequences of the species Homo sapiens, Pongo pygmaeus (orangutan), Pan troglodytes (chimpanzee), Macaca mulatta (rhesus monkey), Macaca fascicularis (crab-eating macaque), Sus scrofa (pig), Mus musculus (mouse), Rattus norvegicus (rat), Monodelphis domestica (opossum), Oryctolagus cuniculus (rabbit), Bos taurus (cattle) and Canis familiaris (dog). In the consensus sequences, the following standard abbreviations are used for nucleotides: m is a or c, r is a or g, w is a or t, s is c or g, y is c or t, k is g or t, v is a or c or g, h is a or c or t, d is a or g or t, b is c or g or t, xin is a or c or g or t.

In some embodiments, a non-mammalian vertebrate PLP and/or DM20 sequence can be used, such as those denoted in GenBank as CAA43839 (chicken), P47790 (zebra finch), AAW79015 (gecko lizard), CAA79582 (frog), or BAA84207 (coelacanth).

DNA sequences encoding these are readily available to one of ordinary skill in the art by searching NCBI Genbank in the Nucleotide menu at the http World Wide Web ncbi.nlm.nih.gov/sites/entrez website. For example, useful DNA sequences include those listed under Genbank accession numbers: AJ006976 (human), CR860432 (orangutan), XM_001140782 (chimpanzee), XM_001088537 (rhesus monkey), AB083324 (crab-eating macaque), NM_213974 (pig), NM_011123 (mouse), NM 030990 (rat), XM 001374446 (opossum), NM_001082328 (rabbit), AJ009913 (cattle), X55317 (dog), X61661 (chicken), NM_001076703 (residues 113-946, zebra finch), AY880400 (gecko lizard), 219522 (frog), and AB025938 (coelacanth).

In certain instances, sequence elements operably linked to the encoded TR element might disrupt the selective translational activity displayed by the TR element or exhibit sub-optimal translational activity. To alleviate any effect on TR activity by the linked ORF, the present invention provides for codon-usage variants of the disclosed nucleotide sequences, that employ alternate codons which do not alter the polypeptide sequence (and thereby do not affect the biological activity) of the expressed polypeptides. These variants are based on the degeneracy of the genetic code, whereby several amino acids are encoded by more than one codon triplet. An example would be the codons CGT, CGG, CGC, and CGA, which all encode the amino acid, arginine (R). Thus, a protein can be encoded by a variant nucleic acid sequence that differs in its precise sequence, but still encodes a polypeptide with an identical amino acid sequence. Based on codon utilization/preference, codons can be selected to optimize the translation efficiency of an ORF without affecting regulated translation from the TR expression cassette.

Site directed mutagenesis is one particularly useful method for producing sequence variants by altering a nucleotide sequence at one or more desired positions. Site directed (or site specific) mutagenesis uses oligonucleotide sequences comprising a DNA sequence with the desired mutation, as well as a sufficient number of adjacent nucleotides to provide a sequence of sufficient size and complexity to form a stable duplex on both sides of the proposed mutation. Typically, a synthetic primer of about 20 to 25 nucleotides in length is preferred, with about 5 to 10 residues on both sides of the proposed mutation of the sequence being altered. Typical vectors useful in site directed mutagenesis include the disclosed vectors, as well as any commercially or academically available plasmid vector. In general, nucleotide substitutions are introduced by annealing the appropriate DNA oligonucleotide sequence with the target DNA and amplifying the target sequence by PCR procedures known in the art.

The present invention contemplates the use of every possible codon in a coding sequence for producing the desired ORF sequence for use in accordance with this invention.

Directed evolution techniques can be used to prepare sequence variants having improved TR function. In a directed evolution technique, at least one round of nucleic acid mutation or nucleic acid splicing or homologous recombination can be performed, starting from a TR-containing polynucleotide. Mutation, splicing, and homologous recombination can be performed in a directed or random manner. For example, one or more oligonucleotides can be designed for site-directed mutagenesis of the TR element, as described above, or one or more randomly generated oligonucleotides can be contacted with the initial TR-containing polynucleotide template. Alternatively, or in addition, PCR amplification of the initial template can be performed under error-permissive conditions and/or an error-prone polymerase to permit introduction of mutations, a technique referred to as “sloppy” PCR.

Similarly, a set of homologous, TR-element-containing polynucleotides can be spliced or recombined in a directed or random manner. For example, one or more restriction endonucleases can be used to digest the homologous polynucleotide templates, randomly or in a predetermined manner, and the resulting fragments can then be ligated together. Alternatively or in addition, the set of TR-element-containing polynucleotides can be pooled and treated under conditions favoring homologous recombination among them, either in vitro or in cyto. In particular, regulatory sequences important for TR-specific translational efficiency could be combined or amplified in number so that sequences containing multiple copies are produced. For this effort, any combination of mutation and splicing or recombination techniques can be employed. One or more than one rounds of any of these can be performed.

After one or more rounds of mutation, splicing, and/or recombination, the resulting polynucleotides are then tested to screen for TR activity. Typically, this can be done by placing a reporter molecule coding sequence under the operative control of one or more of the TR variants that have been produced. The resulting construct(s) are then expressed in a cell that is placed under conditions, such as a condition of stress, for which TR translation can take place. The testing can be used to detect a desired improvement in TR element function. For example, any one of improvement in specificity of TR element translation to a stress condition, sensitivity of TR element activation to a cellular stress response (e.g., a biochemical change antecedent to cell stress and/or death), or efficiency (i.e. magnitude) of translation initiation upon TR element activation can be the focus of the assay).

Based on the assay result, one or more improved TR elements can be selected for use, or for further development; in some embodiments, the selected improved TR element nucleic acids can be used as a starting polynucleotide or as a starting set of polynucleotides for another round, or course of rounds, of directed evolution.

Site directed mutagenesis examined exon 5-7 sequences for SET regulation activity. As expected, deletion of exon 5 (also present in the mouse jimpy mutation) disrupts all of the ORFs associated with exons 5 and 6 (the PIRP-M/PIRP-L ORFs and uORFs 7 and 8) but did not affect SET regulation. This indicated that SET from the full-length TR expression cassette required cis-regulatory elements possibly associated with the single uORF not affected by the exon 5 deletion (uORF9, which maps within exon 7). Unexpectedly, a single base change altering the uORF9 AUG codon (AUG to UUG) diminished SET regulation in growing cells and allowed translation of the reporter ORF in unstressed cells. To further examine whether translation of uORF9 was necessary for SET of the reporter ORF, mutations were introduced into specific amino acids (expressed as single and paired codon changes) that altered the amino acid sequence but did not introduce termination codons into uORF9. These mutants established that specific nucleotide changes exhibited dominant negative regulation of SET. Using the RNA structure analysis software M-fold, each of the uORF9 mutations were examined for RNA structural changes, which found that each dominant negative mutation altered multiple stable RNA structures, including the intact proteolipid mRNA structure. These studies provide strong evidence that a wildtype RNA structure associated with uORF9 is required for efficient SET regulation.

Further comparative sequence analysis of uORF9 identified a short RNA segment that was highly complementary to a second 18S rRNA sequence, but had minimal homology to the plp IRES 18S rRNA sequence. Although both sequences could tentatively hybridize to different segments of the same exposed helix in 18S rRNA, the uORF9 18S rRNA sequence was more similar to a conserved viral sequence (present in many mammalian viruses) that directs cap-independent translation reinitiation of dORFs in infected cells. As with the IRES 18S rRNA sequence, viral translation reinitiation requires mRNA-18S rRNA interactions that control 40S subunit interactions with a mRNA. However, in contrast to the IRES 18S rRNA interaction, translation reinitiation also requires RNA structural elements that interact with the eIF3 complex to align the mRNA in the mRNA exit tunnel. This mRNA-18S rRNA-eIF3 complex retains the 40S subunit on a mRNA so that a fresh ternary complex can be bound and also positions a dORF AUG codon for translation reinitiation.

In a preferred aspect, the TR mRNA directs the SET of a reporter protein in stressed cells using two independent functional elements. The first functional sequence (termed the TR IRES) is a constitutive IRES element located in Exon 4 capable of binding the 80S subunit in growing and stressed cells. The second functional sequence (termed the TR Regulator located in Exon 7) controls TR mRNA interactions between the growth ribosome and the plp IRES in unstressed cells; however, in cells treated with a toxin the TR Regulator controls the interactions between the TR mRNA, the 40S subunit, and the eIF3 complex to position the reporter dORF for translation reinitiation. The ability of the downstream TR Regulator to control an upstream constitutive TR IRES has not been detected in or reported for any other IRES element. This makes the SET process detected by the TR expression system unique and shows that the 80S ribosome directing SET in stressed cells differs from the 80S ribosome producing cap-dependent translation in growing cells.

In one aspect of this invention, a SET “Reference Standard” agent or RefStan is any drug dosing protocol, environmental treatment, or cellular manipulation process that can be performed using a standardized procedure and consistently produces a predictable SET response in a TR “Reference” or “Ref” cell line. In another aspect of this invention, a TR Ref cell line is any cell line stably expressing the TR gene cassette that has been subjected to a comprehensive screen of SET RefStan and stably produces predictable SET responses. In a preferred aspect, this invention describes methods that use TR Ref cell lines to characterize the biological impact of overexpressing the SET signaling pathway. These Ref cell lines are vital for defining the in vitro and in vivo efficacy of drugs that target a primary SET signaling effector (the SET Ribosome).

In some aspects of the present invention, a mammalian cell can be a mammalian cell that is isolated from an animal (i.e., a primary cell) or a mammalian tumor cell line. Methods for cell isolation from animals are well known in the art. In some aspects, a primary cell is isolated from a mouse. In other aspects, a primary cell is isolated from a human. In still other aspects, a mammalian tumor cell line can be used. Exemplary cell lines include HEK293 (human embryonic kidney), HT1080 (human fibrosarcoma), NTera2D (human embryonic teratoma), HeLa (human cervical adenocarcinoma), Caco2 (human colon adenocarcinoma), HepG2 (human liver hepatocellular carcinoma), HCT116 (human colon tumor), MDA231 (human breast cancer), U2 OS (human bone osteosarcoma), DU145 (human prostate carcinoma), LNCaP (human prostate adenocarcinoma), LoVo (human colon cancer), MiaPaCa2 (human pancreatic carcinoma), AsPC1 (human pancreatic adenocarcinoma), MCF-7 (human breast cancer), PC3, Capan-2 (human pancreas adenocarcinoma), COL0201 (human colon cancer), COL0205 (human colon tumor), H4 (human brain neuroglioma), HuTu80 (human duodenum adenocarcinoma), HT1080 (human connective tissue fibrosarcoma), and SK-N-MC (human brain neuroepithelioma). Mammalian tumor cell lines are typically available from, for example, the American Tissue Culture Collection (ATCC) or any approved Budapest treaty site or other biological depository.

One aspect of the invention was the surprising discovery that mammalian cells stably transformed with the TR expression cassette can be divided into three “TR Classes” based upon the level of “SET activation”-dependent protein translation (see below). An entire population of stably transformed cells, in which each cell comprises at least one integration event of the transgene which confers drug resistance, is termed a “cell pool.” The subsequent isolation of individual cell colonies derived from a cell pool is termed a “cell line.” In contrast to the first approach, which provides a mixed population of cells with a wide array of SET-specific expression levels, the second approach requires the selection, isolation, and characterization of distinct clones from thousands of potential cell colonies to purify a select group of colonies which express a unique SET-dependent protein expression level (i.e. a TR Ref cell line).

Cell pools were generated using multiple transfection protocols and plated to recover all drug resistant cells. These primary cell pool cultures (termed a passage 0 culture) contain a comprehensive random set of all potential transfectants. However, as is well known in the art, it is generally desirable to subclone the cells from the cell pool in order to obtain a pure cell isolate. Cell isolates were recovered using selection and purification methods that did not opt for a cell type-specific isolate (e.g. larger colony size, enhanced plating efficiency, or faster isolate growth rate). Colonies were isolated from multiple plates, harvested without any size bias, propagated using a standardized method, and assayed when colony growth reached a defined size and cell number. Each clone surviving this protocol (i.e. the entire cell line population) was screened for a TR Assay response using a SET Agonist RefStan.

To measure a standard SET response, the amount of reporter protein translated from the TR expression cassette in treated cells is assayed and compared to the level of the reporter protein expressed by an untreated cell standard. The ratio of the test sample response to control expression level is expressed as percent of untreated control. At the start of these studies, no RefStan existed that had been experimentally shown to directly or indirectly regulate SET or a SET signaling effector (the SET Ribosome). Therefore, it seemed logical that candidate RefStan agents would include any agent that could damage and/or kill a cell, selectively alter a known metabolic process in a cell without affecting cell viability, or produce a neutral SET response. As an initial test concentration for a candidate RefStan, a concentration or treatment known to absolutely regulate the agent-specific target enzyme or signaling system was used as a preferred test treatment. One skilled in the art will know that if a candidate test treatment involved a specific drug dose, then defining a standard SET response requires the use of a dose response assay. After a standard RefStan protocol had been established, that protocol was used for all subsequent cell based assays.

TR cell lines, characterized for their SET activation potential, were assigned to specific classes using population analysis. All treatment responses were assigned an order using a ranking plot. A trend analysis was used to define at least three SET activation trends that was independent of tumor cell type (all transfected tumor cells contained the same three SET activation responses and could be used to isolate TR Ref cell lines). However, as one skilled in the art will recognize, the most accurate TR Class distribution requires the examination of a statistically significant number of subclones to accurately represent the entire range of SET activation responses. Preferably, to obtain cell line representatives from the three TR Classes required the isolation of at least about 60 independent subclones, more preferably of at least about 100 independent subclones, still more preferably of at least about 250 independent subclones. Once a cell line was identified, it was amplified and either maintained in cell culture or frozen for storage and future use.

The three TR cell Classes were arbitrarily named Class 1, Class 2 and Class 3 cells, and can be classified as follows. Upon treatment with a “SET Agonist” RefStan, Class 1 cells are characterized by the level of a reporter protein ranging from 100% to 500% greater than the level of the reporter protein in the untreated cell standard, wherein the untreated cell standard represents the level of the reporter protein in mammalian cells stably transformed with the nucleic expression cassette and not treated with a reference standard agent(s). Similarly, Class 2 cells are characterized by the level of a reporter protein being more than 500% and not more than 1400% greater than the level of the reporter protein in the untreated cell standard, and Class 3 are characterized by the level of a reporter protein being more than 1400% greater than the level of the reporter protein in the untreated cell standard. In one preferred aspect, the Class 3 cells are characterized by the level of a reporter protein being more than 20,000% and not more than 75,000% greater than the level of the reporter protein in the untreated cell standard. Class designations were assigned to groups of cell lines based upon the mean of a putative Class differing by 2 standard deviations from the adjacent Class grouping. For example, the mean of reporter protein expression in all Class 1 cell lines, following treatment with a SET Agonist, was 2 standard deviations lower than the mean of all recovered Class 2 cell lines.

In a preferred aspect, cell lines are treated with one RefStan, which was delivered at a fixed dose, for a fixed time, in a defined volume, on a specific number of cells at 37° C. and 5% CO₂ (all water insoluble RefStan are dissolved and delivered to cells in DMSO). In other aspects, the cells are treated with multiple RefStan. By way of example and not of limitation, the SET RefStan were developed from the group consisting of cAMP, thapsigargin, TPA, paclitaxel (Taxol), nocodazole, vinblastine, colchicine, Calcium Ionophore A23167, MG132, bortezomib (Velcade), hycamtin (Topotecan), 4-oxoquinoline-3-carboxylic acid derivative antibiotic, ethanol, and methanol. When using at least two RefStan, any combination of RefStan can be used. One skilled in the art can readily determine which RefStan combinations may be particularly useful based on their mechanism of action. Exemplary combinations of two RefStan are detailed below. By way of example, two RefStan combinations include but are not limited to cAMP and TPA; cAMP and paclitaxel, cAMP and thapsigargin, cAMP and nocodazole, cAMP and vinblastin, cAMP and colchicine, cAMP and MG132, cAMP and bortezomib(Velcade), cAMP and Calcium lonophore A23167, cAMP and 4-oxoquinoline-3-carboxylic acid derivative antibiotic, cAMP and hycamtin; TPA and paclitaxel, TPA and thapsigargin, TPA and nocodazole, TPA and vinblastin; TPA and colchicine, TPA and MG132, TPA and bortezomib, TPA and Calcium Ionophore A23167, TPA and 4-oxoquinoline-3-carboxylic acid derivative antibiotic, TPA and hycamtin; paclitaxel and thapsigargin; paclitaxel and nocodazole; paclitaxel and vinblastin; paclitaxel and colchicine, paclitaxel and MG132, paclitaxel and bortezomib, paclitaxel and Calcium lonophore A23167, paclitaxel and 4-oxoquinoline-3-carboxylic acid derivative antibiotic, paclitaxel and hycamtin; MG132 and thapsigargin; MG132 and nocodazole; MG132 and vinblastin; MG132 and colchicine; MG132 and bortezomib, MG132 and Calcium Ionophore A23167, MG132 and 4-oxoquinoline-3-carboxylic acid derivative antibiotic, MG132 and hycamtin; thapsigargin and nocodazole, thapsigargin and vinblastin; thapsigargin and colchicine, thapsigargin and bortezomib, thapsigargin and Calcium Ionophore A23167, thapsigargin and 4-oxoquinoline-3-carboxylic acid derivative antibiotic, thapsigargin and hycamtin; nocodazole and vinblastin; nocodazole and colchicine, nocodazole and Calcium ionophore A23167, nocodazole and 4-oxoquinoline-3-carboxylic acid derivative antibiotic, nocodazole and hycamtin; vinblastin and colchicine, vinblastin and bortezomib, vinblastin and Calcium lonophore A23167, vinblastine and 4-oxoquinoline-3-carboxylic acid derivative antibiotic, vinblastin and hycamtin; colchicine and bortezomib, colchicine and Calcium Ionophore A23167; colchicine and 4-oxoquinoline-3-carboxylic acid derivative antibiotic, colchicine and hycamtin; bortezomib and Calcium lonophore A23167; bortezomib and 4-oxoquinoline-3-carboxylic acid derivative antibiotic, bortezomib and hycamtin; Calcium Ionophore A23167 and 4-oxoquinoline-3-carboxylic acid derivative antibiotic, Calcium lonophore A23167 and hycamtin; and 4-oxoquinoline-3-carboxylic acid derivative antibiotic and hycamtin.

In a preferred aspect, all TR cell lines are screened with a SET activation RefStan to assign each isolate to a TR Class. In another aspect, the SET activation RefStan upregulates the protein kinase C pathway. In another aspect, specific examples of SET Agonist RefStan include the polyoxyl hydrogenated castor oil family, the phorbol ester compound family, and the bryostatin analogs. In other aspects, specific examples of SET activation RefStan include cremophor EL, TPA and bryostatin 1.

The prototypical PKC isozyme contains a conserved COOH-terminal kinase sequence and a variable NEI-terminal regulatory domain, where differences in the regulatory sequences functionally defines three enzyme classes based upon differential modes of activation. For example, the conventional PKCs [(cPKC) PKCα, PKCβI, PKCβII, and PKCγ] are described as lipid-sensitive enzymes activated by the hydrolysis of the membrane bound phosphatidylinositol 4,5-bisphosphase (PIP2) by phospholipase C (PLC) and the release of the second messenger molecules diacylglycerol (DAG) and inositol triphosphate (IP3). Whereas IP3 enters the cytosol and stimulates calcium ion release from the ER, the hydrophobic DAG molecule binds the cPKCs at the plasma membrane surface. Therefore, cPKC requires DAG binding (or a DAG derivative such as the phorbol ester TPA) and calcium ions for activation. In contrast, the novel PKCs [(nPKC) PKCδ/θ and PKCε/β] lack the calcium ion binding sequence and only require DAG (or TPA) for activation. In contrast, the atypical PKCs [(aPKCs) PKCζ, PKC₁/λ] lack the calcium ion binding sequence but contain a modified regulatory sequence so that aPKC activation is regulated by phosphoinositol-3,4,5-triphosphate (PIP3) binding, phosphorylation by various kinases, and autophosphorylation. The aPKC isoforms also contain protein-protein contact sites that direct the inactive and active kinase to subcellular locations close to target substrates to facilitate receptor mediated signal transduction and cytoskeletal/microvesicle reorganization.

As with the prototypical PKC isoforms, a fourth group of lipid-activated PKC-like kinases (PKCμ/PKD1, PKCv/PKD2, PKD3) can regulate TPA-dependent SET activation. These enzymes contain sequences homologous to the PKC regulatory domain but contain a kinase domain similar to the calmodulin-dependent kinase (an enzymatic activity required for cell cycle progression). Upon cellular stimulation, the NH-terminal PKC-like regulatory domain guides the inactive PKD protein to specific subcellular positions (e.g. the plasma or ER membrane) where the inactive kinase binds lipids (or TPA) and is phosphorylated by a PKC-dependent (e.g. nPKC enzymatic activity) or PKC-independent kinases. Autophosphorylation completes the activation of the PKD kinases which allows the PKD kinase to act as a down-stream effector of PKC activation and regulate cellular recovery after cell damage.

A complex pattern of isozyme-specific spatiotemporal movements are required to localize the PKCs close to their intracellular substrates. After activation, the PKC isozymes often move from the site of activation and localize to the plasma membrane, nucleus, ER/Golgi, and/or mitochondria. As with other cellular kinases, maintenance of the activated state, protein turnover, and subcellular localization are regulated by scaffolding proteins that anchor the activated kinase. In this manner, scaffold proteins integrate diverse signal transduction pathways and control cross-talk between different signaling cascades by physically clustering signaling molecules.

Of particular interest to this invention is the PKC activity associated with the Receptor for Activated C Kinase 1 (RACK1) protein, a 36 kDa cytosolic protein containing seven WD40 (Trp-Asp 40) repeats that is a selective anchoring protein for PKC (preferred partners are the PKCβII, PKCε, PKCδ and PKCμ isotypes). Even though RACK1 can be found at the plasma and nuclear membranes, it is of particular interest to this invention, that the RACK1/Protein Kinase complex binds to the eukaryotic ribosome. At this location, the RACK1 protein connects to the 40S Head structure, contacting the 18S rRNA (close to the mRNA exit channel) and the eIF3 complex, as well as a vast array of signaling proteins, such as the Src kinase family, integrin β subunit (CD104), PDE4D5 signal transducers, activators of transcription 1 (STAT1), insulin-like growth factor receptor, E3 Ubiquitin ligases (VHL, Elongin C, etc), and the androgen receptor. By anchoring these proteins, RACK1 complexes control cell cycle progression, anti-apoptotic/stress responses, altered adhesion/motility, protein turnover, cell differentiation, transcription and translation. It is likely that a RACK1 protein complex directs SET ribosome activity on a mRNA IBES and promotes the assembly of functional ribosomes on specific mRNA sequences, that increase the frequency of translation reinitiation.

In one aspect of this invention, TR cell lines overexpressing the SET response (i.e. TR Outlier Class 3 cells, which exhibit SET responses that are 3 standard deviations larger than the mean of all Class 3 cells) can exhibit growth traits that correlate with stem cell-like, metastatic cancer cells. To remain consistent with the earlier defined TR Class designation, any TR Class 3 cell line that exhibits empirically defined in vitro and in vivo growth traits is termed a TR Class 4 cell. Although cancer cells have the capacity for uncontrolled proliferation and resistance to cell death, few cells have the ability to grow in the absence of a growth-supportive substrate. In an aspect of this invention, a TR Class 4 cell must exhibit a Class 3 Outlier SET response and the in vitro ability to grow in suspension cultures as nonadherent 3D structures. The sphere-forming (i.e. tumorsphere) capacity of a TR Class 4 cell line does not reflect cell aggregation but represents an ability to grow from a small number of nonadherent cells.

In another aspect of this invention, a TR Class 4 cell line must exhibit a Class 3 Outlier SET response, the in vitro ability to form tumorspheres, and in vivo tumor initiating and propagating activities. By definition, a small number of TR Class 4 cells implanted into nude mice is sufficient to initiate and grow a primary xenogenic tumor, that can be dissected into subfragments and propagated as a secondary tumor.

In one aspect of this invention, mammalian cells expressing the TR expression vector can be used to isolate stable cell lines that are “addicted” to SET signaling pathways. By definition, tumors become addicted to an oncogene signaling pathway if that pathway is vital for initiating and/or maintaining tumorigenic growth. For these cells, disruption of the addicted signaling pathway blocks tumor proliferation and reduces viability. For example, preclinical studies show that tumors overexpressing c-Myc (i.e. 5-15% of human breast cancers exhibit Myc gene amplification) can be treated by targeting this pathway, which can result in tumor regression independent of other genetic and epigenetic alterations. However, clinical studies show that metastatic breast cancer cannot be treated by any existing therapy. In most cases, tumor regrowth involves the acquired ability of a tumor cell to efficiently proliferate after the reduction in Myc activity (signal transduction crosstalk). Alternatively, tumors can become addicted to signaling pathways (e.g. VEGFR signaling) that are important for structural integrity. The ability of a tumor to form functional blood vasculature is an essential step in tumor growth beyond a size that prevents passive diffusion of nutrients throughout a tumor. By definition, the cells within a VEGFR dependent tumor are addicted to the size-dependent presence of VEGF. As previously described, tumor adapt to abnormal blood or lymph structures by undergoing metastatic spread, which limits cell starvation and necrotic death.

In a preferred aspect of this invention, overexpression of the SET response in TR Class 4 cells equates with SET signaling addiction, meaning that therapeutic intervention of this pathway could regulate drug efficacy. Drugs targeting the major SET effector in TR Class 4 cells (the SET Ribosome) will block cell signaling pathways controlled by mTORC2. Therefore, the development of drugs that inhibit protein synthesis from the SET ribosome should reduce the synthesis of cell recovery proteins, alter the MAM ribosome/mTORC2/mitochondria signaling pathway, facilitate cell cycle checkpoint control of proliferation, disrupt cell recovery, and enhance mitochondria-specific apoptotic death. As shown in the examples, drugs regulating the SET Ribosome enhanced the in vivo therapeutic index of cytotoxic drugs.

The invention is achieved by evaluating the cellular, biochemical, and molecular targets of the cytotoxic drug and therapies in the tumor microenvironment and by exploiting targeted therapeutics that disrupt the key cell signaling systems linked to resistance to cytotoxic drugs by cancer cells. The invention provides methods and compositions that enhance the efficacy of the cytotoxic drug or therapy, while enhancing the safety of the cytotoxic treatment. Most cytotoxic drugs are toxic when administered as monotherapies, but their toxicity can be potentiated or diminished when used in combination with other agents. In the same manner, cytotoxic therapies, such as radiotherapy, damage normal and cancer cells. According to aspects of the present invention, the combination of treatments may be more or less toxic than the sum of the toxicities of the individual components. The invention describes highly unexpected and novel results showing that the best combinatorial therapeutic effect is observed when low (i.e. subtoxic) doses of the targeted therapeutic is delivered with a therapeutic dose of the cytotoxic agent. Based upon these examples, the present invention describes methods, in vitro and in vivo protocols and compositions based upon dilutions to achieve a maximal treatment effect (i.e. a Biologically Effective Dose or BED).

The use of a cytotoxic or other chemotherapeutic agent, described in any cancer therapeutic regimen, is generally well characterized in the cancer therapy art and their use herein falls under the same considerations for monitoring toxicity, tolerance, and efficacy, as well as for controlling the administration route and dosage, with some adjustments. For example, the actual dose of a cytotoxic agent delivered to a patient depends upon a patient's tolerance for chemotherapy. As one skilled in the art knows, any variety of ex vivo assay can be used to define unacceptable histological or molecular metrics indicative of organ damage. For patients exhibiting toxicity, the cytotoxic drug dosage must be reduced compared to the amount used in the absence of negative outcomes. The present invention anticipates the need for patient-dependent dosing of a cytotoxic agent and defines methods to determine optimal dosing and a preferred pharmaceutical composition that maximally enhances cytotoxic drug efficacy when the cytotoxic drug must be administered at a suboptimal therapeutic concentration.

The invention provides a paradigm for (a) selecting a cytotoxic drug for a specific cancer (e.g. the approved standard(s) of care), (b) evaluating the effect of this agent on the in vitro and in vivo cellular, biochemical and molecular responses in the tumor microenvironment, (c) selecting a combinatorial chemotherapy that blocks the target enzymatic activity induced in the tumor microenvironment so that the inhibition blocks or prevents drug resistance produced by the target(s), (d) titration of varying combinations of the cytotoxic drug(s) and the targeted chemotherapy in preclinical toxicology and efficacy studies using in vitro and in vivo tumor models to define a BED, and (e) to establish the human starting dose thereof. This paradigm for development of novel therapeutic regimens aims for an optimum response using a combination of the two or more drugs selected to achieve the maximum efficacy in the targeted therapeutic when administered with the cytotoxic agent.

Capecitabine and 5-Fluorouracil: Pharmaceutical compositions and treatment methods according of treating a proliferative disorder in a subject to aspects of the present invention include administration of a SET Combination drug with capecitabine (pentyl [1-(3,4-dihydroxy-5-methyltetrahydrifuran-2-yl)-5-fluoro-2-oxo-1H-pyrimidine-4-yl]carbamate) or 5-Fluorouracil (5-FU)/leucovorin.

Compositions including a SET Combination drug with capecitabine or 5-FU/leucovorin according to aspects of the present invention are provided.

Capecitabine is an antimetabolite prodrug of fluorouracil or 5-FU, which has been shown to effectively treat a broad range of cancer types (including breast, esophagus, larynx, gastrointestinal and genitourinary tracts) but also exhibits severe toxicity exemplified by neutropenia, stomatitis, and diarrhea. Capecitabine was developed to reduce 5-FU side effects while also increasing the intratumor drug concentration (requiring a tumor cell enzyme to convert a liver metabolite to the active 5-FU drug).

After administration, oral capecitabine is readily absorbed by the gastrointestinal tract and transported to the liver for processing by a carboxylesterase enzyme into 5′-deoxy-5-fluorocytidine (5′DFCR). The liver cytidine deaminase enzyme converts 5′DFCR to 5′-deoxy-5-fluorouridine (5′DFUR) which is delivered to the blood circulatory system. When 5′DFUR diffuses into a tumor cell, the overexpressed thymidine phosphorylase enzyme converts 5′DFUR into 5-fluorouracil (5-FU). This tumor cell-specific conversion step provides a large concentration of 5-FU which irreversibly inhibits the thymidylate synthetase (TS) enzyme and blocks the conversion of deoxyuridine monophosphate (dUMP) to deoxythymidine monophosphate (TMP). By antagonizing TS, the capecitabine metabolite prevents the synthesis of thymidine nucleotides which stops cell growth, DNA synthesis, and replication.

Capecitabine is currently FDA approved for treatment of metastatic colorectal cancer and metastatic breast cancer. It is also approved in other countries for the treatment of low stage colorectal cancers. Standard dosing as a monotherapy is 1,250 mg/m² orally twice daily (BID), morning and evening for 14 consecutive days in a 3-week cycle.

In a preferred aspect of this invention, tumors and/or tumor metastases are treated with the SET Combination drug and capecitabine. A SET Combination drug is administered prior to, in combination with, or after capecitabine to enhance the cell death of replicating cells. In another aspect, a SET Combination drug is administered orally prior to, in combination with, or after capecitabine to enhance the cell death of replicating cells.

In this invention, tumors and/or tumor metastases are treated with the SET Combination drug and 5-FU. A SET Combination drug is administered prior to, in combination with, or after 5-FU to enhance the cell death of replicating cells. In another aspect, a SET Combination drug is administered orally prior to, in combination with, or after intravenous 5-FU to enhance the cell death of replicating cells.

Cyclophosphamide: Methods of treating a proliferative disorder in a subject according to aspects of the present invention include administering a SET Combination drug with cyclophosphamide (RS-N,N-bis(2-chloroethyl)-1,3,2-oxazaphosphinan-2-amine 2-oxide).

Compositions including a SET Combination drug with cyclophosphamide according to aspects of the present invention are provided.

Cyclophosphamide is a nitrogen mustard alkylating agent, from the oxazophorine chemical group, that is used to treat various cancers (e.g. breast, lung, prostate, ovarian, lymphomas and multiple myeloma) and some autoimmune disorders. As a prodrug, cyclophosphamide is converted in the liver by the cytochrome p450 system (i.e. CYP3A5 and CYP2B6 oxidases) to an active metabolite (4-hydroxycyclophosphamide which tautomerizes to aldophosphamide). After delivery to the circulatory system, aldophosphamide can be transported to tumor cells where it is dephosphorylated by intracellular phosphatase to the two cytotoxically active metabolites, phosphoramide mustard and acrolein (a systemic toxin). Phosphoramide mustard irreversibly alkylates the number 7 nitrogen of guanine, which interferes with DNA replication by forming intrastrand and interstrand DNA crosslinks. However, cyclophosphamide modification of cellular DNA is independent of the mitotic phase and activates DNA repair at multiple cell cycle checkpoints.

Cyclophosphamide is available in both oral (coated tablets) and parental formulations. During development of pediatric oncology drugs, an oral formulation of cyclophosphamide was developed that can be consumed orally after dissolving the powder in water. Oral cyclophosphamide is rapidly absorbed with a bioavailability of >75% with an elimination half-life of 3-12 hrs. It is eliminated primarily as metabolites but 5-25% of the dose is excreted in the urine as unchanged drug. In contrast, intravenous capecitabine results in a maximal metabolite concentration in the plasma 2-3 hr after administration even though infusion rates vary from 30 min to over 24 hr.

In a preferred aspect of this invention, tumors and/or tumor metastases are treated with a SET Combination drug and cyclophosphamide. A SET Combination drug is administered prior to, in combination with, or after cyclophosphamide to enhance the cell death of replicating cells. In another aspect, a SET Combination drug is administered orally prior to, in combination with, or after oral cyclophosphamide to enhance the cell death of replicating cells. A SET Combination drug can be administered orally prior to, in combination with, or after cyclophosphamide injection or infusion to enhance the cell death of replicating cells.

Topotecan and Irinotecan: Methods of treating a proliferative disorder in a subject according to aspects of the present invention include administering a SET Combination drug with topotecan [(S)-10-[(dimethylam ino)methyl]-4-ethyl-4,9-dihydroxy-1H-pyranol [3′,4′,6,7] indolizinol[1,2-b]quinoline-3,14(4H,12H)-dione monohydrochloride] or irinotecan. Camptothecin, which was originally isolated from an extract of the Chinese tree Camptotheca acuminata, is a potent poison of topoisomerase 1, a protein required for DNA synthesis. The camptothecin drug analogs (Camptothecin, Irinotecan, Rubitecan, and Topotecan; CPT, IRT, RBT, and TPT), exhibit two dose dependent modes of action. At low doses (˜20 nM), camptothecin and its derivatives elicit a stress response that includes the activation and synthesis of stress proteins, such as PKCδ, ATR kinase, CIP2/Kap1, p16Ink4a, Nek2, p21 and cdc2, and a transient G2/M checkpoint. In contrast, high doses (>1pM) result in an irreversible Topol-drug complex, permanent Intra-S phase arrest due to DNA strand breakage, cell senescence, and increased apoptosis.

Topotecan is a water soluble, semi-synthetic derivative of camptothecin. As a selective inhibitor of topoisomerase I, high dose topotecan can eliminate DNA supercoiling by preventing religation of single-stranded DNA breaks, but has no effect on topoisomerase II. Topotecan was developed as an alternative to camptothecin which exhibits unacceptable dose limiting toxicity, poor aqueous solubility, and undesirable shelf life stability. Oral topotecan (a capsule) is delivered as the water soluble hydrochloride salt with the remainder of the excipients being gelatin, glyceryl monostearate, hydrogenated vegetable oil, and titanium dioxide (and red iron oxide). The recommended topotecan dose is 1.2-3.1 mg/m² administered daily for 5 days in cancer patients. Topotecan is rapidly absorbed with an oral bioavailability of ˜40% and a peak plasma concentration occurring between 1-2 hr post-administration.

Irinotecan is a water insoluble prodrug derivative of camptothecin that is converted to a biologically active metabolite 7-ethyl-10-hydroxy-camptothecin (SN-38) by a carboxylesterase-converting enzyme that is 1000X more potent than irinotecan. SN-38 inhibits topoisomerase I (topoI) activity by stabilizing the cleavable complex between topoI and DNA, resulting in DNA double-strand breaks that inhibit DNA replication, repair, and trigger apoptotic cell death during S phase.

In a preferred aspect of this invention, tumors and/or tumor metastases are treated with a SET Combination drug and topotecan. A SET Combination drug is administered prior to, in combination with, or after topotecan to enhance the cell death of replicating cells. In another aspect, a SET Combination drug is administered orally prior to, in combination with, or after topotecan to enhance the cell death of replicating cells.

In a preferred aspect of this invention, tumors and/or tumor metastases are treated with a SET Combination drug and irinotecan. A SET Combination drug is administered prior to, in combination with, or after irinotecan to enhance the cell death of replicating cells. In another aspect, a SET Combination drug is administered orally prior to, in combination with, or after intravenous irinotecan to enhance the cell death of replicating cells.

Paclitaxel and Docetaxel: Methods of treating a proliferative disorder in a subject according to aspects of the present invention include administering a SET Combination drug with paclitaxel (5β,20-epoxy-1,2α,4,7β,10β,13α-hexahydroxytax-1-1-en-9-14,10-diacetate 2-benzoate 13-ester with (2R,3S)-N-benzoyl-3-phenylisoserine) or docetaxel. Paclitaxel is a diterpene anticancer compound originally derived from the bark of the Pacific Yew tree. A crude extract of the bark demonstrated antineoplastic activity in preclinical tumor assays, as part of the National Cancer Institute's large-scale screening program. Paclitaxel is one of several cytoskeletal drugs that target tubulin function. Unlike other tubulin-targeting drugs (i.e. colchicine, vincristine, and vinblastine) which disrupt microtubule assembly, paclitaxel prevents microtubule disassembly during metaphase and disrupts mitotic spindle assembly, chromosome segregation, and cell division. As a result of a prolonged activation of the M phase checkpoint, cells become senescent and undergo apoptosis or revert to the G1 phase with cell division (i.e. formation of multinucleated cells).

Docetaxel is a semi-synthetic, second generation taxane derived from a compound found in the European yew tree Taxus baccata. As with paclitaxel, docetaxel binds and stabilizes tubulin, inhibits microtubule disassembly, arrests the cell cycle in late G2/M, and promotes cell senescence and death. However, when compared to paclitaxel, docetaxel exhibited greater affinity for the tubulin binding site, a distinct microtubule polymerization pattern, longer intracellular retention and higher intracellular concentration in target cells. This makes docetaxel a potent and broad anticancer drug that also regulates the expression of pro-angiogenic factors, displays immunomodulatory and pro-inflammatory properties by controlling the expression of inflammatory response mediators.

Paclitaxel is poorly soluble in water (less than 0.01 mg/ml) and other common vehicles used for the parenteral drug administration. While organic solvents can partially dissolve paclitaxel, when a water-miscible organic solvent containing paclitaxel at its saturation solubility is diluted into aqueous infusion fluid, the drug will precipitate. Solubilization with surfactants allows emulsions that can be stably delivered to patients, so paclitaxel is commonly formulated using 50% cremophor, 50% dehydrated alcohol (USP, United States Pharmacopoeia) and diluted in normal saline or 5% dextrose in water to a final concentration of 5% cremophor and 5% dehydrated alcohol or less, for intravenous administration to humans.

In a preferred aspect of this invention, tumors and/or tumor metastases are treated with a SET Combination drug and paclitaxel. A SET Combination drug is administered prior to, in combination with, or after paclitaxel to enhance the cell death of replicating cells. A SET Combination drug can be administered orally prior to, in combination with, or after paclitaxel injection or infusion to enhance the cell death of replicating cells.

In a preferred aspect of this invention, tumors and/or tumor metastases are treated with a SET Combination drug and docetaxel. A SET Combination drug is administered prior to, in combination with, or after docetaxel to enhance the cell death of replicating cells. A SET Combination drug can be administered orally prior to, in combination with, or after docetaxel injection or infusion to enhance the cell death of replicating cells.

Oxaliplatin: Methods of treating a proliferative disorder in a subject according to aspects of the present invention include administering a SET Combination drug with oxaliplatin [oxalato(trans-L-1,2-diaminocyclohexane)platinum]. As an advanced generation platinum(II) analog, oxaliplatin is similar to cisplatin and carboplatin in that it functions by forming Pt-DNA adducts that produce replication damage and enhance cell death. However, the oxaliplatin pro-drug exhibits distinct synergistic interactions, unique pharmacodynamics, reduced toxicity, and activated immunologic responses which differentiate it from the other analogs. The structure of oxaliplatin with oxalate and 1,2-diaminocyclohexane carrier ligands allow the rapid non-enzymatic hydrolysis and displacement of the oxalate group to generate reactive intermediates that modify proteins, RNA and DNA.

In a preferred aspect of this invention, tumors and/or tumor metastases are treated with a SET Combination drug and oxaliplatin. A SET Combination drug is administered prior to, in combination with, or after oxaliplatin to enhance the cell death of replicating cells. A SET Combination drug can be administered orally prior to, in combination with, or after oxaliplatin injection or infusion to enhance the cell death of replicating cells.

Adjunct Therapeutic Treatment—Radiotherapy

Radiation therapy is a standard treatment for controlling unresectable or inoperable tumors and tumor metastases. Improved results have been seen when radiation therapy is combined with chemotherapy. Radiation therapy is based upon the principle that high-dose radiation delivered to a target area will result in the death of replicating cells. The radiation dosage regimen is generally defined in terms of a radiation absorbed dose (Gy), time, and fractionation. The amount of radiation a patient receives will depend upon various factors but the two most important are the location of the tumor in relation to unaffected critical structures or organs and the extent of tumor metastasis. A typical course of treatment for a patient undergoing radiation therapy will be a schedule extending over 1-6 week period, with a total dose of between 10-80Gy administered in a single daily fraction of 1.8-2.0Gy, 5 days a week.

According to aspects of this invention, a tumor and/or a tumor metastasis is treated with a SET Combination drug and radiation. In a preferred aspect, a SET Combination drug is administered prior to, during, or after radiotherapy to enhance the cell death of replicating cells. In another aspect, a SET Combination drug is administered with a cytotoxic agent prior to, during, or after radiotherapy to enhance the cell death of replicating cells.

The radiation source can be either external or internal to the patient being treated. When the source is external to the patient, the therapy can be known as external beam radiation therapy. When the radiation source is internal to the patient, the treatment can be called brachytherapy. Radioactive atoms for use in the context of this invention can be selected from the group including, but not limited to, radium, cesium-137, iridium-192, americium-241, gold-198, cobalt-57, copper-67, technetium-99, iodine-123, iodine-131, and indium-111.

In an aspect of this invention, a SET Combination drug can be administered with a therapeutic antibody component, such that the antibody is labeled with a radioactive isotope to enhance the targeted death of tumor cells. In a preferred aspect, a SET Combination drug is administered with a cytotoxic agent and a therapeutic antibody component, wherein the antibody is labeled with a radioactive isotope to enhance the targeted death of tumor cells.

Adjunct Therapeutics—Chemotherapeutic Drugs

In certain aspects, a SET Combination drug and a cytotoxic agent are co-administered with one or more additional chemotherapeutic drugs, such as, but not limited to, lapatinib, docetaxel, and herceptin. The production, formulation, and use of lapatinib, docetaxel, and herceptin are well known.

Examples of additional chemotherapeutic drugs optionally administered according to aspects of the present invention include, but are not limited to, allopurinol, altretamine, amifostine, nastrozole, arsenic trioxide, bexarotene, bleomycin, busulfan, carboplatin, cisplatin, cisplatin-epinepherine gel, celecoxib, chlorabucil, cladribine, cytarabine liposomal, daunorubicin liposomal, daunorubicin, dexrazoxane, doxorubicin, chlorambucil, cladribine, daunomycin, dexrazorane, epirubicin, estramustine, etoposide phosphate, etoposide, exemestane, goserelin acetate, hydroxyurea, idarubicin, idamycin, ifosfamide, imatinib mesylate, letrozole, leucovorin, leucovorin levamisole, melphalan, mesna, methotrexate, methoxsalen, mitomycin C, mitoxantrone, paclitaxel, pedademase, pentostatin, talc, tamoxifen, temozolomide, teniposide, topotecan, tretinoin, valrubicin, vinorelbine, and zoledronate.

Chemotherapeutic drugs optionally administered according to aspects of the present invention with a SET Therapeutic may be chosen from small molecules, peptides, saccharides, steroids, antibodies (including fragments or variants thereof), fusion proteins, antisense polynucleotides, ribozymes, small interfering RNAs, peptidomimetics, and the like. Examples of antibodies include, but not limited to, antibodies against prostate-specific membrane antigens (such as MLN-591, MLN591RL, and MLN2704), bevacizumab (or other anti VEGF antibodies), alemtuzmab, MLN576 (XR11576), gemtuzumab-ozogamicin, rituximab, and trastuzumab.

SET Combination Drugs

The mechanistic target of rapamycin (i.e. the mammalian target of rapamycin) or mTOR kinase is a serine/threonine kinase, a member of the phosphatidylinositol 3-kinase-related kinase family, that regulates cell growth, cell proliferation, cell motility, cell survival, protein synthesis and transcriptional activation. The mTOR protein is the catalytic subunit of two structurally distinct complexes, mTORC1 and mTORC2, which regulate distinct signaling processes. However, the signaling processes controlled by each complex crosstalk so that mTORC1 prevents mTORC2 activity in growing cells and mTORC2 activates mTORC1 when a cell can reinitiate growth.

In one aspect of this invention, mTORC1 kinase activity indirectly regulates cap-dependent translation by activating a series of 5′ cap recognition proteins that position the 40S ribosomal subunit immediately proximal to a genic ORF. The cap-dependent, rapamycin-sensitive 80S ribosome is responsible for the synthesis of all proteins required for cell cycle progression. Given the cellular functions regulated by this translational activity, this 80S ribosome is termed the “growth ribosome” herein.

In an aspect of this invention, mTORC2 is only activated when mTORC1 is inactive. In a preferred aspect of this invention, mTORC2 is activated by a direct bond to an 80S ribosome localized to the MAM membrane structure. In this subcellular position, the 80S/mTORC2 complex directs the synthesis of injury recovery proteins, controls cellular cytostasis by limiting progression to senescence, and blocks cell death processes. Given that 80S/mTORC2 complex preferentially uses sequence-specific translational mechanisms (i.e. IRES translation initiation and translation reinitiation of dORFs) that are only possible for a subset of mRNA species, termed the 80S/mTORC2 ribosome the “Selective Translation” or SET Ribosome.

The included examples provide evidence that the rapamycin-resistant 80S/mTORC2 complex exhibits unique stress-resistant activity. For example, the 80S/mTORC2 is heat- and cold-resistant whereas the Cap-dependent ribosome is rapidly inactivated by both treatments. In a preferred aspect, the 80S/mTORC2 directs protein synthesis in damaged cells during a cell cycle checkpoint which increases cell viability, promotes injury repair, and promotes the resumption of proliferation.

In a preferred aspect of this invention, tumors treated are composed of a mixture of proliferative and nonproliferative cells, with proliferative cells controlled by mTORC1 activity and nonproliferative cells responding to mTORC2 action. Agents that selectively regulate either mTORC1 or mTORC2 cannot prevent signaling crosstalk by the mTOR catalytic subunit. Therefore, a therapeutic agent of the present invention that blocks mTOR activity in a tumor first inactivates mTORC1 by inducing SET activity via a SET agonist in proliferative cells, producing a cytostatic checkpoint and a second agent, a SET ribosome antagonist, blocks 80S/mTORC2-specific translation to prevent cell recovery and cell cycle progression. The combination of these two drug actions will increase the efficacy of a DNA damaging chemotherapy drug by enhancing the progression from a cytostatic state to a senescent state, which enhances cell death.

In a preferred aspect, the drug combination capable of regulating mTOR activity in tumors is called a “SET Combination drug” and is composed of a “SET Agonist” and a “SET Ribosome Antagonist”. When a SET Combination Drug is combined with a cytotoxic chemotherapeutic, the regimen is particularly well suited to treat drug resistant cancers, metastases and/or recurrent cancers.

In one aspect, the three components are the sole anti-cancer components in the regimen. In another embodiment, the regimen further involves delivery of other active ingredients, which are non-antineoplastic. As used herein, a SET Combination Drug refers, in one aspect, to a first compound or derivative or pharmaceutically acceptable salt thereof, that activates the cellular stress response program that is exemplified by the Selective Translation process and a second compound or derivative or pharmaceutically acceptable salt thereof, that blocks protein synthesis from the Selective Translation Ribosome.

In a preferred embodiment, the SET Agonist is a compound of the invention that can be used to activate protein kinase C function or stimulates cell cycle progression to G2 which induces Selective Translation in a mammalian subject, wherein a compound of the invention, termed a SET Agonist, is administered to the subject in an amount sufficient to increase one or more components of Selective Translation for which modulation of SET signaling respond to activation. In a preferred aspect of this invention, the active pharmaceutical ingredient termed the SET Agonist of a SET Combination Drug activates protein kinase C function or stimulates cell cycle progression to G2 which activates the SET process during cancer therapy and acts with the SET Ribosome Antagonist to improve the efficacy of cytotoxic therapeutics. In another aspect of this invention, the active pharmaceutical ingredient termed the SET Agonist in a SET Combination drug activates protein kinase C function or stimulates cell cycle progression to G2 which induces the SET process systemically in vivo and improves cell recovery after injury which prevents cytotoxic death and increases the safety of cytotoxic therapeutics.

SET Agonist—Polyoxyl hydrogenated castor oils

According to aspects of the present invention, polyoxyl hydrogenated castor oil (PHCO, an accepted commercial excipient) is included in SET Combination drug formulations and administered to a subject.

As shown in the examples of this invention, PHCO included in a SET Combination Drug, polyoxyl 35 castor oil or cremophorEL, was unexpectedly effective as a SET Agonist that stimulates cell cycle progression to G2.

One or more components of a SET Therapeutic is optionally treated to enhance material solubility, by methods illustratively including cosolvency, emulsification, microemulsification, drug complexation with cyclodextrins, carrier mediation using liposomes and nanoparticles, as well as chemical modification to obtain a water soluble derivative or prodrug.

Oral drug formulations, containing lipophilic drugs, can be suspended in an emulsion that can be mixed with an aqueous medium. For effective oral delivery, the emulsion must form droplets consisting of two immiscible liquids that are stabilized by a surfactant agent. Upon arrival at the lumen of the gut, these droplets will disperse into fine droplets that allow a hydrophobic drug to remain in a liquid state. Therefore, the surfactant or emulsifying agent stabilizes and solubilizes, possibly in conjunction with the other components, the active drug or pharmaceutical agent. The surfactant or emulsifying agent used in a formulation can be a single product, or a combination of two or more of products. Examples of surfactant and emulsifying agents include, but are not limited to, polyoxyethlene sorbitan fatty acid esters, polyoxyethlene alkyl ethers, polyoxyethylene castor oil derivatives, polyoxyethlene stearates, and saturated polyglycolized glycerides. These pharmaceutically acceptable surfactants are well known in the art and are available from commercial sources.

PHCO included in compositions and administered according to aspects of this invention is a non-ionic surfactant prepared by converting castor oil to hard oil by hydrogenation, and condensing the hard oil with ethylene oxide. PHCO is classified according to the average mole of added ethylene oxide, with the average mole of added ethylene oxide being preferably 30, 35, 40, 50, and 60. For example, if each mole of castor oil is reacted with an average of 35 moles of ethylene oxide, the resulting mixture is termed polyoxyl 35 castor oil (or cremophorEL). Similarly, the use of 40 moles of ethylene oxide results in a product termed polyoxyl 40 hydrogenated castor oil (or cremophorRH). In each case, the resulting product is a mixture of polyethylene glycol ethers, polyethylene glycol esters of ricinoleic acid, polyethylene glycols, and polyethyelene glycol ethers of glycerol. While chromatography is used to reduce the water soluble ionic, metallic, and oxidizing impurities in a PHCO (which catalyze the decomposition of pharmaceutical agents) an unresolved lipophilic mixture remains in the commercial product.

In an aspect of this invention, PHCO is included as a SET Agonist which activates the SET process during cancer therapy and acts with the SET Ribosome Antagonist to improve the efficacy of cytotoxic therapeutics. Additionally, the PHCO serves as a nonionic detergent-like surfactant that improves the solubility of hydrophilic compounds and as a SET Agonist which acts with the SET Ribosome Antagonist to improve the efficacy of cytotoxic therapeutics.

In a preferred aspect, polyoxyl 35 castor oil (cremophorEL) serves as the nonionic surfactant for hydrophobic drugs and as a SET Agonist. The detergent-like polyoxyl 35 castor oil micelles (cmc is 0.0095 w/v%) act by enhancing cell membrane fluidity. In addition to activating SET, polyoxyl 35 castor oil may inhibit P-glycoprotein transporter function (blocking multidrug resistance and enhancing intestinal absorption of certain hydrophobic agents) and disrupt lipoprotein complexes in the blood (altered HDL and LDL physical traits). In another aspect, polyoxyl 40 castor oil (cremophorRH) serves as the nonionic surfactant for hydrophobic drugs and as a SET Agonist.

A preferred aspect of this invention is the inclusion of a PHCO in a SET Combination Drug to activate a systemic in vivo SET process which improves cell recovery after injury by preventing cytotoxic death and increasing the safety of cytotoxic therapeutics. According to an aspect of this invention, polyoxyl 35 castor oil is included as the SET Agonist in a SET Combination Drug to activate the SET process which improves in vivo cell cycle progression. According to an aspect of this invention, polyoxyl 40 castor oil is included as the SET Agonist in a SET Combination Drug to activate the SET process which improves in vivo cell cycle progression.

SET Agonist—Phorbol esters

Phorbol is a natural plant-derived organic compound of the tigliane family of diterpenes, which acts as a molecular mimic of diacylglycerol (DAG). As with DAG, phorbol esters modulate cell signaling pathways by directly activating a family of serine/threonine protein kinases, collectively known as the protein kinase C (PKC) family.

As shown in the examples of this invention, SET activation stimulates protein synthesis controlled by the SET Ribosome and activates an innate immune response in a mouse xenogenic tumor model. Consistent with these examples, the phorbol ester TPA has been shown to induce phenotypic changes in the epidermis similar to those observed in a cutaneous inflammatory response. In this system, TPA directly mimics the natural response of the skin to injury, including the induction of both IL-1α release and de novo IL-1 gene expression (localized inflammation). This in vivo response is consistent with cell responses regulated by the MAM/mitochondria/MAVS complex when bound to a Nod-like receptor family pyrin domain containing protein (NLRPs) activated by pathogen or damage signal binding. Formation of the complex results in signaling to the inflammasome producing an innate immune inflammatory response to the pathogenic signal.

According to aspects of the present invention, a SET Agonist component of a SET Combination Drug is a phorbol ester which activates SET during cancer therapy and acts with the SET Ribosome Antagonist to improve the efficacy of cytotoxic therapeutics. According to aspects of the present invention, a preferred compound, phorbol-12-myristate-13-actate (PMA or TPA) is included in a SET Combination Drug to activate the SET process during cancer therapy and work with the SET Ribosome Antagonist to improve the efficacy of a cytotoxic therapeutic.

Given that PKC activation can block many cytotoxic processes, a preferred aspect of this invention is the use of a phorbol ester in a SET Combination drug to activate a systemic in vivo SET process which improves cell recovery after injury by preventing cytotoxic death and increasing the safety of cytotoxic therapeutics. One aspect of this invention is the inclusion of phorbol-12-myristate-13-actate (PMA or TPA) as a SET Agonist in a SET Combination drug to activate the SET process which improves in vivo cell recovery responses.

SET Agonist—Bryostatin Compounds

The bryostatins are a group of macrolide lactones first isolated from extracts of a species of bryozoan, Bulula neritina. The bryostatin compounds are potent modulators of protein kinase C (PKC) activity. To date, at least 20 different bryostatin analogs have been identified. As with other PKC activators, bryostatin 1 exhibits a broad range of conditional in vitro and in vivo responses. Bryostatin 1 is a non-typical activator of the classic and novel PKCs when given in short exposures; however, extended exposure results in isoform-specific PKC inactivation that inhibits cell growth (resulting in differentiation and/or apoptotic death). While preclinical animal studies indicated that a bryostatin might treat cancer, phase II human clinical trials did not detect any therapeutic activity for bryostatin 1 when given as a monotherapy or in combination with other chemotherapeutic agents. These results support the theory that bryostatin activation of SET is not sufficient to block mTORC2 kinase function which permits tumor recovery after injury by cytotoxic agents.

Further support for a role of SET induction in enhanced cell recovery, bryostatin 1 has been shown to activate the a-secretase enzyme which cleaves the amyloid precursor protein (APP), generating non-toxic protein fragments. On the basis of this result, bryostatin 1 was tested for an ability to prevent neurodegeneration associated with APP processing (i.e. Alzheimer's disease or AD). Preclinical testing in AD transgenic animals (three rodent lines containing different human AD-causing mutations) showed that bryostatin 1 reduced amyloid-β plaques and neurofibrillary tangles, restored neuronal synapses, and protected against memory loss. In related preclinical work, bryostatin 1 also enhanced and restored memory by regenerating synapses previously destroyed by stroke, head trauma, or aging. These activities supporting the theory that PKC-mediated SET activation enhances injury recovery processes which increase cell viability by limiting cytotoxicity.

Bryostatin 2 is a structurally distinct bryostatin analog that associates with the phorbol ester binding site of PKC and exhibits an enzyme binding constant that is 10 times the magnitude of bryostatin 1 (reflects a greater affinity of bryostatin 2 for PKC). Preclinical studies show that bryostatin 2 inhibits DNA synthesis (and cell growth), induces the release of arachidonic acid from treated cells, and acts synergistically with B cell stimulatory factor-1 to cause differentiation of naïve, resting lymph node T cells into cytotoxic T lymphocytes.

In an aspect of this invention, the SET Agonist in the SET Combination drug is a bryostatin derivative that activates SET during cancer therapy and acts with the SET Ribosome Antagonist to improve the efficacy of a cytotoxic therapeutic. In other aspects, bryostatin 1 is the preferred compound used in a SET Combination Drug to activate the SET process during cancer therapy and acts with the SET Ribosome Antagonist to improve the efficacy of a cytotoxic therapeutic. In other aspects, bryostatin 2 is the compound used in a SET Combination Drug to activate the SET process during cancer therapy and acts with the SET Ribosome Antagonist to improve the efficacy of a cytotoxic therapeutic.

In an aspect of this invention, a SET Agonist enhances cell recovery, reduces side effects, and improves drug safety by activating the SET process. Given that PKC activation can block many cytotoxic responses, a preferred aspect of this invention is the use of a bryostatin in a SET Combination Drug to activate a systemic in vivo SET process which improves cell recovery after injury by preventing cytotoxic death and increasing the safety of cytotoxic therapeutics. One aspect of this invention is the use of bryostatin 1 as the SET Agonist in a SET Combination Drug to activate the SET process which improves in vivo cell recovery responses. In another aspect of this invention, bryostatin 2 is used as the SET Agonist in a SET Combination Drug to activate the SET process which improves in vivo cell recovery responses.

SET Ribosome Antagonist—Anisomycin

It is contemplated that candidate molecules for inhibiting SET Ribosome protein synthesis can be designed de novo or may be identified by functional assays using pre-existing ribosome inhibitors. It is contemplated that many of the approaches useful for designing de novo molecules may also be useful for modifying existing molecules after functional activity on the SET Ribosome has been empirically determined. A variety of agents bind the 80S ribosome and disrupt protein synthesis including for example, but not limited to: chloramphenicols, macrolides, lincosamides, streptogramins, althiomycins, oxazolidinones, nucleotide analogs, thiostreptons (e.g. the micrococcin family), peptides, glutarimides, trichothecenes, TAN-1057, pleuromutilins, hygromycins, betacins, eveminomicins, boxazomycins and fusidanes.

Anisomycin or (2R,3S,4S)-4-hydroxy-2-(4-methoxybenzyl)-pyrrolidin-3-yl acetate) is an antibiotic produced by Streptomyces griseolus that inhibits eukaryotic protein synthesis. The pyrrolidine ring of anisomycin is important for interaction with the 60S ribosomal subunit, binding at the junction of the aminoacyl (A site) and peptidyl (P site). In this site, anisomycin blocks peptide bond formation and suppresses the peptidyltransferase reaction (preventing elongation and disrupting polysome stability).

Anisomycin has been used extensively as a neuromodulator that regulates memory retention and recovery. In addition to its ability to block translation, anisomycin has also been shown to be a potent activator of the mitogen-activated protein kinase (MAPK) signaling system, in particular, the stress-activated p38 mitogen activated protein kinase (p38MAPK) and c-Jun NH2-terminal kinase (JNK) at doses that do not significantly impact protein synthesis.

The present invention describes a SET Combination Drug composed of a SET Agonist and a SET Ribosome Antagonist, in which the combination of the SET affective drugs inactivate mTOR kinase activity and improves the efficacy of a cytotoxic therapeutic. In an aspect of this invention, the SET Ribosome Antagonist is an inhibitor of ribosomal activity for which the “Biologically Effective Dose” (BED) produces 100% SET ribosome inhibition but is well below lethal concentrations in animals.

As shown in the examples, anisomycin selectively blocks translation from the activated 80S/mTORC2 ribosome (the SET Ribosome) with a 50% inhibitory concentration or IC50 of <50 nM and an IC100 (100% SET Ribosome inhibition) of 1 μM. In this example, absolute inhibition of the SET Ribosome at a 1 μM dose is well below the LD50 of 35 μM (intramuscular) and 500 μM (oral) for mice and LD50 of 200 μM (intramuscular) and 1 mM (oral) for monkeys.

In an aspect of this invention, the SET Ribosome Antagonist is a compound of the invention that can be used to block protein synthesis from the SET Ribosome in a mammalian subject, wherein a compound of the invention, termed a SET Ribosome Antagonist, is administered to the subject in an amount sufficient to eliminate all SET after which activation of the SET Ribosome produces recovery protein synthesis. In a preferred aspect, anisomycin is included as a SET Ribosome Antagonist in a SET Combination Drug that blocks protein synthesis from the SET Ribosome during cancer therapy and acts with the SET Agonist to improve the efficacy of cytotoxic therapeutics.

SET Ribosome Antagonist—Emetine

Emetine or (2S, 3R, 11bS)-2-{{(1R)-6,7-dimethoxy-1,2,3,4-tetrahydroisoquinolin-1-yl]methyl}-3-ethyl-9,10-dimethoxy-2,3,4,6,7,11b-hexahydro-1H-pyrido[2,1-a] isoquinoline) is the principal alkaloid of ipecac, isolated from the ground roots of Cephaelis ipecacuanha. Some of the earliest uses of emetine were as an emetic, an expectorant, an antiparasitic drug, and as an antibacterial/antiviral agent. However, at physiological pH, emetine irreversibly inhibits mammalian, yeast and plant protein synthesis in a concentration and time-dependent manner by binding to the rpS14 protein in the 40S subunit.

The rpS14 protein is a vital ribosome maturation factor that is involved in the processing of the 20S pre-rRNA to 18S rRNA and maturation of 43S preribosomes to 40S. In the 80S ribosome, rpS14 promotes mRNA assembly on the 40S subunit by binding to a conserved helix structure in the 18S rRNA and to mRNA sequence elements. At the 40S platform structure, near the mRNA exit tunnel, rpS14 also controls the conformational changes in the 40S subunit needed to align various viral IRES RNA elements in the 40S decoding groove.

Upon exposure to the 80S ribosome, emetine binds rpS14 on an exposed basic carboxy-terminal sequence which blocks 40S subunit binding of the mRNA. As an antiparasitic drug, emetine blocked growth and induced apoptosis at sub-cytotoxic concentrations. As an antiviral, emetine blocked assembly of the dengue virus IRES RNA structure on the 40S subunit which prevented cap-independent viral protein synthesis and replication.

The present invention describes a SET Combination Drug composed of a SET Agonist and a SET Ribosome Antagonist, in which the combination of the SET affective drugs inactivate all mTOR kinase activity and improves the efficacy of a cytotoxic therapeutic. In an aspect of this invention, the SET Ribosome Antagonist is an inhibitor of ribosomal activity for which the “Biologically Effective Dose” (BED) produces 100% SET ribosome inhibition but is well below lethal concentrations in animals. As shown in the examples, emetine selectively blocks translation from the activated 80S/mTORC2 ribosome (the SET ribosome) with a 50% inhibitory concentration or IC50 of 175 nM and an IC100 (100% SET ribosome inhibition) of 2.5 μM. In this example, absolute inhibition of the SET Ribosome at a 2.5 μM dose is well below the LD50 of 5 μM (intravenous) and 35 μM (oral) for rabbits, LD50 of 58 μM (subcutaneous) for mice, and LD50 of 216 μM (oral, 120 mg/kg) and 174 uM (subcutaneous, 95 mg/kg) for rats.

In an aspect of this invention, the SET Ribosome Antagonist is a compound of the invention that can be used to block protein synthesis from the SET Ribosome in a mammalian subject, wherein a compound of the invention, termed a SET Ribosome Antagonist, is administered to the subject in an amount sufficient to eliminate all SET after which activation of the SET Ribosome produces recovery protein synthesis. In a preferred aspect, emetine is the active pharmaceutical ingredient, termed the SET Ribosome Antagonist, in a SET Combination drug that blocks protein synthesis from the SET Ribosome during cancer therapy and acts with the SET Agonist to improve the efficacy of cytotoxic therapeutics.

Agent Administration

An effective amount of one or more pharmaceutical compositions of the present invention may be contained in one aspect, such as a single pill, capsule, premeasured intravenous dose, or pre-filled syringe for injection. Alternatively, the composition will be prepared in individual dose forms where one unit, such as a pill, will contain a suboptimal dose but the patient may be instructed to take two or more unit doses per treatment. Concentrates for later dilution by the end user may also be prepared, for instance for intravenous (IV) formulations and multi-dose injectable formulations.

A variety of administration routes are available for use in the treatment of a human or animal patient. The particular mode selected will depend upon the particular condition being treated, the dosage required for therapeutic efficacy, and composition of the combinatorial formulation. The methods of this invention may be practiced using any mode of administration that is medically acceptable (i.e. a mode that provides an optimal therapeutic activity from the pharmaceutical active compounds without enhancing any clinically unacceptable adverse reactions).

Preferred administration routes include orally, parentally (e.g. subcutaneous, injection, intravenous, intramuscular, intrasternal or infusion), by inhalation spray, topically, by absorption through a mucous membrane, or rectally. More preferably, the compounds of the present invention are administered orally. In another aspect, the administration route is parenterally (i.e. intravenously, intraperitoneally, infusion or injection). In one aspect of the invention, the compounds are administered directly to a tumor by tumor injection. In another aspect, the compounds are administered systemically.

For oral administration as a suspension or emulsion, the compositions can be prepared according to techniques well-known in the art of pharmaceutical formulation. The compositions can contain microcrystalline cellulose for bulk, alginic acid or sodium alginate as a suspending agent, methylcellulose as a viscosity enhancer, sweeteners, or flavoring agents. As immediate release tablets, the compositions can contain microcrystalline cellulose, starch, magnesium stearate, lactose, or other excipients, binders, extenders, disintegrants, diluents, and lubricants known in the art.

For parenteral administration as an injectable solution or suspension, the compositions can be formulated according to techniques well-known in the art, using suitable dispersing or wetting and suspending agents. Solutions or suspensions are prepared in water, isotonic saline (PBS), or mixed with an inert surfactant (PCHO). Dispersions can also be prepared in glycerol, liquid polyethylene, glycols, DNA, vegetable oils, triacetin, and mixtures thereof.

Under ordinary conditions of storage and use, injectable preparations contain an inert preservative to prevent the growth of microorganisms. A preservative can be a substance or process added to or applied to a pharmaceutical composition to prevent decomposition by microbial growth or undesirable chemical reactions. In general, preservation is implemented by either chemical additives or physical processing. In a preferred aspect, methods and compositions are described for identifying inert chemical additives that can be used as preservatives that do not regulate the SET process by either stimulating or suppressing mTOR-specific translation.

As a prophylaxis to treat post-chemotherapy infections due to immunosuppression, antimicrobial treatment (i.e. antibiotics) will be used after chemotherapy. Similarly, targeting cancer associated viruses and bacteria can prevent the initiation of gastric, cervical, hematopoietic, liver, and brain cancer. In a preferred aspect of this invention, antimicrobials and antivirals are administered to control the SET process in the subject by either stimulating or suppressing mTOR-regulated translation. In another aspect, methods and compositions are described for identifying inert antibiotics that do not control the SET process by either stimulating or suppressing mTOR-regulated translation and can be safely added to the pharmaceutical formulations described in this invention to prevent microbial infections.

A wide variety of pharmaceutical forms can be employed. Thus, if a solid carrier is used the preparation can be tableted, placed in a hard gelatin capsule, a powder, pellet form, in the form of a troche, or lozenge. The amount of solid carrier will vary widely but preferably will be in the form of a syrup, emulsion, soft gelatin capsule, sterile injectable solution, or suspension in an ampule, vial, or nonaqueous liquid suspension. To obtain a stable water soluble dose form, a pharmaceutically acceptable salt of the SET combination drug and cytotoxic agent can be dissolved in an aqueous solution of an organic or inorganic acid or base. If a soluble salt form is not available, the SET combination drug and cytotoxic agent may be dissolved in a suitable co-solvent or combinations thereof. Examples of such suitable cosolvents include, but are not limited to, alcohol, propylene glycol, polyethylene gycol 300, polysorbate 80, glycerin and the like in concentration ranging from 0-60% of the total volume.

Excipients, diluents, or carriers contemplated for use in these compositions are generally known in the pharmaceutical formulary arts. Reference to useful materials can be found in well-known compilations such as Remington's Pharmaceutical Sciences, Mack Publishing Co., Easton, PA. The nature of the composition and the pharmaceutical excipient, diluent, or carrier will depend upon the intended route of administration, for example by intravenous and intramuscular injection, parenterally, topically, orally or by inhalation. For parenteral administration, the pharmaceutical composition will be in the form of a sterile injectable liquid such as an ampule or an aqueous or nonaqueous liquid suspension. For topical administration the composition will be in the form of a cream, ointment, lotion, paste, spray or drops suitable for administration to the skin, eye, ear, nose or genitalia. For oral administration the pharmaceutical composition will be in the form of a tablet, capsule, powder, pellet, troche, lozenge, syrup, liquid, or emulsion.

The pharmaceutical excipient, diluent, or carrier employed may be either a solid or liquid. When the pharmaceutical composition is employed in the form of a solution or suspension, examples of appropriate carriers or diluents include: for aqueous systems, water; for non-aqueous systems: ethanol, glycerin, propylene glycol, olive oil, corn oil, cottonseed oil, peanut oil, sesame oil, liquid paraffins, and mixture of water; for solid systems: lactose, terra alba, sucrose, talc, gelatin, agar, pectin, acacia, magnesium stearate, stearic acid, kaolin and mannitol; and for aerosol systems: dichlorodifluoromethane, chlorotrifluoroethane and compressed carbon dioxide. Also, in addition to the pharmaceutical carrier or diluent, the instant compositions may include other ingredients such as stabilizers, antioxidants, preservatives, lubricants, suspending agents, viscosity modifiers and the like, provided that the additional ingredients do not have a detrimental effect on the pharmacodynamics, pharmacokinetics or therapeutic action of the instant compositions. Similarly, the carrier or diluent may include time delay material well known in the art, such as glyceryl monostearate or glyceryl distearate alone or with a wax, ethylcellulose, hydroxypropylmethylcellulose, methylmethacrylate and the like.

The compounds of the invention are capable of forming both pharmaceutically acceptable acid addition and/or base salts. Base salts are formed with metals or amines, such as alkali and alkaline earth metals or organic amines. Examples of metals used as cations are sodium, potassium, magnesium, calcium and the like. Also included are heavy metal salts such as, for example, silver, zinc, cobalt, and cerium. Examples of suitable amines are N,N′-dibenzylethylenediamine, chloroprocaine, choline, diethanolamine, ethylene-diamine, N-methylglucamine, and procaine. Pharmaceutically acceptable acid addition salts are formed with organic and inorganic acids. Examples of suitable acids for salt formation are hydrochloric, sulfuric, phosphoric, acetic, citric, oxalic, malonic, salicylic, malic, gluconic, fumaric, succinic, ascorbic, maleic, methane-sulfonic, and the like. The acid salt is prepared by contacting the free base form with a sufficient amount of the desired acid to produce either a mono or di, etc salt in the conventional manner. The free base forms may be regenerated, as needed, by treating the salt form with a base. The free base forms may differ from their respective salt forms in certain physical properties such as solubility in polar solvents, but the pharmaceutical salt forms should be otherwise equivalent to the respective free base forms for the practice of this invention.

The pharmaceutically active compounds will be administered in therapeutically effective amounts. A therapeutically effective amount means that amount necessary to attain the desired response, such as to delay the onset of, inhibit the progression of, or halt altogether, the onset or progression of the proliferative disease being treated. Such therapeutic administration, in particular for the cytotoxic agent, will depend upon the particular condition being treated, the severity of the condition (e.g. tumor stage), and individual patient parameters such as age, physical condition, size, weight, concurrent disease states, and concurrent treatments. These factors are well known in the art and can be addressed with no more than routine clinical evaluation. For the cytotoxic agent, it is preferred that a maximum tolerated dose be used, that is, the highest safe dose according to sound medical judgment and empirical human trials. It will be understood by those with ordinary skill in the art, that a lower dose or tolerable dose may be administered for technical, psychological, or for virtually any justifiable medical reason.

It will be appreciated that the actual preferred dosage of the SET combination drug and cytotoxic agent used in the compositions and methods of treatment of the present invention will vary according to the particular components being used, the particular composition formulated, the mode of administration, and the particular site, host, and proliferative condition being treated. Optimal dosages for a specific pathological condition in a particular patient may be ascertained by those of ordinary skill in the antineoplastic art using conventional dosage determination tests in view of the above experimental data. For example, the dose administered by parenteral delivery may range from 2-50 mg/m² of body surface area per day for one to five days, preferably repeated every three to four weeks for four courses of treatment. For continuous IV administration, the dose may be about 0.5 mg/m²/day for 5 to 21 days. For oral administration, the dose may range from 20-150 mg/m² of body surface area for one to five days, with courses of treatment repeated for appropriate intervals.

Molecular biological techniques, biochemical techniques, and microorganism techniques as used herein are well known in the art and commonly used, and are described in, for example, Sambrook J. et al. (1989), Molecular Cloning: A Laboratory Manual, Cold Spring Harbor and its 3rd Ed. (2001); Ausubel, F. M. (1987), Current Protocols in Molecular Biology, Greene Pub. Associates and Wiley-Interscience; Ausubel, F. M. (1989), Short Protocols in Molecular Biology: A Compendium of Methods from Current Protocols in Molecular Biology, Greene Pub. Associates and Wiley-Interscience; Innis, M. A. (1990), PCR Protocols: A Guide to Methods and Applications, Academic Press; Ausubel, F. M. (1992), Short Protocols in Molecular Biology: A Compendium of Methods from Current Protocols in Molecular Biology, Greene Pub. Associates; Ausubel, F. M. (1995), Short Protocols in Molecular Biology: A Compendium of Methods from Current Protocols in Molecular Biology, Greene Pub. Associates; Innis, M. A. et al. (1995), PCR Strategies, Academic Press; Ausubel, F. M. (1999), Short Protocols in Molecular Biology: A Compendium of Methods from Current Protocols in Molecular Biology, Wiley, and annual updates; Sninsky, J. J. et al. (1999), PCR Applications: Protocols for Functional Genomics, Academic Press; Special issue, Jikken Igaku [Experimental Medicine] “Idenshi Donyu & Hatsugenkaiseki Jikkenho [Experimental Method for Gene introduction & Expression Analysis]”, Yodo-sha, 1997. Relevant portions (or possibly the entirety) of each of these publications are herein incorporated by reference.

Amino acid or nucleotide deletion, substitution or addition of the polypeptide of the present invention can be carried out by site-specific mutagenesis methods which are well-known techniques. One or several amino acid or nucleotide deletions, substitutions or additions can be carried out in accordance with methods described in Molecular Cloning, A Laboratory Manual, Second Edition, Cold Spring Harbor Laboratory Press (1989); Current Protocols in Molecular Biology, Supplement 1 to 38, John Wiley & Sons (1987-1997); Nucleic Acids Research, 10, 6487 (1982); Proc. Natl. Acad. Sci., USA, 79, 6409 (1982); Gene, 34, 315 (1985); Nucleic Acids Research, 13, 4431 (1985); Proc. Natl. Acad. Sci. USA, 82, 488 (1985); Proc. Natl. Acad. Sci., USA, 81, 5662 (1984); Science, 224, 1431 (1984); PCT W085/00817(1985); Nature, 316, 601 (1985); and the like.

Having described the invention in detail, it will be apparent that modifications and variations are possible without departing the scope of the invention defined in the appended claims. Furthermore, it should be appreciated that all examples in the present disclosure, while illustrating the invention, are provided as non-limiting examples and are, therefore, not to be taken as limiting the various aspects of the invention so illustrated.

### EXAMPLES

The following non-limiting examples demonstrate that regulation of the SET Ribosome creates a chronic stress state in a tumor that enhances the therapeutic activity of a first-line oncology drug and are provided to further illustrate the present invention.

### Example 1

1A. Defining the Characteristics of a TR Metastatic Cancer Cell Model.

Advanced and aggressive tumors are thought to contain a unique population of cancer cells that exhibit stem cell traits, such as an ability for self-renewal, the capacity to evolve and give rise to novel stem cell progeny, enhanced resistance to cell damage, and a tumor initiating capacity. Although cancer stem cells (CSCs) represent a small fraction of any tumor, they constitute the population needed to create distant, heterogeneous metastases. Because high TR Class number (and elevated SET Ribosome activity) correlates with increased G2/M damage repair potential, improved cell viability, and drug resistance; multiple mTR and hTR cell lines were used to compare SET Ribosome responses with established in vitro and in vivo CSC properties. By example, a TR Metastatic Cancer Cell model will exhibit a series of measurable traits including: (1) it was derived from a small outlier population of a parental TR cell line (top 1-5% SET induction), (2) it demonstrated drug and stress resistance that correlated with a statistically elevated SET Ribosome activity in cell-based TR assays (termed a Class 4 response), (3) it exhibited Clonal Evolution that resulted in highly significant changes in SET Ribosome activity (creating a novel TR Outlier response) as a result of low density selective growth, such as repeated single cell colony formation and the generation of nonadherent tumorspheres from a small number of cells, (4) it displayed in vivo tumor initiating activity following serial xenotransplantation into nude mice, (5) it formed xenogenic tumors that exhibited in vivo regulation of SET-specific translation from the TR expression cassette, and (6) it formed xenogenic tumors with an elevated growth rate and resistance to cytotoxic drug treatment. For example, a TR metastatic colorectal cancer (CRC) cell model clone would be isolated from a parental CRC cell line (such as HCTZ 16) and exhibit each of these traits. As shown in subsequent sections, one example of a TR metastatic CRC cell model is hTRdm-fLUC#32.

1B. Identifying and Isolating TR Cell Lines Derived from a Small Outlier Population

(a) Cell Culture Materials

All mammalian cells were maintained at 37° C., 5% CO2 in appropriate complete growth medium (specified below for each cell line).

DMEM: 1 packet/L DMEM powder (Invitrogen Life Technologies); 3.7 g/L sodium bicarbonate; 30-50 mg/L gentamicin sulfate; 10% Fetal Bovine Serum (FBS) or DMEM, high glucose liquid (ThermoFisher Scientific); gentamicin sulfate; 10% FBS.

MEM: 1 packet/L MEM powder with Earle's salts (Invitrogen Life Technologies); 1.5 g/L sodium bicarbonate; 10 mL/L 100 mM sodium pyruvate solution (Invitrogen Life Technologies); 10 mL/L 10 mM MEM nonessential amino acid solution (Invitrogen Life Technologies); 30-50 mg/L gentamicin sulfate; 10% FBS or MEM liquid (ThermoFisher Scientific); gentamicin sulfate; sodium pyruvate; MEM nonessential amino acids; 10% FBS.

RPMI: 1 packet/L RPMI 1640 powder (Invitrogen Life Technologies); 10 mL/L 100 mM sodium pyruvate solution (Invitrogen Life Technologies); 30-50 mg/L gentamicin sulfate; 10% FBS or RPMI liquid (ThermoFisher Scientific); sodium pyruvate; gentamicin sulfate; 10% FBS.

(b) Transfection of Mammalian Cells

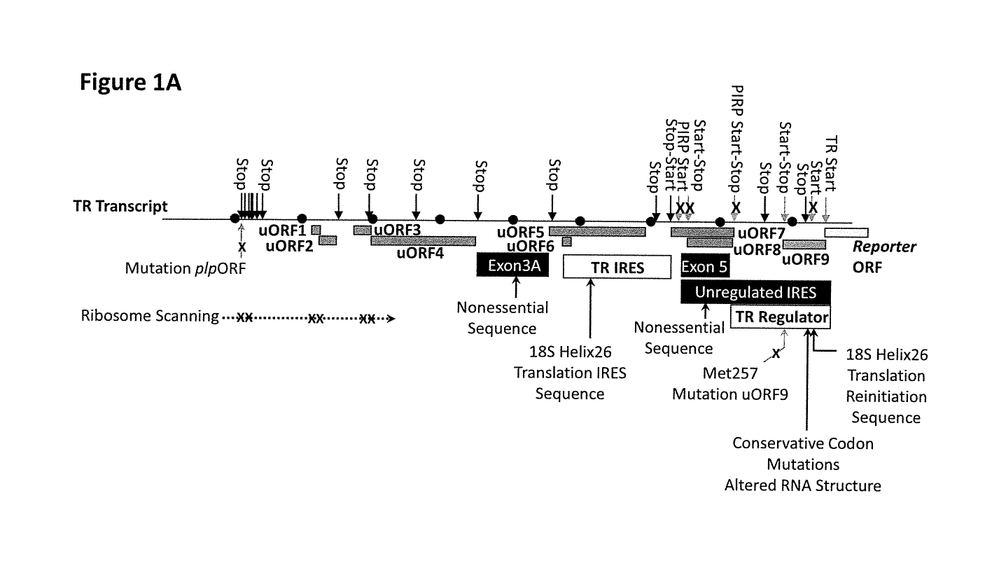

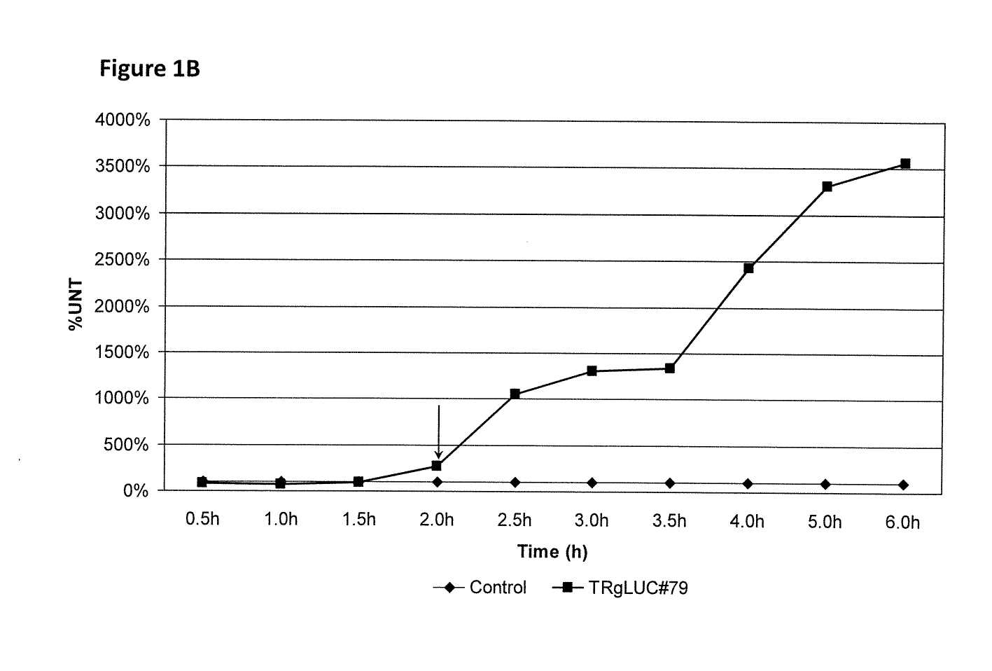

The following expression plasmids were used to transfect mammalian cells: pCMV-fLUC, pmTRdm-fLUC, phTRdm-fLUC, pmTRdm-gLUC, and phTRdm-gLUC. These plasmids contain the TR expression cassette (FIG. 1A) which controls protein synthesis by the SET Ribosome using a rare translation control system. Polysomal profiling demonstrates that 3% of mammalian mRNAs (220-350 species) are actively translated after suppression of Cap-dependent translation. Internal translation initiation from these transcripts is commonly detected using transgenes that are concurrently translated by Cap-dependent and Cap-independent processes. Since these translational activities map to distinct cell cycle phases, existing technology can only qualitatively measure a translation event. The TR expression sequence contains an abundance of upstream open reading frames (uORFs) and translation termination codons that prevent Cap-dependent ribosome scanning to a downstream ORF of a reporter gene. This requires that TR translation occurs by an internal initiation process.

To define internal regulatory elements, deletion mapping identified an Internal Ribosome Entry Sequence (TR IRES) in Exon 4 that controls the translation of two internal ORFS (iORFs) during G2/M from the parental gene (site directed mutagenesis was used to inactivate these ORFs in the TR sequence). The specificity of this process was shown by the fact that the Exon 4 IRES was flanked by nonessential sequences (exons 3b and 5) that could be deleted without affecting SET. However, deleting Exons 5-7 disrupted SET and produced constitutive translation initiation from Exon 4. Subsequent sequence analysis identified a putative TR IRES element in Exon 4 with sequence identity to an IRES element in the GTX gene and homology to an 18S rRNA helix 26 sequence required for IRES function (Table 1). Since Exons 5-7 did not exhibit transcriptional or translational activity when cloned into mammalian expression vectors, this indicates that Exons 6-7 must contain a negative regulator of the Exon 4 TR IRES.

Sequence analysis of Exon 7 identified a segment with identity to the translation “Reinitiation” sequence present in multiple viral genomes (TR Regulator, FIG. 1 and Table 1). In viruses, this sequence functions during G2/M to direct translation from ORFs that are normally blocked during Cap-dependent translation. Moreover, mRNA secondary structures in the vial RNAs (with minimal cross-species structural homology) are needed to position this sequence and an initiation codon on the 40S ribosome subunit. To examine translational regulation by the TR Exon 7 sequence, site directed mutagenesis was used to introduce mutations in RNA structures encompassing this Reinitiation element and any mutation affecting the native Exon 7 RNA structure dysregulated TR SET (Table 2). This proved that a constitutive TR IRES in Exon 4 is controlled by a TR Regulator in Exon 7 to produce G2-specific SET and reporter protein expression (fLUC, gLUC) from the TR expression cassette. No other mammalian mRNA is known to contain this bifunctional regulatory system.

Mammalian transfections were performed using the nonlipidic Transfectol transfection reagent (Continental Lab Products) or FuGENE6 lipid-based transfection reagent (Roche Applied Science) as instructed by the vendor. Prior to a Transfectol transfection, mammalian cells were grown in 100mm dishes to 50% confluence and fed with the appropriate growth medium supplemented with 2.5-5% FBS 1-3 hrs prior to addition of the DNA/transfection reagent mixture. The mixtures were prepared by first combining 1 mL Diluent with 15ps plasmid DNA and vortexing, then adding 60 μL Transfectol and vortexing for 5sec. Each DNA/transfection reagent mixture was incubated at RT for 15 min, then added dropwise to cells. Cells were grown in the presence of the DNA/Transfectol mixtures for 2-16 hr. At this time, the culture medium was replaced with complete growth medium, 10% FBS and cells were grown for additional 24 hr prior to addition of G418 selective medium.

Prior to transfection with FuGENE6, mammalian cells were grown in T-25 flasks to 50% confluence and fed with the appropriate complete growth medium, 10% FBS 1-3 hrs prior to addition of the DNA/transfection reagent mixture. Three DNA/transfection reagent mixtures were prepared for each plasmid DNA using 1:3, 2:3, and 1:6 DNA:FuGENE6 ratios. To set up the DNA/FuGENE6 mixtures, FuGENE6 was diluted in the appropriate serum free growth medium as follows: 1:3 Ratio Mix: 242.5 μL SFM+7.5 μL FuGENE6, 2:3 Ratio Mix: 242.5 μL SFM+7.5 μL FuGENE6, 1:6 Ratio Mix: 235 μL SFM+15 μL FuGENE6

FuGENE6 dilutions were vortexed and incubated for 5 min at RT. Then the plasmid DNA was added as follows: 1:3 Ratio Mix: 2.5 μg, 2:3 Ratio Mix: 5 μg, 1:6 Ratio Mix: 2.5 μg. The DNA/FuGENE6 mixtures were vortexed and incubated at RT for 15 min prior to their addition to cells. Cells were grown in the presence of the DNA/FuGENE6 mixtures overnight. At this time, the culture medium was replaced with complete growth medium, 10% FBS and cells were grown for additional 24 hr prior to addition of G418 selective medium.

(c) Selection for Stably Transformed Cells

To isolate stable subclones, transfectants were selected for the 6418 resistance factor encoded by the expression plasmids. The G418 selective medium (complete growth medium supplemented with 500 μg/mL G418) was applied about 48 hrs post transfection. The selective medium was changed every second day for 2-3 weeks until the nonresistant cells detached and G418 resistant “primary” colonies emerged. Depending upon the number and density of colonies, plates were grown in G418-free medium until the plate was 50-60% confluent. All of the “primary” colonies on a selection plate were collected together in one sample, transferred to 100mm dish, fed 24 hrs after plating, and grown until ˜80% confluent. This collection of colonies was termed a cell pool or passage 1 (P1) pool.

(d) Measuring SET Ribosome Activity in Stably Transformed Mammalian Cell Pools

Each P1 pool was tested for SET Ribosome activity using one or more TR SET Reference Standard Reagents (Table 3) and measured using either of two assay procedures (e.g. a Cell Count or Confluence Assay).

All quantitative TR SET Assays were performed using a Cell Count protocol. For subclone analysis, cells from the P1 pool were counted and passed into a white clear bottom 96-well microtiter tray at a density of 25,000 cells per well (triplicate wells). Leftover cells were placed into a passage 2 (P2) dish to maintain a stock culture. Cells in the microtiter plate were allowed to grow for 18-40 hr to achieve a ˜75% cell confluence prior to incubation with a Reference Standard Reagent in complete growth medium and assayed for fLUC activity. In contrast, quantitative analysis of established cell lines required that cells must be grown using culture conditions (e.g. cell number, frequent media changes) that insured logarithmic growth (resulting in a high proportion of cells undergoing DNA replication or S phase cells). These cultures were counted and processed as before. This culture system reduced G2-specific SET Ribosome background and improved the response ratio of untreated to treated cells.

For the Confluence Assay, a confluent P1 culture was processed for passage and a fixed volume of the cell suspension (approximately 1% of the total or ˜60,000 cells per well) was passed into a white clear bottom 96-well microtiter tray. Cells in the microtiter plate were allowed to grow for 24-40 hr until all sample wells had reached confluence (i.e. the maximum number of cells per square centimeter) prior to incubation with a Reference Standard Reagent in complete growth medium and assayed for fLUC activity.

After incubation for 6 hr, cells were examined by phase contrast microscopy for signs of detachment. If more than ˜10% of cells were detached, the 96-well plates were centrifuged at 1200 rpm for 3 min to pellet the detached cells. The media were removed and replaced with 50 μL of Cell Lysis Buffer (25 mM Tris-phosphate (pH7.8), 10% glycerol, 1% Triton X-100, 1 mg/ml BSA, 2 mM EGTA and 2 mM DTT). Cells were incubated with the Cell Lysis Buffer for 10min at RT and cell lysis was verified using a phase contrast microscope. To ensure complete lysis, each sample well was vigorously agitated. Air bubbles manually disrupted with a syringe needle. To develop luminescence, wells were injected with 5 μL of the D-luciferin solution dissolved in Reaction Buffer (25 mM Glycylglycine (pH 7.8), 15 mM MgSO4, 4 mM EDTA, 15 mM Potassium phosphate, 1 mM DTT, 1 mM Coenzyme A, 6.7 mM ATP and 3.35 mM D-luciferin). After 4 sec with shaking, luminescence values were measured using the FLUOstar Optima (BMG Labtech) microplate reader with the appropriate lens filter at gains of 2500-4000.

Light values were converted to Fold Induction using the ratio of values produced by treated and untreated cells. Initially, the maximal SET value produced by any given Reference Standard Reagent was used to define an optimal Reference Standard Assay (i.e. composition and dosage of the Reference Standard that elicits the highest TR SET response) for each cell type (Table 3). This assay was subsequently used to screen subclones derived from this pool and to assign a Class designation.

(e) Isolation of Clonal Cell Lines Containing Distinct TR SET Classes and Construction of a TR Cell Panel.

Although dependent upon the cell line pool, cells transformed with the constitutive CMV-fLUC vector were judged to be responsive if the total relative light units (RLU) at a gain of 3500 were no lower than a value of 50,000 in the toxin untreated wells and the induction in the treated cells was 1.5 to 2.5 fold. Similarly, cells containing a TR fLuc expression vector were judged to be responsive when the total RLU at a gain of 3500 were no lower than a value of 1000 in the toxin untreated and treated wells and the induction in the treated cells was 3 to 1000 fold.

To prepare clonal isolates from responsive cell pools, cells from P3-P4 cultures were collected, diluted, counted, and replated. Plating densities ranged from 500 to 10,000 cells per 100mm dish, depending on cell type. Slow growing, low density sensitive cell types were plated at higher cell numbers, while a fast growing, density insensitive cell type was plated at lower cell numbers. Colony formation took 1-4 weeks, dependent upon the cell type. Once colonies were visible with the naked eye, individual colonies were marked for subcloning. Flame sterilized cloning rings were treated with a light coating of high vacuum grease and attached to the plate surrounding a colony. The cloning ring was filled with 1X trypsin-EDTA (Invitrogen) and incubated to release the cells which were passaged as a P1 colony into 24-well trays. Sufficient colonies per pool (150-400 independent subclones) were processed to recover >75% of all translationally responsive isolates. As each subclone reached confluence, each isolate was passed into a T-25 flask (marked as P2), grown to confluence, and analyzed using the cell-specific optimal Reference Standard Reagent assay in a Confluence Assay protocol.

Based upon the luminescence readout, a Fold Induction value was calculated for each subclone. These values were rank ordered from lowest to highest value and plotted as a function of rank order versus Fold Induction value (i.e. a Ranking Plot). To assign a TR Class designation, statistical analysis was used to group subclones into subsets that varied by at least 2 standard deviations from the mean of a lower Class response group. Based upon the lowest ranking series (lowest translational response), cell subclones were classified as a TR Class 1. Using an analogous procedure, TR Class 2 and 3 subclones were identified. The compilation of all Class responses were used to establish Class 1, 2 and 3 definitions and identify subclones for detailed analysis using the Cell Counting Assay.

The results of the quantitative Cell Counting Assay was used to identify sufficient subclones to construct a TR Cell Panel which contained: Class 1: no fewer than 2 to 3 representatives plus all boundary clones (Class designations that border Class 1 and 2); Class 2: no fewer than 3 to 4 representatives including all boundary clones; Class 3: all subclones were retained. Based upon the Class 3 response definition, an “Outlier” would be any subclone that exhibited a TR SET response that was >3 standard deviations outside the mean of the collective Class 3 subclones. Each TR Cell Panel subclone was placed in cryogenic storage. Cryopreserved stocks were generally prepared using low passage subclone stocks that had been grown to confluence in 100mm dishes, washed at least twice with 1X trypsin-EDTA (1 min, RT), collected in 2mL freezing medium (90% fetal bovine serum, 10% DMSO) per 100mm dish, and transferred to cryovials (1 ml cells per vial; lx10e7 cells per vial). Cryovials were placed in a −70/−80C freezer in a slow freeze container for 16-24 hr, then transferred to liquid nitrogen for preservation.

(f) Characterizing and Maintaining a TR Cell Panel

To define a SET expression pattern for each TR Cell Panel member, every cell in a Panel would be examined using a Cell Counting Assay with Reference Standard Reagents and subjected to Time Course (spanning no more than 6 hours), Dose-dependent (doses defined by known biochemical or enzymatic properties), and various Dose-dependent Modifier assays (testing the ability of a test compound or treatment to modify a Reference Standard response). These assays could be performed using single or combination treatments (containing 2 test agents/conditions) and dependent upon the total number of reagents/conditions in the assay were termed the 3- , 15- , or 21-reagent assay formats.

Occasionally, TR Class cell lines can be damaged by improper maintenance or poor cryopreservation. To recover a specific isolate, secondary subcloning and repurification of a Class defined subclone would be required. To purify secondary subclones, cell lines would be subjected to serial dilutions (plating 100-1000 viable cells/100mm dish) to recover individual colonies that were subcloned, propagated, and re-tested using the Cell Counting Assay as described. For TR Class 2 and 3 cells, this secondary subcloning procedure often resulted in subclones with lower and higher SET values than the parental clone (e.g. FIG. 7A).

1C. Establishing Specific SET Ribosome Responses Associated with a Distinct Outlier Population

(a) Measuring the Temporal Activation of the SET Ribosome During S Phase

The logarithmic or exponential cell growth protocol used for the TR Assay produces a high proportion of S phase cells, with a minimal fraction of G1 and G2 phase cells and low SET Ribosome background activity. Translation induction by various SET Agonists (Table 3) were shown to activate the SET Ribosome by an unexpectedly fast time course. As shown in FIG. 1B, an excellent system for measuring temporal regulation of the SET Ribosome involved the TR gLUC expression vector (secreted gLUC protein). In this study, TPA induced a statistically significant SET increase within 2 hr of treatment. Since the S phase cell cycle segment covers 6-8 hr, these results indicate that the SET Ribosome becomes active in late S phase cells and increased in magnitude as cells enter G2. This means that SET Ribosome activation correlates with DNA replication and any agent capable of inducing an Intra-S checkpoint should non-selectively produce an immediate block of SET Ribosome translation (Non-selective SET Antagonist). In contrast, compounds or treatments capable of stimulating DNA replication and G2 progression would activate the SET Ribosome and exhibit SET Agonist activity.

(b) Detecting and Defining the Thermal Regulation of the SET Ribosome

Cells damaged during DNA replication can activate an Intra-S checkpoint, induce senescence, and increase apoptotic cell death. Alternatively, a resistant cell can respond to DNA damage by activating a G2/M cell cycle checkpoint which provides sufficient time to synthesize materials needed to repair cell damage and induce cell cycle progression. Heat stress, one of the best studied cellular stressors, shows a temperature-dependent ability to stop DNA synthesis, induce DNA strand breaks, sequester mRNAs into stress granules, and enhance the SET of the heat shock proteins while inactivating the Cap-dependent ribosome. Earlier work showed that short-term exposure (1-2 hr) to low (41° C.) or moderate (43° C.) temperature does not significantly increase cell death or an Intra-S checkpoint but does inactivate replication enzymes (e.g. topoisomerases) and Cap-dependent translation while stopping mitosis at a G2/M checkpoint.

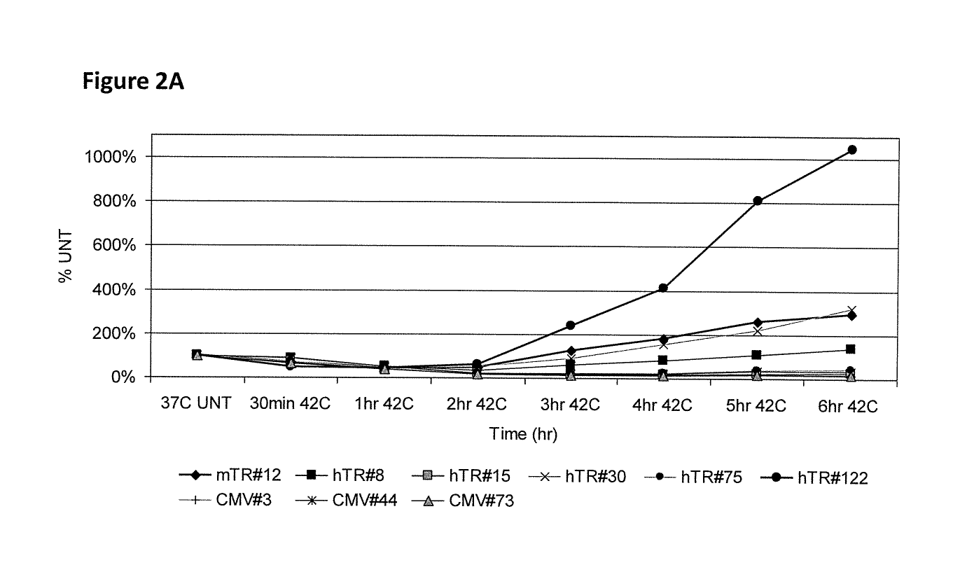

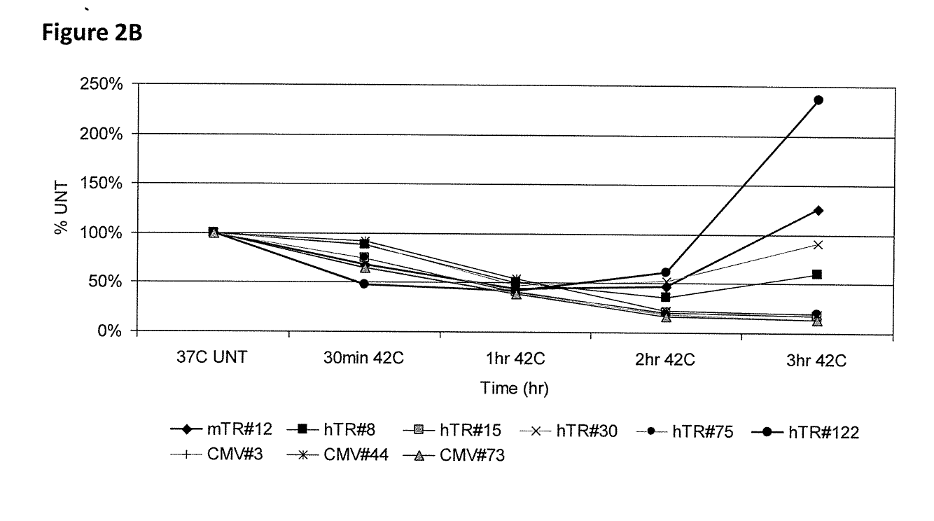

As shown in FIGS. 2A and 2B, the HEK293 TR Cell Panel was subjected to continuous heat shock (42° C.) and assayed for SET Ribosome responses. Each panel line was plated into a series of 96-well microtiter plates, as described for a Cell Count protocol using 25,000 cells per well, and grown for about 40 hr. Each microtiter plate was heated at 42° C. and plates were removed hourly, the samples processed, and assayed for firefly luciferase activity, as described. Each time point represents the average of triplicate wells. As expected, Cap-dependent translation (exemplified by the CMV expression vectors) declined significantly within lhr and continued to decline for 6 hr. However, beginning at 2 hr (a time consistent with the earlier gLUC temporal assay of SET Ribosome activation) and continuing through the treatment period, SET of the fLUC reporter protein increased linearly. Unexpectedly, at 6 hr, the magnitude of the SET Ribosome response in each TR cell line correlated with the previously assigned TR Class designation. These results show that activation of the SET Ribosome during S phase and SET are both heat resistant.

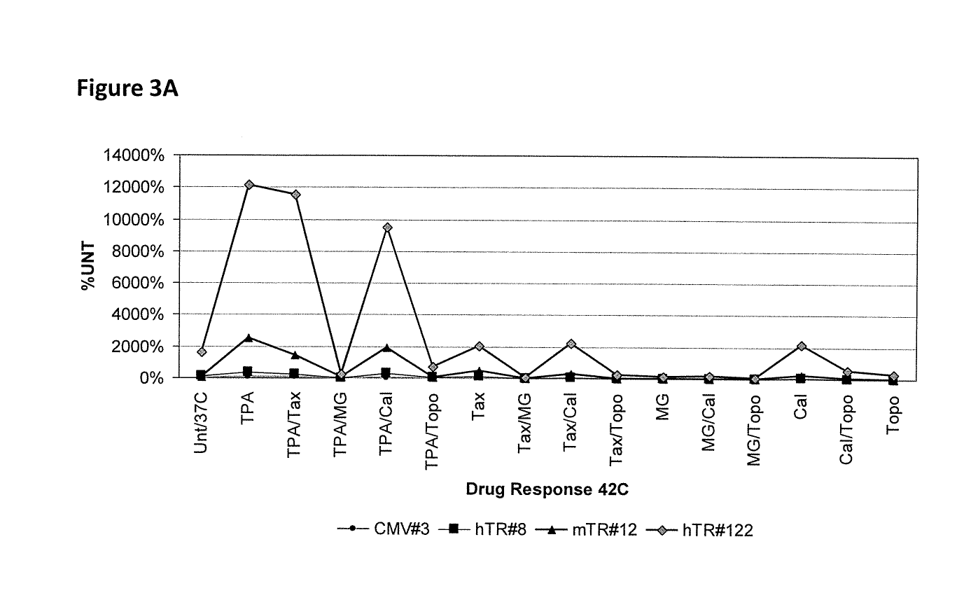

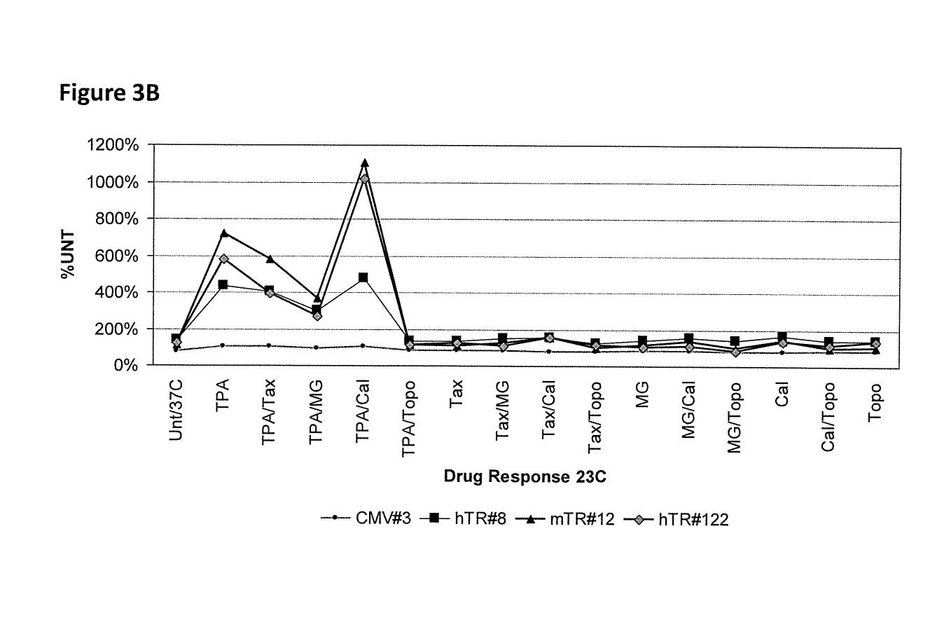

As with heat shock, treating cells at ambient temperature (cold shock) results in the rapid SET of cold shock proteins and inactivation of the Cap-dependent ribosome. To further examine the thermal properties of the SET Ribosome (FIGS. 3A and 3B), a 15-assay study was performed on representative cell lines from the HEK293 TR Cell Panel at 42° C. and 23° C. using 5 reference standards (Table 3). The HEK293 TR Cell Panel lines were plated into 96-well microtiter plates and processed as described for a Cell Count protocol. Cells were treated with Reference Standard Reagents (summarized in Table 3) as single or pairwise combinations and incubated at ambient (23° C.), physiological (37° C.), and high (42° C.) temperatures for 6 hours. Cells were processed and assayed for firefly luciferase activity as described.

As expected, Cap-dependent translation in the CMV cell line did not show any responses at either temperature. In contrast, comparing high and low temperature results found that TR Class 3 cells exhibited a synergistic SET activation when a preferred SET Agonist (TPA) was applied with heat. This enhanced SET activity was abrogated by the proteasome inhibitor and topoisomerase I poison which are known to stop early DNA synthesis. In contrast, cold regulated SET displayed a TR Class independent response so that all SET responses to the TPA SET Agonist were equivalent at 6 hr. These results demonstrate that heat stimulates S phase progression and the initiation of a G2/M checkpoint, whereas cold slows SET Ribosome activation and/or cell progression to G2.

(c) Detecting and Defining SET Ribosome Responses in Cells Treated with a Cap-Dependent Translational Inhibitor

Throughout early interphase, the mammalian target of rapamycin (mTOR) kinase is a component of the multiprotein mTOR Complex 1. During GO/G1 Cap-dependent translation initiation, mTORC1 never directly binds the ribosome but enzymatically activates regulatory proteins that enhance 40S subunit assembly on the 5′ mRNA Cap structure and induce ribosome scanning to an adjacent ORF. In contrast, during G2/M, an mTOR Complex 2 is formed that contains a distinct group of accessory proteins that must bind the 80S ribosome to activate mTOR kinase. This unique protein complex alters 80S ribosome function so that the G2/M ribosome-mTORC2 hybrid can selectively translate the TR mRNA (the SET Ribosome).

Translational regulation pathways can be distinguished by their sensitivity to mTOR kinase inhibition. Low doses of rapamycin, a macrocyclic lactone antibiotic, will bind the FKBP12 protein in mTORC1, downregulate GI phase Cap-dependent ribosome activity, and induce a GUS checkpoint. For mTORC2, changes in the accessory proteins make the mTOR kinase insensitive to low dose rapamycin; however, the effect of this process on the SET Ribosome remained undefined. Select members of the MCF7 TR Cell Panel were plated into 96-well microtiter plates and processed as described for a Cell Count Dose-dependent Modifier protocol. Rapamycin concentrations were tested for an ability to alter the Reference Standard responses produced by a 100 nM TPA/500 nM paclitaxel combination. Rapamycin was applied at doses ranging from 1 nM to 1 μM concentrations. All dilutions were prepared in complete growth media. Cells were incubated for 6 hrs, processed, and assayed for luciferase activity as described.

As shown in FIG. 4, MCF7 cells respond to low dose rapamycin (1 nM-50 nM) by activating the SET Ribosome. At doses that inhibit the mTORC2 kinase (>50 nM), the magnitude of SET is reduced but not eliminated. These results show that the SET ribosome is not regulated by a standard GI translational inhibitor.

(d) Defining SET Ribosome Responses in Cells treated with DNA Replication Toxins

Select members of the MCF7 and HEK293 TR Cell Panels were plated into 96-well microtiter plates to perform a Cell Count Dose-dependent and/or Dose-dependent Modifier (tested for an ability to alter the SET Agonist response produced by 100 nM TPA) protocol on cobalt chloride and topotecan. Cobalt chloride doses ranged from 2 μM to 2 mM. Topotecan doses ranged from 2 nM to 25 μM. Each test dose, prepared in complete growth media, was mixed with the appropriate Reference Standard reagent and applied to cells for 6 hours, the samples were processed, and assayed for luciferase activity as described.

Environmentally ubiquitous metals are recognized as human health hazards in applications involving prolonged occupational exposure during mining, industry, medicine, or agriculture. In mammals, metals such as the soluble cobalt(II) salts can cause dose dependent acute toxicity, DNA damage, increased mutation frequency, and chromosomal aberrations. At doses as low as 50 μM-100 μM, cultured cells can exhibit S phase defects, such as DNA strand breaks and unwinding. As shown in FIGS. 5A and 5B, a fixed dose of a TR Standard Reagent (SET Agonist and/or Antagonist) was mixed with increasing concentrations of cobalt(II) chloride to test for a combinatorial increase or decrease in SET. At low doses (2 μM-50 μM), cobalt(II) had no affect on SET; however, doses >50 μM were able to inhibit SET Ribosome activation by paclitaxel (IC50 of about 200 μM). In contrast, cells treated with TPA required doses >200 μM to inhibit SET activity (IC50 of about 1 mM). These results confirm that DNA damage produced by environmental toxins can inhibit G2 progression and that SET Ribosome activation may show drug-specific regulation.

Eukaryotic DNA topoisomerase I (topoI) is an enzyme that relaxes DNA supercoils generated during transcription and replication. Topol regulates DNA relaxation by forming a covalent enzyme-DNA complex that stimulates the production of transient single-strand breaks which can rotate around the intact DNA strand. After DNA unwinding, the topoI-DNA covalent bond is reversed and the free DNA end is religated. A variety of drugs (such as camptothecin, topotecan, and irinotecan) have been shown to interfere with this process by stabilizing the enzyme-DNA complex and preventing DNA ligation. At low doses, these drugs induce single strand breaks that stimulate cell cycle progression to a G2/M checkpoint. In contrast, higher concentrations produce sufficient numbers of trapped topoI-DNA complexes that replication fork collisions result in double strand DNA breaks, cell senescence, and death. As shown in FIG. 6A, select members of the MCF7 TR Cell Panel treated with topotecan displayed a dose dependent SET Agonist activity (2-100 nM, maximal SET response at 10 nM) that transitioned to a SET Antagonist response at higher doses (100 nM-10 μM, IC100 at >5 μM). Similarly, FIG. 6B shows that treating HEK293 Class 3 cell lines with a fixed dose of TPA and variable topotecan doses produced a similar biphasic SET response profile. In FIG. 6A-6B, topotecan had no detectable effect on the CMV Cap-dependent ribosome. FIG. 6C correlates the topotecan-specific SET responses with known cell/animal responses and toxicity. Of particular importance is the observation that doses at the transition from SET Agonist to Antagonist activity correlated with human clinical doses and the maximum tolerated dose. SET Antagonist doses induced DNA damage, spontaneously killed mice, and stopped the cell cycle. These results show that TR cell lines respond to mild DNA damage (e.g. single stranded breaks) and G2 cell cycle progression by rapidly increasing SET Ribosome activity. In contrast, agents capable of severe DNA damage (double-strand breaks) promote an early S phase checkpoint which prevents SET Ribosome activation.

(e) In Vitro Growth Assays that Define Unique Properties in TR Class 3 Outlier Cell Lines

Clearly, the magnitude of the SET Ribosome response correlates with an increased ability to synthesize late S and G2/M-specific proteins that are needed to repair cell damage and cell cycle progression. Based upon the CSC Model, these traits are commonly associated with drug and stress resistant tumor cells. If the TR Class 3 Outlier cell lines are candidates for a TR Metastatic Cancer Cell Model, these cells must exhibit growth characteristics consistent with metastatic potential. This example uses adherent and nonadherent growth assays to test for enhanced in vitro growth ability and an ability to evolve and produce more differentiated progeny.

The first assay employs a repeated Colony Formation protocol to test for enhanced plating efficiency in single cells. In this assay, putative Class 3 Outliers from the MCF7, HEK293 and HCT116 TR Cell Panels were established in exponentially growing cultures and 250-500 cells plated into two 100mm tissue culture dishes (Corning, cell culture treated). Colony formation in G418 selective medium was performed as previously described. Colonies were harvested into a single pool, transferred to a single T75 flask for stock maintenance, and subcloning repeated for at least 3 cycles. Based upon the Fold Induction, cell responses were ordered into a rank from lowest to highest and plotted as rank order versus Fold Induction.

In a second assay, the putative TR Class 3 Outliers from the MCF7, HEK293 and HCT116 TR Cell Panels were established in exponentially growing cultures and 10,000 cells were transferred to two 100 mm tissue culture dishes (Fisherbrand polystyrene Petri dish) that were not cell culture treated. Cells were allowed to aggregate and adapted to nonadherent growth by passage in complete medium for a week. Cell aggregates were manually disrupted to single cells and transferred to fresh petri dishes. It was not unusual for early cultures to contain a number of dead cells in the cell aggregates. Since these cells would not attach to attach to fresh cell aggregates, they could be removed by allowing the viable cell clumps to settle and repeated medium changes. Microscopic examination was used to verify cell viability, growth rate (clump size), and to determine whether cultures contained small cell spheres. After 2 weeks as nonadherent cultures, the capacity to form tumorspheres was measured by using Trypan Blue to determine the number of viable cells and plating 100-200 viable cells into a fresh petri dish with complete medium. In essence, selection for the ability to form nonadherent cell colonies. Dishes were carefully transferred to an incubator and grown for >5 days. Microscopic examination was used to identify cultures that contained small tumorspheres (FIG. 7B). This study established that nonadherent growth correlated with a tumor cell type and not a TR Class response. For example, the majority of isolates in the HCT116 TR Cell Panel demonstrated the ability to grow nonadherently (Table 4). Cell pools were transferred to standard tissue cultures dishes and adapted to adherent growth for at least 2 passages prior to measuring SET responses.

As shown in FIG. 7A, subclones isolated from a HEK293 TR Class 3 Outlier cell line subjected to the Colony Formation protocol exhibited Clonal Evolution (a change in cellular growth displayed by a single cell clone) exemplified in this study by decreased or increased SET responses compared to the parental TR Cell Panel clone. In particular, colony formation was able to select for a novel outlier subclone with a significantly higher TR assay response compared to other subclones. Similarly, testing the TR assay responses produced by the HCT116 TR Class Panel, which contains a number of TR Class 3 Outlier clones, after 4 weeks of nonadherent growth established that only the putative TR Metastatic Cancer Cell Models exhibited significant increases in SET response (mTRdm-fLUC#25, #28, and #75, Table 4). These results are consistent with the ability of the TR Class 3 Outlier cell lines to respond to stressful growth conditions by altering their SET magnitude which produces enhanced resistance and viability. These traits are consistent with the expected response of a metastatic tumor cell and supports the concept that the TR Class 3 Outlier subclones can adapt to growth stress and alter the parental SET response so that a new outlier cell is generated with an elevated SET activity. We term these candidate cells Class 4 cells, which only require specific in vivo tumor traits to become an accepted TR Metastatic Cancer Cell Model.

### Example 2

2A. Establishing that a HCT116 TR Metastatic Cancer Cell Model Exhibits a Tumor Initiating Activity During Serial Transplantation in Nude Mice

(a) Implanting Cells and Tumor Fragments from the TR Metastatic Tumor Cell Model and the HCT116 Parental Cells

Female mice (Crl:NU-Foxnlnu) obtained from Charles River Laboratories were 7 weeks old on Day 1 of the experiment. The mice were fed irradiated Rodent Diet 5053 (LabDiet) and water ad libitum. Mice were housed in static cages with Bed-O'Cobs bedding inside Biobubble Clean Rooms that provide HEPA filtered air into the bubble environment at 100 complete air changes per hour. The environment was controlled to a temperature range of 70°±2° F. and a humidity range of 30-70%.

HCT116 parental cells and the Class 4 HCT116 hTRdm-fLUC#32 cell line were expanded using RPMI 1640 media modified with L-Glutamine (Cell Gro) supplemented with 10% fetal bovine serum, 1% penicillin-streptomycin-glutamine, 1% Sodium Pyruvate, and 25 mM Hepes in a 5% CO2 atmosphere at 37° C. Prior to implantation, each cell type was collected, pooled, and viable cell number determined using a trypan blue exclusion assay. Cell suspensions were centrifuged at 1500 rpm (300×g) for 5 minutes at 4° C.

A 25×10e6 cells/rill suspension (serum-free RPMI) was prepared for the HCT116 and HCT116 hTRdm-fLUC#32 cells and 5×10e6 cells/mouse (0.2 ml) were implanted subcutaneously into twenty mice (10 animals in each test Arm) on Day 0 using a 27-gauge needle. Each cell suspension was maintained on wet ice to minimize the loss of cell viability and inverted frequently to maintain a uniform cell suspension. At 21 days post-implantation (tumor mean size of 750 mg), animals were euthanized, tumors harvested, and sectioned into 30 to 60 mg fragments (average size of 45 mg). Chunks from an HCT116 parental and hTRdm-fLUC#32 tumor were implanted subcutaneously and bilaterally (Day 0) using a 12-gauge trocar needle into twelve nude mice (6 animals per test arm). Animals were sacrificed on day 22 when one tumor in each Arm had grown to >2 g. (00429) All mice were observed for clinical signs at least once daily. Body weights and tumor measurements were recorded twice weekly. Tumor burden (mg) was estimated from caliper measurements using the formula for the volume of a prolate ellipsoid assuming unit density as: Tumor burden (mg)=(L×W2)/2, where L and W are the respective orthogonal tumor length and width measurements (mm). All treatments, body weight determinations, and tumor measurements were carried out in the bubble environment.

(b) Serial Tumor Growth in Mice Implanted with Cultured Cells and Tumor Fragments

Nine of ten test animals implanted with the parental HCT116 cell line produced tumors. Tumors reached a mean size of 650 mg in 17 days (tumor volume doubling time was 5.8 days). All ten animal implanted with the hTRdm-fLUC#32 cells produced tumors, which reached a mean size of 650 mg in 17.4 days (tumor volume doubling time of 6.5 days). In both Arms, mice exhibited minimal weight loss and no spontaneous regressions. As shown in Table 5, tumor fragment growth was highly variable. Although each tumor exhibited positive size increases over 21 days (HCT116 size increases of 3.2×-47.5× compared to hTRdm-fLUC#32 size increases of 4×-30×), the distribution of tumor sizes were not random. In both Arms, the top four tumor sizes were statistically larger than the remaining eight tumors; however, the rank distribution of large sizes favored the hTRdm-fLUC#32 tumor with 6 of 12 tumors exceeding 600 mg compared to 4 of 12 in the HCT116 parental tumor. These results show that the hTRdm-fLUC#32 cell line exhibits a serial in vivo tumor initiation activity and also support an enhanced tumor growth rate.

2B. Noninvasive Imaging of the Putative TR Metastatic Cancer Cell Tumor Showing regulated translation from the TR Expression Cassette

(a) Producing tumors from the TR Metastatic Tumor Cell Model and the HCT116 Parental Cells

Female mice were obtained from Charles River Laboratories (Crl:NU-Foxn1nu) or Harlan Laboratories (Hsd:Athymic Nude-Foxl nu) which were 6-7 weeks old on Day 1 of the study. The mice were fed irradiated Rodent Diet 5053 (LabDiet) and water ad libitum, housed in static cages with Bed-O′Cobs bedding inside Biobubble Clean Rooms that provide H.E.P.A filtered air into the bubble environment at 100 complete air changes per hour. All treatments, body weight determinations, and tumor measurements were carried out in the bubble environment. The environment was controlled to a temperature range of 70°±2° F. and a humidity range of 30-70%. All mice were observed for clinical signs at least once daily. Mice with tumors in excess of 2 g, with ulcerated tumors, in obvious distress, or in a moribund condition were euthanized.

HCT116 parental and HCT116 hTRdm-fLUC#32 cells were grown in RPMI1640 medium supplemented with 10% (heat-inactivated) fetal bovine serum, 1% penicillin-streptomycin-glutamine, 25 mM HEPES and 1% sodium pyruvate in a 5% CO2 atmosphere at 37° C. Cells were collected and pooled for implantation after determining cell viability using a trypan blue exclusion assay. The cell suspension was centrifuged and a 50×10e6 cells/ml suspension was prepared in 50% Serum-Free RPMI and 50% Matrigel. A total of 29 mice were implanted subcutaneously (Day 0) with 5x10e6 cells/mouse HCT116 hTRdm-fLUC#32 cells (Arms 1, 2 and 3), and 29 mice were implanted with HCT116 parental cells (Arms 4, 5 and 6). Treatments began on Day 8 (animals triaged into 3 groups of 6 animals each), when the mean estimated tumor mass for all groups was 125 mg (range of group means, 119-129 mg). All mice weighed ≧19.2 g at the start of treatment. Mean group body weights at first treatment were well-matched (range of group means, 22.4-23.5 g). All mice were dosed according to individual body weight on the day of treatment (0.2 ml/20 g). To repeat the noninvasive imaging study, the remaining HCT116 hTRdm-fLUC#32 animals were placed in 3 Arms containing 3 animals (mean tumor weight was about 500 mg).

(b) Bioluminescent Imaging Results for Tumors Containing the TR Metastatic Tumor Cell Model

As shown in FIGS. 8A-8D and Table 6, bioluminescence images of the TR reporter protein (fLUC) activity expressed by the HCT116 hTRdm-fLUC#32 tumors were taken prior to treatment and 6 hours after treatment. Arm #1 was treated with cremophorEL (0.5 mg/kg/day). Arm #2 was treated with cremophorEL (0.5 mg/kg/day), paclitaxel (20 mg/kg/day). Arm #3 was treated with cyclophosphamide (120 mg/kg/day). The fLUC enzyme was detected using D-Luciferin powder obtained from Molecular Imaging Products Company (MIP). Saline was added to the luciferin powder to produce a 15 mg/ml suspension. The suspension was vortexed for approximately 1 minute to produce a clear, yellow solution. D-Luciferin was prepared immediately prior to each bioluminescence imaging session and stored on wet ice during use. In vivo bioluminescence imaging was performed using an IVIS 50 optical imaging system (Xenogen, Alameda, CA). Animals were imaged (three at a time) under 2% isoflurane gas anesthesia. Each mouse was injected IP with 150 mg/kg luciferin and imaged with the tumors facing the camera, 10 minutes after the injection. Large binning of the CCD chip was used and the exposure time was adjusted (5 seconds to 5 minutes) to obtain at least several hundred counts from the tumors and to avoid saturation of the CCD chip. Images were analyzed using Living Image (Xenogen, Alameda, CA) software and each unique signal was circled manually and labeled by group and mouse number.

FIGS. 8A-8D and Table 6 show that the bioluminescence level expressed by the untreated HCT116 hTRdm-fLUC#32 tumors was highly variable (ranging from 0.2×10e6 to 60×10e6 photons/sec). However, tumor responses could be separated into two classes, with the lowest pre-treatment expression level ranging from 0.2×10e6−1.2×10e6 photons/sec and the highest spanning 13.2×10e6−60.4×10e6 photons/sec (more than 10× the greatest low pre-treatment response). While the low pre-treatment tumors exhibited highly significant SET increases following treatment (10,540%-46,600% increase in treated over untreated tumors), tumors expressing a higher level were unable to induce significant SET activity (70.7%-381.8%). To rule out that these responses were produced by residual luciferin 6 hr after the pre-treatment measurement, a separate cohort of tumor bearing animals were imaged 24 hr after pre-treatment (Table 6). For these larger tumors (average tumor size of about 500 mg), only 1 tumor in Arm #2 exhibited a low endogenous expression level (0.5×10e6 photons/sec) but it produced the expected SET increase (20,500% induction). These results show that SET can be activated in HCT116 hTRdm-fLUC#32 tumors by paclitaxel/cremophorEL producing an in vivo response consistent with an in vitro TR Class 3 outlier activity. Second, the presence of pre-treatment SET activity indicates that small tumor morphology produced an unexpected stress response in mitotic cells (a previously unknown G2-related cell cycle checkpoint) that becomes more common during tumor growth. Moreover, the presence of an inducible tumor response in each treatment group shows that SET from the TR expression cassette responds to some compound in each drug/vehicle formulation. Of particular note was the large SET induction produced by cremophorEL (an excipient commonly used to dissolve paclitaxel in aqueous solutions).

2C. Unexpected Tumor- and SET-Dependent Drug Resistance in the TR Metastatic Cancer Cell Model Tumors

(a) Drug Formulations and Animal Treatments

Test Arms #1 and #4 were treated with the vehicle control (cremophorEL 0.5 mg/kg/day; Q2Dx5), Arms #2 and #5 were treated with paclitaxel/cremophorEL (20 mg/kg/day and 0.5 mg/kg/day, respectively; Q2Dx5), and Arms #3 and #6 were treated with cyclophosphamide (120 mg/kg/day, Q4Dx3). Each drug (0.2 ml/animal) was delivered by intravenous (IV) delivery. The vehicle control contained 12.5% ethanol, 12.5% Cremophor EL and 75% saline. Reagent grade paclitaxel was obtained from Hauser Pharmaceutical Services as a dry yellow powder and stored protected from light at room temperature. On each day of treatment, the compound was dissolved in absolute ethanol (12.5% of the final volume), followed by sequential addition of cremophorEL (12.5% of the final volume) and saline (75% of the final volume) with thorough mixing after each addition. The resulting solution was clear and colorless. Cyclophosphamide was obtained from McKessen Specialty Products as a white powder and was dissolved fresh prior to each treatment in saline to create a clear, colorless solution with a pH of 4.0.

All mice were observed for clinical signs at least once daily. Mice were weighed on each day of treatment and at least twice weekly thereafter. Tumor measurements were recorded twice weekly for 63 days. Tumor burden (mg) was estimated from caliper measurements using the previously described formula. Mice with tumor burdens in excess of 2 g or with ulcerated tumors were euthanized, as were those found in obvious distress or in a moribund condition. Individual tumor weights were plotted over time for each animal group (FIG. 9A). Animal survival was assessed using a Kaplan-Meier graph, where the animal number (% Survival) is plotted versus day of trial (time) and provides an estimate of the Survival Function for each treatment arm (FIG. 9B).

(b) Unexpected Drug Resistance Activity in TR Metastatic Tumor Cell Model Tumors

All treatments began on Day 8 when the average tumor was 125 mg and average animal weight was 19.2 g (range 22.4-23.5 g). Although tumor sizes in Vehicle Arm #1 and #4 did not differ on Day 8, a statistically significant tumor size increase was detected in the hTRdm-fLUC#32 animals as early as Day 11 (p=0.043), which continued through Day 22 (p=0.0093). Animal sacrifice on Day 25 (Arm #1, 3 of 6 animals sacrificed for tumor burden compared to 0 of 6 animals in Arm #4) prevented further tumor comparisons. In contrast, tumors in Arm #2 and #6 only displayed a significant size difference on Day 11 (p=0.023), which means that subsequent cyclophosphamide treatments suppressed tumor growth for the remainder of the study. These results show that hTRdm-fLUC#32 tumors exhibit enhanced growth in vivo.

Although Arms #2 and #5 established paclitaxel/cremophorEL efficacy in each cell line, there was remarkable individual tumor variation. As a group, the hTRdm-fLUC#32 tumors (Arm #2) displayed an average time to 750 mg of >63 days (tumor growth delay of >48.7 days) compared to 50.4 days for the HCTZ 16 parental tumors (growth delay of 33.7 days). However, as shown in FIG. 9A, the 3 tumors that were sensitive to paclitaxel/cremophorEL also exhibited low pre-treatment SET activity, a significant SET induction after treatment, minimal tumor regression, and high resistance to chemotherapy (resulting in tumor regrowth between Days 29-36). In contrast, the high pre-treatment SET tumors were exceptionally sensitive to paclitaxel/cremophorEL and exhibited minimal regrowth potential.

Further support of an enhanced growth rate for the hTRdm-fLUC#32 tumors is shown in the Survival Plot of FIG. 9B. For example, the last Arm #1 animal was sacrificed for tumor burden on Day 29 (survival mean of 24 days) compared to Day 43 in Arm #4 (survival mean of 29 days). Although tumor size and survival means did not vary significantly in Arms #3 and #6, the last animal in Arm #3 was sacrificed for tumor burden on Day 39 compared to Day 43. Since paclitaxel/cremophorEL reduced tumor regrowth in Arms #2 and #5, animal sacrifice for tumor burden was lowered; however, two hTRdm-fLUC#32 animals were sacrificed significantly earlier than the HCT116 animals (Days 50 and 57 compared to Days 57 and 62). This cell line-dependent reduction in animal survival, coupled with an enhanced tumor growth rate, proves that the hTRdm-fLUC#32 tumors grew faster than the HCT116 parental cells. Moreover, the ability of 4 of 6 hTRdm-fLUC#32 tumors to regrow after paclitaxel/cremophorEL treatment shows that these tumors exhibit in vivo drug resistance consistent with the TR Class 3 response observed in vitro. Therefore, the hTRdm-fLUC#32 cell line exhibits each of the properties associated with a TR HCT116 CSC and as such represents an enabling example of a TR Metastatic Cancer Cell Model. To date, 15 candidate Class 3 Outlier cell lines have been identified in 6 cancer cell types that exhibit Class 4 drug and stress resistance (HCT116 mTRdm-fLUC#25, #28, #75 and hTRdm-fLUC#32, #69, #122; MCF-7 mTRplp-fLUC#118 and mTRdm-fLUC#111, #217; HepG2 hTRdm-fLUC#16; HEK293 hTRdm-fLUC#122 and hTRdm-gLUC#79; DU145 hTRdm-fLUC#27 and mTRdm-fLUC#194; HT1080 mTRdm-fLUC#99, #122). Of this group,

HCT116 mTRdm-fLUC#75 and hTRdm-fLUC#32 completed all in vitro studies and the hTRdm-fLUC#32 cell line was chosen for in vivo tumor validation described in this example.

### Example 3

3A. Examining the In Vitro Translational Activity Produced By SET Agonists and Antagonists

(a) Unexpected Cell Based SET Response Produced by an In Vivo SET Agonist

As shown in Arm #1 of Table 6, intravenous delivery of cremophorEL (0.5 mg/kg/day) produced significant SET activation in pre-treatment tumors with low SET activity. As a non-ionic surfactant, cremophorEL is commonly used to solubilize hydrophobic drugs, but it has also been shown to produce multiple in vivo side effects. Given that the activation pattern and magnitude of the tumor SET response was consistent with a cell based SET Assay, a Cell Count Dose Response Assay was used to examine the ability of cremophorEL to induce in vitro SET. As shown in FIG. 10A, cremophorEL (dose range 2.5 mg/ml to 100 mg/ml) reduced fLUC expression in the CMV cell line demonstrating that cremophorEL inhibits Cap-dependent translation. Unexpectedly, a 6 hr treatment of HEK293 mTRdm-fLUC#12 (a TR Class 3) and mTRdm-fLUC#122 (a TR Class 4) produced no significant SET increase at any dose. Only cells incubated for 24 hours exhibited a modest 160% SET increase at 10 mg/ml. These results show that cremophorEL produces a cell type-specific mitotic effect. Since low dose cremophorEL can stop cell cycle progression in S phase, it appears that cremophorEL inhibits Gl Cap-dependent translation in cultured cells but does not induce mitosis and SET activation. In contrast, tumors respond to cremophorEL by entering G2 and activating the SET Ribosome, which shows that cell culture systems may not effectively model in vivo tumor responses.

(b) Examining SET Antagonist Activity Produced by ribosome Binding Translation Inhibitors

As shown in Table 3, a number of SET Antagonists have been identified. However, the majority of these agents simply stop cell cycle progression in the S phase which prevents activation of the G2 SET Ribosome. To replicate the Pre-treatment SET expressed by drug sensitive tumors, a therapeutic must activate G2 progression but also block G2 translation to prevent SET super-induction which is responsible for cell recovery. FIG. 10B shows the Cell Count Dose-dependent Modifier Assay results that examine SET Antagonist activity associated with a set of translational inhibitors that bind directly to the SET Ribosome. To determine the IC100 dose (drug amount that produces an immediate and complete inhibition of SET Ribosome activity), TR Class 3 cells HEK293 hIRdm-fLUC#13 and mTRdm-fLUC#45 were treated with a combination of 100 nM TPA (SET Agonist) and varying concentrations of the translational inhibitors; anisomycin, emetine, cycloheximide, and puromycin (dose range 10 nM to 25 μM). Based upon the reported half-life of firefly luciferase, the IC100 treatment must completely stop all SET (block the SET Agonist response and produce an apparent decrease in fLuc activity to about 85% of the activity in an untreated control sample). Therefore, a SET blocking dose stops the SET Agonist induction but exhibits residual translational activity (95-150% fLUC activity), an IC100 concentration stops all SET activity, and any treatment that results in <85% activity likely affects protein synthesis and degradation.

As shown in FIG. 10B, each drug could block the SET Agonist activity; however, the SET blocking and IC100 doses exhibited drug-specific variation. For example, SET Agonist induction could be stopped by 250 nM-500 nM anisomycin, 1 μM-2.5 μM emetine, 2.5 μM-5 μM cycloheximide, and 10 μM-25 μM puromycin. As expected, the IC100 dose for each drug increased to 500 nM-1 μM for anisomycin, 2.5-10 μM for emetine, >10 μM for cycloheximide, and >25 μM for puromycin. Therefore, anisomycin exhibited the lowest SET blocking and IC100 doses, followed by emetine, cycloheximide and puromycin, respectively.

B. Testing the Safety and Efficacy of Oral SET Combination Drugs (Animal Study 1)

(a) Preparing and testing SET Combination Drug formulations in Nude Mice Containing TR Metastatic Cancer Cell Model tumors

A total of 45 mice were implanted with HCT116 mTRdm-fLUC#32 cells and triaged into 5 Arms (8 animals each). All treatments began on Day 7, when the mean tumor weight was 125 mg (range of group means, 152-169 mg). All mice weights averaged >18.6 g at the start of therapy (range of group means, 20.5-22.4 g). All mice were dosed orally and treatments were applied daily for 18 days. All mice were weighed at treatment and at least twice weekly and mice whose body weight dropped below 20% of their starting weight on the first day of treatment were euthanized. Tumor burden (mg) was estimated as previously described and mice with tumors in excess of 2 g were sacrificed, tumors were excised, snap frozen in liquid nitrogen, and stored at −80 C for histopathological and immunostaining analysis. Body weights and tumor measurements were recorded twice weekly for 70 days.

The Group average tumor weights, as well as individual tumor weights within each group were plotted over time to determine the effects of treatment on tumor growth and regression. Individual animal body weights measured on any given day were normalized by subtracting the tumor weights on that day and converting them to percentages of the initial body weights as measured on the first day of treatment. The normalized weights were plotted over time to assess the effects of treatment. Animal survival was assessed using a Kaplan-Meier graph, where the animal number (% Survival) is plotted versus day of trial (time) and provides an estimate of the Survival Function for each treatment arm.

(b) Highly Significant TR Metastatic Cancer Cell Model Tumor Responses Produced by a SET Combination Drug

As shown in Table 7, various drug formulations were given to a total of 40 mice (containing HCT116 mTRdm-fLUC#32 tumors) that were organized into 5 test Arms of 8 animals each. Each SET Combination drug contained a SET Agonist, a SET Antagonist and a cytotoxic S Phase toxin. For this study (termed the First Xenogenic

Animal Study), the cytotoxic S Phase drug was capecitabine (a First Line therapeutic used to treat metastatic colon cancer). Although the 500 mg/kg/day capecitabine (18 days of treatment) was equivalent to 78% of a standard human dose, this high dose will produce mouse toxicity. The SET Agonist selected for this study was cremophorEL (0.5 mg/kg/day), a dose previously shown to activate SET in vivo. Given that the LD50 for cremophorEL in fish and rats is 450-6400 mg/kg, the test dose is >900× lower than a toxic concentration. The SET Antagonist selected for this study was anisomycin, which exhibited the lowest SET blocking and IC100 doses. As described, SET blocking activity was observed in cell based assays using 250 nM-500 nM anisomycin (equivalent oral dose of 0.000027 mg/kg/day) and the IC100 was 500 nM-1 μM (equivalent oral dose of 0.000054 mg/kg/day). Given that the mouse LD50 for anisomycin is 75-200 mg/kg, the IC100 dose would be >14,000× lower than a toxic concentration. Arm #1 was treated with a solution of 10% ethanol, 10% cremophorEL (0.5 mg/kg/day) and 80% saline; Arm #2 was treated with 500 mg/kg/day capecitabine; Arm #3 was treated with 0.5 mg/kg/day cremophorEL and 0.000054 mg/kg/day anisomycin; Arm #4 (Low Dose anisomycin) was treated with cremophorEL, capecitabine, and 0.000027 mg/kg/day anisomycin; and Arm #5 (High Dose anisomycin) was treated with cremophorEL, capecitabine, and 0.000054 mg/kg/day anisomycin.

As shown in FIG. 11A and Table 8, the IC100 or High Dose anisomycin SET Combination Drug produced highly significant tumor regression (Arm #5; average 73.7% tumor size regression) compared to animals treated with capecitabine (Arm #2; 53.2% regression) or a Low Dose anisomycin SET Combination Drug (Arm #4; 45.3%). As shown in FIG. 11B, animals treated with capecitabine or the Low Dose anisomycin SET Combination Drug immediately suppressed tumor growth but within 2 weeks of stopping treatment, regrowth was evident in all tumors. In contrast, FIG. 11C shows that the High Dose anisomycin SET Combination Drug produced 4 of 6 tumors with insignificant tumor regrowth and 3 of 6 tumors with no postmortem tumor at 70 days.

As detailed in FIG. 13, the only significant survival increase was evident in animals treated with the High Dose anisomycin SET Combination Drug. The survival mean for Arms #1 (28 days), #2 (24 days) and #3 (28 days) were not significantly different. In contrast, the animals sacrificed for weight loss in Arm #4 lowered the survival mean (15 days) compared to Arm #5 (>70 days). Together, these results show that the Low Dose SET Combination Drug produced no positive tumor or survival responses compared to the High Dose anisomycin SET Combination Drug which was very effective in combination with high dose capecitabine.

(c) Reversible Animal Weight Loss Produced By the SET Combination Drugs

As shown in FIGS. 12A, 12B, and Table 9, the SET Combination Drugs produced distinct animal weight loss patterns. Surprisingly, the SET drug components in Arm #3 produced a modest weight increase during early drug treatment (Day 10), that was not obvious at later times. As expected, the toxic capecitabine treatment in Arm #2 produced one spontaneous death and four weight loss sacrifices by Day 24 (>20% weight loss) but the weight of the surviving animals did not increase or decrease significantly throughout the trial (FIG. 12B). As shown in FIG. 12A, animals treated with both SET Combination Drugs resolved into two animal groups, with one group showing significant weight loss and a second group that did not differ significantly from control animals. For many of the animals in the significant weight loss group, weight loss exceeded 20% and animals were sacrificed (Table 9; Arm #4, 5 of 8 animals by Day 15, Arm #5, 2 of 8 animals by Day 24). In contrast, before the end of treatment (Day 25), the surviving animals began to rapidly recover lost weight and exhibited a statistically significant weight gain by Days 31 to 38 (Table 9). Although this weight gain correlated with the maximal tumor regression (FIG. 11C), gaining weight before the end of drug treatment and the significant difference in weight-dependent animal sacrifice in Arms #2 and #4 compared to Arm #5 is surprising. This result shows that the SET Drug components provided a protective effect that diminished the toxicity of high dose capecitabine.

(d) Unexpected Immune Responses Produced By the SET Combination Drugs

FIGS. 17A-17J and Table 12 show immunostaining studies on hTRdm-fLUC#32 tumors treated with the SET Combination Drugs. Tumors were dissected from animals sacrificed for weight loss (Arm #2 animals #1 on day 24 and #7 on day 22; Arm #4 animal #5 on day 18; Arm #5 animals #2 on day 24 and #8 on day 22), flash frozen, fixed in PBS buffered 4% paraformaldehyde, cut into 3p.m frozen sections, mounted onto slides, and stained with a mixture of fluorescently labeled and unlabeled antibodies to detect macrophage marker proteins (biotin-labeled anti-mouse MHC class II molecules IA/IE, Alexa-647-labeled anti-mouse CD11b/Mac-1, Alexa-488-labeled anti-mouse F4/80, and Alexa-647-labeled anti-mouse CD68) and the TR reporter protein (anti-firefly luciferase). To detect unlabeled primary antibodies, an Alexa-555-labeled secondary antibody or PE-labeled streptavidin were used. Nuclear DNA staining with the DAN dye is used to detect viable tumor cells. Slides were photographed (Nikon 90i Eclipse) and images analyzed using NIS Elements 3.2 or the Image) software.

Correlating the nuclear staining of FIG. 17A with the G2-specific fLUC expression in FIG. 17B (Arm #2 animal #1 tumor treated with capecitabine for 16 days) confirmed that capecitabine induced a G2/M checkpoint and activated SET Ribosome translation (fLUC expression) in a narrow strip of peripheral mitotic cells (white arrow FIG. 17B, termed Layer 1). By counting the number of sequential nuclei extending from the tumor surface, Layer 1 was shown to have an average thickness of 3.4 cells (Table 12). Surprisingly, Layer 1 cells exhibited minimal staining for macrophage epitopes but was bordered by an inner cell layer (termed Layer 2) that contained a dense concentration of F4/80 stained macrophages (6.4 cells thick). The F4/80+ macrophages in this layer did not stain for the other immune or fLUC proteins and appeared to be contained within and established a boundary for the tumor mitotic cell layer (9.8 cells thick). Individual F4/80+ cells penetrated into the tumor for an average depth of 16.6 cells (termed Layer 3). The total depth of immunostained cells extended into the tumor for 26.4 cells. While the border of Layers 2/3 contained a modest number of fLUC positive cell bodies, minimal staining was observed between Layer 3 and the necrotic core (dead cells with minimal nuclear DAPI staining). Identical tumor and immune cell responses were observed in a second tumor processed from Arm #2 animal #7 that had been treated for 14 days. These results are consistent with the capecitabine mode of action, the expected multi-layer structure of solid tumors, and activation of a specific subclass of F4/80+ innate immune cells by dying cells.

FIGS. 17C and 17D show a tumor from Arm #4 animal #5, treated with a Low Dose anisomycin SET Combination Drug for 10 days. In this tumor, the SET Combination drug activated uniform, G2-specific fLUC expression in tumor cells extending from Layer 3 to the necrotic core (white arrow FIG. 17D). In FIG. 17C, the Layer 2 macrophages were exemplified by bright, small nuclei that do not stain for the fLUC antigen. In contrast to the capecitabine tumor, the majority of the Layer 2 immune cells displayed selective staining for the CD68 marker protein (CD68+F4/80−) and a minor fraction of macrophages co-stained or lightly stained for F4/80 (CD68+F4/80+). Moreover, the CD68+F4/80− immune cells penetrated throughout the entire tumor, including the necrotic core. Since the tumors in Arm #4 did not display significant tumor responses or improved animal survival, these results showed that the SET Agonist stimulated G2-specific SET throughout the tumor (forcing non-mitotic cells to reenter the cell cycle) and activated a distinct CD68+F4/80− macrophage subtype.

FIGS. 17E-17F and Table 12 show a tumor isolated from Arm #5 animal #2 treated with a High Dose anisomycin SET Combination Drug for 16 days. FIG. 17F confirmed that G2-specific translation of the fLUC reporter protein was present in Layer 1 (white arrow); however, the average thickness of Layer 1 increased significantly to 7.8 cells (Layer 1 thickness, p=0.008, Table 12). Furthermore, this Layer was highly disorganized and contained small, subcellular fLUC+ bodies that mapped to the tumor periphery. Significantly, internal tumor cells did not display significant fLUC staining except in the necrotic core. Similar size increases were also observed in Layer 2 (average thickness of 7.8 cells) and Layer 3 (average thickness of 18.6 cells). The total depth of the Arm #5 immunoreactive cell layer was 15.6 cells (_{>}50% size increase). As in the earlier SET drug tumor, the majority of macrophages were CD68+F4/80− and had penetrated to the necrotic core. These results confirmed the ability of the High Dose anisomycin SET Combination Drug to enhance apoptotic cell death (the appearance of small fLUC+ bodies adjacent to dying mitotic cells) and induce an invasive CD68+F^{4/80}− macrophage response while also reducing G2-specific translation in non-mitotic cells.

FIGS. 17G and 17H show a tumor isolated from Arm #5 animal #8 treated with the High Dose anisomycin SET Combination Drug for 14 days. FIG. 17G shows DAPI staining produced by a tumor section spanning from the proximal necrotic layer (detectable DAPI stained nuclei) to the necrotic core (minimal DAPI staining). FIG. 17H demonstrates that this section contains a high density of fLUC+bodies that localize to cells containing no detectable DAPI staining (white arrows in FIG. 17G and 17H). Surprisingly, this data shows that the High Dose anisomycin SET Combination Drug stimulated cell cycle progression and enhanced cell death at the center of a tumor (an unexpectedly high metabolic activity in supposedly dead cells).

FIGS. 17I and 17J show a tumor from Arm #5 animal #8 and the quantitation of fLUC+ fluorescence across the interior of a tumor using the ImageJ software. A fluorescence density map was produced by drawing 15 boxes (35×695 pixels, 0.64 um/px) on FIG. 17I and measuring the fluorescence intensity for each of the 695 pixels. The darkest necrotic cell layer pixel was adjusted to 100% background and the total fluorescence for each pixel was compared to background (FIG. 17J). This density map shows that fLUC staining intensity increased by about 600% in cells with minimal DAPI staining compared to adjacent DAPI+cells. This result is consistent with a highly significant and selective increase in G2-specific apoptotic cell death in cells that are commonly assumed to be nonmitotic and metabolically inactive.

3B. Testing the Safety and Eefficacy of Oral SET Combination Drugs (Animal Study 2)

(a) Preparing and Testing SET Combination Drug Formulations in Nude Mice Containing TR Metastatic Cancer Cell Model tumors

A total of 45 mice were implanted with HCT116 mTRdm-fLUC#32 cells and triaged into 5 Arms (8 animals each). For this study (termed the Second Xenogenic Animal Study), the concentration of capecitabine was reduced to 400 mg/kg/day capecitabine (10 days of treatment) which was equivalent to 35% of a standard human dose. This low dose treatment should reduce mouse toxicity. The SET Antagonists selected for this study were anisomycin and emetine. As described, the anisomycin IC100 dose was 500 nM-1 μM (equivalent oral dose of 0.000054 mg/kg/day) and the emetine IC100 dose was 2.5-10 μM (equivalent oral dose of 0.00013 mg/kg/day). Given that the rat LD50 for emetine is 68 mg/kg, the IC100 dose is >5,231× lower than the maximum test dose. As shown in Table 10, Arm #1 was treated with vehicle; Arm #2 was treated with 400 mg/kg/day capecitabine; Arm #3 was treated with cremophorEL, capecitabine, and an IC100 concentration of emetine (0.00013 mg/kg/day), Arm #4 was treated with cremophorEL, capecitabine, and 0.000054 mg/kg/day anisomycin (High Dose); and Arm #5 was treated with cremophorEL, capecitabine, and 0.00013 mg/kg/day anisomycin (Very High Dose). All treatments began on Day 6, when the mean estimated tumor mass for all groups in the experiment was 125 mg (range of group means, 152-172 mg). All mice weighed >16.9 g at the initiation of therapy (range of means, 19.9-21.2 g). All mice were dosed orally, according to individual body weight on the day of treatment. Treatments were applied daily for 10 days, after which the animals were monitored for a total of 72 days. All mice were weighed and tumor measurements recorded as previously described. As mice are euthanized for tumor burden >2 g, the tumors were excised, fixed in PBS buffered 4% paraformaldehyde, and stored at 4° C. Group average tumor weights, as well as individual tumor weights within each group were plotted over time to determine the effects of treatment on tumor growth and regression. Individual animal body weights measured on any given day were normalized by subtracting the tumor weights on that day and converting them to percentages of the initial body weights as measured on the first day of treatment. The normalized weights were plotted over time to assess the effects of treatment on the overall animal health. Animal survival was assessed using a Kaplan-Meier graph, where the animal number (% Survival) is plotted versus day of trial (time) and provides an estimate of the Survival Function for each treatment arm.

(b) Highly Significant TR Metastatic Cancer Cell Model Tumor Responses Produced By SET Combination Drugs

As shown in FIG. 14A and Table 11, the IC100 or High Dose emetine (Arm #3; 66.4% average tumor regression), the IC100 or High Dose anisomycin (Arm #4; 51.6% tumor regression) and the Very High Dose anisomycin (Arm #5; 46.2% tumor regression) SET Combination Drugs produced highly significant tumor responses compared to animals treated with capecitabine (Arm #2; 35.2% tumor regression). As shown in FIG. 14A, capecitabine containing drugs immediately suppressed tumor growth but within 2 weeks of stopping treatment, regrowth was evident in all Arms. Detailed analysis of the maximal tumor regression observed in Arm #3 found that individual tumors exhibited significant tumor regressions in 7 of 8 animals (FIG. 14B).

As detailed in FIG. 16, the highest survival increases were evident in animals treated with the High Dose emetine (Arm #3; survival mean 62 days) and anisomycin SET Combination Drug (Arm #5; survival mean 54 days) compared to Arms #1 (26 days), #2 (47 days) and #5 (21 days). Together, these results show that the High Dose anisomycin and High Dose emetine SET Combination Drug were very effective when combined with low dose capecitabine.

(c) Reversible Animal Weight Loss Produced By the SET Combination Drugs

As shown in FIG. 15, drugs containing 400 mg/kg/day capecitabine produced a distinct biphasic weight change pattern. For each drug, animal weight declined during treatment (days 6-16) but recovered rapidly after stopping treatment. For the anisomycin SET Combination Drugs, the weight loss resulted in a number of animals being sacrificed for >20% weight loss and not tumor regrowth; Arm #4 (2 of 8 animals by Day 14) and Arm #5 (4 of 8 animals by Day 21), which contrasted with no animal sacrifices in Arms #2 and #3. Although the average weight change produced by the High Dose emetine SET Combination Drug was statistically greater than capecitabine treated animals (Days 12-14, p=0.02), animal weights rapidly recovered and exhibited a statistically significant weight gain by Days 29 (p=0.0006). As in animal study 1, this weight gain correlated with the maximal tumor regression (FIG. 14B). These results showed that the SET Combination Drug formulations enhanced capecitabine-induced animal weight loss and the anisomycin containing drugs may not be as effective as an emetine containing drug for lowering animal sacrifice due to >20% weight loss. However, animal weight increased for each SET Combination Drug within 10 days of stopping treatment and reached statistical significance in the emetine SET Combination Drug.

Taken together, Example 3 proves that the anisomycin and emetine SET Combination Drugs improved capecitabine drug action by killing mitotic cells at the tumor surface and non-mitotic cells in the necrotic core (FIGS. 17A-17J), produced extensive tumor regression (Tables 8 and 11), significantly improved tumor responses in combination with high and low dose capecitabine (FIGS. 11A and 14A), reduced/reversed animal weight changes (FIGS. 12A and 15), and significantly increased animal survival (FIGS. 13 and 16).

TABLE 1
18S rRNA Complementary Elements

| Name | Alignment |
|---|---|
| 18S rRNA | REINITIATION IRES |
| HELIX26 | 5' GCGAUGCGGCGGCGUUAUUCCCAUG [symbol] GCAGCUUCCGGGA 3' |
| SEQ ID NO: 10 | |
| Influenza | 3' CUCGACUUAAAGGGUAUCUCGAGAC 5' |
| SEQ ID NO: 11 | |
| FCV | 3' GGUUAACAUAAGGGUACAUCCUCCG 5' |
| SEQ ID NO: 12 | |
| RHDV | 3' GACUUAAAGGGUAUCUCGAGAC 5' |
| SEQ ID NO: 13 | |
| GTX IRES | 3' CGGGGGCG [symbol] GAGCCC 5' |
| SEQ ID NO: 14 | |
| TR IRES | 3' ACAUGUGUCCAU [symbol] U [symbol] U [symbol] CGUUUGU 5' |
| SEQ ID NO: 15 | |
| TR REGULATOR | 3' CUUGAACCACGGAGCCGGGUACUCAAAUUCCUGCCGCUUCAA 5' |
| SEQ ID NO: 16 | |

TABLE 2
Preferred RNA Structures (Most stable secondary structures)

| Mutation | | | | SET |
|---|---|---|---|---|
| (Isoform) | Structure 1 | Structure 2 | Structure 3 | Regulation |
| Wildtype | dG = −11.40 | dG = −11.20 | dG = −11.0 | Native |
| A701G (DM20) | dG = −11.40 | dG = −11.20 | dG = −11.0 | Native |
| A722G (DM20) | dG = −8.68 | dG = −7.92 | dG = −11.90 | Disrupted |
| A701G | dG = −8.68 | dG = −7.92 | dG = −11.90 | Disrupted |
| A722G (DM20) | | | | |
| A667T (DM20) | dG = −11.00 | dG = −10.7 | dG = −10.5 | Disrupted |
| A706T (DM20) | dG = −11.40 | dG = −11.20 | dG = −10.6 | Native |
| A667T | dG = −11.00 | dG = −10.7 | dG = −10.5 | Disrupted |
| A706T (DM20) | | | | |

TABLE 3
TR SET Reference Standard Reagents

| | | Concentra- | | SET |
|---|---|---|---|---|
| | Toxin Name | tion(s) | | Response |
| 1 | dbcAMP¹ | 5 | mM | Antagonist/ |
| | | | | Agonist |
| 2 | TPA² | 100 | nM | Agonist |
| 3 | Paclitaxel | 500 | nM | Agonist |
| 4 | MG132³ | 50 | uM | Antagonist/ |
| | | | | Agonist |
| 5 | High dose (HD) Calon⁴ | 10 | uM | Antagonist |
| 6 | Low dose (LD) Calon⁴ | 1 | uM | Agonist |
| 7 | Low Dose (LD) Topotecan⁵ | 100 | nM | Agonist |
| 8 | High Dose (HD) Topotecan⁵ | 10 | uM | Antagonist |
| 9 | Colchicine⁶ | 1 | uM | Agonist |
| 10 | MRA⁷ | 150 | nM | Antagonist |
| 11 | Bortezomib (Velcade)⁸ | 50 | nM | Antagonist/ |
| | | | | Agonist |

¹Dibutyryl-cyclic AMP
²12-O-tetradecanoylphorbol-13-acetate
³Z-Leu-Leu-Leu-aldehyde
⁴Calcium Ionophore A23187, Calcimycin
⁵(S)-10-[(dimethylamino)methyl]-4-ethyl-4,9-dihydroxy-1H-pyrano[3′,4′:6,7]indolizino[1,2-b]quinoline-3,14(4H,12H)-dione monohydrochloride; Topetecan
⁶N-[(7S)-1,2,3,10-tetramethoxy-9-oxo-5,6,7,9-tetrahydrobenzo[a]heptalen-7-yl]acetamide
⁷Mycoplasma Removal Agent, 4-oxoquinoline-3-carboxylic acid derivative
⁸[(1R)-3-methyl-1-[[(2S)-1-oxo-3-phenyl-2-[(pyrazinylcarbonyl) amino]propyl]amino]butyl] boronic acid; Velcade

TABLE 4
SET Activity following Nonadherent
Growth of the HCT116 TR Cell Panel

| | SET Activity | SET Activity | |
|---|---|---|---|
| Cell Line Name | (day 0) | (post-growth) | % change |
| mTRdm-fLUC#17 | 226.3% | 366.0% | 161.7% |
| mTRdm-fLUC#25** | 336.6% | 26,675.6% | 7,925.0% |
| mTRdm-fLUC#28** | 1,365.5% | 20,492.8% | 1,500.8% |
| mTRdm-fLUC#36 | 875.3% | 3,926.9% | 448.6% |
| mTRdm-fLUC#47 | 237.5% | 1,434.3% | 603.9% |
| mTRdm-fLUC#49 | 329.4% | 2,015.4% | 611.8% |
| mTRdm-fLUC#52 | 261.1% | 920.0% | 352.4% |
| mTRdm-fLUC#58 | 324.4% | 636.6% | 196.2% |
| mTRdm-fLUC#75** | 693.1% | 19,413.0% | 2,800.9% |
| mTRdm-fLUC#139 | 114.8% | 311.9% | 271.7% |
| mTRdm-fLUC#156 | 462.3% | 3,673.3% | 794.6% |
| mTRdm-fLUC#190 | 204.4% | 178.3% | 87.2% |
| mTRdm-fLUC#220 | 115.5% | 81.8% | 70.8% |

**Outlier cell lines that produce tumorsphere with enhanced SET induction and are TR metastatic cancer cell line candidates

TABLE 5
Enhanced Tumor Growth in the Metastatic Cancer Cell Model

| HCT116 hTRdm-fLUC#32 Cells | | | | | HCT116 Parental Cell Line | | | | |
|---|---|---|---|---|---|---|---|---|---|
| Implant | Tumor Size | | | | Implant | Tumor Size | | | |
| site | Day 15 | | Day 21 | | site | Day 15 | | Day 21 | |
| 6R* | 162 | mg | 1368 | mg | 3R | 100 | mg | 2138 | mg |
| 1R | 100 | mg | 1271 | mg | 5R | 113 | mg | 1210 | mg |
| 5R | 162 | mg | 1200 | mg | 4L | 100 | mg | 750 | mg |
| 2R | 162 | mg | 1150 | mg | 4R | 144 | mg | 726 | mg |
| 6L | 100 | mg | 666 | mg | 6R | 126 | mg | 550 | mg |
| 3R | 100 | mg | 650 | mg | 6L | 113 | mg | 544 | mg |
| 5L | 75 | mg | 448 | mg | 1R | 144 | mg | 527 | mg |
| 1L | 88 | mg | 352 | mg | 1L | 113 | mg | 416 | mg |
| 4R | 113 | mg | 288 | mg | 5L | 75 | mg | 365 | mg |
| 4L | 100 | mg | 221 | mg | 3L | 75 | mg | 138 | mg |
| 3L | 75 | mg | 162 | mg | 2L | 88 | mg | 144 | mg |
| 2L | 88 | mg | 180 | mg | 2R | 144 | mg | 144 | mg |

Day 21 Tumor Size Distribution

| | |
|---|---|
| Tumor Size Category | Tumor Size Category |

| | | | | | |
|---|---|---|---|---|---|
| 0-200 | mg | n = 2 | 0-200 | mg | n = 3 |
| 201-600 | mg | n = 4 | 201-600 | mg | n = 5 |
| 601-2200 | mg | n = 6 | 601-2200 | mg | n = 4 |

*Nomenclature - 6R refers to animal #6 implanted on the right side

TABLE 6
Pre- and Post-treament Bioluminescence
Signal Intensity (Table 7 animals)
Aminals with an Average Tumor Size of 125 mg

| | Pre- | 6 hr Post- | |
|---|---|---|---|
| | treatment Value | treatment Value | |
| | (×10e6 | (×10e6 | |
| Arm-#Animal | photons) | photons) | % Induction |
| 1 - #1 | 58.8 | 41.6 | 70.7% |
| 1 - #2 | 1.2* | 152.5 | 12,708.3% *** |
| 1 - #3 | 0.2* | 57.8 | 28,900% *** |
| 1 - #4 | 18.8 | 44.1 | 234.6% |
| 1 - #5 | 32.2 | 45.8 | 142.2% |
| 1 - #6 | 28.3 | 29.5 | 104.2% |
| 2 - #1 | 29.6 | 37.9 | 128.0% |
| 2 - #2 | 24.7 | 55.7 | 225.5% |
| 2 - #3 | 31.8 | 37.4 | 117.6% |
| 2 - #4 | 0.2* | 93.2 | 46,600.0% *** |
| 2 - #5 | 1.2* | 86.3 | 7,191.7% *** |
| 2 - #6 | 0.5* | 52.7 | 10,540.0% *** |
| 3 - #1 | 0.3* | 38.6 | 12,866.7% *** |
| 3 - #2 | 13.2 | 50.4 | 381.8% |
| 3 - #3 | 60.4 | 101.0 | 167.2% |
| 3 - #4 | 0.3* | 39.3 | 13,100.0% *** |
| 3 - #5 | 22.1 | 82.5 | 373.3% |
| 3 - #6 | 0.3* | 56.8 | 18,933.3% *** |

Animals with an Average Tumor Size of 500 mg

| | Pre- | 6 hr Post- | |
|---|---|---|---|
| Arm-#Animal | treatment Value | treatment Value | % Induction |
| 1 - #1 | 34.7 (×10e6 | 79.5 (×10e6 | 229.1% |
| | photons) | photons) | |
| 1 - #2 | 20.7 | 82.5 | 398.6% |
| 1 - #3 | 18.7 | 76.0 | 406.4% |
| 2 - #1 | 40.4 | 61.1 | 151.2% |
| 2 - #2 | 0.5 * | 102.5 | 20,500.0% *** |
| 2 - #3 | 16.0 | 28.1 | 175.6% |
| 3 - #1 | 36.6 | 75.0 | 204.9% |
| 3 - #2 | 22.1 | 81.5 | 368.8% |
| 3 - #3 | 44.5 | 48.8 | 109.7% |

Arm 1 - CremophorEL;
Arm 2 - Paclitaxel/CremophorEL;
Arm 3 - Cyclophosphamide
* Denotes animals with a signifiantly lower Pre-treatment biolumiscence level
*** Denotes animals with a highly significant increase in Post-treatment bioluminescence (translation of the fLUC reporter protein by the SET Ribosome)

TABLE 7
First Xenogenic Animal Study

| | Test Arm Name | Drug & Concentrations |
|---|---|---|
| Arm #1 | Vehicle (SET | 0.5 mg/kg/day or 75.8 mg/sq m/day |
| | Agonist) | CremophorEL |
| Arm #2 | Capecitabine | 500 mg/kg/day or 1500 mg/sq m/day |
| | (Cytotoxic) | Capecitabine |
| | 78% of a human | |
| | cycle dose | |
| Arm #3 | CaCy Components | 0.5 mg/kg/day or 75.8 mg/sq m/day |
| | (SET Agonist & | CremophorEL, 0.000054 mg/kg/day or |
| | Antagonist) | 0.00016 mg/sq m/day Anisomycin |
| Arm #4 | Low Dose | 500 mg/kg/day or 1500 mg/sq m/day |
| | Anisomycin | Capecitabine, 0.5 mg/kg/day or |
| | (Combination) | 75.8 mg/sq m/day CremophorEL, |
| | | 0.000027 mg/kg/day or 0.00008 mg/sq |
| | | m/day Anisomycin |
| Arm #5 | High Dose | 500 mg/kg/day or 1500 mg/sq m/day |
| | Anisomycin | Capecitabine, 0.5 mg/kg/day or |
| | (Combination) | 75.8 mg/sq m/day CremophorEL, |
| | | 0.000054 mg/kg/day or 0.00016 mg/sq |
| | | m/day Anisomycin |

TABLE 8
First Xenogenic Animal Study (Ranking Tumor
Regression in animals during Treatment)

| | Arm #2 | Arm #4 | Arm #5 |
|---|---|---|---|
| | Drug | | |
| | Cape | Anisomycin | Anisomycin |
| | (Cytotoxic) | (L) | (H) |
| | 9.1% | 11.3% | 33.9% |
| | 40.7% | 20.0% | 59.8% |
| | 53.5% | 45.1% | 69.4% |
| | 55.8% | 50.0% | 75.3% |
| | 56.1% | 57.5% | 78.1% |
| | 64.0% | 87.9% | 81.5% |
| | 67.9% | | 91.5% |
| | 78.1% | | 99.9% |
| Mean | 56.0% | 47.6% | 76.7% |
| Average | 53.2% | 45.3% | 73.7% |
| SD | 20.9% | 27.5% | 20.3% |

TABLE 9
Statistical Analysis of Animal Weight Changes in First Xenogenic Animal Study
Animal weight averages (g) per day of study

| | day 7 | day 10 | day 13 | day 15 | day 18 | day 24 | day 31 | day 35 | day 38 |
|---|---|---|---|---|---|---|---|---|---|
| Arm1 | 22.25 | 22.39 | 22.23 | 21.60 | 21.93 | 21.59 | 20.22 | | |
| Arm2 | 20.88 | 20.88 | 20.36 | 19.38 | 18.50 | 19.56 | 21.17 | 22.32 | 22.99 |
| Arm3 | 21.29 | 22.23+ | 21.65 | 21.06 | 21.33 | 20.39 | 21.05 | | |
| Arm4 | 20.33 | 19.99* | 18.20* | 16.39* | 18.58 | 19.19 | 22.47+ | 23.27+ | 23.77+ |
| Arm5 | 21.44 | 21.94 | 21.01 | 19.78* | 20.29 | 21.72 | 23.55+ | 24.36+ | 24.60+ |

+Significant (p < 0.05; 2-tailed tTest) weight gain compared to day 7
*Significant (p < 0.05; 2-tailed tTest) weight loss compared to day 7

TABLE 10
Second Xenogenic Animal Study

| | Test Arm Name | Drug Concentrations |
|---|---|---|
| Arm #1 | Vehicle (SET | 0.5 mg/kg/day or 75.8 mg/sq m/day |
| | Agonist) | CremophorEL |
| Arm #2 | Capecitabine | 400 mg/kg/day or 1200 mg/sq m/day |
| | (Cytostatic) | Capecitabine |
| | 35% of a human | |
| | cycle dose | |
| Arm #3 | Emetine | 400 mg/kg/day or 1200 mg/sq m/day |
| | (Combination) | Capecitabine, 0.5 mg/kg/day or |
| | | 75.8 mg/sq m/day CremophorEL, |
| | | 0.00013 mg/kg/day or |
| | | 0.0004 mg/sq m/day Emetine |
| Arm #4 | High Dose | 400 mg/kg/day or 1200 mg/sq m/day |
| | Anisomycin | Capecitabine, 0.5 mg/kg/day or |
| | (Combination) | 75.8 mg/sq m/day CremophorEL, |
| | | 0.000054 mg/kg/day or |
| | | 0.00016 mg/sq m/day Anisomycin |
| Arm #5 | Very High Dose | 400 mg/kg/day or 1200 mg/sq m/day |
| | Anisomycin | Capecitabine, 0.5 mg/kg/day or |
| | (Combination) | 75.8 mg/sq m/day CremophorEL, |
| | | 0.00013 mg/kg/day or |
| | | 0.0004 mg/sq m/day Anisomycin |

TABLE 11
Second Xenogenic Animal Study (Ranking Tumor
Regression in animals during Treatment)

| | Arm #2 | Arm #3 | Arm #4 | Arm #5 |
|---|---|---|---|---|
| | Drug | | | |
| | Cape | | Anisomycin | Anisomycin |
| | (Cytostatic) | Emetine | (H) | (VH) |
| | 0.0% | 9.9% | 10.0% | 9.9% |
| | 9.2% | 51.1% | 38.8% | 28.9% |
| | 19.0% | 65.6% | 50.8% | 51.6% |
| | 33.6% | 68.9% | 50.8% | 56.4% |
| | 36.8% | 73.3% | 75.9% | 62.5% |
| | 42.4% | 81.9% | 83.7% | 67.9% |
| | 45.4% | 90.0% | | |
| | 48.8% | 90.6% | | |
| Mean | 35.2% | 71.1% | 50.8% | 54.0% |
| Average | 29.4% | 66.4% | 51.6% | 46.2% |
| SD | 18.0% | 26.4% | 26.5% | 22.3% |

TABLE 12
Analyzing Cell Distribution in Treated Xenogenic
Tumors from First Animal Study

| | Arm 2 Animal #1 Tumor | | | Arm 5 Animal #2 Tumor | | |
|---|---|---|---|---|---|---|
| | Layer 1 | Layer 2 | Layer 3 | Layer 1 | Layer 2 | Layer 3 |
| Antibody | fLUC | F4/80 | F4/80 | fLUC | F4/80 | F4/80 |
| Marker | | | | | | |
| Avg. Cell | 3.43 | 6.43 | 16.6 | 7.8 | 7.8 | 18.6 |
| Thickness | | | | | | |
| Standard | 1.1 | 2.6 | 2.9 | 5.5 | 5.8 | 7.8 |
| Deviation | | | | | | |
| Mean | 3.0 | 5.5 | 15.0 | 5.5 | 5.0 | 20.0 |
| 2-tailed | | | | 0.008 | 0.430 | 0.439 |
| tTest | | | | | | |
| Counted | n = 14 | n = 14 | n = 11 | n = 12 | n = 11 | n = 7 |
| Sections | | | | | | |

TABLE 13
Drug Combinations

| Ref. No | SET Agonist | Cytotoxic Drug | SET Antagonist |
|---|---|---|---|
| 1 | polyoxyl 35 castor oil* | Capecitabine | Anisomycin |
| 2 | polyoxyl 35 castor oil | Capecitabine | Emetine |
| 3 | polyoxyl 35 castor oil | Capecitabine | Cycloheximide |
| 4 | polyoxyl 35 castor oil | 5-FU/leucovorin | Anisomycin |
| 5 | polyoxyl 35 castor oil | 5-FU/leucovorin | Emetine |
| 6 | polyoxyl 35 castor oil | 5-FU/leucovorin | Cycloheximide |
| 7 | polyoxyl 35 castor oil | paclitaxel | Anisomycin |
| 8 | polyoxyl 35 castor oil | paclitaxel | Emetine |
| 9 | polyoxyl 35 castor oil | paclitaxel | Cycloheximide |
| 10 | polyoxyl 35 castor oil | docetaxel | Anisomycin |
| 11 | polyoxyl 35 castor oil | docetaxel | Emetine |
| 12 | polyoxyl 35 castor oil | docetaxel | Cycloheximide |
| 13 | polyoxyl 35 castor oil | cyclophosphamide | Anisomycin |
| 14 | polyoxyl 35 castor oil | cyclophosphamide | Emetine |
| 15 | polyoxyl 35 castor oil | cyclophosphamide | Cycloheximide |
| 16 | polyoxyl 35 castor oil | topotecan | Anisomycin |
| 17 | polyoxyl 35 castor oil | topotecan | Emetine |
| 18 | polyoxyl 35 castor oil | topotecan | Cycloheximide |
| 19 | polyoxyl 35 castor oil | irinotecan | Anisomycin |
| 20 | polyoxyl 35 castor oil | irinotecan | Emetine |
| 21 | polyoxyl 35 castor oil | irinotecan | Cycloheximide |
| 22 | polyoxyl 35 castor oil | oxaliplatin | Anisomycin |
| 23 | polyoxyl 35 castor oil | oxaliplatin | Emetine |
| 24 | polyoxyl 35 castor oil | oxaliplatin | Cycloheximide |
| 25 | polyoxyl 40 castor oil** | Capecitabine | Anisomycin |
| 26 | polyoxyl 40 castor oil | Capecitabine | Emetine |
| 27 | polyoxyl 40 castor oil | Capecitabine | Cycloheximide |
| 28 | polyoxyl 40 castor oil | 5-FU/leucovorin | Anisomycin |
| 29 | polyoxyl 40 castor oil | 5-FU/leucovorin | Emetine |
| 30 | polyoxyl 40 castor oil | 5-FU/leucovorin | Cycloheximide |
| 31 | polyoxyl 40 castor oil | paclitaxel | Anisomycin |
| 32 | polyoxyl 40 castor oil | paclitaxel | Emetine |
| 33 | polyoxyl 40 castor oil | paclitaxel | Cycloheximide |
| 34 | polyoxyl 40 castor oil | docetaxel | Anisomycin |
| 35 | polyoxyl 40 castor oil | docetaxel | Emetine |
| 36 | polyoxyl 40 castor oil | docetaxel | Cycloheximide |
| 37 | polyoxyl 40 castor oil | cyclophosphamide | Anisomycin |
| 38 | polyoxyl 40 castor oil | cyclophosphamide | Emetine |
| 39 | polyoxyl 40 castor oil | cyclophosphamide | Cycloheximide |
| 40 | polyoxyl 40 castor oil | topotecan | Anisomycin |
| 41 | polyoxyl 40 castor oil | topotecan | Emetine |
| 42 | polyoxyl 40 castor oil | topotecan | Cycloheximide |
| 43 | polyoxyl 40 castor oil | irinotecan | Anisomycin |
| 44 | polyoxyl 40 castor oil | irinotecan | Emetine |
| 45 | polyoxyl 40 castor oil | irinotecan | Cycloheximide |
| 46 | polyoxyl 40 castor oil | oxaliplatin | Anisomycin |
| 47 | polyoxyl 40 castor oil | oxaliplatin | Emetine |
| 48 | polyoxyl 40 castor oil | oxaliplatin | Cycloheximide |
| 49 | phorbol-12-myristate-13-acetate*** | Capecitabine | Anisomycin |
| 50 | phorbol-12-myristate-13-acetate | Capecitabine | Emetine |
| 51 | phorbol-12-myristate-13-acetate | Capecitabine | Cycloheximide |
| 52 | phorbol-12-myristate-13-acetate | 5-FU/leucovorin | Anisomycin |
| 53 | phorbol-12-myristate-13-acetate | 5-FU/leucovorin | Emetine |
| 54 | phorbol-12-myristate-13-acetate | 5-FU/leucovorin | Cycloheximide |
| 55 | phorbol-12-myristate-13-acetate | paclitaxel | Anisomycin |
| 56 | phorbol-12-myristate-13-acetate | paclitaxel | Emetine |
| 57 | phorbol-12-myristate-13-acetate | paclitaxel | Cycloheximide |
| 58 | phorbol-12-myristate-13-acetate | docetaxel | Anisomycin |
| 59 | phorbol-12-myristate-13-acetate | docetaxel | Emetine |
| 60 | phorbol-12-myristate-13-acetate | docetaxel | Cycloheximide |
| 61 | phorbol-12-myristate-13-acetate | cyclophosphamide | Anisomycin |
| 62 | phorbol-12-myristate-13-acetate | cyclophosphamide | Emetine |
| 63 | phorbol-12-myristate-13-acetate | cyclophosphamide | Cycloheximide |
| 64 | phorbol-12-myristate-13-acetate | topotecan | Anisomycin |
| 65 | phorbol-12-myristate-13-acetate | topotecan | Emetine |
| 66 | phorbol-12-myristate-13-acetate | topotecan | Cycloheximide |
| 67 | phorbol-12-myristate-13-acetate | irinotecan | Anisomycin |
| 68 | phorbol-12-myristate-13-acetate | irinotecan | Emetine |
| 69 | phorbol-12-myristate-13-acetate | irinotecan | Cycloheximide |
| 70 | phorbol-12-myristate-13-acetate | oxaliplatin | Anisomycin |
| 71 | phorbol-12-myristate-13-acetate | oxaliplatin | Emetine |
| 72 | phorbol-12-myristate-13-acetate | oxaliplatin | Cycloheximide |
| 73 | Bryostatin1**** | Capecitabine | Anisomycin |
| 74 | Bryostatin1 | Capecitabine | Emetine |
| 75 | Bryostatin1 | Capecitabine | Cycloheximide |
| 76 | Bryostatin1 | 5-FU/leucovorin | Anisomycin |
| 77 | Bryostatin1 | 5-FU/leucovorin | Emetine |
| 78 | Bryostatin1 | 5-FU/leucovorin | Cycloheximide |
| 79 | Bryostatin1 | paclitaxel | Anisomycin |
| 80 | Bryostatin1 | paclitaxel | Emetine |
| 81 | Bryostatin1 | paclitaxel | Cycloheximide |
| 82 | Bryostatin1 | docetaxel | Anisomycin |
| 83 | Bryostatin1 | docetaxel | Emetine |
| 84 | Bryostatin1 | docetaxel | Cycloheximide |
| 85 | Bryostatin1 | cyclophosphamide | Anisomycin |
| 86 | Bryostatin1 | cyclophosphamide | Emetine |
| 87 | Bryostatin1 | cyclophosphamide | Cycloheximide |
| 88 | Bryostatin1 | topotecan | Anisomycin |
| 89 | Bryostatin1 | topotecan | Emetine |
| 90 | Bryostatin1 | topotecan | Cycloheximide |
| 91 | Bryostatin1 | irinotecan | Anisomycin |
| 92 | Bryostatin1 | irinotecan | Emetine |
| 93 | Bryostatin1 | irinotecan | Cycloheximide |
| 94 | Bryostatin1 | oxaliplatin | Anisomycin |
| 95 | Bryostatin1 | oxaliplatin | Emetine |
| 96 | Bryostatin1 | oxaliplatin | Cycloheximide |

*Also known as CremophorEL
**Also known as CremophorRH
***Delivered in CremophorEL or CremophorRH
****Delivered in CremophorEL or CremophorRH

### Items

Item 1. A pharmaceutical composition, comprising:

a SET agonist and a SET ribosome antagonist.

Item 2. The pharmaceutical composition of item 1, wherein the SET agonist is a stimulator of G2 phase progression.

Item 3. The pharmaceutical composition of item 1 or 2, wherein the SET agonist is selected from the group consisting of: a polyoxyl hydrogenated castor oil; a phorbol ester; a bryostatin; a pharmaceutically acceptable salt of any thereof; and a combination of any two or more thereof.

Item 4. The pharmaceutical composition of any of items 1-3, wherein the polyoxyl hydrogenated castor oil is selected from the group consisting of: polyoxyl 30 hydrogenated castor oil; polyoxyl 35 hydrogenated castor oil; polyoxyl 40 hydrogenated castor oil; polyoxyl 50 hydrogenated castor oil; polyoxyl 60 hydrogenated castor oil; and a combination of any two or more thereof.

Item 5. The pharmaceutical composition of any of items 1-4, wherein the polyoxyl hydrogenated castor oil is selected from the group consisting of: polyoxyl 35 hydrogenated castor oil; polyoxyl 40 hydrogenated castor oil; and a combination thereof.

Item 6. The pharmaceutical composition of any of items 1-5, wherein the bryostatin is selected from the group consisting of: bryostatin 1; bryostatin 2; a pharmaceutically acceptable salt of either thereof; and a combination of any two or more thereof.

Item 7. The pharmaceutical composition of any of items 1-6, wherein the phorbol ester is 12-O-tetradecanoylphorbol-13-acetate or a pharmaceutically acceptable salt thereof.

Item 8. The pharmaceutical composition of any of items 1-7, wherein the SET ribosome antagonist inhibits protein synthesis by SET Ribosomes.

Item 9. The pharmaceutical composition of any of items 1-8, wherein the SET ribosome antagonist is selected from the group consisting of: anisomycin; emetine; cycloheximide; a pharmaceutically acceptable salt of any thereof; and a combination of any two or more thereof.

Item 10. The pharmaceutical composition of any of items 1-9, wherein the SET agonist comprises polyoxyl 35 hydrogenated castor oil and the SET ribosome antagonist comprises anisomycin or a pharmaceutically acceptable salt thereof.

Item 11. The pharmaceutical composition of any of items 1-10, wherein the SET agonist comprises polyoxyl 35 hydrogenated castor oil and the SET ribosome antagonist comprises emetine or a pharmaceutically acceptable salt thereof.

Item 12. The pharmaceutical composition of any of items 1-11, formulated for oral administration to a subject.

Item 13. A method of identifying an agent effective to promote or inhibit G2 progression in vivo are provided according to aspects of the present invention which include providing a cell of a TR Class 4 cell line characterized by a TR Class 3 outlier SET response, wherein the cell comprises a TR nucleic acid expression cassette encoding a TR element and a reporter; wherein the expression cassette is stably integrated into the genome of the cells; administering the cell to a non-human animal, producing a xenograft tumor in the non-human animal; administering a test substance to the non-human animal; and measuring the effect of the test substance on the SET response, wherein an increase in a SET response identifies the agent as a SET agonist effective to promote G2 progression in vivo.

Item 14. The method of item 13, further comprising administering a SET agonist to the non-human animal to promote G2 progression in vivo, wherein a decrease in the SET response identifies the agent as a SET antagonist effective to inhibit G2 progression in vivo.

Item 15. The method of item 13 or 14, further comprising measuring the effect of the test substance on the xenograft tumor.

Item 16. The method of any of items 13-15, wherein the non-human animal is a rat or mouse.

Item 17. A method of identifying an agent effective as a component of a SET Combination drug for treatment a proliferative disease, comprising:

providing a cell characterized by a TR Class 3 SET response or a TR Class 3 SET outlier response, wherein the cell comprises an expression construct encoding a TR element and a reporter stably integrated in the genome of the cell;

contacting the cell with a test substance; and

measuring the effect of the test substance on protein synthesis from a SET ribosome compared to a control, wherein inhibition of protein synthesis from a SET ribosome by the test substance identifies the substance as an agent effective as a component of a SET Combination drug for treatment a proliferative disease.

Item 18. The method of item 17, wherein the cell is further characterized by in vitro ability to grow in suspension cultures as nonadherent 3D structures and the ability to initiate and grow into a primary xenogenic tumor in vivo, that can be dissected into subfragments and propagated as a secondary tumor.

Item 19. A method of generating a metastatic cancer cell line model, comprising:

introducing an expression cassette encoding a TR element and a reporter into a cell, producing a parental population of cells wherein the expression cassette is stably integrated into the genome of the cells;

isolating subclones of the parental population;

administering a SET agonist to a population of cells of each subclone to induce a SET TR response in the population of cells of each subclone;

assaying the TR SET response in the population of cells of each subclone by detecting expression of the reporter;

ranking the TR SET response of each subclone compared to each other subclone, establishing a range of TR SET responses characterized by an average response;

selecting the subclones characterized by detectable increases in expression of the reporter of at least two standard deviations greater than the mean response, thereby defining the selected subclones as TR Class 3 TR SET response subclones;

administering a SET agonist to a population of cells of each TR Class 3 TR SET response subclone to induce a SET TR response in the population of cells of each TR Class 3 TR SET response subclone;

assaying the TR SET response in the population of cells of each TR Class 3 SET response subclone by detecting expression of the reporter;

ranking the TR SET response of each TR Class 3 SET response subclone compared to each other TR Class 3 SET response subclone, establishing a range of TR SET responses characterized by an average response;

selecting the TR Class 3 SET response subclones characterized by detectable increases in expression of the reporter of at least two standard deviations greater than the mean response, thereby defining the selected TR Class 3 SET response subclones as TR Class 3 SET response outliers;

administering one or more toxins to cells of one or more subclones characterized as a TR Class 3 SET response outliers; and

detecting a response of the cells of the one or more subclones characterized as a TR

Class 3 SET response outliers indicative of drug and stress resistance due to elevated SET ribosome activity in the cells of the subclone, thereby determining that the cells are TR Class 4 cells; and thereby generating a metastatic cancer cell line model.

Item 20. The method of item 19, further comprising:

culturing the TR Class 4 cells under low density conditions for at least 50 cell cycles, generating TR Class 4 subclones and capable of low density colony formation;

selecting the TR Class 4 subclones capable of low density colony formation;

administering a SET agonist to a population of cells of each TR Class 4 subclone capable of low density colony formation to induce a TR SET response;

assaying the SET response in the population of cells of each TR Class 4 subclone capable of low density colony formation to induce a TR SET response by detecting expression of the reporter;

ranking the TR SET response of each TR Class 4 subclone capable of low density colony formation compared to each other TR Class 4 subclone capable of low density colony formation establishing a range of SET responses characterized by an average response; and

selecting the TR Class 4 subclones capable of low density colony formation and characterized by detectable increases in expression of the reporter of at least two standard deviations greater than the mean response.

Item 21. The method of item 19 or 20, further comprising:

culturing the TR Class 4 cells under nonadherent low density culture conditions;

selecting subclones of the TR Class 4 cells that grow as suspended aggregates, thereby selecting subclones of TR Class 4 cells capable of ex vivo tumorsphere formation with 10 or fewer cells initiating the tumorsphere;

administering one or more toxins to cells of the TR Class 4 subclones capable of ex vivo tumorsphere formation with 10 or fewer cells initiating the tumorsphere response; and

detecting a response of the cells of the TR Class 4 subclones capable of ex vivo tumorsphere formation with 10 or fewer cells initiating the tumorsphere indicative of drug and stress resistance due to elevated SET ribosome activity in the cells of the subclone, thereby determining that the cells of the TR Class 4 subclones are capable of ex vivo tumorsphere formation with 10 or fewer cells, characterized by a TR Class 4 SET response.

Item 22. An isolated, non-naturally occurring, cell characterized by a class 3 outlier SET response, wherein the cell comprises an expression cassette encoding a TR element and a reporter stably integrated in the genome of the cell.

Item 23. The cell of item 22, further characterized by in vitro ability to grow in suspension cultures as nonadherent 3D structures and the ability to initiate and grow into a primary xenogenic tumor in vivo, that can be dissected into subfragments and propagated as a secondary tumor.

Item 24. A method for treatment of a proliferative disorder characterized by abnormal cells in a mammalian subject, comprising:

administering a pharmaceutically effective amount of a combination of: a cytotoxic agent, a SET agonist and a SET ribosome antagonist.

Item 25. The method of item 24, wherein the abnormal cells comprise both mitotic abnormal cells and non-mitotic abnormal and wherein both abnormal cells and non-mitotic abnormal induced to die due to the administering of the pharmaceutically effective amount of a combination of: a cytotoxic agent, a SET agonist and a SET ribosome antagonist.

Item 26. The method of item 24 or 25, wherein the combination of a cytotoxic agent, a SET agonist and a SET ribosome antagonist is effective such that a lower dose of the cytotoxic agent is required to kill the abnormal cells compared to treatment by administering the cytotoxic agent without the SET agonist and the SET ribosome antagonist.

Item 27. The method of any of items 24-26, wherein the cytotoxic agent is selected from the group consisting of: 5-fluorouracil, leucovorin, capecitabine, cyclophosphamide, irinotecan, topotecan, paclitaxel, docetaxel, oxaliplatin, a pharmaceutically acceptable salt thereof and a combination of any two or more thereof.

Item 28. The method of any of items 24-27, wherein the SET agonist is a stimulator of G2 phase progression.

Item 29. The method of any of items 24-28, wherein the SET agonist is selected from the group consisting of: a polyoxyl hydrogenated castor oil; a phorbol ester; a bryostatin; a pharmaceutically acceptable salt of any thereof; and a combination of any two or more thereof.

Item 30. The method of any of items 24-29, wherein the polyoxyl hydrogenated castor oil is selected from the group consisting of: polyoxyl 30 hydrogenated castor oil; polyoxyl 35 hydrogenated castor oil; polyoxyl 40 hydrogenated castor oil; polyoxyl 50 hydrogenated castor oil; polyoxyl 60 hydrogenated castor oil; and a combination of any two or more thereof.

Item 31. The method of any of items 24-30, wherein the polyoxyl hydrogenated castor oil is selected from the group consisting of: polyoxyl 35 hydrogenated castor oil; polyoxyl 40 hydrogenated castor oil; and a combination thereof.

Item 32. The method of any of items 24-31, wherein the bryostatin is selected from the group consisting of: bryostatin 1; bryostatin 2; a pharmaceutically acceptable salt of either thereof; and a combination of any two or more thereof.

Item 33. The method of any of items 24-32, wherein the phorbol ester is 12-O-tetradecanoylphorbol-13-acetate or a pharmaceutically acceptable salt thereof.

Item 34. The method of any of items 24-33, wherein the SET ribosome antagonist inhibits protein synthesis by SET Ribosomes.

Item 35. The method of any of items 24-34, wherein the SET ribosome antagonist is selected from the group consisting of: anisomycin; cycloheximide; emetine; a pharmaceutically acceptable salt of any thereof; and a combination of any two or more thereof.

Item 36. The method of any of items 24-35, wherein the cytotoxic agent comprises capecitabine or a pharmaceutically acceptable salt thereof; the SET agonist comprises polyoxyl 35 hydrogenated castor oil and the SET ribosome antagonist comprises anisomycin or a pharmaceutically acceptable salt thereof.

Item 37. The method of any of items 24-36, wherein the cytotoxic agent comprises capecitabine or a pharmaceutically acceptable salt thereof; the SET agonist comprises polyoxyl 35 hydrogenated castor oil and the SET ribosome antagonist comprises emetine or a pharmaceutically acceptable salt thereof.

Item 38. The method of any of items 24-37, wherein the subject is human.

Item 39. The method of any of items 24-38, wherein the proliferative disorder is drug-resistant cancer and/or metastatic cancer.

Item 40. The method of any of items 24-39, wherein the cytotoxic agent, the SET agonist and the SET ribosome antagonist are administered simultaneously.

Item 41. The method of any of items 24-40, wherein the cytotoxic agent, the SET agonist and the SET ribosome antagonist are administered at different times.

Item 42. The method of any of items 24-41, wherein the SET agonist and the SET ribosome antagonist are administered together in a pharmaceutical formulation.

Item 43. The method of any of items 24-42, wherein the SET agonist and the SET ribosome antagonist are administered orally together in a pharmaceutical formulation.

Item 44. The method of any of items 24-43, further comprising an adjunct therapeutic treatment.

Item 45. The method of any of items 24-44, wherein the adjunct therapeutic treatment comprises radiation treatment of the subject.

Item 46. The method of any of items 24-45, wherein the adjunct therapeutic treatment comprises administration of one or more additional cytotoxic agents.

Item 47. The method of any of items 24-46, wherein the cytotoxic agent is administered by injection.

Item 48. The method of any of items 24-47, wherein the cytotoxic agent is administered intravenously.

Item 49. The method of any of items 24-48, wherein an abnormal cell of the subject having the proliferative disorder characterized by abnormal cells is contacted with the cytotoxic agent prior to being contacted with the SET agonist or a SET ribosome antagonist.

Item 50. The method of any of items 24-49, wherein the abnormal cell is a cancer cell.

Item 51. The method or cell according to any of items 13-23, wherein expression cassette encodes a TR element selected from: a human and a mouse TR element.

Item 52. The method or cell according to any of items 13-23, wherein expression cassette encodes a TR element selected from those encoded by: SEQ ID NO:1, SEQ ID NO:2, SEQ ID NO:3, SEQ ID NO:4, SEQ ID NO:17, SEQ ID NO:18, SEQ ID NO:19, SEQ ID NO:20 or a variant of any thereof, wherein the encoded TR element confers selective translation on an operably linked coding sequence in an mRNA.

Item 53. The method or cell according to any of items 13-23 and 52, wherein the expression cassette encodes a reporter selected from: an antigenic epitope, a bioluminescent protein, an enzyme, a fluorescent protein, a receptor, and a transporter.

Item 54. The method or cell according to any of items 13-23 and 52-53, wherein the expression cassette encodes a reporter selected from: luciferase, GFP, EYFP, mRFP1, β-Gal, and CAT.

Item 55. A method of treatment substantially as described herein.

Item 56. A pharmaceutical composition substantially as described herein.

Item 57. A method of identifying an agent effective as a component of a SET Combination drug for treatment a proliferative disease substantially as described herein.

Item 58. An isolated, non-naturally occurring, cell characterized by a class 3 outlier SET response as described herein.

Item 59. A method of generating a metastatic cancer cell line model substantially as described herein.

Item 60. A method of identifying an agent effective to promote or inhibit G2 progression in vivo substantially as described herein.

Sequences

SEQ ID NO: 1
MurineTRdm
ttgagtgagttagagtagtgagctagttgtctggtaggggccccctttgcttccctggtggccactggattg
tgtttctttggagtggcactgttctgtggatgtggacatgaagctctcactggtacagaaaagctaattgag
acctatttctccaaaaactaccaggactatgagtatctcattaatgtgattcatgctttccagtatgtcatc
tatggaactgcctctttcttcttcctttatggggccctcctgctggctgagggcttctacaccaccggcgct
gtcaggcagatctttggcgactacaagaccaccatctgcggcaagggcctgagcgcaacgtttgtgggcatc
acctatgccctgactgttgtatggctcctggtgtttgcctgctcggctgtacctgtgtacatttacttcaat
acctggaccacctgtcagtctattgccttccctagcaagacctctgccagtataggcagtctctgcgctgat
gccagattgtatggtgttctcccatggaatgctttccctggcaaggtttgtggctccaaccttctgtccatc
tgcaaaacagctgagttccaattgaccttccacctgtttattgctgcgtttgtgggtgctgcggccacacta
gtttccctgctcaccttcatgattgctgccacttacaacttcgccgtccttaaactcatgggccgaggcacc
aagttc
SEQ ID NO: 2
Murine TRplp
ttgagtgagttagagtagtgagctagttgtctggtaggggccccctttgcttccctggtggccactggattg
tgtttctttggagtggcactgttctgtggatgtggacatgaagctctcactggtacagaaaagctaattgag
acctatttctccaaaaactaccaggactatgagtatctcattaatgtgattcatgctttccagtatgtcatc
tatggaactgcctctttcttcttcctttatggggccctcctgctggctgagggcttctacaccaccggcgct
gtcaggcagatctttggcgactacaagaccaccatctgcggcaagggcctgagcgcaacggtaacagggggc
cagaaggggaggggttccagaggccaacatcaagctcattctttggagcgggtgtgtcattgtttgggaaaa
tggctaggacatcccgacaagtttgtgggcatcacctatgccctgactgttgtatggctcctggtgtttgcc
tgctcggctgtacctgtgtacatttacttcaatacctggaccacctgtcagtctattgccttccctagcaag
acctctgccagtataggcagtctctgcgctgatgccagattgtatggtgttctcccatggaatgctttccct
ggcaaggtttgtggctccaaccttctgtccatctgcaaaacagctgagttccaattgaccttccacctgttt
attgctgcgtttgtgggtgctgcggccacactagtttccctgctcaccttcatgattgctgccacttacaac
ttcgccgtccttaaactcatgggccgaggcaccaagttc
SEQ ID NO: 3
Human TRdm
ttgagtgagttagagtagtgagctagttgtctggtaggggccccctttgcttccctggtggccactggattg
tgtttctttggggtggcactgttctgtggctgtggacatgaagccctcactggcacagaaaagctaattgag
acctatttctccaaaaactaccaagactatgagtatctcatcaatgtgattcatgctttccagtatgtcatc
tatggaactgcctctttcttcttcctttatggggccctcctgctggctgagggcttctacaccaccggcgca
gtcaggcagatctttggcgactacaagaccaccatctgcggcaagggcctgagcgcaacgtttgtgggcatc
acctatgccctgaccgttgtgtggctcctggtgtttgcctgctctgctgtgcccgtgtacatttacttcaac
acctggaccacctgcgactctattgccttccccagcaagacctctgccagtataggcagtctctgtgctgac
gccagattgtatggtgttctcccatggaatgctttccctggcaaggtttgtggctccaaccttctgtccatc
tgcaaaacagctgagttccaattgaccttccacctgtttattgctgcatttgtgggggctgcagccacactg
gtttccctgctcaccttcatgattgctgccacttacaactttgccgtccttaaactcatgggccgaggcacc
aagttc
SEQ ID NO: 4
Human TRplp
ttgagtgagttagagtagtgagctagttgtctggtaggggccccctttgcttccctggtggccactggattg
tgtttctttggggtggcactgttctgtggctgtggacatgaagccctcactggcacagaaaagctaattgag
acctatttctccaaaaactaccaagactatgagtatctcatcaatgtgattcatgctttccagtatgtcatc
tatggaactgcctctttcttcttcctttatggggccctcctgctggctgagggcttctacaccaccggcgca
gtcaggcagatctttggcgactacaagaccaccatctgcggcaagggcctgagcgcaacggtaacagggggc
cagaaggggaggggttccagaggccaacatcaagctcattctttggagcgggtgtgtcattgtttgggaaaa
tggctaggacatcccgacaagtttgtgggcatcacctatgccctgaccgttgtgtggctcctggtgtttgcc
tgctctgctgtgcccgtgtacatttacttcaacacctggaccacctgcgactctattgccttccccagcaag
acctctgccagtataggcagtctctgtgctgacgccagattgtatggtgttctcccatggaatgctttccct
ggcaaggtttgtggctccaaccttctgtccatctgcaaaacagctgagttccaattgaccttccacctgttt
attgctgcatttgtgggggctgcagccacactggtttccctgctcaccttcatgattgctgccacttacaac
tttgccgtccttaaactcatgggccgaggcaccaagttc
SEQ ID NO: 5
Mus musculus
atgggcttgttagagtgttgtgctagatgtctggtaggggccccctttgcttccctggtggccactggattg
tgtttctttggagtggcactgttctgtggatgtggacatgaagctctcactggtacagaaaagctaattgag
acctatttctccaaaaactaccaggactatgagtatctcattaatgtgattcatgctttccagtatgtcatc
tatggaactgcctctttcttcttcctttatggggccctcctgctggctgagggcttctacaccaccggcgct
gtcaggcagatctttggcgactacaagaccaccatctgcggcaagggcctgagcgcaacggtaacagggggc
cagaaggggaggggttccagaggccaacatcaagctcattctttggagcgggtgtgtcattgtttgggaaaa
tggctaggacatcccgacaagtttgtgggcatcacctatgccctgactgttgtatggctcctggtgtttgcc
tgctcggctgtacctgtgtacatttacttcaatacctggaccacctgtcagtctattgccttccctagcaag
acctctgccagtataggcagtctctgcgctgatgccagaatgtatggtgttctcccatggaatgctttccct
ggcaaggtttgtggctccaaccttctgtccatctgcaaaacagctgagttccaaatgaccttccacctgttt
attgctgcgtttgtgggtgctgcggccacactagtttccctgctcaccttcatgattgctgccacttacaac
ttcgccgtccttaaactcatgggccgaggcaccaagttctga
SEQ ID NO: 6
Mus musculus
atgggcttgttagagtgttgtgctagatgtctggtaggggccccctttgcttccctggtggccactggattg
tgtttctttggagtggcactgttctgtggatgtggacatgaagctctcactggtacagaaaagctaattgag
acctatttctccaaaaactaccaggactatgagtatctcattaatgtgattcatgctttccagtatgtcatc
tatggaactgcctctttcttcttcctttatggggccctcctgctggctgagggcttctacaccaccggcgct
gtcaggcagatctttggcgactacaagaccaccatctgcggcaagggcctgagcgcaacgtttgtgggcatc
acctatgccctgactgttgtatggctcctggtgtttgcctgctcggctgtacctgtgtacatttacttcaat
acctggaccacctgtcagtctattgccttccctagcaagacctctgccagtataggcagtctctgcgctgat
gccagaatgtatggtgttctcccatggaatgctttccctggcaaggtttgtggctccaaccttctgtccatc
tgcaaaacagctgagttccaaatgaccttccacctgtttattgctgcgtttgtgggtgctgcggccacacta
gtttccctgctcaccttcatgattgctgccacttacaacttcgccgtccttaaactcatgggccgaggcacc
aagttctga
SEQ ID NO: 7
Homo sapiens
atgggcttgttagagtgctgtgcaagatgtctggtaggggccccctttgcttccctggtggccactggattg
tgtttctttggggtggcactgttctgtggctgtggacatgaagccctcactggcacagaaaagctaattgag
acctatttctccaaaaactaccaagactatgagtatctcatcaatgtgatccatgccttccagtatgtcatc
tatggaactgcctctttcttcttcctttatggggccctcctgctggctgagggcttctacaccaccggcgca
gtcaggcagatctttggcgactacaagaccaccatctgcggcaagggcctgagcgcaacgtttgtgggcatc
acctatgccctgaccgttgtgtggctcctggtgtttgcctgctctgctgtgcccgtgtacatttacttcaac
acctggaccacctgcgactctattgccttccccagcaagacctctgccagtataggcagtctctgtgctgac
gccagaatgtatggtgttctcccatggaatgctttccctggcaaggtttgtggctccaaccttctgtccatc
tgcaaaacagctgagttccaaatgaccttccacctgtttattgctgcatttgtgggggctgcagctacactg
gtttccctgctcaccttcatgattgctgccacttacaactttgccgtccttaaactcatgggccgaggcacc
aagttctga
SEQ ID NO: 8
Homo sapiens
atgggcttgttagagtgctgtgcaagatgtctggtaggggccccctttgcttccctggtggccactggattg
tgtttctttggggtggcactgttctgtggctgtggacatgaagccctcactggcacagaaaagctaattgag
acctatttctccaaaaactaccaagactatgagtatctcatcaatgtgatccatgccttccagtatgtcatc
tatggaactgcctctttcttcttcctttatggggccctcctgctggctgagggcttctacaccaccggcgca
gtcaggcagatctttggcgactacaagaccaccatctgcggcaagggcctgagcgcaacggtaacagggggc
cagaaggggaggggttccagaggccaacatcaagctcattctttggagcgggtgtgtcattgtttgggaaaa
tggctaggacatcccgacaagtttgtgggcatcacctatgccctgaccgttgtgtggctcctggtgtttgcc
tgctctgctgtgcccgtgtacatttacttcaacacctggaccacctgcgactctattgccttccccagcaag
acctctgccagtataggcagtctctgtgctgacgccagaatgtatggtgttctcccatggaatgctttccct
ggcaaggtttgtggctccaaccttctgtccatctgcaaaacagctgagttccaaatgaccttccacctgttt
attgctgcatttgtgggggctgcagctacactggtttccctgctcaccttcatgattgctgccacttacaac
tttgccgtccttaaactcatgggccgaggcaccaagttctgatacactggtttccctg
SEQ ID NO: 9
Mammalian PLP consensus sequence
atgggcytgttagagtgytgygcnagatgyctsgtaggggccccctttgcttccytggtggccactggattn
tgtttctttggngtggcactsttctgtggmtgtggacatgaagchytmactggyacagaaaagytaattgag
acmtatttctccaaaaaytaccaagactaygagtatctcatyaatgtgatycatgcyttccagtatgtcatc
tatggaactgcctctttcttcttcctttatggggccctcctgctggcygagggcttctacaccaccggygcw
gtcaggcagatctttggcgactacaagaccaccatctgcggsaagggcctgagygcaacggtaacagggggc
cagaaggggaggggttccagaggccaacatcaagctcattctttggagcgggtgtgtcattgtttgggaaaa
tggctaggacatcccgacaagtttgtgggcatcacctatgccytgacygttgtntggctcctngtgtttgcc
tgctckgctgtncctgtgtacatttayttcaayacctggaccacytgycagtctattgcckycccyagcaag
acytctgccagyataggcastctctgygctgatgccagaatgtatggtgttctcccatggaatgctttyccw
ggcaangtktgtggctccaaccttctgtccatctgcaaaacagctgagttccaaatgacsttccayctgttt
attgctgcvttygtgggkgctgcngcyacactngtktccctgctcaccttcatgattgctgccacttacaac
ttygccgtcctkaaactcatgggccgaggcaccaagttctga
PLP generic consensus sequence including exon 5 SEQ ID NO: 17
btgagtgagttagagtagtgagcnagttgyctsgtaggggccccctttgcttccytggtggccactggattn
tgtttctttggngtggcactsttctgtggmtgtggacatgaagchytmactggyacagaaaagytaattgag
acmtatttctccaaaaaytaccaagactaygagtatctcatyaatgtgatycatgcyttccagtatgtcatc
tatggaactgcctctttcttcttcctttatggggccctcctgctggcygagggcttctacaccaccggygcw
gtcaggcagatctttggcgactacaagaccaccatctgcggsaagggcctgagygcaacggtaacagggggc
cagaaggggaggggttccagaggccaacatcaagctcattctttggagcgggtgtgtcattgtttgggaaaa
tggctaggacatcccgacaagtttgtgggcatcacctatgccytgacygttgtntggctcctngtgtttgcc
tgctckgctgtncctgtgtacatttayttcaayacctggaccacytgycagtctattgcckycccyagcaag
acytctgccagyataggcastctctgygctgatgccagabtgtatggtgttctcccatggaatgctttyccw
ggcaangtktgtggctccaaccttctgtccatctgcaaaacagctgagttccaabtgacsttccayctgttt
attgctgcvttygtgggkgctgcngcyacactngtktccctgctcaccttcatgattgctgccacttacaac
ttygccgtcctkaaactcatgggccgaggcaccaagttc
DM20 generic consensus sequence including exon 5 SEQ ID NO: 18
btgagtgagttagagtagtgagcnagttgyctsgtaggggccccctttgcttccytggtggccactggattc
tgtttctttggngtggcactsttctgtggmtgtggacatgaagchytmactggyacagaaaagytaattgag
acmtatttctccaaaaaytaccaagactaygagtatctcatyaatgtgatycatgcyttccagtatgtcatc
tatggaactgcctctttcttcttcctttatggggccctcctgctggcygagggcttctacaccaccggygcw
gtcaggcagatctttggcgactacaagaccaccatctgcggsaagggcctgagygcaacgtttgtgggcatc
acctatgccytgacygttgtntggctcctngtgtttgcctgctckgctgtncctgtgtacatttayttcaay
acctggaccacytgycagtctattgcckycccyagcaagacytctgccagyataggcastctctgygctgat
gccagabtgtatggtgttctcccatggaatgctttyccwggcaangtktgtggctccaaccttctgtccatc
tgcaaaacagctgagttccaabtgacsttccayctgtttattgctgcvttygtgggkgctgcngcyacactn
gtktccctgctcaccttcatgattgctgccacttacaacttygccgtcctkaaactcatgggccgaggcacc
aagttc
PLP generic consensus sequence exon 5 deleted SEQ ID NO: 19
btgagtgagttagagtagtgagcnagttgyctsgtaggggccccctttgcttccytggtggccactggattn
tgtttctttggngtggcactsttctgtggmtgtggacatgaagchytmactggyacagaaaagytaattgag
acmtatttctccaaaaaytaccaagactaygagtatctcatyaatgtgatycatgcyttccagtatgtcatc
tatggaactgcctctttcttcttcctttatggggccctcctgctggcygagggcttctacaccaccggygcw
gtcaggcagatctttggcgactacaagaccaccatctgcggsaagggcctgagygcaacggtaacagggggc
cagaaggggaggggttccagaggccaacatcaagctcattctttggagcgggtgtgtcattgtttgggaaaa
tggctaggacatcccgacaagtttgtgggcatcacctatgccytgacygttgtntggctcctngtgtttgcc
tgctckgctgtncctgtgtacatttayttcaayacctggaccacytgycagtctattgcckycccyagcaag
acytctgccagyataggcastctctgygctgatgccagabtgtatgttccaabtgacsttccayctgtttat
tgctgcvttygtgggkgctgcngcyacactngtktccctgctcaccttcatgattgctgccacttacaactt
ygccgtcctkaaactcatgggccgaggcaccaagttc
DM20 generic consensus sequence exon 5 deleted SEQ ID NO: 20
btgagtgagttagagtagtgagcnagttgyctsgtaggggccccctttgcttccytggtggccactggattn
tgtttctttggngtggcactsttctgtggmtgtggacatgaagchytmactggyacagaaaagytaattgag
acmtatttctccaaaaaytaccaagactaygagtatctcatyaatgtgatycatgcyttccagtatgtcatc
tatggaactgcctctttcttcttcctttatggggccctcctgctggcygagggcttctacaccaccggygcw
gtcaggcagatctttggcgactacaagaccaccatctgcggsaagggcctgagygcaacgtttgtgggcatc
acctatgccytgacygttgtntggctcctngtgtttgcctgctckgctgtncctgtgtacatttayttcaay
acctggaccacytgycagtctattgcckycccyagcaagacytctgccagyataggcastctctgygctgat
gccagabtgtatgttccaabtgacsttccayctgtttattgctgcvttygtgggkgctgcngcyacactngt
ktccctgctcaccttcatgattgctgccacttacaacttygccgtcctkaaactcatgggccgaggcaccaa
gttc

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1.-12. (canceled)

13. A method of identifying an agent effective to promote or inhibit G2 progression in vivo are provided according to aspects of the present invention which include providing a cell of a TR Class 4 cell line characterized by a TR Class 3 outlier SET response, wherein the cell comprises a TR nucleic acid expression cassette encoding a TR element and a reporter; wherein the expression cassette is stably integrated into the genome of the cells;
administering the cell to a non-human animal, producing a xenograft tumor in the non-human animal; administering a test substance to the non-human animal; and measuring the effect of the test substance on the SET response, wherein an increase in a SET response identifies the agent as a SET agonist effective to promote G2 progression in vivo.

14. The method of claim 13, further comprising administering a SET agonist to the non-human animal to promote G2 progression in vivo, wherein a decrease in the SET response identifies the agent as a SET antagonist effective to inhibit G2 progression in vivo.

15. The method of claim 13, further comprising measuring the effect of the test substance on the xenograft tumor.

16. The method of claim 13 any of claims 13, wherein the non-human animal is a rat or mouse.

17. A method of identifying an agent effective as a component of a SET Combination drug for treatment a proliferative disease, comprising:
providing a cell characterized by a TR Class 3 SET response or a TR Class 3 SET outlier response, wherein the cell comprises an expression construct encoding a TR element and a reporter stably integrated in the genome of the cell;
contacting the cell with a test substance; and
measuring the effect of the test substance on protein synthesis from a SET ribosome compared to a control, wherein inhibition of protein synthesis from a SET ribosome by the test substance identifies the substance as an agent effective as a component of a SET Combination drug for treatment a proliferative disease.

18. The method of claim 17, wherein the cell is further characterized by in vitro ability to grow in suspension cultures as nonadherent 3D structures and the ability to initiate and grow into a primary xenogenic tumor in vivo, that can be dissected into subfragments and propagated as a secondary tumor.

19. A method of generating a metastatic cancer cell line model, comprising:
introducing an expression cassette encoding a TR element and a reporter into a cell, producing a parental population of cells wherein the expression cassette is stably integrated into the genome of the cells;
isolating subclones of the parental population;
administering a SET agonist to a population of cells of each subclone to induce a SET TR response in the population of cells of each subclone;
assaying the TR SET response in the population of cells of each subclone by detecting expression of the reporter;
ranking the TR SET response of each subclone compared to each other subclone, establishing a range of TR SET responses characterized by an average response;
selecting the subclones characterized by detectable increases in expression of the reporter of at least two standard deviations greater than the mean response, thereby defining the selected subclones as TR Class 3 TR SET response subclones;
administering a SET agonist to a population of cells of each TR Class 3 TR SET response subclone to induce a SET TR response in the population of cells of each TR Class 3 TR SET response subclone;
assaying the TR SET response in the population of cells of each TR Class 3 SET response subclone by detecting expression of the reporter;
ranking the TR SET response of each TR Class 3 SET response subclone compared to each other TR Class 3 SET response subclone, establishing a range of TR SET responses characterized by an average response;
selecting the TR Class 3 SET response subclones characterized by detectable increases in expression of the reporter of at least two standard deviations greater than the mean response, thereby defining the selected TR Class 3 SET response subclones as TR Class 3 SET response outliers;
administering one or more toxins to cells of one or more subclones characterized as a TR Class 3 SET response outliers; and
detecting a response of the cells of the one or more subclones characterized as a TR Class 3 SET response outliers indicative of drug and stress resistance due to elevated SET ribosome activity in the cells of the subclone, thereby determining that the cells are TR Class 4 cells; and
thereby generating a metastatic cancer cell line model.

20. The method of claim 19, further comprising:
culturing the TR Class 4 cells under low density conditions for at least 50 cell cycles, generating TR Class 4 subclones and capable of low density colony foiniation;
selecting the TR Class 4 subclones capable of low density colony formation;
administering a SET agonist to a population of cells of each TR Class 4 subclone capable of low density colony formation to induce a TR SET response;
assaying the SET response in the population of cells of each TR Class 4 subclone capable of low density colony formation to induce a TR SET response by detecting expression of the reporter;
ranking the TR SET response of each TR Class 4 subclone capable of low density colony formation compared to each other TR Class 4 subclone capable of low density colony formation establishing a range of SET responses characterized by an average response; and
selecting the TR Class 4 subclones capable of low density colony formation and characterized by detectable increases in expression of the reporter of at least two standard deviations greater than the mean response.

21. The method of claim 19, further comprising:
culturing the TR Class 4 cells under nonadherent low density culture conditions;
selecting subclones of the TR Class 4 cells that grow as suspended aggregates, thereby selecting subclones of TR Class 4 cells capable of ex vivo tumorsphere formation with 10 or fewer cells initiating the tumorsphere;
administering one or more toxins to cells of the TR Class 4 subclones capable of ex vivo tumorsphere formation with 10 or fewer cells initiating the tumorsphere response; and
detecting a response of the cells of the TR Class 4 subclones capable of ex vivo tumorsphere formation with 10 or fewer cells initiating the tumorsphere indicative of drug and stress resistance due to elevated SET ribosome activity in the cells of the subclone, thereby determining that the cells of the TR Class 4 subclones are capable of ex vivo tumorsphere formation with 10 or fewer cells, characterized by a TR Class 4 SET response.

22.-60. (canceled)


SEQUENCE LISTING (20 sequences)

SEQ ID NO: 1 - DNA, 726 bp, Artificial Sequence
  mutated version of murine DM20 gene encoding a TR element
ttgagtgagttagagtagtgagctagttgtctggtaggggccccctttgcttccctggtggccactggattgtgtttctttggagtggcactgttctgtggatgtggacatgaagctctcactggtacagaaaagctaattgagacctatttctccaaaaactaccaggactatgagtatctcattaatgtgattcatgctttccagtatgtcatctatggaactgcctctttcttcttcctttatggggccctcctgctggctgagggcttctacaccaccggcgctgtcaggcagatctttggcgactacaagaccaccatctgcggcaagggcctgagcgcaacgtttgtgggcatcacctatgccctgactgttgtatggctcctggtgtttgcctgctcggctgtacctgtgtacatttacttcaatacctggaccacctgtcagtctattgccttccctagcaagacctctgccagtataggcagtctctgcgctgatgccagattgtatggtgttctcccatggaatgctttccctggcaaggtttgtggctccaaccttctgtccatctgcaaaacagctgagttccaattgaccttccacctgtttattgctgcgtttgtgggtgctgcggccacactagtttccctgctcaccttcatgattgctgccacttacaacttcgccgtccttaaactcatgggccgaggcaccaagttc

SEQ ID NO: 2 - DNA, 831 bp, Artificial Sequence
  mutated version of murine proteolipid protein gene encoding a TR element
ttgagtgagttagagtagtgagctagttgtctggtaggggccccctttgcttccctggtggccactggattgtgtttctttggagtggcactgttctgtggatgtggacatgaagctctcactggtacagaaaagctaattgagacctatttctccaaaaactaccaggactatgagtatctcattaatgtgattcatgctttccagtatgtcatctatggaactgcctctttcttcttcctttatggggccctcctgctggctgagggcttctacaccaccggcgctgtcaggcagatctttggcgactacaagaccaccatctgcggcaagggcctgagcgcaacggtaacagggggccagaaggggaggggttccagaggccaacatcaagctcattctttggagcgggtgtgtcattgtttgggaaaatggctaggacatcccgacaagtttgtgggcatcacctatgccctgactgttgtatggctcctggtgtttgcctgctcggctgtacctgtgtacatttacttcaatacctggaccacctgtcagtctattgccttccctagcaagacctctgccagtataggcagtctctgcgctgatgccagattgtatggtgttctcccatggaatgctttccctggcaaggtttgtggctccaaccttctgtccatctgcaaaacagctgagttccaattgaccttccacctgtttattgctgcgtttgtgggtgctgcggccacactagtttccctgctcaccttcatgattgctgccacttacaacttcgccgtccttaaactcatgggccgaggcaccaagttc

SEQ ID NO: 3 - DNA, 726 bp, Artificial Sequence
  mutated version of human DM20 gene encoding a TR element
ttgagtgagttagagtagtgagctagttgtctggtaggggccccctttgcttccctggtggccactggattgtgtttctttggggtggcactgttctgtggctgtggacatgaagccctcactggcacagaaaagctaattgagacctatttctccaaaaactaccaagactatgagtatctcatcaatgtgattcatgctttccagtatgtcatctatggaactgcctctttcttcttcctttatggggccctcctgctggctgagggcttctacaccaccggcgcagtcaggcagatctttggcgactacaagaccaccatctgcggcaagggcctgagcgcaacgtttgtgggcatcacctatgccctgaccgttgtgtggctcctggtgtttgcctgctctgctgtgcccgtgtacatttacttcaacacctggaccacctgcgactctattgccttccccagcaagacctctgccagtataggcagtctctgtgctgacgccagattgtatggtgttctcccatggaatgctttccctggcaaggtttgtggctccaaccttctgtccatctgcaaaacagctgagttccaattgaccttccacctgtttattgctgcatttgtgggggctgcagccacactggtttccctgctcaccttcatgattgctgccacttacaactttgccgtccttaaactcatgggccgaggcaccaagttc

SEQ ID NO: 4 - DNA, 831 bp, Artificial Sequence
  mutated version of human proteolipid protein gene encoding a TR element
ttgagtgagttagagtagtgagctagttgtctggtaggggccccctttgcttccctggtggccactggattgtgtttctttggggtggcactgttctgtggctgtggacatgaagccctcactggcacagaaaagctaattgagacctatttctccaaaaactaccaagactatgagtatctcatcaatgtgattcatgctttccagtatgtcatctatggaactgcctctttcttcttcctttatggggccctcctgctggctgagggcttctacaccaccggcgcagtcaggcagatctttggcgactacaagaccaccatctgcggcaagggcctgagcgcaacggtaacagggggccagaaggggaggggttccagaggccaacatcaagctcattctttggagcgggtgtgtcattgtttgggaaaatggctaggacatcccgacaagtttgtgggcatcacctatgccctgaccgttgtgtggctcctggtgtttgcctgctctgctgtgcccgtgtacatttacttcaacacctggaccacctgcgactctattgccttccccagcaagacctctgccagtataggcagtctctgtgctgacgccagattgtatggtgttctcccatggaatgctttccctggcaaggtttgtggctccaaccttctgtccatctgcaaaacagctgagttccaattgaccttccacctgtttattgctgcatttgtgggggctgcagccacactggtttccctgctcaccttcatgattgctgccacttacaactttgccgtccttaaactcatgggccgaggcaccaagttc

SEQ ID NO: 5 - DNA, 729 bp, Mus musculus
atgggcttgttagagtgttgtgctagatgtctggtaggggccccctttgcttccctggtggccactggattgtgtttctttggagtggcactgttctgtggatgtggacatgaagctctcactggtacagaaaagctaattgagacctatttctccaaaaactaccaggactatgagtatctcattaatgtgattcatgctttccagtatgtcatctatggaactgcctctttcttcttcctttatggggccctcctgctggctgagggcttctacaccaccggcgctgtcaggcagatctttggcgactacaagaccaccatctgcggcaagggcctgagcgcaacgtttgtgggcatcacctatgccctgactgttgtatggctcctggtgtttgcctgctcggctgtacctgtgtacatttacttcaatacctggaccacctgtcagtctattgccttccctagcaagacctctgccagtataggcagtctctgcgctgatgccagaatgtatggtgttctcccatggaatgctttccctggcaaggtttgtggctccaaccttctgtccatctgcaaaacagctgagttccaaatgaccttccacctgtttattgctgcgtttgtgggtgctgcggccacactagtttccctgctcaccttcatgattgctgccacttacaacttcgccgtccttaaactcatgggccgaggcaccaagttctga

SEQ ID NO: 6 - DNA, 834 bp, Mus musculus
atgggcttgttagagtgttgtgctagatgtctggtaggggccccctttgcttccctggtggccactggattgtgtttctttggagtggcactgttctgtggatgtggacatgaagctctcactggtacagaaaagctaattgagacctatttctccaaaaactaccaggactatgagtatctcattaatgtgattcatgctttccagtatgtcatctatggaactgcctctttcttcttcctttatggggccctcctgctggctgagggcttctacaccaccggcgctgtcaggcagatctttggcgactacaagaccaccatctgcggcaagggcctgagcgcaacggtaacagggggccagaaggggaggggttccagaggccaacatcaagctcattctttggagcgggtgtgtcattgtttgggaaaatggctaggacatcccgacaagtttgtgggcatcacctatgccctgactgttgtatggctcctggtgtttgcctgctcggctgtacctgtgtacatttacttcaatacctggaccacctgtcagtctattgccttccctagcaagacctctgccagtataggcagtctctgcgctgatgccagaatgtatggtgttctcccatggaatgctttccctggcaaggtttgtggctccaaccttctgtccatctgcaaaacagctgagttccaaatgaccttccacctgtttattgctgcgtttgtgggtgctgcggccacactagtttccctgctcaccttcatgattgctgccacttacaacttcgccgtccttaaactcatgggccgaggcaccaagttctga

SEQ ID NO: 7 - DNA, 729 bp, Homo sapiens
atgggcttgttagagtgctgtgcaagatgtctggtaggggccccctttgcttccctggtggccactggattgtgtttctttggggtggcactgttctgtggctgtggacatgaagccctcactggcacagaaaagctaattgagacctatttctccaaaaactaccaagactatgagtatctcatcaatgtgatccatgccttccagtatgtcatctatggaactgcctctttcttcttcctttatggggccctcctgctggctgagggcttctacaccaccggcgcagtcaggcagatctttggcgactacaagaccaccatctgcggcaagggcctgagcgcaacgtttgtgggcatcacctatgccctgaccgttgtgtggctcctggtgtttgcctgctctgctgtgcccgtgtacatttacttcaacacctggaccacctgcgactctattgccttccccagcaagacctctgccagtataggcagtctctgtgctgacgccagaatgtatggtgttctcccatggaatgctttccctggcaaggtttgtggctccaaccttctgtccatctgcaaaacagctgagttccaaatgaccttccacctgtttattgctgcatttgtgggggctgcagctacactggtttccctgctcaccttcatgattgctgccacttacaactttgccgtccttaaactcatgggccgaggcaccaagttctga

SEQ ID NO: 8 - DNA, 850 bp, Homo sapiens
atgggcttgttagagtgctgtgcaagatgtctggtaggggccccctttgcttccctggtggccactggattgtgtttctttggggtggcactgttctgtggctgtggacatgaagccctcactggcacagaaaagctaattgagacctatttctccaaaaactaccaagactatgagtatctcatcaatgtgatccatgccttccagtatgtcatctatggaactgcctctttcttcttcctttatggggccctcctgctggctgagggcttctacaccaccggcgcagtcaggcagatctttggcgactacaagaccaccatctgcggcaagggcctgagcgcaacggtaacagggggccagaaggggaggggttccagaggccaacatcaagctcattctttggagcgggtgtgtcattgtttgggaaaatggctaggacatcccgacaagtttgtgggcatcacctatgccctgaccgttgtgtggctcctggtgtttgcctgctctgctgtgcccgtgtacatttacttcaacacctggaccacctgcgactctattgccttccccagcaagacctctgccagtataggcagtctctgtgctgacgccagaatgtatggtgttctcccatggaatgctttccctggcaaggtttgtggctccaaccttctgtccatctgcaaaacagctgagttccaaatgaccttccacctgtttattgctgcatttgtgggggctgcagctacactggtttccctgctcaccttcatgattgctgccacttacaactttgccgtccttaaactcatgggccgaggcaccaagttctgatacactggtttccctg

SEQ ID NO: 9 - DNA, 834 bp, Artificial Sequence
  Mammalian proteolipid protein gene consensus sequence
  misc_feature (24)..(24): n is a, c, g, or t
  misc_feature (72)..(72): n is a, c, g, or t
  misc_feature (84)..(84): n is a, c, g, or t
  misc_feature (486)..(486): n is a, c, g, or t
  misc_feature (495)..(495): n is a, c, g, or t
  misc_feature (516)..(516): n is a, c, g, or t
  misc_feature (654)..(654): n is a, c, g, or t
  misc_feature (744)..(744): n is a, c, g, or t
  misc_feature (753)..(753): n is a, c, g, or t
atgggcytgttagagtgytgygcnagatgyctsgtaggggccccctttgcttccytggtggccactggattntgtttctttggngtggcactsttctgtggmtgtggacatgaagchytmactggyacagaaaagytaattgagacmtatttctccaaaaaytaccaagactaygagtatctcatyaatgtgatycatgcyttccagtatgtcatctatggaactgcctctttcttcttcctttatggggccctcctgctggcygagggcttctacaccaccggygcwgtcaggcagatctttggcgactacaagaccaccatctgcggsaagggcctgagygcaacggtaacagggggccagaaggggaggggttccagaggccaacatcaagctcattctttggagcgggtgtgtcattgtttgggaaaatggctaggacatcccgacaagtttgtgggcatcacctatgccytgacygttgtntggctcctngtgtttgcctgctckgctgtncctgtgtacatttayttcaayacctggaccacytgycagtctattgcckycccyagcaagacytctgccagyataggcastctctgygctgatgccagaatgtatggtgttctcccatggaatgctttyccwggcaangtktgtggctccaaccttctgtccatctgcaaaacagctgagttccaaatgacsttccayctgtttattgctgcvttygtgggkgctgcngcyacactngtktccctgctcaccttcatgattgctgccacttacaacttygccgtcctkaaactcatgggccgaggcaccaagttctga

SEQ ID NO: 10 - RNA, 47 nt, Artificial Sequence
  helix 26 18s rRNA, partial
gcgaugcggcggcguuauucccaugacccgccgggcagcuuccggga

SEQ ID NO: 11 - RNA, 25 nt, Artificial Sequence
  influenzavirus B, partial
cagagcucuaugggaaauucagcuc

SEQ ID NO: 12 - RNA, 25 nt, Artificial Sequence
  feline calicivirus, partial
gccuccuacaugggaauacaauugg

SEQ ID NO: 13 - RNA, 25 nt, Artificial Sequence
  rabbit hemorrhagic disease virus, partial
cagagcucuaugggaaauucagcuc

SEQ ID NO: 14 - RNA, 23 nt, Artificial Sequence
  GTX IRES
cccgagccggcgggugcgggggc

SEQ ID NO: 15 - RNA, 30 nt, Artificial Sequence
  murine TR IRES
uguuugccugcucggcuguaccuguguaca

SEQ ID NO: 16 - RNA, 42 nt, Artificial Sequence
  human TR regulator
aacuucgccguccuuaaacucaugggccgaggcaccaaguuc

SEQ ID NO: 17 - DNA, 834 bp, Artificial Sequence
  proteolipid protein generic consensus including exon 5
  misc_feature (24)..(24): n is a, c, g, or t
  misc_feature (72)..(72): n is a, c, g, or t
  misc_feature (84)..(84): n is a, c, g, or t
  misc_feature (486)..(486): n is a, c, g, or t
  misc_feature (495)..(495): n is a, c, g, or t
  misc_feature (516)..(516): n is a, c, g, or t
  misc_feature (654)..(654): n is a, c, g, or t
  misc_feature (663)..(663): n is a, c, g, or t
  misc_feature (747)..(747): n is a, c, g, or t
  misc_feature (756)..(756): n is a, c, g, or t
btgagtgagttagagtagtgagcnagttgyctsgtaggggccccctttgcttccytggtggccactggattntgtttctttggngtggcactsttctgtggmtgtggacatgaagchytmactggyacagaaaagytaattgagacmtatttctccaaaaaytaccaagactaygagtatctcatyaatgtgatycatgcyttccagtatgtcatctatggaactgcctctttcttcttcctttatggggccctcctgctggcygagggcttctacaccaccggygcwgtcaggcagatctttggcgactacaagaccaccatctgcggsaagggcctgagygcaacggtaacagggggccagaaggggaggggttccagaggccaacatcaagctcattctttggagcgggtgtgtcattgtttgggaaaatggctaggacatcccgacaagtttgtgggcatcacctatgccytgacygttgtntggctcctngtgtttgcctgctckgctgtncctgtgtacatttayttcaayacctggaccacytgycagtctattgcckycccyagcaagacytctgccagyataggcastctctgygctgatgccagabtgtatggtgttctcccatggaatgctttyccwggcaangtktgtsdnggctccaaccttctgtccatctgcaaaacagctgagttccaabtgacsttccayctgtttattgctgcvttygtgggkgctgcngcyacactngtktccctgctcaccttcatgattgctgccacttacaacttygccgtcctkaaactcatgggccgaggcaccaagttc

SEQ ID NO: 18 - DNA, 726 bp, Artificial Sequence
  DM20 generic consensus sequence including exon 5
  misc_feature (24)..(24): n is a, c, g, or t
  misc_feature (72)..(72): n is a, c, g, or t
  misc_feature (84)..(84): n is a, c, g, or t
  misc_feature (381)..(381): n is a, c, g, or t
  misc_feature (390)..(390): n is a, c, g, or t
  misc_feature (411)..(411): n is a, c, g, or t
  misc_feature (549)..(549): n is a, c, g, or t
  misc_feature (639)..(639): n is a, c, g, or t
  misc_feature (648)..(648): n is a, c, g, or t
btgagtgagttagagtagtgagcnagttgyctsgtaggggccccctttgcttccytggtggccactggattntgtttctttggngtggcactsttctgtggmtgtggacatgaagchytmactggyacagaaaagytaattgagacmtatttctccaaaaaytaccaagactaygagtatctcatyaatgtgatycatgcyttccagtatgtcatctatggaactgcctctttcttcttcctttatggggccctcctgctggcygagggcttctacaccaccggygcwgtcaggcagatctttggcgactacaagaccaccatctgcggsaagggcctgagygcaacgtttgtgggcatcacctatgccytgacygttgtntggctcctngtgtttgcctgctckgctgtncctgtgtacatttayttcaayacctggaccacytgycagtctattgcckycccyagcaagacytctgccagyataggcastctctgygctgatgccagabtgtatggtgttctcccatggaatgctttyccwggcaangtktgtggctccaaccttctgtccatctgcaaaacagctgagttccaabtgacsttccayctgtttattgctgcvttygtgggkgctgcngcyacactngtktccctgctcaccttcatgattgctgccacttacaacttygccgtcctkaaactcatgggccgaggcaccaagttc

SEQ ID NO: 19 - DNA, 757 bp, Artificial Sequence
  proteolipid protein generic consensus sequence exon 5 deleted
  misc_feature (24)..(24): n is a, c, g, or t
  misc_feature (72)..(72): n is a, c, g, or t
  misc_feature (84)..(84): n is a, c, g, or t
  misc_feature (486)..(486): n is a, c, g, or t
  misc_feature (495)..(495): n is a, c, g, or t
  misc_feature (516)..(516): n is a, c, g, or t
  misc_feature (670)..(670): n is a, c, g, or t
  misc_feature (679)..(679): n is a, c, g, or t
btgagtgagttagagtagtgagcnagttgyctsgtaggggccccctttgcttccytggtggccactggattntgtttctttggngtggcactsttctgtggmtgtggacatgaagchytmactggyacagaaaagytaattgagacmtatttctccaaaaaytaccaagactaygagtatctcatyaatgtgatycatgcyttccagtatgtcatctatggaactgcctctttcttcttcctttatggggccctcctgctggcygagggcttctacaccaccggygcwgtcaggcagatctttggcgactacaagaccaccatctgcggsaagggcctgagygcaacggtaacagggggccagaaggggaggggttccagaggccaacatcaagctcattctttggagcgggtgtgtcattgtttgggaaaatggctaggacatcccgacaagtttgtgggcatcacctatgccytgacygttgtntggctcctngtgtttgcctgctckgctgtncctgtgtacatttayttcaayacctggaccacytgycagtctattgcckycccyagcaagacytctgccagyataggcastctctgygctgatgccagabtgtatgttccaabtgacsttccayctgtttattgctgcvttygtgggkgctgcngcyacactngtktccctgctcaccttcatgattgctgccacttacaacttygccgtcctkaaactcatgggccgaggcaccaagttc

SEQ ID NO: 20 - DNA, 652 bp, Artificial Sequence
  DM20 generic consensus sequence exon 5 deleted
  misc_feature (24)..(24): n is a, c, g, or t
  misc_feature (72)..(72): n is a, c, g, or t
  misc_feature (84)..(84): n is a, c, g, or t
  misc_feature (381)..(381): n is a, c, g, or t
  misc_feature (390)..(390): n is a, c, g, or t
  misc_feature (411)..(411): n is a, c, g, or t
  misc_feature (565)..(565): n is a, c, g, or t
  misc_feature (574)..(574): n is a, c, g, or t
btgagtgagttagagtagtgagcnagttgyctsgtaggggccccctttgcttccytggtggccactggattntgtttctttggngtggcactsttctgtggmtgtggacatgaagchytmactggyacagaaaagytaattgagacmtatttctccaaaaaytaccaagactaygagtatctcatyaatgtgatycatgcyttccagtatgtcatctatggaactgcctctttcttcttcctttatggggccctcctgctggcygagggcttctacaccaccggygcwgtcaggcagatctttggcgactacaagaccaccatctgcggsaagggcctgagygcaacgtttgtgggcatcacctatgccytgacygttgtntggctcctngtgtttgcctgctckgctgtncctgtgtacatttayttcaayacctggaccacytgycagtctattgcckycccyagcaagacytctgccagyataggcastctctgygctgatgccagabtgtatgttccaabtgacsttccayctgtttattgctgcvttygtgggkgctgcngcyacactngtktccctgctcaccttcatgattgctgccacttacaacttygccgtcctkaaactcatgggccgaggcaccaagttc